(19) United States
(12) Patent Application Publication
(10) Pub. No.: US 2024/0217957 A1
(43) Pub. Date: Jul. 4, 2024

(54) HETEROCYCLIC COMPOUND AND USE THEREOF

(71) Applicant: TAKEDA PHARMACEUTICAL COMPANY LIMITED, Osaka (JP)

(72) Inventors: Asato KINA, Fujisawa (JP); Zenichi IKEDA, Fujisawa (JP); Mitsunori KONO, Fujisawa, Kanagawa (JP); Yuya OGURO, Fujisawa (JP); Osamu KUBO, Fujisawa (JP); Toru YAMASHITA, Fujisawa (JP); Kazuaki TAKAMI, Fujisawa (JP); Takafumi YUKAWA, Fujisawa (JP); Masaki SETO, Fujisawa (JP); Makoto KAMATA, Fujisawa (JP); Kenjiro SATO, Fujisawa (JP); Masataka MURAKAMI, Fujisawa (JP); Yusuke SASAKI, Fujisawa (JP); Matthew Thomas REYNOLDS, Fujisawa (JP); Minoru NAKAMURA, Fujisawa (JP); Taku KAMEI, Fujisawa (JP); Hiroyuki KAKEI, Fujisawa (JP); Fumie YAMAGUCHI, Fujisawa (JP); Tomohiro OHASHI, Fujisawa (JP); Takuto KOJIMA, Fujisawa (JP); Keiko KAKEGAWA, Fujisawa (JP); Florian PÜNNER, Fujisawa (JP); Shinji NAKAMURA, Fujisawa (JP); Nao MORISHITA, Fujisawa (JP); Tadashi HIDAKA, Fujisawa (JP); Sachie TAKASHIMA, Fujisawa (JP); Takahiko TANIGUCHI, Fujisawa (JP)

(21) Appl. No.: 18/522,917

(22) Filed: Nov. 29, 2023

Related U.S. Application Data
(60) Provisional application No. 63/385,598, filed on Nov. 30, 2022.

(51) Int. Cl. C07D 413/04; A61P 25/00; C07D 413/14

(52) U.S. Cl. CPC C07D 413/04; A61P 25/00; C07D 413/14

(57) ABSTRACT

The present invention provides a heterocyclic compound having an orexin type 2 receptor agonist activity. Provided is a compound of formula (I):

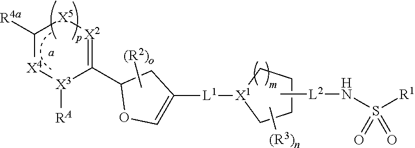

wherein each symbol is as described in the specification, or a salt thereof has an orexin type 2 receptor agonist activity, and is useful as an agent for the prophylaxis or treatment of narcolepsy.

### TECHNICAL FIELD

The present invention relates to a heterocyclic compound, particularly, a heterocyclic compound having an orexin type 2 receptor agonist activity.

### BACKGROUND OF THE INVENTION

Orexin is a neuropeptide specifically produced in particular neurons located sparsely in the lateral hypothalamus and its surrounding area, and consists of two subtypes, orexin A and orexin B. Both orexin A and orexin B are endogenous ligands of the orexin receptors, which are G protein-coupled receptors mainly present in the brain, and two types of subtypes, type 1 and type 2, are known for the orexin receptors (non-patent document 1).

Since orexin-producing neurons (orexin neurons) are localized in the vicinity of the feeding center, and intraventricular administration of orexin peptide results in an increase in food intake, orexin initially attracted attention as a neuropeptide having a feeding behavioral regulation. Thereafter, however, it was reported that the cause of dog narcolepsy is genetic variation of orexin type 2 receptor (non-patent document 2), and the role of orexin in controlling sleep and wakefulness has been also attracted.

From the studies using a transgenic mouse having denatured orexin neurons and a double transgenic mouse obtained by crossing this mouse with orexin overexpressing transgenic mouse, it was clarified that narcolepsy-like symptoms that appear by degeneration of orexin neurons disappear due to sustained expression of orexin. Similarly, when orexin peptide was intraventricularly administered to a transgenic mouse having denatured orexin neuron, improvement of narcolepsy-like symptoms was also observed (non-patent document 3). Studies of orexin type 2 receptor knockout mice have suggested that orexin type 2 receptor is important for maintaining arousal (non-patent document 4, non-patent document 5). Such background suggests that orexin type 2 receptor agonists become therapeutic drugs for narcolepsy or therapeutic drugs for other sleep disorders exhibiting excessive sleepiness (non-patent document 6).

In addition, it is suggested that a peptidic agonist that selectively acts on the orexin type 2 receptor improves obesity due to high fat diet load in mice (non-patent document 7).

In addition, it is suggested that intraventricular administration of orexin peptide shortens the systemic anesthetic time of rat (non-patent document 8).

In addition, it is suggested that patients with sleep apnea syndrome show low orexin A concentration levels in plasma (non-patent document 9).

In addition, it is suggested that intraventricular administration of orexin peptide improves memory retention of senescence-accelerated model mouse (SAMP8) with cognitive dysfunction (non-patent document 10).

In addition, it is suggested that Orexin type 2 receptor agonist will be a therapeutic drug for cardiac failure (patent document 1, non-patent document 11).

In addition, it is suggested that the daytime sleepiness of Parkinson's disease patients is caused by orexin nerve fallout (non-patent document 12).

In addition, it is suggested that orexin regulates bone formation and bone loss, and orexin type 2 receptor agonist will be a therapeutic drug for diseases related to bone loss such as osteoporosis, rheumatoid arthritis and the like (patent document 2).

In addition, it is suggested that orexin receptor agonist is useful for the prophylaxis or treatment of sepsis, severe sepsis and septic shock, since the mortality was significantly improved by mere continuous administration of orexin from the periphery in septic shock model mouse (patent document 3).

Therefore, a compound having an orexin type 2 receptor agonist activity is expected to be useful as a novel therapeutic drug for narcolepsy, idiopathic hypersomnia, hypersomnia, sleep apnea syndrome, disturbance of consciousness such as coma and the like, narcolepsy syndrome accompanied by narcolepsy-like symptoms, hypersomnia syndrome accompanied by daytime hypersomnia (e.g., Parkinson's disease, Guillain-Barre syndrome and Kleine Levin syndrome), Alzheimer, obesity, insulin resistance syndrome, cardiac failure, diseases related to bone loss, sepsis and the like, further, anesthetic antagonist, a prophylactic or therapeutic drug for side effects and complications due to anesthesia.

As sulfonamide derivatives, a compound represented by the formula

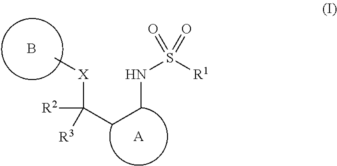

wherein each symbol is as described in the document (Patent Document 4) has been reported.

In addition, as compounds having an orexin type 2 receptor agonist activity, the following compounds have been reported.

A compound represented by the formula

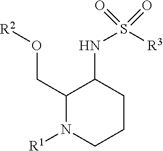

wherein each symbol is as described in the document (Patent Document 5).

A compound represented by the formula

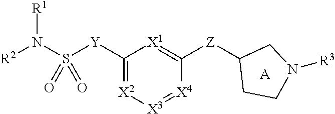

wherein each symbol is as described in the document (Patent Document 6).

A compound represented by the formula

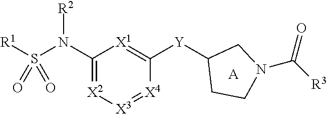

wherein each symbol is as described in the document (Patent Document 7).

A compound represented by the formula

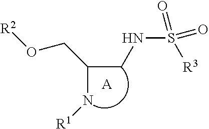

wherein each symbol is as described in the document (Patent Document 8).

A compound represented by the formula

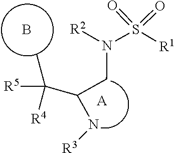

wherein each symbol is as described in the document (Patent Document 9).

A compound represented by the formula

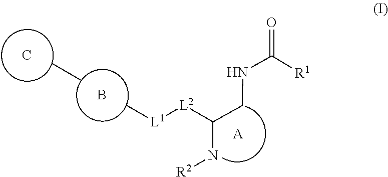

wherein each symbol is as described in the document (Patent Document 10).

A compound represented by the formula

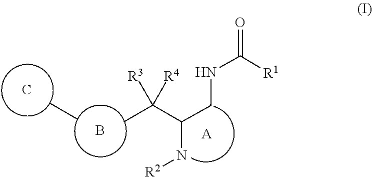

wherein each symbol is as described in the document (Patent Document 11).

A compound represented by the formula

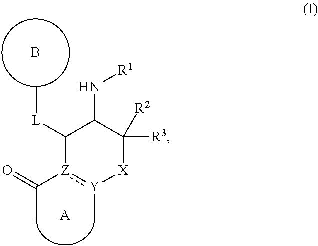

wherein each symbol is as described in the document (Patent Document 12).

A compound represented by the formula

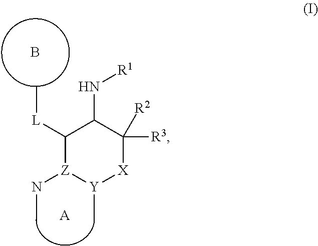

wherein each symbol is as described in the document (Patent Document 13).

A compound represented by the formula

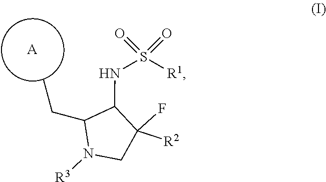

wherein each symbol is as described in the document (Patent Document 14).

A compound represented by the formula

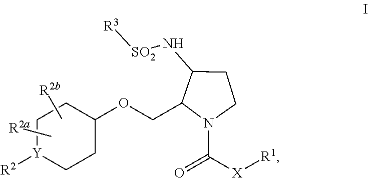

wherein each symbol is as described in the document (Patent Document 15).

A compound represented by the formula

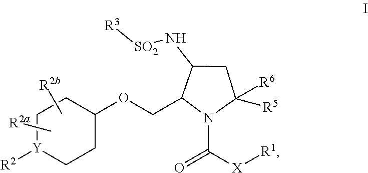

wherein each symbol is as described in the document (Patent Document 16).

A compound represented by the formula

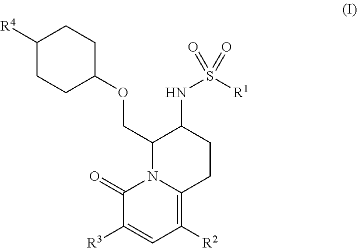

wherein each symbol is as described in the document (Patent Document 17).

Development of a novel compound having an orexin type 2 receptor agonist activity is desired.

### DOCUMENT LIST

### Patent Document

[Patent Document 1] WO 2015/073707 A1
[Patent Document 2] WO 2015/048091 A1
[Patent Document 3] WO 2015/147240 A1
[Patent Document 4] WO 2012/137982 A9
[Patent Document 5] WO 2017/135306 A1
[Patent Document 6] WO 2018/164191 A1
[Patent Document 7] WO 2018/164192 A1
[Patent Document 8] WO 2019/027003 A1
[Patent Document 9] WO 2019/027058 A1
[Patent Document 10] WO 2020/004536 A1
[Patent Document 11] WO 2020/004537 A1
[Patent Document 12] WO 2020/122092 A1
[Patent Document 13] WO 2020/122093 A1
[Patent Document 14] WO 2020/158958 A1
[Patent Document 15] WO 2020/167701 A1
[Patent Document 16] WO 2020/167706 A1
[Patent Document 17] WO 2021/106975 A1

### Non-Patent Document

[Non-Patent Document 1] Cell, Vol. 92, 573-585, 1998
[Non-Patent Document 2] Cell, Vol. 98, 365-376, 1999
[Non-Patent Document 3] Proc. Natl. Acad. Sci. USA, Vol. 101, 4649-4654, 2004
[Non-Patent Document 4] Cell, Vol. 98, 437-451, 1999
[Non-Patent Document 5] Neuron, Vol. 38, 715-730, 2003
[Non-Patent Document 6] CNS Drugs, Vol. 27, 83-90, 2013
[Non-Patent Document 7] Cell Metabolism, Vol. 9, 64-76, 2009
[Non-Patent Document 8] Neuroscience, Vol. 121, 855-863, 2003
[Non-Patent Document 9] Respiration, Vol. 71, 575-579, 2004
[Non-Patent Document 10] Peptides, Vol. 23, 1683-1688, 2002
[Non-Patent Document 11] Journal of the American College of Cardiology. Vol. 66, 2015, Pages 2522-2533
[Non-Patent Document 12] Brain. Vol. 130, 2007, Pages 1586-1595

### SUMMARY OF THE INVENTION

### Problems to be Solved by the Invention

The present invention aims to provide a heterocyclic compound having an orexin type 2 receptor agonist activity.

### Means of Solving the Problems

The present inventors have found that a compound represented by the following formula (I) or a salt thereof (sometimes to be referred to as compound (I) in the present specification) has an orexin type 2 receptor agonist activity. As a result of further studies, they have completed the present invention.

Accordingly, the present invention relates to the following.

[1] A compound of formula (I)

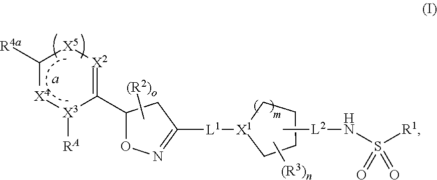

wherein
R¹ is a C₁₋₆ alkyl group, a C₃₋₇ cycloalkyl group, or a mono- or di-C₁₋₆ alkylamino group, wherein each of the C₁₋₆ alkyl group, C₃₋₇ cycloalkyl group, and mono- or di-C₁₋₆ alkylamino group is optionally substituted;
each R² is the same or different, and is an optionally substituted C₁₋₆ alkyl group;
each R³ is the same or different, and is an optionally substituted C₁₋₆ alkyl group, or a halogen atom;
R^{A} is an optionally substituted 3- to 8-membered non-aromatic heterocyclic group, an optionally substituted 5- to 6-membered aromatic heterocyclic group, an optionally substituted C₆₋₁₄ aryl group, or an optionally substituted C₃₋₁₀ cycloalkyl group;
L¹ is a bond, —NR⁶—, or —O—; wherein R⁶ is a hydrogen atom, or an optionally substituted C₁₋₆ alkyl group;
L² is a bond or —CH₂—;
X¹ is N or CR^{3′};
X² is N or CR^{4b};
X³ is N or C;
X⁴ is N or CR^{4c};
X⁵ is N or CR^{4d};
provided that no more than two of X², X³, X⁴, and X⁵ are N;
R^{3′} is a hydrogen atom, an optionally substituted C₁₋₆ alkyl group, or a halogen atom;
R^{4a}, R^{4b}, R^{4c}, and R^{4d} are the same or different and each is a hydrogen atom, an optionally substituted C₁₋₆ alkyl group, an optionally substituted C₃₋₇ cycloalkyl group, a halogen atom, or an optionally substituted C₁₋₆ alkoxy group;
m is an integer of 0 to 3;
n is an integer of 0 to 3;
o is an integer of 0 to 3;
p is an integer of 0 or 1; and
[symbol] comprises one single bond and one double bond when p is 0 or one single bond and two double bonds when p is 1 to provide an aromatic ring structure;
   or a salt thereof (hereinafter referred to as “compound (I)”).

[2] The compound according to [1], wherein R^{A} has the following structure:

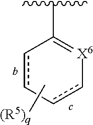

wherein
X⁶ is N or CR^{5′};
each R⁵ is the same or different, and is a halogen atom, or a C₁₋₆ alkoxy group;
R^{5′} is a hydrogen atom, or a halogen atom;
q is an integer of 0 to 7; and
[symbol] and [symbol] are the same and are both single bonds or both double bonds;
or a salt thereof.

[3] The compound according to [1] or [2], wherein X¹ is CR^{3′}, m is an integer of 0 to 2, and n is 0; or a salt thereof.

[4] The compound according to [1] or [2], wherein X¹ is N; or a salt thereof.

[5] The compound according to any one of [2]-[4], wherein
R¹ is a C₁₋₆ alkyl group, a C₁₋₆ alkoxy-C₁₋₆ alkyl group, a halo-C₁₋₆ alkyl group, a cyclopropyl group, a halo-cyclopropyl group, or a mono- or di-C₁₋₆ alkylamino group;
each R² is the same or different, and is a C₁₋₆ alkyl group, a halo-C₁₋₆ alkyl group, a C₁₋₆ alkoxy-C₁₋₆ alkyl group, or a hydroxy-C₁₋₆ alkyl group;
each R³ is the same or different, and is a C₁₋₆ alkyl group, a C₁₋₆ alkoxy-C₁₋₆ alkyl group, or a halogen atom;
R^{4a} is a hydrogen atom, a C₁₋₆ alkyl group, a cyclopropyl group, a C₁₋₆ alkoxy group, a halo-C₁₋₆ alkyl group, or a halogen atom; and
q is an integer of 0 to 4;
or a salt thereof.

[6] The compound according to any one of [1]-[5], wherein either
(i) X² is CR^{4b}, X³ is N, X⁴ is N, and p is 0; or
(ii) X² is N, X³ is N, X⁴ is CR^{4c}, and p is 0;
or a salt thereof.

[7] The compound according to any one of [1]-[5], wherein X² is CR^{4b}, X³ is C, X⁴ is CR^{4c}, X⁵ is CR^{4d}, and p is 1; or a salt thereof.

[8] The compound according to any one of [1]-[5], wherein X² is N, X³ is C, X⁴ is CR^{4c}, X⁵ is CR^{4d}, and p is 1; or a salt thereof.

[9] The compound according to any one of [1]-[5], wherein X² is CR^{4b}, X³ is C, X⁴ and X⁵ are N, and p is 1; or a salt thereof.

[10] The compound according to any one of [1]-[5], wherein X² is CR^{4b}, X³ is C, X⁴ is CR^{4c}, X⁵ is N, and p is 1; or a salt thereof.

[11] The compound according to any one of [1]-[5], wherein X² and X⁴ are N, X³ is C, X⁵ is CR^{4d}, and p is 1; or a salt thereof.

[12] The compound according to any one of [1]-[5], wherein X² is CR^{4b}, X³ is C, X⁴ is N, X⁵ is CR^{4d}, and p is 1; or a salt thereof.

[13] The compound according to any one of [2]-[12], wherein X⁶ is CR^{5′}, and [symbol] and [symbol] are both single bonds; or a salt thereof.

[14] The compound according to any one of [2]-[12], wherein X⁶ is CR^{5′}, and [symbol] and [symbol] are both double bonds; or a salt thereof.

[15] The compound according to any one of [2]-[12], wherein X is N′, and [symbol] and [symbol] are both double bonds; or a salt thereof.

[16] The compound according to any one of [1]-[5], wherein the moiety:

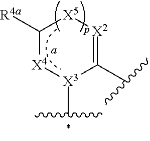

is selected from the group consisting of:

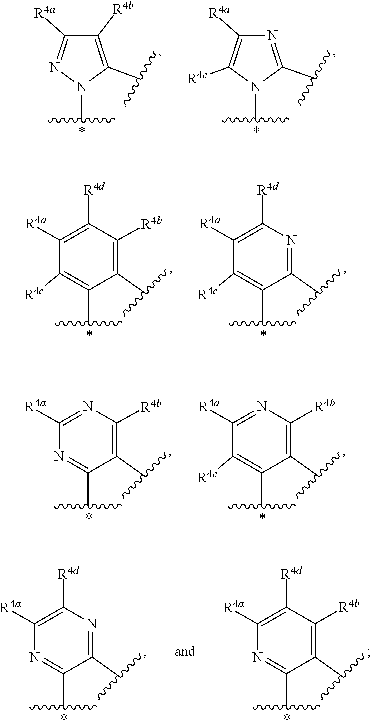

wherein
* indicates the bonding site to R^{A}; and
R^{4a}, R^{4b}, R^{4c}, and R^{4d} are as defined in claim 1;
or a salt thereof.

[17] The compound according to any one of [2]-[5], wherein the moiety:

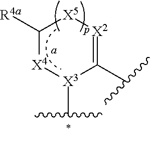

is selected from the group consisting of:

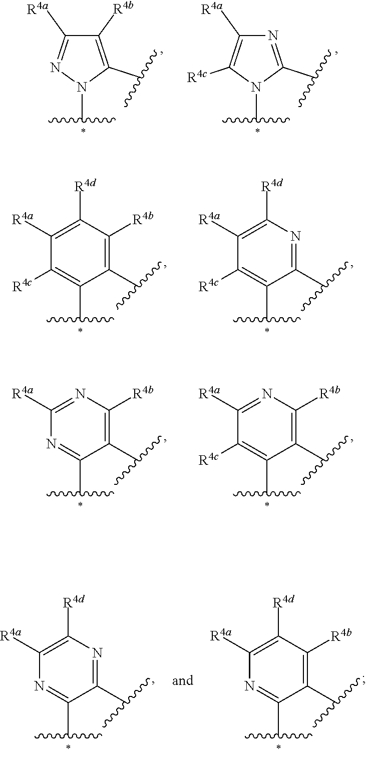

wherein * indicates the bonding site to R^{A};
R¹ is a C₁₋₆ alkyl group, a C₁₋₆ alkoxy-C₁₋₆ alkyl group, a halo-C₁₋₆ alkyl group, a cyclopropyl group, a halo-cyclopropyl group, or a mono- or di-C₁₋₆ alkylamino group;
each R² is the same or different, and is a C₁₋₆ alkyl group, a halo-C₁₋₆ alkyl group, a C₁₋₆ alkoxy-C₁₋₆ alkyl group, or a hydroxy-C₁₋₆ alkyl group;
each R³ is the same or different, and is a C₁₋₆ alkyl group, a C₁₋₆ alkoxy-C₁₋₆ alkyl group, or a halogen atom;
L¹ is a bond, —NR⁶—, or —O—; wherein R⁶ is a hydrogen atom, or a C₁₋₆ alkyl group;
L² is a bond or —CH₂—;
X¹ is N or CR^{3′}; wherein R^{3′} is a hydrogen atom;

R^{4a} is a hydrogen atom, a C₁₋₆ alkyl group, a cyclopropyl group, a C₁₋₆ alkoxy group, a halo-C₁₋₆ alkyl group, or a halogen atom;
X² is N or CR^{4b}; wherein R^{4b} is a hydrogen atom, or a halogen atom;
X⁴ is N or CR^{4c}; wherein R^{4c} is a hydrogen atom, or a halogen atom;
X⁵ is N or CR^{4d}; wherein R^{4d} is a hydrogen atom;
m is an integer of 0 to 3;
n is an integer of 0 to 3;
is an integer of 0 to 3; and
the moiety:

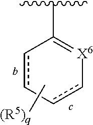

is selected from the group consisting of:

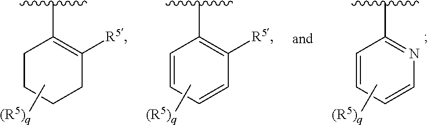

wherein
each R⁵ is the same or different, and is a halogen atom, or a C₁₋₆ alkoxy group;
R^{5′} is a hydrogen atom, or a halogen atom; and
q is an integer of 0 to 4;
   or a salt thereof.

[18] The compound according to any one of [2]-[5], wherein the compound is a compound of formula (Ia):

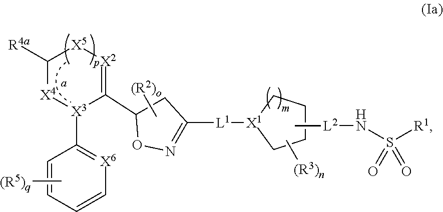

wherein
q is an integer of 0 to 4; and
the other symbols are as defined in [2];
   or a salt thereof.

[19] The compound according to any one of [2]-[5], wherein the compound is a compound of formula (Ib):

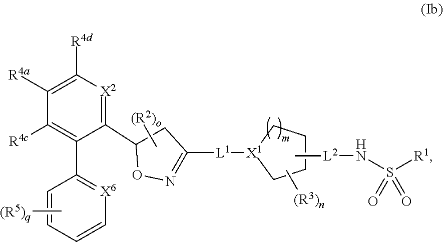

wherein
q is an integer of 0 to 4; and
the other symbols are as defined in [2];
or a salt thereof.

[20] The compound according to any one of [2]-[5], wherein the compound is a compound of formula (Ic):

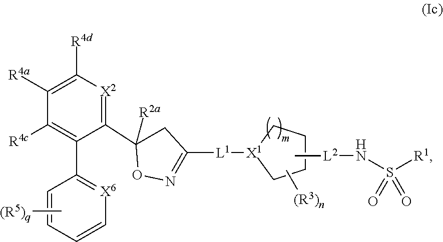

wherein
R^{2a} is a hydrogen atom, or an optionally substituted C₁₋₆ alkyl group;
q is an integer of 0 to 4; and
the other symbols are as defined in [2];
   or a salt thereof.

[21] The compound according to any one of [2]-[5], wherein the compound is a compound of formula (Id):

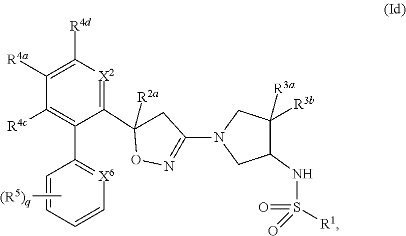

wherein
R^{2a} is a hydrogen atom, or an optionally substituted C₁₋₆ alkyl group;
R^{3a} and R^{3b} are the same or different and each is a hydrogen atom, an optionally substituted C₁₋₆ alkyl group, or a halogen atom;
q is an integer of 0 to 4; and
the other symbols are as defined in [2];
   or a salt thereof.

[22] The compound according to any one of [1]-[5], wherein the compound is a compound of formula (Ie):

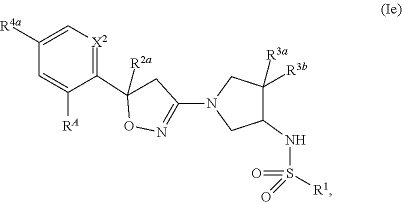

wherein
R¹ is a C₁₋₆ alkyl group, or a halo-C₁₋₆ alkyl group;
R^{2a} is a hydrogen atom, a C₁₋₆ alkyl group, or a halo-C₁₋₆ alkyl group;
R^{3a} and R^{3b} are the same or different and each is a C₁₋₆ alkyl group, or a halogen atom;
R^{4a} is a C₁₋₆ alkyl group, or a halogen atom;
X² is N or CR^{4b}; wherein R^{4b} is a halogen atom; and
R^{A} is a phenyl group, or a pyridyl group, wherein each of the phenyl group and pyridyl group is optionally substituted by one to three halogen atoms;
   or a salt thereof.

[23] The compound according to [22], wherein
R¹ is a C₁₋₆ alkyl group, or a halo-C₁₋₆ alkyl group;
R^{2a} is a hydrogen atom, a C₁₋₆ alkyl group, or a halo-C₁₋₆ alkyl group;
R^{3a} and R^{3b} are each a halogen atom;
R^{4a} is a C₁₋₆ alkyl group, or a halogen atom;
X² is N or CR^{4b}; wherein R^{4b} is a halogen atom; and
R^{A} is a phenyl group, or a pyridyl group, wherein each of the phenyl group and pyridyl group is optionally substituted by one to three halogen atoms;
   or a salt thereof.

[24] The compound according to [22], wherein
R¹ is a C₁₋₆ alkyl group, or a halo-C₁₋₆ alkyl group;
R^{2a} is a C₁₋₆ alkyl group, or a halo-C₁₋₆ alkyl group;
R^{3a} and R^{3b} are each a halogen atom;
R^{4a} is a halogen atom;
X² is N; and
R^{A} is a phenyl group optionally substituted by one to three halogen atoms;
   or a salt thereof.

[25] The compound according to any one of [1]-[5] and [22]-[24], wherein
R¹ is a C₁₋₆ alkyl group;
R^{2a} is a halo-C₁₋₆ alkyl group;
R^{3a} and R^{3b} are each a halogen atom;
R^{4a} is a halogen atom;
X² is N; and
R^{A} is a phenyl group optionally substituted by one to three halogen atoms;
   or a salt thereof.

The compound according to [1], wherein the compound is selected from the group consisting of:
N-[(3R)-4,4-difluoro-1-{(5S)-5-[5-methyl-3-(2,4,6-trifluorophenyl)pyridin-2-yl]-4,5-dihydro-1,2-oxazol-3-yl}pyrrolidin-3-yl]methanesulfonamide;
N-[(3R)-1-{(5S)-5-[3-(2,6-difluorophenyl)-5-fluoropyridin-2-yl]-5-(fluoromethyl)-4,5-dihydro-1,2-oxazol-3-yl}-4,4-difluoropyrrolidin-3-yl]methanesulfonamide;
N-[(3R)-1-{(5S)-5-[3-(2,6-difluorophenyl)-5-fluoropyridin-2-yl]-5-(fluoromethyl)-4,5-dihydro-1,2-oxazol-3-yl}-4,4-difluoropyrrolidin-3-yl]-1-fluoromethanesulfonamide; and
N-[(3R)-1-{(5S)-5-[3-(2,6-difluorophenyl)-5-fluoropyridin-2-yl]-5-methyl-4,5-dihydro-1,2-oxazol-3-yl}-4,4-difluoropyrrolidin-3-yl]methanesulfonamide, or a salt thereof.

[27] A pharmaceutical composition comprising the compound as defined in any one of [1]-[26] or a salt thereof, and a pharmacologically acceptable carrier.

[28] A medicament comprising the compound as defined in any one of [1]-[26] or a salt thereof, or the pharmaceutical composition as defined in [27].

[29] The medicament according to [28], which is an orexin type 2 receptor agonist.

[30] The medicament according to [28], which is an agent for the prophylaxis or treatment of narcolepsy, idiopathic hypersomnia, hypersomnia, sleep apnea syndrome, narcolepsy syndrome accompanied by narcolepsy-like symptoms, hypersomnia syndrome accompanied by daytime hypersomnia, Alzheimer's disease, obesity, insulin resistance syndrome, cardiac failure, diseases related to bone loss, sepsis, disturbance of consciousness, or side effects and complications due to anesthesia.

[31] The medicament according to [28], which is an agent for the prophylaxis or treatment of narcolepsy, idiopathic hypersomnia, hypersomnia, or sleep apnea syndrome.

[32] The medicament according to [28], which is an agent for the prophylaxis or treatment of narcolepsy.

[33] A method for the prophylaxis or treatment of a disease or disorder associated with an orexin type 2 receptor in a mammal in need thereof, comprising administering to the mammal a therapeutically effective amount of the compound as defined in any one of [1]-[26] or a salt thereof, or the pharmaceutical composition as defined in [27].

[34] The method according to [33], wherein the disease or disorder is selected from the group consisting of narcolepsy, idiopathic hypersomnia, hypersomnia, sleep apnea syndrome, narcolepsy syndrome accompanied by narcolepsy-like symptoms, hypersomnia syndrome accompanied by daytime hypersomnia, Alzheimer's disease, obesity, insulin resistance syndrome, cardiac failure, diseases related to bone loss, sepsis, disturbance of consciousness, and side effects and complications due to anesthesia.

[35] The method according to [33], wherein the disease or disorder is selected from the group consisting of narcolepsy, idiopathic hypersomnia, hypersomnia, and sleep apnea syndrome.

[36] The method according to [33], wherein the disease or disorder is narcolepsy.

[37] The compound as defined in any one of [1]-[26], or a salt thereof, for use in therapy.

[38] The compound or salt according to [37], wherein the therapy comprises treatment of a disease or disorder associated with an orexin type 2 receptor.

[39] The compound or salt according to [38], wherein the disease or disorder is selected from the group consisting of narcolepsy, idiopathic hypersomnia, hypersomnia, sleep apnea syndrome, narcolepsy syndrome accompanied by narcolepsy-like symptoms, hypersomnia syndrome accompanied by daytime hypersomnia, Alzheimer's disease, obesity, insulin resistance syndrome, cardiac failure, diseases related to bone loss, sepsis, disturbance of consciousness, and side effects and complications due to anesthesia.

[40] The compound or salt according to [38], wherein the disease or disorder is selected from the group consisting of narcolepsy, idiopathic hypersomnia, hypersomnia, and sleep apnea syndrome.

[41] The compound or salt according to [38], wherein the disease or disorder is narcolepsy.

[42] Use of the compound as defined in any one of [1]-[26] or a salt thereof, in the manufacture of a medicament for the treatment of a disease or disorder associated with an orexin type 2 receptor.

[43] Use according to [42], wherein the disease or disorder is selected from the group consisting of narcolepsy, idiopathic hypersomnia, hypersomnia, sleep apnea syndrome, narcolepsy syndrome accompanied by narcolepsy-like symptoms, hypersomnia syndrome accompanied by daytime hypersomnia, Alzheimer's disease, obesity, insulin resistance syndrome, cardiac failure, diseases related to bone loss, sepsis, disturbance of consciousness, and side effects and complications due to anesthesia.

[44] Use according to [42], wherein the disease or disorder is selected from the group consisting of narcolepsy, idiopathic hypersomnia, hypersomnia, and sleep apnea syndrome.

[45] Use according to [42], wherein the disease or disorder is narcolepsy.

### Effect of the Invention

The compound of the present invention has an orexin type 2 receptor agonist activity, and is useful as an agent for the prophylaxis or treatment of certain diseases, such as narcolepsy.

### DETAILED DESCRIPTION OF THE INVENTION

The definition of each substituent used in the present specification is described in detail in the following. Unless otherwise specified, each substituent has the following definition.

In the present specification, examples of the “halogen atom” include fluorine, chlorine, bromine and iodine.

In the present specification, examples of the “C₁₋₆ alkyl group” include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl and 2-ethylbutyl.

In the present specification, examples of the “optionally halogenated C₁₋₆ alkyl group” include a C₁₋₆ alkyl group optionally having 1 to 7, preferably 1 to 5, halogen atoms. Specific examples thereof include methyl, chloromethyl, difluoromethyl, trichloromethyl, trifluoromethyl, ethyl, 2-bromoethyl, 2,2,2-trifluoroethyl, tetrafluoroethyl, pentafluoroethyl, propyl, 2,2-difluoropropyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl and 6,6,6-trifluorohexyl.

In the present specification, examples of the “C₂₋₆ alkenyl group” include ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl and 5-hexenyl.

In the present specification, examples of the “C₂₋₆ alkynyl group” include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and 4-methyl-2-pentynyl.

In the present specification, examples of the “C₃₋₁₀ cycloalkyl group” include cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl and adamantyl.

In the present specification, examples of the “optionally halogenated C₃₋₁₀ cycloalkyl group” include a C₃₋₁₀ cycloalkyl group optionally having 1 to 7, preferably 1 to 5, halogen atoms. Specific examples thereof include cyclopropyl, 2,2-difluorocyclopropyl, 2,3-difluorocyclopropyl, cyclobutyl, difluorocyclobutyl, cyclopentyl, cyclohexyl, cycloheptyl and cyclooctyl.

In the present specification, examples of the “C₃₋₁₀ cycloalkenyl group” include cyclopropenyl, cyclobutenyl, cyclopentenyl, cyclohexenyl, cycloheptenyl and cyclooctenyl.

In the present specification, examples of the “C₆₋₁₄ aryl group” include phenyl, 1-naphthyl, 2-naphthyl, 1-anthryl, 2-anthryl and 9-anthryl.

In the present specification, examples of the “C₇₋₁₆ aralkyl group” include benzyl, phenethyl, naphthylmethyl and phenylpropyl.

In the present specification, examples of the “C₁₋₆ alkoxy group” include methoxy, ethoxy, propoxy, isopropoxy, butoxy, isobutoxy, sec-butoxy, tert-butoxy, pentyloxy and hexyloxy.

In the present specification, examples of the “optionally halogenated C₁₋₆ alkoxy group” include a C₁₋₆ alkoxy group optionally having 1 to 7, preferably 1 to 5, halogen atoms. Specific examples thereof include methoxy, difluoromethoxy, trifluoromethoxy, ethoxy, 2,2,2-trifluoroethoxy, propoxy, isopropoxy, butoxy, 4,4,4-trifluorobutoxy, isobutoxy, sec-butoxy, pentyloxy and hexyloxy.

In the present specification, examples of the “C₃₋₁₀ cycloalkyloxy group” include cyclopropyloxy, cyclobutyloxy, cyclopentyloxy, cyclohexyloxy, cycloheptyloxy and cyclooctyloxy.

In the present specification, examples of the “C₁₋₆ alkylthio group” include methylthio, ethylthio, propylthio, isopropylthio, butylthio, sec-butylthio, tert-butylthio, pentylthio and hexylthio.

In the present specification, examples of the “optionally halogenated C₁₋₆ alkylthio group” include a C₁₋₆ alkylthio group optionally having 1 to 7, preferably 1 to 5, halogen atoms. Specific examples thereof include methylthio, difluoromethylthio, trifluoromethylthio, ethylthio, propylthio, isopropylthio, butylthio, 4,4,4-trifluorobutylthio, pentylthio and hexylthio.

In the present specification, examples of the “C₁₋₆ alkyl-carbonyl group” include acetyl, propanoyl, butanoyl, 2-methylpropanoyl, pentanoyl, 3-methylbutanoyl, 2-methylbutanoyl, 2,2-dimethylpropanoyl, hexanoyl and heptanoyl.

In the present specification, examples of the “optionally halogenated C₁₋₆ alkyl-carbonyl group” include a C₁₋₆ alkyl-carbonyl group optionally having 1 to 7, preferably 1 to 5, halogen atoms. Specific examples thereof include acetyl, chloroacetyl, trifluoroacetyl, trichloroacetyl, propanoyl, butanoyl, pentanoyl and hexanoyl.

In the present specification, examples of the “C₁₋₆ alkoxy-carbonyl group” include methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl and hexyloxycarbonyl.

In the present specification, examples of the “C₆₋₁₄ aryl-carbonyl group” include benzoyl, 1-naphthoyl and 2-naphthoyl. In the present specification, examples of the “C₇₋₁₆ aralkyl-carbonyl group” include phenylacetyl and phenylpropionyl.

In the present specification, examples of the “5- to 14-membered aromatic heterocyclylcarbonyl group” include nicotinoyl, isonicotinoyl, thenoyl and furoyl.

In the present specification, examples of the “3- to 14-membered non-aromatic heterocyclylcarbonyl group” include morpholinylcarbonyl, piperidinylcarbonyl and pyrrolidinylcarbonyl.

In the present specification, examples of the “mono- or di-C₁₋₆ alkyl-carbamoyl group” include methylcarbamoyl, ethylcarbamoyl, dimethylcarbamoyl, diethylcarbamoyl and N-ethyl-N-methylcarbamoyl.

In the present specification, examples of the “mono- or di-C₇₋₁₆ aralkyl-carbamoyl group” include benzylcarbamoyl and phenethylcarbamoyl.

In the present specification, examples of the “C₁₋₆ alkylsulfonyl group” include methylsulfonyl, ethylsulfonyl, propylsulfonyl, isopropylsulfonyl, butylsulfonyl, sec-butylsulfonyl and tert-butylsulfonyl.

In the present specification, examples of the “optionally halogenated C₁₋₆ alkylsulfonyl group” include a C₁₋₆ alkylsulfonyl group optionally having 1 to 7, preferably 1 to 5, halogen atoms. Specific examples thereof include methylsulfonyl, difluoromethylsulfonyl, trifluoromethylsulfonyl, ethylsulfonyl, propylsulfonyl, isopropylsulfonyl, butylsulfonyl, 4,4,4-trifluorobutylsulfonyl, pentylsulfonyl and hexylsulfonyl.

In the present specification, examples of the “C₆₋₁₄ arylsulfonyl group” include phenylsulfonyl, 1-naphthylsulfonyl and 2-naphthylsulfonyl.

In the present specification, examples of the “substituent” include a halogen atom, a cyano group, a nitro group, an optionally substituted hydrocarbon group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group and an optionally substituted silyl group.

In the present specification, examples of the “hydrocarbon group” (including “hydrocarbon group” of “optionally substituted hydrocarbon group”) include a C₁₋₆ alkyl group, a C₂₋₆ alkenyl group, a C₂₋₆ alkynyl group, a C₃₋₁₀ cycloalkyl group, a C₃₋₁₀ cycloalkenyl group, a C₆₋₁₄ aryl group and a C₇₋₁₆ aralkyl group.

In the present specification, examples of the “optionally substituted hydrocarbon group” include a hydrocarbon group optionally having substituent (s) selected from the following Substituent group A.

### [Substituent Group A]

(1) a halogen atom,
(2) a nitro group,
(3) a cyano group,
(4) an oxo group,
(5) a hydroxy group,
(6) an optionally halogenated C₁₋₆ alkoxy group,
(7) a C₆₋₁₄ aryloxy group (e.g., phenoxy, naphthoxy),
(8) a C₇₋₁₆ aralkyloxy group (e.g., benzyloxy),
(9) a 5- to 14-membered aromatic heterocyclyloxy group (e.g., pyridyloxy),
(10) a 3- to 14-membered non-aromatic heterocyclyloxy group (e.g., morpholinyloxy, piperidinyloxy),
(11) a C₁₋₆ alkyl-carbonyloxy group (e.g., acetoxy, propanoyloxy),
(12) a C₆₋₁₄ aryl-carbonyloxy group (e.g., benzoyloxy, 1-naphthoyloxy, 2-naphthoyloxy),
(13) a C₁₋₆ alkoxy-carbonyloxy group (e.g., methoxycarbonyloxy, ethoxycarbonyloxy, propoxycarbonyloxy, butoxycarbonyloxy),
(14) a mono- or di-C₁₋₆ alkyl-carbamoyloxy group (e.g., methylcarbamoyloxy, ethylcarbamoyloxy, dimethylcarbamoyloxy, diethylcarbamoyloxy),
(15) a C₆₋₁₄ aryl-carbamoyloxy group (e.g., phenylcarbamoyloxy, naphthylcarbamoyloxy),
(16) a 5- to 14-membered aromatic heterocyclylcarbonyloxy group (e.g., nicotinoyloxy),
(17) a 3- to 14-membered non-aromatic heterocyclylcarbonyloxy group (e.g., morpholinylcarbonyloxy, piperidinylcarbonyloxy),
(18) an optionally halogenated C₁₋₆ alkylsulfonyloxy group (e.g., methylsulfonyloxy, trifluoromethylsulfonyloxy),
(19) a C₆₋₁₄ arylsulfonyloxy group optionally substituted by a C₁-6 alkyl group (e.g., phenylsulfonyloxy, toluenesulfonyloxy),
(20) an optionally halogenated C₁₋₆ alkylthio group,
(21) a 5- to 14-membered aromatic heterocyclic group,
(22) a 3- to 14-membered non-aromatic heterocyclic group,
(23) a formyl group,
(24) a carboxy group,
(25) an optionally halogenated C₁₋₆ alkyl-carbonyl group,
(26) a C₆₋₁₄ aryl-carbonyl group,
(27) a 5- to 14-membered aromatic heterocyclylcarbonyl group,
(28) a 3- to 14-membered non-aromatic heterocyclylcarbonyl group,
(29) a C₁₋₆ alkoxy-carbonyl group,
(30) a C₆₋₁₄ aryloxy-carbonyl group (e.g., phenyloxycarbonyl, 1-naphthyloxycarbonyl, 2-naphthyloxycarbonyl),
(31) a C₇₋₁₆ aralkyloxy-carbonyl group (e.g., benzyloxycarbonyl, phenethyloxycarbonyl),
(32) a carbamoyl group,
(33) a thiocarbamoyl group,
(34) a mono- or di-C₁₋₆ alkyl-carbamoyl group,
(35) a C₆₋₁₄ aryl-carbamoyl group (e.g., phenylcarbamoyl),
(36) a 5- to 14-membered aromatic heterocyclylcarbamoyl group (e.g., pyridylcarbamoyl, thienylcarbamoyl),
(37) a 3- to 14-membered non-aromatic heterocyclylcarbamoyl group (e.g., morpholinylcarbamoyl, piperidinylcarbamoyl),
(38) an optionally halogenated C₁₋₆ alkylsulfonyl group,
(39) a C₆₋₁₄ arylsulfonyl group,
(40) a 5- to 14-membered aromatic heterocyclylsulfonyl group (e.g., pyridylsulfonyl, thienylsulfonyl),
(41) an optionally halogenated C₁₋₆ alkylsulfinyl group,
(42) a C₆₋₁₄ arylsulfinyl group (e.g., phenylsulfinyl, 1-naphthylsulfinyl, 2-naphthylsulfinyl),
(43) a 5- to 14-membered aromatic heterocyclylsulfinyl group (e.g., pyridylsulfinyl, thienylsulfinyl),
(44) an amino group,
(45) a mono- or di-C₁₋₆ alkylamino group (e.g., methylamino, ethylamino, propylamino, isopropylamino, butylamino, dimethylamino, diethylamino, dipropylamino, dibutylamino, N-ethyl-N-methylamino),
(46) a mono- or di-C₆₋₁₄ arylamino group (e.g., phenylamino),
(47) a 5- to 14-membered aromatic heterocyclylamino group (e.g., pyridylamino),
(48) a C₇₋₁₆ aralkylamino group (e.g., benzylamino),
(49) a formylamino group,
(50) a C₁₋₆ alkyl-carbonylamino group (e.g., acetylamino, propanoylamino, butanoylamino),
(51) a (C₁₋₆ alkyl) (C₁₋₆ alkyl-carbonyl) amino group (e.g., N-acetyl-N-methylamino),
(52) a C₆₋₁₄ aryl-carbonylamino group (e.g., phenylcarbonylamino, naphthylcarbonylamino),
(53) a C₁₋₆ alkoxy-carbonylamino group (e.g., methoxycarbonylamino, ethoxycarbonylamino, propoxycarbonylamino, butoxycarbonylamino, tert-butoxycarbonylamino),
(54) a C₇₋₁₆ aralkyloxy-carbonylamino group (e.g., benzyloxycarbonylamino),
(55) a C₁₋₆ alkylsulfonylamino group (e.g., methylsulfonylamino, ethylsulfonylamino),
(56) a C₆₋₁₄ arylsulfonylamino group optionally substituted by a C₁₋₆ alkyl group (e.g., phenylsulfonylamino, toluenesulfonylamino),
(57) an optionally halogenated C₁₋₆ alkyl group,
(58) a C₂₋₆ alkenyl group,
(59) a C₂₋₆ alkynyl group,
(60) a C₃₋₁₀ cycloalkyl group,
(61) a C₃₋₁₀ cycloalkenyl group, and
(62) a C₆₋₁₄ aryl group.

The number of the above-mentioned substituents in the “optionally substituted hydrocarbon group” is, for example, 1 to 5, preferably 1 to 3. When the number of the substituents is two or more, the respective substituents may be the same or different.

In the present specification, examples of the “heterocyclic group” (including “heterocyclic group” of “optionally substituted heterocyclic group”) include (i) an aromatic heterocyclic group, (ii) a non-aromatic heterocyclic group and (iii) a 7- to 10-membered bridged heterocyclic group, each containing, as a ring-constituting atom besides carbon atom, 1 to 4 heteroatoms selected from a nitrogen atom, a sulfur atom and an oxygen atom.

In the present specification, examples of the “aromatic heterocyclic group” (including “5- to 14-membered aromatic heterocyclic group”) include a 5- to 14-membered (preferably 5-to 10-membered) aromatic heterocyclic group containing, as a ring-constituting atom besides carbon atom, 1 to 4 heteroatoms selected from a nitrogen atom, a sulfur atom and an oxygen atom.

Preferable examples of the “aromatic heterocyclic group” include 5- or 6-membered monocyclic aromatic heterocyclic groups such as thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl, triazinyl and the like; and 8- to 14-membered fused polycyclic (preferably bi- or tri-cyclic) aromatic heterocyclic groups such as benzothiophenyl, benzofuranyl, benzimidazolyl, benzoxazolyl, benzisoxazolyl, benzothiazolyl, benzisothiazolyl, benzotriazolyl, imidazopyridinyl, thienopyridinyl, furopyridinyl, pyrrolopyridinyl, pyrazolopyridinyl, oxazolopyridinyl, thiazolopyridinyl, imidazopyrazinyl, imidazopyrimidinyl, thienopyrimidinyl, furopyrimidinyl, pyrrolopyrimidinyl, pyrazolopyrimidinyl, oxazolopyrimidinyl, thiazolopyrimidinyl, pyrazolotriazinyl, naphtho[2,3-b]thienyl, phenoxathiinyl, indolyl, isoindolyl, 1H-indazolyl, purinyl, isoquinolyl, quinolyl, phthalazinyl, naphthyridinyl, quinoxalinyl, quinazolinyl, cinnolinyl, carbazolyl, β-carbolinyl, phenanthridinyl, acridinyl, phenazinyl, phenothiazinyl, phenoxazinyl and the like.

In the present specification, examples of the “non-aromatic heterocyclic group” (including “3- to 14-membered non-aromatic heterocyclic group”) include a 3- to 14-membered (preferably 4- to 10-membered) non-aromatic heterocyclic group containing, as a ring-constituting atom besides carbon atom, 1 to 4 heteroatoms selected from a nitrogen atom, a sulfur atom and an oxygen atom.

Preferable examples of the “non-aromatic heterocyclic group” include 3- to 8-membered monocyclic non-aromatic heterocyclic groups such as aziridinyl, oxiranyl, thiiranyl, azetidinyl, oxetanyl, thietanyl, tetrahydrothienyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydroisothiazolyl, tetrahydrooxazolyl, tetrahydroisooxazolyl, piperidinyl, piperazinyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, thiomorpholinyl, azepanyl, diazepanyl, azepinyl, oxepanyl, azocanyl, diazocanyl and the like; and 9- to 14-membered fused polycyclic (preferably bi- or tri-cyclic) non-aromatic heterocyclic groups such as dihydrobenzofuranyl, dihydrobenzimidazolyl, dihydrobenzoxazolyl, dihydrobenzothiazolyl, dihydrobenzisothiazolyl, dihydronaphtho[2,3-b] thienyl, tetrahydroisoquinolyl, tetrahydroquinolyl, 4H-quinolizinyl, indolinyl, isoindolinyl, tetrahydrothieno[2,3-c]pyridinyl, tetrahydrobenzazepinyl, tetrahydroquinoxalinyl, tetrahydrophenanthridinyl, hexahydrophenothiazinyl, hexahydrophenoxazinyl, tetrahydrophthalazinyl, tetrahydronaphthyridinyl, tetrahydroquinazolinyl, tetrahydrocinnolinyl, tetrahydrocarbazolyl, tetrahydro-β-carbolinyl, tetrahydroacrydinyl, tetrahydrophenazinyl, tetrahydrothioxanthenyl, octahydroisoquinolyl and the like.

In the present specification, preferable examples of the “7- to 10-membered bridged heterocyclic group” include quinuclidinyl and 7-azabicyclo[2.2.1]heptanyl.

In the present specification, examples of the “nitrogen-containing heterocyclic group” include a “heterocyclic group” containing at least one nitrogen atom as a ring-constituting atom.

In the present specification, examples of the “optionally substituted heterocyclic group” include a heterocyclic group optionally having substituent (s) selected from the above-mentioned Substituent group A.

The number of the substituents in the “optionally substituted heterocyclic group” is, for example, 1 to 3. When the number of the substituents is two or more, the respective substituents may be the same or different.

In the present specification, examples of the “acyl group” include a formyl group, a carboxy group, a carbamoyl group, a thiocarbamoyl group, a sulfino group, a sulfo group, a sulfamoyl group and a phosphono group, each optionally having “1 or 2 substituents selected from a C₁₋₆ alkyl group, a C₂₋₆ alkenyl group, a C₃₋₁₀ cycloalkyl group, a C₃₋₁₀ cycloalkenyl group, a C₆₋₁₄ aryl group, a C₇₋₁₆ aralkyl group, a 5- to 14-membered aromatic heterocyclic group, a 3- to 14-membered non-aromatic heterocyclic group, an amino group and a mono- or di-C₁₋₆ alkyl-amino group, each of which optionally has 1 to 3 substituents selected from a halogen atom, an optionally halogenated C₁₋₆ alkoxy group, a hydroxy group, a nitro group, a cyano group, an amino group and a carbamoyl group”.

Examples of the “acyl group” also include a hydrocarbon-sulfonyl group, a heterocyclylsulfonyl group, a hydrocarbon-sulfinyl group and a heterocyclylsulfinyl group.

Here, the hydrocarbon-sulfonyl group means a hydrocarbon group-bonded sulfonyl group, the heterocyclylsulfonyl group means a heterocyclic group-bonded sulfonyl group, the hydrocarbon-sulfinyl group means a hydrocarbon group-bonded sulfinyl group and the heterocyclylsulfinyl group means a heterocyclic group-bonded sulfinyl group.

Preferable examples of the “acyl group” include a formyl group, a carboxy group, a C₁₋₆ alkyl-carbonyl group, a C₂₋₆ alkenyl-carbonyl group (e.g., crotonoyl), a C₃₋₁₀ cycloalkyl-carbonyl group (e.g., cyclobutanecarbonyl, cyclopentanecarbonyl, cyclohexanecarbonyl, cycloheptanecarbonyl), a C₃₋₁₀ cycloalkenyl-carbonyl group (e.g., 2-cyclohexenecarbonyl), a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a C₆₋₁₄ aryloxy-carbonyl group (e.g., phenyloxycarbonyl, naphthyloxycarbonyl), a C₇₋₁₆ aralkyloxy-carbonyl group (e.g., benzyloxycarbonyl, phenethyloxycarbonyl), a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl), a mono- or di-C₆₋₁₄ aryl-carbamoyl group (e.g., phenylcarbamoyl), a mono- or di-C₇₋₁₆ aralkyl-carbamoyl group, a 5- to 14-membered aromatic heterocyclylcarbamoyl group (e.g., pyridylcarbamoyl), N—C₁₋₆ alkyl-N′,N′-di-C₁₋₆ alkylhydrazine-carbonyl group, a thiocarbamoyl group, a mono- or di-C₁₋₆ alkyl-thiocarbamoyl group (e.g., methylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a 5- to 14-membered aromatic heterocyclylthiocarbamoyl group (e.g., pyridylthiocarbamoyl), a sulfino group, a C₁₋₆ alkylsulfinyl group (e.g., methylsulfinyl, ethylsulfinyl), a sulfo group, a C₁₋₆ alkylsulfonyl group, a C₆₋₁₄ arylsulfonyl group, a phosphono group and a mono- or di-C₁₋₆ alkylphosphono group (e.g., dimethylphosphono, diethylphosphono, diisopropylphosphono, dibutylphosphono).

In the present specification, examples of the “optionally substituted amino group” include an amino group optionally having “1 or 2 substituents selected from a C₁₋₆ alkyl group, a Ce-6 alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from Substituent group A”.

Preferable examples of the optionally substituted amino group include an amino group, a mono- or di-(optionally halogenated C₁₋₆ alkyl) amino group (e.g., methylamino, trifluoromethylamino, dimethylamino, ethylamino, diethylamino, propylamino, dibutylamino), a mono- or di-C₂₋₆ alkenylamino group (e.g., diallylamino), a mono- or di-C₃₋₁₀ cycloalkylamino group (e.g., cyclopropylamino, cyclohexylamino), a mono- or di-C₆₋₁₄ arylamino group (e.g., phenylamino), a mono- or di-C₇₋₁₆ aralkylamino group (e.g., benzylamino, dibenzylamino), a mono- or di-(optionally halogenated C₁₋₆ alkyl)-carbonylamino group (e.g., acetylamino, propionylamino), a mono- or di-C₆₋₁₄ aryl-carbonylamino group (e.g., benzoylamino), a mono- or di-C₇₋₁₆ aralkyl-carbonylamino group (e.g., benzylcarbonylamino), a mono- or di-5- to 14-membered aromatic heterocyclylcarbonylamino group (e.g., nicotinoylamino, isonicotinoylamino), a mono- or di-3- to 14-membered non-aromatic heterocyclylcarbonylamino group (e.g., piperidinylcarbonylamino), a mono- or di-C₁₋₆ alkoxy-carbonylamino group (e.g., tert-butoxycarbonylamino), a 5- to 14-membered aromatic heterocyclylamino group (e.g., pyridylamino), a carbamoylamino group, a (mono- or di-C₁₋₆ alkyl-carbamoyl) amino group (e.g., methylcarbamoylamino), a (mono- or di-C₇₋₁₆ aralkyl-carbamoyl) amino group (e.g., benzylcarbamoylamino), a C₁₋₆ alkylsulfonylamino group (e.g., methylsulfonylamino, ethylsulfonylamino), a C₆₋₁₄ arylsulfonylamino group (e.g., phenylsulfonylamino), a (C₁₋₆ alkyl) (C₁₋₆ alkyl-carbonyl) amino group (e.g., N-acetyl-N-methylamino) and a (C₁₋₆ alkyl) (C₆₋₁₄ aryl-carbonyl) amino group (e.g., N-benzoyl-N-methylamino).

In the present specification, examples of the “optionally substituted carbamoyl group” include a carbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from Substituent group A”.

Preferable examples of the optionally substituted carbamoyl group include a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl, cyclohexylcarbamoyl), a mono- or di-C₆₋₁₄ aryl-carbamoyl group (e.g., phenylcarbamoyl), a mono- or di-C₇₋₁₆ aralkyl-carbamoyl group, a mono- or di-C₁₋₆ alkyl-carbonyl-carbamoyl group (e.g., acetylcarbamoyl, propionylcarbamoyl), a mono- or di-C₆₋₁₄ aryl-carbonyl-carbamoyl group (e.g., benzoylcarbamoyl) and a 5- to 14-membered aromatic heterocyclylcarbamoyl group (e.g., pyridylcarbamoyl).

In the present specification, examples of the “optionally substituted thiocarbamoyl group” include a thiocarbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from Substituent group A”.

Preferable examples of the optionally substituted thiocarbamoyl group include a thiocarbamoyl group, a mono- or di-C₁₋₆ alkyl-thiocarbamoyl group (e.g., methylthiocarbamoyl, ethylthiocarbamoyl, dimethylthiocarbamoyl, diethylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a mono- or di-C₁₋₆ alkyl-carbonyl-thiocarbamoyl group (e.g., acetylthiocarbamoyl, propionylthiocarbamoyl), a mono- or di-C₆₋₁₄ aryl-carbonyl-thiocarbamoyl group (e.g., benzoylthiocarbamoyl) and a 5- to 14-membered aromatic heterocyclylthiocarbamoyl group (e.g., pyridylthiocarbamoyl).

In the present specification, examples of the “optionally substituted sulfamoyl group” include a sulfamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from Substituent group A”.

Preferable examples of the optionally substituted sulfamoyl group include a sulfamoyl group, a mono- or di-C₁₋₆ alkyl-sulfamoyl group (e.g., methylsulfamoyl, ethylsulfamoyl, dimethylsulfamoyl, diethylsulfamoyl, N-ethyl-N-methylsulfamoyl), a mono- or di-C₂₋₆ alkenyl-sulfamoyl group (e.g., diallylsulfamoyl), a mono- or di-C₃₋₁₀ cycloalkyl-sulfamoyl group (e.g., cyclopropylsulfamoyl, cyclohexylsulfamoyl), a mono- or di-C₆₋₁₄ aryl-sulfamoyl group (e.g., phenylsulfamoyl), a mono- or di-C₇₋₁₆ aralkyl-sulfamoyl group (e.g., benzylsulfamoyl, phenethylsulfamoyl), a mono- or di-C₁₋₆ alkyl-carbonyl-sulfamoyl group (e.g., acetylsulfamoyl, propionylsulfamoyl), a mono- or di-C₆₋₁₄ aryl-carbonyl-sulfamoyl group (e.g., benzoylsulfamoyl) and a 5- to 14-membered aromatic heterocyclylsulfamoyl group (e.g., pyridylsulfamoyl).

In the present specification, examples of the “optionally substituted hydroxy group” include a hydroxy group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from Substituent group A”. Preferable examples of the optionally substituted hydroxy group include a hydroxy group, a C₁₋₆ alkoxy group, a C₂₋₆ alkenyloxy group (e.g., allyloxy, 2-butenyloxy, 2-pentenyloxy, 3-hexenyloxy), a C₃₋₁₀ cycloalkyloxy group (e.g., cyclohexyloxy), a C₆₋₁₄ aryloxy group (e.g., phenoxy, naphthyloxy), a C₇₋₁₆ aralkyloxy group (e.g., benzyloxy, phenethyloxy), a C₁₋₆ alkyl-carbonyloxy group (e.g., acetyloxy, propionyloxy, butyryloxy, isobutyryloxy, pivaloyloxy), a C₆₋₁₄ aryl-carbonyloxy group (e.g., benzoyloxy), a C₇₋₁₆ aralkyl-carbonyloxy group (e.g., benzylcarbonyloxy), a 5- to 14-membered aromatic heterocyclylcarbonyloxy group (e.g., nicotinoyloxy), a 3- to 14-membered non-aromatic heterocyclylcarbonyloxy group (e.g., piperidinylcarbonyloxy), a C₁₋₆ alkoxy-carbonyloxy group (e.g., tert-butoxycarbonyloxy), a 5- to 14-membered aromatic heterocyclyloxy group (e.g., pyridyloxy), a carbamoyloxy group, a C₁₋₆ alkyl-carbamoyloxy group (e.g., methylcarbamoyloxy), a C₇-16 aralkyl-carbamoyloxy group (e.g., benzylcarbamoyloxy), a C₁₋₆ alkylsulfonyloxy group (e.g., methylsulfonyloxy, ethylsulfonyloxy) and a C₆₋₁₄ arylsulfonyloxy group (e.g., phenylsulfonyloxy).

In the present specification, examples of the “optionally substituted sulfanyl group” include a sulfanyl group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group and a 5- to 14-membered aromatic heterocyclic group, each of which optionally has 1 to 3 substituents selected from Substituent group A” and a halogenated sulfanyl group.

Preferable examples of the optionally substituted sulfanyl group include a sulfanyl (—SH) group, a C₁₋₆ alkylthio group, a C₂₋₆ alkenylthio group (e.g., allylthio, 2-butenylthio, 2-pentenylthio, 3-hexenylthio), a C₃₋₁₀ cycloalkylthio group (e.g., cyclohexylthio), a C₆₋₁₄ arylthio group (e.g., phenylthio, naphthylthio), a C₇₋₁₆ aralkylthio group (e.g., benzylthio, phenethylthio), a C₁₋₆ alkyl-carbonylthio group (e.g., acetylthio, propionylthio, butyrylthio, isobutyrylthio, pivaloylthio), a C₆₋₁₄ aryl-carbonylthio group (e.g., benzoylthio), a 5- to 14-membered aromatic heterocyclylthio group (e.g., pyridylthio) and a halogenated thio group (e.g., pentafluorothio).

In the present specification, examples of the “optionally substituted silyl group” include a silyl group optionally having “1 to 3 substituents selected from a C₁₋₆ alkyl group, a C₂₋₆ alkenyl group, a C₃₋₁₀ cycloalkyl group, a C₆₋₁₄ aryl group and a C₇₋₁₆ aralkyl group, each of which optionally has 1 to 3 substituents selected from Substituent group A”.

Preferable examples of the optionally substituted silyl group include a tri-C₁₋₆ alkylsilyl group (e.g., trimethylsilyl, tert-butyl(dimethyl) silyl).

In the present specification, examples of the “hydrocarbon ring” include a C₆₋₁₄ aromatic hydrocarbon ring, C₃₋₁₀ cycloalkane and C₃₋₁₀ cycloalkene.

In the present specification, examples of the “C₆₋₁₄ aromatic hydrocarbon ring” include benzene and naphthalene.

In the present specification, examples of the “C₃₋₁₀ cycloalkane” include cyclopropane, cyclobutane, cyclopentane, cyclohexane, cycloheptane and cyclooctane.

In the present specification, examples of the “C₃₋₁₀ cycloalkene” include cyclopropene, cyclobutene, cyclopentene, cyclohexene, cycloheptene and cyclooctene.

In the present specification, examples of the “heterocycle” include an aromatic heterocycle and a non-aromatic heterocycle, each containing, as a ring-constituting atom besides carbon atom, 1 to 4 hetero atoms selected from a nitrogen atom, a sulfur atom and an oxygen atom.

In the present specification, examples of the “aromatic heterocycle” include a 5- to 14-membered (preferably 5- to 10-membered) aromatic heterocycle containing, as a ring-constituting atom besides carbon atom, 1 to 4 hetero atoms selected from a nitrogen atom, a sulfur atom and an oxygen atom. Preferable examples of the “aromatic heterocycle” include 5- or 6-membered monocyclic aromatic heterocycles such as thiophene, furan, pyrrole, imidazole, pyrazole, thiazole, isothiazole, oxazole, isoxazole, pyridine, pyrazine, pyrimidine, pyridazine, 1,2,4-oxadiazole, 1,3,4-oxadiazole, 1,2,4-thiadiazole, 1,3,4-thiadiazole, triazole, tetrazole, triazine and the like; and 8- to 14-membered fused polycyclic (preferably bi- or tri-cyclic) aromatic heterocycles such as benzothiophene, benzofuran, benzimidazole, benzoxazole, benzisoxazole, benzothiazole, benzisothiazole, benzotriazole, imidazopyridine, thienopyridine, furopyridine, pyrrolopyridine, pyrazolopyridine, oxazolopyridine, thiazolopyridine, imidazopyrazine, imidazopyrimidine, thienopyrimidine, furopyrimidine, pyrrolopyrimidine, pyrazolopyrimidine, oxazolopyrimidine, thiazolopyrimidine, pyrazolopyrimidine, pyrazolotriazine, naphtho[2,3-b]thiophene, phenoxathiin, indole, isoindole, 1H-indazole, purine, isoquinoline, quinoline, phthalazine, naphthyridine, quinoxaline, quinazoline, cinnoline, carbazole, β-carboline, phenanthridine, acridine, phenazine, phenothiazine, phenoxazine and the like.

In the present specification, examples of the “non-aromatic heterocycle” include a 3- to 14-membered (preferably 4-to 10-membered) non-aromatic heterocycle containing, as a ring-constituting atom besides carbon atom, 1 to 4 hetero atoms selected from a nitrogen atom, a sulfur atom and an oxygen atom. Preferable examples of the “non-aromatic heterocycle” include 3-to 8-membered monocyclic non-aromatic heterocycles such as aziridine, oxirane, thiirane, azetidine, oxetane, thietane, tetrahydrothiophene, tetrahydrofuran, pyrroline, pyrrolidine, imidazoline, imidazolidine, oxazoline, oxazolidine, pyrazoline, pyrazolidine, thiazoline, thiazolidine, tetrahydroisothiazole, tetrahydrooxazole, tetrahydroisoxazole, piperidine, piperazine, tetrahydropyridine, dihydropyridine, dihydrothiopyran, tetrahydropyrimidine, tetrahydropyridazine, dihydropyran, tetrahydropyran, tetrahydrothiopyran, morpholine, thiomorpholine, azepane, diazepane, azepine, azocane, diazocane, oxepane and the like; and 9- to 14-membered fused polycyclic (preferably bi- or tri-cyclic) non-aromatic heterocycles such as dihydrobenzofuran, dihydrobenzimidazole, dihydrobenzoxazole, dihydrobenzothiazole, dihydrobenzisothiazole, dihydronaphtho[2,3-b] thiophene, tetrahydroisoquinoline, tetrahydroquinoline, 4H-quinolizine, indoline, isoindoline, tetrahydrothieno[2,3-c]pyridine, tetrahydrobenzazepine, tetrahydroquinoxaline, tetrahydrophenanthridine, hexahydrophenothiazine, hexahydrophenoxazine, tetrahydrophthalazine, tetrahydronaphthyridine, tetrahydroquinazoline, tetrahydrocinnoline, tetrahydrocarbazole, tetrahydro-β-carboline, tetrahydroacridine, tetrahydrophenazine, tetrahydrothioxanthene, octahydroisoquinoline and the like.

In the present specification, examples of the “nitrogen-containing heterocycle” include a heterocycle containing at least one nitrogen atom as a ring-constituting atom, from among the “heterocycle”.

In the present specification, examples of the “C₃₋₇ cycloalkyl group” include cyclopropyl, cyclobutyl, cyclopentyl, cyclohexyl, and cycloheptyl.

In the present specification, examples of the “mono- or di-C₁₋₆ alkylamino group” include methylamino, dimethylamino, ethylamino, diethylamino, propylamino, isopropylamino, butylamino, isobutylamino, sec-butylamino, tert-butylamino, pentylamino, isopentylamino, neo-pentylamino, 1-ethylpropylamino, hexylamino, isohexylamino, 1,1-dimethylbutylamino, 2,2-dimethylbutylamino, 3,3-dimethylbutylamino, 2-ethylbutylamino and the like.

In the present specification, examples of the “3- to 8-membered non-aromatic heterocyclic group” in the definition of “optionally substituted 3- to 8-membered non-aromatic heterocyclic group” include a 3- to 8-membered (preferably 5- to 6-membered) non-aromatic heterocyclic group containing, as a ring-constituting atom besides carbon atom, 1 to 4 heteroatoms selected from a nitrogen atom, a sulfur atom and an oxygen atom.

Preferable examples of the “3- to 8-membered non-aromatic heterocyclic group” include 3- to 8-membered monocyclic non-aromatic heterocyclic groups such as aziridinyl, oxiranyl, thiiranyl, azetidinyl, oxetanyl, thietanyl, tetrahydrothienyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydroisothiazolyl, tetrahydrooxazolyl, tetrahydroisooxazolyl, piperidinyl, piperazinyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, thiomorpholinyl, azepanyl, diazepanyl, azepinyl, oxepanyl, azocanyl, diazocanyl and the like.

In the present specification, examples of the “5- to 6-membered aromatic heterocyclic group” in the definition of “optionally substituted 5- to 6-membered aromatic heterocyclic group” include a 5- to 6-membered aromatic heterocyclic group containing, as a ring-constituting atom besides carbon atom, 1 to 4 heteroatoms selected from a nitrogen atom, a sulfur atom and an oxygen atom.

Preferable examples of the “5- to 6-membered aromatic heterocyclic group” include monocyclic aromatic heterocyclic groups such as thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl, triazinyl and the like.

The definition of each symbol in formula (I) is explained in detail in the following.

In formula (I), R¹ is a C₁₋₆ alkyl group, a C₃₋₇ cycloalkyl group, or a mono- or di-C₁₋₆ alkylamino group, wherein each of the C₁₋₆ alkyl group, C₃₋₇ cycloalkyl group, and mono- or di-C₁₋₆ alkylamino group is optionally substituted.

Examples of the substituent of the above-mentioned “optionally substituted C₁₋₆ alkyl group”, “optionally substituted C₃₋₇ cycloalkyl group”, and “optionally substituted mono- or di-C₁₋₆ alkylamino group” include substituents selected from Substituent group A. The number of the substituents is preferably 1 to 3. When the number of the substituents is 2 or more, the respective substituents may be the same or different.

In some embodiments of formula (I), R¹ is
(1) an optionally substituted C₁₋₆ alkyl group (e.g., methyl, ethyl, a halo-C₁₋₆ alkyl group (e.g., fluoromethyl, difluoromethyl, fluoroethyl), or a C₁₋₆ alkoxy-C₁₋₆ alkyl group (e.g., methoxymethyl)),
(2) an optionally substituted C₃₋₇ cycloalkyl group (e.g., cyclopropyl or a halo-C₃₋₇ cycloalkyl group (e.g., fluorocyclopropyl)), or
(3) an optionally substituted mono- or di-C₁₋₆ alkylamino group (e.g., dimethylamino).

In some embodiments of formula (I), R¹ is preferably
(1) a C₁₋₆ alkyl group (e.g., methyl, ethyl),
(2) a C₁₋₆ alkoxy-C₁₋₆ alkyl group (e.g., methoxymethyl),
(3) a halo-C₁₋₆ alkyl group (e.g., fluoromethyl, difluoromethyl, fluoroethyl),
(4) a cyclopropyl group,
(5) a halo-cyclopropyl group (e.g., fluorocyclopropyl), or
(6) a mono- or di-C₁₋₆ alkylamino group (e.g., dimethylamino).

In some embodiments of formula (I), R¹ is more preferably
(1) a C₁₋₆ alkyl group (e.g., methyl, ethyl), or
(2) a halo-C₁₋₆ alkyl group (e.g., fluoromethyl).

In some embodiments of formula (I), R¹ is further more preferably a C₁₋₆ alkyl group (e.g., methyl).

In formula (I), each R² is the same or different and each is an optionally substituted C₁₋₆ alkyl group.

Examples of the substituent of the above-mentioned “optionally substituted C₁₋₆ alkyl group” include substituents selected from Substituent group A. The number of the substituents is preferably 1 to 3. When the number of the substituents is 2 or more, the respective substituents may be the same or different.

In some embodiments of formula (I), each R² is the same or different, and is an optionally substituted C₁₋₆ alkyl group (e.g., methyl, ethyl, a halo-C₁₋₆ alkyl group (e.g., fluoromethyl, difluoromethyl), a C₁₋₆ alkoxy-C₁₋₆ alkyl group (e.g., methoxymethyl), or a hydroxyl-C₁₋₆ alkyl group (e.g., hydroxymethyl)).

In some embodiments of formula (I), each R² is the same or different, and is preferably
(1) a C₁₋₆ alkyl group (e.g., methyl, ethyl),
(2) a halo-C₁₋₆ alkyl group (e.g., fluoromethyl, difluoromethyl),
(3) a C₁₋₆ alkoxy-C₁₋₆ alkyl group (e.g., methoxymethyl), or
(4) a hydroxyl-C₁₋₆ alkyl group (e.g., hydroxymethyl).

In some embodiments of formula (I), each R² is the same or different, and is more preferably
(1) a C₁₋₆ alkyl group (e.g., methyl), or
(2) a halo-C₁₋₆ alkyl group (e.g., fluoromethyl).

In some embodiments of formula (I), each R² is the same or different, and is further more preferably a halo-C₁₋₆ alkyl group (e.g., fluoromethyl).

The symbol “o” means the number of substituent R² and is an integer of 0 to 3 (i.e., 0, 1, 2, or 3).

In some embodiments of formula (I), the symbol “o” is preferably 0 or 1.

In some embodiments of formula (I), the symbol “o” is more preferably an integer of 1.

In some embodiments of formula (I), (R²), is preferably R^{2a} when “o” is an integer of 0 or 1.

In formula (I), or formula (Ic), (Id), or (Ie) described herein, R^{2a} is a hydrogen atom, or an optionally substituted C₁₋₆ alkyl group.

Examples of the substituent of the above-mentioned “optionally substituted C₁₋₆ alkyl group” include substituents selected from Substituent group A. The number of the substituents is preferably 1 to 3. When the number of the substituents is 2 or more, the respective substituents may be the same or different.

In some embodiments of formula (I), (Ic) or (Id), R^{2a} is
(1) a hydrogen atom, or
(2) an optionally substituted C₁₋₆ alkyl group (e.g., methyl, ethyl, a halo-C₁₋₆ alkyl group (e.g., fluoromethyl, difluoromethyl), a C₁₋₆ alkoxy-C₁₋₆ alkyl group (e.g., methoxymethyl), or a hydroxyl-C₁₋₆ alkyl group (e.g., hydroxymethyl)).

In some embodiments of formula (I), (Ic) or (Id), R^{2a} is preferably
(1) a hydrogen atom,
(2) a C₁₋₆ alkyl group (e.g., methyl, ethyl),
(3) a halo-C₁₋₆ alkyl group (e.g., fluoromethyl, difluoromethyl),
(4) a C₁₋₆ alkoxy-C₁₋₆ alkyl group (e.g., methoxymethyl), or
(5) a hydroxyl-C₁₋₆ alkyl group (e.g., hydroxymethyl).

In some embodiments of formula (I), (Ic), (Id), or (Ie), R^{2a} is more preferably
(1) a hydrogen atom,
(2) a C₁₋₆ alkyl group (e.g., methyl, ethyl), or
(3) a halo-C₁₋₆ alkyl group (e.g., fluoromethyl, difluoromethyl).

In some embodiments of formula (I), (Ic), (Id), or (Ie), R^{2a} is further more preferably
(1) a C₁₋₆ alkyl group (e.g., methyl), or
(2) a halo-C₁₋₆ alkyl group (e.g., fluoromethyl).

In some embodiments of formula (I), (Ic), (Id), or (Ie), R^{2a} is still more preferably a halo-C₁₋₆ alkyl group (e.g., fluoromethyl).

In formula (I), each R³ is the same or different, and is an optionally substituted C₁₋₆ alkyl group, or a halogen atom.

Examples of the substituent of the above-mentioned “optionally substituted C₁₋₆ alkyl group” include substituents selected from Substituent group A. The number of the substituents is preferably 1 to 3. When the number of the substituents is 2 or more, the respective substituents may be the same or different.

In some embodiments of formula (I), each R³ is the same or different, and is
(1) an optionally substituted C₁₋₆ alkyl group (e.g., methyl, ethyl, or a C₁₋₆ alkoxy-C₁₋₆ alkyl group (e.g., methoxymethyl)), or
(2) a halogen atom (e.g., fluorine).

In some embodiments of formula (I), each R³ is the same or different, and is preferably
(1) a C₁₋₆ alkyl group (e.g., methyl, ethyl),
(2) a C₁₋₆ alkoxy-C₁₋₆ alkyl group (e.g., methoxymethyl), or
(3) a halogen atom (e.g., fluorine).

The symbol “n” means the number of substituent R³ and is an integer of 0 to 3 (i.e., 0, 1, 2, or 3).

In some embodiments of formula (I), the symbol “n” is preferably an integer of 2.

In some embodiments of formula (I), (R³), is preferably R^{3a} and R^{3b} when “n” is an integer of 2.

In formula (I), or formula (Id) described herein, R^{3a} and R^{3b} are the same or different and each is a hydrogen atom, an optionally substituted C₁₋₆ alkyl group, or a halogen atom.

Examples of the substituent of the above-mentioned “optionally substituted C₁₋₆ alkyl group” include substituents selected from Substituent group A. The number of the substituents is preferably 1 to 3. When the number of the substituents is 2 or more, the respective substituents may be the same or different.

In some embodiments of formula (I) or (Id), R^{3a} and R^{3b} are the same or different and each is
(1) a hydrogen atom,
(2) an optionally substituted C₁₋₆ alkyl group (e.g., methyl, ethyl, a C₁₋₆ alkoxy-C₁₋₆ alkyl group (e.g. methoxymethyl)), or
(3) a halogen atom (e.g., fluorine).

In some embodiments of formula (I) or (Id), R^{3a} and R^{3b} are the same or different and each is preferably
(1) a hydrogen atom,
(2) a C₁₋₆ alkyl group (e.g., methyl, ethyl)
(3) a C₁₋₆ alkoxy-C₁₋₆ alkyl group (e.g. methoxymethyl), or
(4) a halogen atom (e.g., fluorine).

In some embodiments of formula (I), (Id), or (Ie), R^{3a} and
R^{3b} are the same or different and each is more preferably
(1) a C₁₋₆ alkyl group (e.g., methyl), or
(2) a halogen atom (e.g., fluorine).

In some embodiments of formula (I), (Id), or (Ie), R^{3a} and R^{3b} are more preferably each a halogen atom (e.g., each fluorine).

In formula (I), R^{4a} is a hydrogen atom, an optionally substituted C₁₋₆ alkyl group, an optionally substituted C₃₋₇ cycloalkyl group, a halogen atom, or an optionally substituted C₁₋₆ alkoxy group.

Examples of the substituent of the above-mentioned “optionally substituted C₁₋₆ alkyl group”, “optionally substituted C₃₋₇ cycloalkyl group”, and “optionally substituted C₁₋₆ alkoxy” include substituents selected from Substituent group A. The number of the substituents is preferably 1 to 3. When the number of the substituents is 2 or more, the respective substituents may be the same or different.

In some embodiments of formula (I), (Ia), (Ib), (Ic), or (Id), R^{4a} is
(1) a hydrogen atom,
(2) an optionally substituted C₁₋₆ alkyl group (e.g., methyl, a halo-C₁₋₆ alkyl group (e.g., fluoromethyl, difluoromethyl, trifluoromethyl)),
(3) an optionally substituted C₃₋₇ cycloalkyl group (e.g., cyclopropyl),
(4) a halogen atom (e.g., fluorine, chlorine, bromine, iodine), or
(5) an optionally substituted C₁₋₆ alkoxy group (e.g., methoxy).

In some embodiments of formula (I), (Ia), (Ib), (Ic), or (Id), R^{4a} is preferably
(1) a hydrogen atom,
(2) a C₁₋₆ alkyl group (e.g., methyl),
(3) a cyclopropyl group,
(4) a C₁₋₆ alkoxy group (e.g., methoxy),
(5) a halo-C₁₋₆ alkyl group (e.g., fluoromethyl, difluoromethyl, trifluoromethyl), or
(6) a halogen atom (e.g., fluorine, chlorine, bromine).

In some embodiments of formula (I), (Ia), (Ib), (Ic), (Id), or (Ie), R^{4a} is more preferably
(1) a C₁₋₆ alkyl group (e.g., methyl), or
(2) a halogen atom (e.g., fluorine or chlorine).

In some embodiments of formula (I), (Ia), (Ib), (Ic), (Id), or (Ie), R^{4a} is further more preferably a halogen atom (e.g., fluorine).

In formula (I), R^{A} is an optionally substituted 3- to 8-membered non-aromatic heterocyclic group, an optionally substituted 5- to 6-membered aromatic heterocyclic group, an optionally substituted C₆₋₁₄ aryl group, or an optionally substituted C₃₋₁₀ cycloalkyl group.

Examples of the substituent of the above-mentioned “optionally substituted 3- to 8-membered non-aromatic heterocyclic group”, “optionally substituted 5- to 6-membered aromatic heterocyclic group”, “optionally substituted C₆₋₁₄ aryl group”, and “optionally substituted C₃₋₁₀ cycloalkyl group” include substituents selected from Substituent group A. The number of the substituents is preferably 1 to 3. When the number of the substituents is 2 or more, the respective substituents may be the same or different.

In some embodiments of formula (I), R^{A} preferably has the following structure:

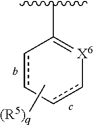

wherein
X⁶ is N or CR^{5′};
each R⁵ is the same or different, and is a halogen atom (e.g., fluorine, chlorine);
R^{5′} is a hydrogen atom, or a halogen atom (e.g., fluorine);
q is an integer of 0 to 7 (e.g., 0, 1, 2, 3, 4, 5, 6, or 7, preferably 1, 2, or 3); and [symbol] and [symbol] are the same and are both single bonds or both double bonds.

In some embodiments of formula (I), R^{A} preferably has the following structure:

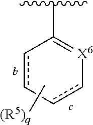

wherein
X⁶ is N or CR^{5′};
each R⁵ is the same or different, and is a halogen atom (e.g., fluorine, chlorine), or a C₁₋₆ alkoxy group (e.g., methoxy);
R^{5′} is a hydrogen atom, or a halogen atom (e.g., fluorine);
q is an integer of 0 to 7 (e.g., 0, 1, 2, 3, 4, 5, 6, or 7, preferably 1, 2, or 3); and
[symbol] and [symbol] are the same and are both single bonds or both double bonds.

In some embodiments of formula (I), R^{A} is preferably a 6-membered cyclic group having the structure in which
(1) X⁶ is CR^{5′}, and [symbol] and [symbol] are both single bonds (i.e., R^{A} is an optionally substituted 1-cyclohexyl group),
(2) X⁶ is CR^{5′}, and [symbol] and [symbol] are both double bonds (i.e., R^{A} is an optionally substituted phenyl group), or
(3) X⁶ is N, and [symbol] and [symbol] are both double bonds (i.e., R^{A} is an optionally substituted 2-pyridyl group).

In some embodiments of formula (I), the structure:

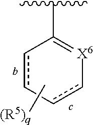

is preferably selected from the group consisting of:

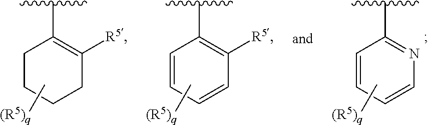

wherein
each R⁵ is the same or different, and is a halogen atom (e.g., fluorine, chlorine), or a C₁₋₆ alkoxy group (e.g., methoxy);
R^{5′} is a hydrogen atom, or a halogen atom (e.g., fluorine); and
q is an integer of 0 to 4.

In some embodiments of formula (I), R^{A} is more preferably
(1) a phenyl group optionally substituted by 1 to 3 substituents selected from a halogen atom (e.g., fluorine, chlorine) and a C₁₋₆ alkoxy group (e.g., methoxy) that are the same or different, or
(2) a 2-pyridyl group optionally substituted by 1 to 3 halogen atoms (e.g., fluorine) that are the same or different.

In some embodiments of formula (I), R^{A} is further more preferably
(1) a phenyl group optionally substituted by 1 to 3 halogen atoms that are the same or different (e.g., fluorine, chlorine, preferably fluorine), or
(2) a 2-pyridyl group optionally substituted by 1 to 3 halogen atoms that are the same or different (e.g., fluorine).

In some embodiments of formula (I), R^{A} is still more preferably a phenyl group optionally substituted by 1 to 3 halogen atoms that are the same or different (e.g., fluorine).

In some embodiments of formula (I), R^{A} is still more preferably a phenyl group substituted by 1 to 3 fluorines, or a 2-pyridyl group substituted by 1 to 3 fluorines.

In some embodiments of formula (I), R^{A} is even more preferably 2,6-difluorophenyl, 2,4,6-trifluorophenyl, or 4,6-difluoropyridin-2-yl. In some embodiments of formula (I), R^{A} is even more preferably 2,6-difluorophenyl or 2,4,6-trifluorophenyl. In some embodiments of formula (I), R^{A} is particularly preferably 2,6-difluorophenyl.

In formula (I), L¹ is a bond, —NR⁶—, or —O—; wherein R⁶ is a hydrogen atom, or an optionally substituted C₁₋₆ alkyl group.

Examples of the substituent of the above-mentioned “optionally substituted C₁₋₆ alkyl group” include substituents selected from Substituent group A. The number of the substituents is preferably 1 to 3. When the number of the substituents is 2 or more, the respective substituents may be the same or different.

In some embodiments of formula (I), (Ia), (Ib), or (Ic), L¹ is preferably
(1) a bond,
(2) —NR⁶—, wherein R⁶ is a hydrogen atom, or a C₁₋₆ alkyl group (e.g., methyl), or
(3) —O—.

In some embodiments of formula (I), (Ia), (Ib), or (Ic), L¹ is more preferably a bond.

In formula (I), L² is a bond or —CH₂—.

In some embodiments of formula (I), (Ia), (Ib), or (Ic), L² is preferably a bond.

In formula (I), X¹ is N or CR^{3′}, wherein R^{3′} is a hydrogen atom, an optionally substituted C₁₋₆ alkyl group, or a halogen atom.

Examples of the substituent of the above-mentioned “optionally substituted C₁₋₆ alkyl group” include substituents selected from Substituent group A. The number of the substituents is preferably 1 to 3. When the number of the substituents is 2 or more, the respective substituents may be the same or different.

In some embodiments of formula (I), (Ia), (Ib), or (Ic) R^{3′} is
(1) a hydrogen atom,
(2) an optionally substituted C₁₋₆ alkyl group (e.g., methyl, ethyl, or a C₁₋₆ alkoxy-C₁₋₆ alkyl group (e.g., methoxymethyl)), or
(3) a halogen atom (e.g., fluorine).

In some embodiments of formula (I), (Ia), (Ib), or (Ic), R^{3′} is preferably
(1) a hydrogen atom,
(2) a C₁₋₆ alkyl group (e.g., methyl, ethyl),
(3) a C₁₋₆ alkoxy-C₁₋₆ alkyl group (e.g., methoxymethyl), or
(4) a halogen atom (e.g., fluorine).

In some embodiments of formula (I), (Ia), (Ib), or (Ic), R^{3′} is more preferably a hydrogen atom.

The symbol “m” means the number of a methylene group (—CH₂—) and is an integer of 0 to 3 (i.e., 0, 1, 2, or 3) to form a 4- to 7-membered carbocyclic or N-containing ring.

In some embodiments of formula (I), (Ia), (Ib), or (Ic), X¹ is
(1) N (i.e., the 4- to 7-membered N-containing ring is azetidine, pyrrolidine, piperidine, or azepane, each of which is optionally substituted by R³), or
(2) CR^{3′} (i.e., the 4- to 7-membered carbocyclic ring is cyclobutane, cyclopentane, cyclohexane, or cycloheptane, each of which is substituted by R^{3′} and optionally substituted by R³).

In some embodiments of formula (I), (Ia), (Ib), or (Ic), X¹ is preferably
(1) N (i.e., the 4- to 7-membered N-containing ring is azetidine, pyrrolidine, piperidine, or azepane, each of which is optionally substituted by R³), or
(2) CH (i.e., the 4- to 7-membered carbocyclic ring is preferably cyclobutane, cyclopentane, cyclohexane, or cycloheptane, each of which is optionally substituted by R³).

In some embodiments of formula (I), (Ia), (Ib), or (Ic), X¹ is more preferably N (i.e., the 4- to 7-membered N-containing ring is azetidine, pyrrolidine, piperidine, or azepane, each of which is optionally substituted by R³, particularly preferably pyrrolidine optionally substituted by R^{3a} and R^{3b}).

In formula (I), X² is N or CR^{4b}, wherein R^{4b} is a hydrogen atom, an optionally substituted C₁₋₆ alkyl group, an optionally substituted C₃₋₇ cycloalkyl group, a halogen atom, or an optionally substituted C₁₋₆ alkoxy group.

Examples of the substituent of the above-mentioned “optionally substituted C₁₋₆ alkyl group”, “optionally substituted C₃₋₇ cycloalkyl group”, and “optionally substituted C₁₋₆ alkoxy” include substituents selected from Substituent group A. The number of the substituents is preferably 1 to 3. When the number of the substituents is 2 or more, the respective substituents may be the same or different.

In some embodiments of formula (I), (Ia), (Ib), (Ic), or (Id), X² is
(1) N, or
(2) CR^{4b}, wherein R^{4b} is
   (1) a hydrogen atom,
   (2) an optionally substituted C₁₋₆ alkyl group (e.g., methyl, a halo-C₁₋₆ alkyl group (e.g., fluoromethyl, trifluoromethyl)),
   (3) an optionally substituted C₃₋₇ cycloalkyl group (e.g., cyclopropyl),
   (4) a halogen atom (e.g., fluorine, chlorine, bromine, iodine), or
   (5) an optionally substituted C₁₋₆ alkoxy group (e.g., methoxy).

In some embodiments of formula (I), R^{4b} is preferably
(1) a hydrogen atom, or
(2) a halogen atom (e.g., fluorine).

In some embodiments of formula (I), (Ia), (Ib), (Ic), or (Id), X² is preferably
(1) N, or
(2) CR^{4b}, wherein R^{4b} is a hydrogen atom, or a halogen atom (e.g., fluorine).

In some embodiments of formula (I), (Ia), (Ib), (Ic), (Id), or (Ie), X² is more preferably
(1) N, or
(2) CR^{4b}, wherein R^{4b} is a halogen atom (e.g., fluorine).

In some embodiments of formula (I), (Ia), (Ib), (Ic), (Id), or (Ie), X² is further more preferably N or —CF.

In some embodiments of formula (I), (Ia), (Ib), (Ic), (Id), or (Ie), X² is still more preferably N.

In formula (I), X³ is N or C.

In some embodiments of formula (I) or (Ia), X³ is preferably C.

In formula (I), X⁴ is N or CR^{4c}, wherein R^{4c} is a hydrogen atom, an optionally substituted C₁₋₆ alkyl group, an optionally substituted C₃₋₇ cycloalkyl group, a halogen atom, or an optionally substituted C₁₋₆ alkoxy group.

Examples of the substituent of the above-mentioned “optionally substituted C₁₋₆ alkyl group”, “optionally substituted C₃₋₇ cycloalkyl group”, and “optionally substituted C₁₋₆ alkoxy” include substituents selected from Substituent group A. The number of the substituents is preferably 1 to 3. When the number of the substituents is 2 or more, the respective substituents may be the same or different.

In some embodiments of formula (I) or (Ia), X⁴ is
(1) N, or
(2) CR^{4c}, wherein R^{4c} is
   (1) a hydrogen atom,
   (2) an optionally substituted C₁₋₆ alkyl group (e.g., methyl, a halo-C₁₋₆ alkyl group (e.g., fluoromethyl, trifluoromethyl)),
   (3) an optionally substituted C₃₋₇ cycloalkyl group (e.g., cyclopropyl),
   (4) a halogen atom (e.g., fluorine, chlorine, bromine, iodine), or
   (5) an optionally substituted C₁₋₆ alkoxy group (e.g., methoxy).

In some embodiments of formula (I) or (Ia), R^{4c} is preferably
(1) a hydrogen atom, or
(2) a halogen atom (e.g., fluorine).

In some embodiments of formula (I) or (Ia), X⁴ is preferably
(1) N, or
(2) CR^{4c}, wherein R^{4c} is a hydrogen atom, or a halogen atom (e.g., fluorine).

In some embodiments of formula (I) or (Ia), X⁴ is more preferably
(1) N, or
(2) CR^{4c}, wherein R^{4c} is a hydrogen atom.

In some embodiments of formula (I) or (Ia), X⁴ is preferably CR^{4c}, wherein R^{4c} is as defined above.

In some embodiments of formula (I) or (Ia), X⁴ is more preferably CR^{4c}, wherein R^{4c} is a hydrogen atom, or a halogen atom (e.g., fluorine).

In some embodiments of formula (I) or (Ia), X⁴ is further more preferably —CH.

In formula (I), X⁵ is N or CR^{4d}, wherein R^{4d} is a hydrogen atom, an optionally substituted C₁₋₆ alkyl group, an optionally substituted C₃₋₇ cycloalkyl group, a halogen atom, or an optionally substituted C₁₋₆ alkoxy group.

Examples of the substituent of the above-mentioned “optionally substituted C₁₋₆ alkyl group”, “optionally substituted C₃₋₇ cycloalkyl group”, and “optionally substituted C₁₋₆ alkoxy” include substituents selected from Substituent group A. The number of the substituents is preferably 1 to 3. When the number of the substituents is 2 or more, the respective substituents may be the same or different.

In some embodiments of formula (I) or (Ia), X⁵ is
(1) N, or
(2) CR^{4d}, wherein R^{4d} is
   (1) a hydrogen atom,
   (2) an optionally substituted C₁₋₆ alkyl group (e.g., methyl, a halo-C₁₋₆ alkyl group (e.g., fluoromethyl, trifluoromethyl)),
   (3) an optionally substituted C₃₋₇ cycloalkyl group (e.g., cyclopropyl),
   (4) a halogen atom (e.g., fluorine, chlorine, bromine, iodine), or
   (5) an optionally substituted C₁₋₆ alkoxy group (e.g., methoxy).

In some embodiments of formula (I), R^{4d} is preferably
(1) a hydrogen atom, or
(2) a halogen atom.

In some embodiments of formula (I), (Ib), (Ic), or (Id), R^{4d} is more preferably a hydrogen atom.

In some embodiments of formula (I) or (Ia), X⁵ is preferably
(1) N, or
(2) CR^{4d}, wherein R^{4d} is a hydrogen atom.

In some embodiments of formula (I) or (Ia), X⁵ is preferably CR^{4d}, wherein R^{4d} is as defined above.

In some embodiments of formula (I) or (Ia), X⁵ is more preferably —CH.

In formula (I), the moiety:

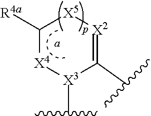

is such that no more than two of X², X³, X⁴, and X⁵ are N.

In some embodiments of formula (I), the moiety is preferably such that no more than one of X², X³, X⁴, and X⁵ are N.

In some embodiments of formula (I), the moiety is more preferably such that one of X², X³, X⁴, and X⁵ are N.

In formula (I), the symbol “p” means the number of X⁵ and is an integer of 0 or 1, and [symbol] comprises one single bond and one double bond when p is 0 or one single bond and two double bonds when p is 1 to provide an aromatic ring structure. When p is 0, then X⁵ is absent to provide a 5-membered aromatic ring. When p is 1, then X⁵ is present to provide a 6-membered aromatic ring.

In some embodiments of formula (I) or (Ia), a combination of X², X³, X⁴, X⁵, and p is
(1) X² is CR^{4b}, X³ is N, X⁴ is N, and p is 0;
(2) X² is N, X³ is N, X⁴ is CR^{4c}, and p is 0;
(3) X² is CR^{4b}, X³ is C, X⁴ is CR^{4c}, X⁵ is CR^{4d}, and p is 1;
(4) X² is N, X³ is C, X⁴ is CR^{4c}, X⁵ is CR^{4d}, and p is 1;
(5) X² is CR^{4b}, X³ is C, X⁴ and X⁵ are N, and p is 1;
(6) X² is CR^{4b}, X³ is C, X⁴ is CR^{4c}, X⁵ is N, and p is 1;
(7) X and X are N, X is C, X is CR, and p is 1; or
(8) X is CR, X is C, X is N, X is CR, and p is 1.

In some embodiments of formula (I) or (Ia), the moiety

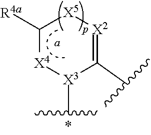

is preferably selected from the group consisting of:

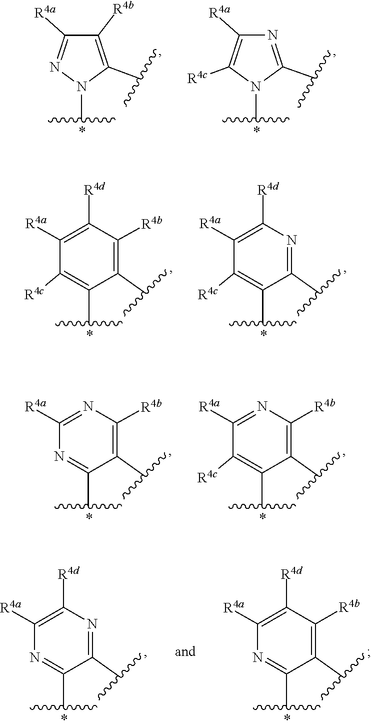

wherein
* indicates the bonding site to R^{A}; and
R^{4a}, R^{4b}, R^{4c}, and R^{4d} are as defined above.

In this embodiment, a combination of R^{4a}, R^{4b}, R^{4c}, and R^{4d} is preferably as follows:
R^{4a} is
(1) a hydrogen atom,
(2) a C₁₋₆ alkyl group (e.g., methyl),
(3) a cyclopropyl group,
(4) a C₁₋₆ alkoxy group (e.g., methoxy),
(5) a halo-C₁₋₆ alkyl group (e.g., fluoromethyl, difluoromethyl, trifluoromethyl), or
(6) a halogen atom (e.g., fluorine, chlorine, bromine);
R^{4b} is
(1) a hydrogen atom, or
(2) a halogen atom (e.g., fluorine);
R^{4c} is
(1) a hydrogen atom, or
(2) a halogen atom (e.g., fluorine); and
R^{4d} is a hydrogen atom.

In some embodiments of formula (I), a combination of R¹, R², R³, R^{4a}, and R^{A} is preferably as follows:
R¹ is
(1) a C₁₋₆ alkyl group (e.g., methyl, ethyl),
(2) a C₁₋₆ alkoxy-C₁₋₆ alkyl group (e.g., methoxymethyl),
(3) a halo-C₁₋₆ alkyl group (e.g., fluoromethyl, difluoromethyl, fluoroethyl),
(4) a cyclopropyl group,
(5) a halo-cyclopropyl group (e.g., fluorocyclopropyl), or
(6) a mono- or di-C₁₋₆ alkylamino group (e.g., dimethylamino);
each R² is the same or different, and is
(1) a C₁₋₆ alkyl group (e.g., methyl, ethyl),
(2) a halo-C₁₋₆ alkyl group (e.g., fluoromethyl, difluoromethyl),
(3) a C₁₋₆ alkoxy-C₁₋₆ alkyl group (e.g., methoxymethyl), or
(4) a hydroxyl-C₁₋₆ alkyl group (e.g., hydroxymethyl);
each R³ is the same or different, and is
(1) a C₁₋₆ alkyl group (e.g., methyl, ethyl),
(2) a C₁₋₆ alkoxy-C₁₋₆ alkyl group (e.g., methoxymethyl), or
(3) a halogen atom (e.g., fluorine);
R^{4a} is
(1) a hydrogen atom,
(2) a C₁₋₆ alkyl group (e.g., methyl),
(3) a cyclopropyl group,
(4) a C₁₋₆ alkoxy group (e.g., methoxy),
(5) a halo-C₁₋₆ alkyl group (e.g., fluoromethyl, difluoromethyl, trifluoromethyl), or
(6) a halogen atom (e.g., fluorine, chlorine, bromine); and
R^{A} has the following structure:

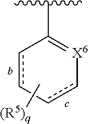

which is selected from the group consisting of:

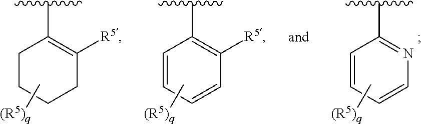

wherein
q is an integer of 0 to 4 (e.g., 0, 1, 2, 3 or 4); and
   the other symbols are as defined above.

In some embodiments, the preferred compound (I) is a compound wherein
R¹ is
(1) an optionally substituted C₁₋₆ alkyl group (e.g. methyl, ethyl, a halo-C₁₋₆ alkyl group (e.g., fluoromethyl, difluoromethyl, fluoroethyl), or a C₁₋₆ alkoxy-_{C1-6} alkyl group (e.g., methoxymethyl)),
(2) an optionally substituted C₃₋₇ cycloalkyl group (e.g., cyclopropyl or halo-C₃₋₇ cycloalkyl group (e.g., fluorocyclopropyl)), or
(3) an optionally substituted mono- or di-C₁₋₆ alkylamino group (e.g., dimethylamino);
each R² is the same or different, and is an optionally substituted C₁₋₆ alkyl group (e.g., methyl, ethyl, a halo-C₁₋₆ alkyl group (e.g., fluoromethyl, difluoromethyl), a C₁₋₆ alkoxy-C₁₋₆ alkyl group (e.g., methoxymethyl), or a hydroxyl-C₁₋₆ alkyl group (e.g., hydroxymethyl));
each R³ is the same or different, and is
(1) an optionally substituted C₁₋₆ alkyl group (e.g., methyl, ethyl, or a C₁₋₆ alkoxy-C₁₋₆ alkyl group (e.g., methoxymethyl)), or
(2) a halogen atom (e.g., fluorine);
R^{A} is an optionally substituted 3- to 8-membered non-aromatic heterocyclic group, an optionally substituted 5- to 6-membered aromatic heterocyclic group, an optionally substituted C₆₋₁₄ aryl group, or an optionally substituted C₃₋₁₀ cycloalkyl group;
L¹ is a bond, —NR⁶—, or —O—; wherein R⁶ is a hydrogen atom, or an optionally substituted C₁₋₆ alkyl group (e.g., methyl);
L² is a bond or —CH₂—;
X¹ is N or CR^{3′};
X² is N or CR^{4b};
X³ is N or C;
X⁴ is N or CR^{4c};
X⁵ is N or CR^{4d};
provided that no more than two of X², X³, X⁴, and X⁵ are N;
R^{3′} is
(1) a hydrogen atom,
(2) a C₁₋₆ alkyl group (e.g., methyl, ethyl),
(3) a C₁₋₆ alkoxy-C₁₋₆ alkyl group (e.g., methoxymethyl), or
(4) a halogen atom (e.g., fluorine);
R^{4a}, R^{4b}, R^{4c}, and R^{4d} are the same or different and each is
(1) a hydrogen atom,
(2) an optionally substituted C₁₋₆ alkyl group (e.g., methyl, a halo-C₁₋₆ alkyl group (e.g., fluoromethyl, difluoromethyl, trifluoromethyl)),
(3) an optionally substituted C₃₋₇ cycloalkyl group (e.g., cyclopropyl),
(4) a halogen atom (e.g., fluorine, chlorine, bromine), or
(5) an optionally substituted C₁₋₆ alkoxy group (e.g., methoxy);
m is an integer of 0 to 3 (i.e., 0, 1, 2, or 3);
n is an integer of 0 to 3 (i.e., 0, 1, 2, or 3);
o is an integer of 0 to 3 (i.e., 0, 1, 2, or 3);
p is an integer of 0 or 1; and
[symbol] comprises one single bond and one double bond when p is 0 or one single bond and two double bonds when p is 1 to provide an aromatic ring structure.

In some embodiments, the more preferred compound (I) is a compound wherein
R¹ is
(1) a C₁₋₆ alkyl group (e.g., methyl, ethyl),
(2) a C₁₋₆ alkoxy-C₁₋₆ alkyl group (e.g., methoxymethyl),
(3) a halo-C₁₋₆ alkyl group (e.g., fluoromethyl, difluoromethyl, fluoroethyl),
(4) a cyclopropyl group,
(5) a halo-cyclopropyl group (e.g., fluorocyclopropyl), or
(6) a mono- or di-C₁₋₆ alkylamino group (e.g., dimethylamino).
each R² is the same or different, and is
(1) a C₁₋₆ alkyl group (e.g., methyl, ethyl),
(2) a halo-C₁₋₆ alkyl group (e.g., fluoromethyl, difluoromethyl),
(3) a C₁₋₆ alkoxy-C₁₋₆ alkyl group (e.g., methoxymethyl), or
(4) a hydroxyl-C₁₋₆ alkyl group (e.g., hydroxymethyl);
each R³ is the same or different, and is
(1) a C₁₋₆ alkyl group (e.g., methyl, ethyl),
(2) a C₁₋₆ alkoxy-C₁₋₆ alkyl group (e.g., methoxymethyl), or
(3) a halogen atom (e.g., fluorine);
R^{A} has the following structure:

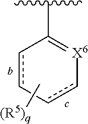

wherein
X⁶ is N or CR^{5′};
each R⁵ is the same or different, and is a halogen atom (e.g., fluorine, chlorine), or a C₁₋₆ alkoxy group (e.g., methoxy);
R^{5′} is a hydrogen atom, or a halogen atom (e.g., fluorine);
q is an integer of 0 to 7 (e.g., 0, 1, 2, 3, 4, 5, 6, or 7, preferably 1 to 3); and
[symbol] and [symbol] are the same and are both single bonds or both double bonds;
L¹ is a bond, —NR⁶—, or —O—; wherein R⁶ is a hydrogen atom, or an optionally substituted C₁₋₆ alkyl group (e.g., methyl);
L² is a bond or —CH₂—;
X¹ is N or CR^{3′};
X² is N or CR^{4b};
X³ is N or C;
X⁴ is N or CR^{4c};
X⁵ is N or CR^{4d};
provided that no more than two of X², X³, X⁴, and X⁵ are N;
R^{3′} is
(1) a hydrogen atom,
(2) a C₁₋₆ alkyl group (e.g., methyl, ethyl),
(3) a C₁₋₆ alkoxy-C₁₋₆ alkyl group (e.g., methoxymethyl), or
(4) a halogen atom (e.g., fluorine);
R^{4a}, R^{4b}, R^{4c}, and R^{4d} are the same or different and each is
(1) a hydrogen atom,
(2) a C₁₋₆ alkyl group (e.g., methyl),
(3) a cyclopropyl group,
(4) a C₁₋₆ alkoxy group (e.g., methoxy),
(5) a halo-C₁₋₆ alkyl group (e.g., fluoromethyl, difluoromethyl, trifluoromethyl), or
(6) a halogen atom (e.g., fluorine, chlorine, bromine);
m is an integer of 0 to 3 (i.e., 0, 1, 2, or 3);
n is an integer of 1 to 3 (i.e., 1, 2, or 3);
o is an integer of 1 to 3 (i.e., 1, 2, or 3); and
p is an integer of 0 or 1; and
[symbol] comprises one single bond and one double bond when p is 0 or one single bond and two double bonds when p is 1 to provide an aromatic ring structure.

In some embodiments, the further more preferred compound (I) is a compound wherein

the moiety:

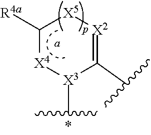

is selected from the group consisting of:

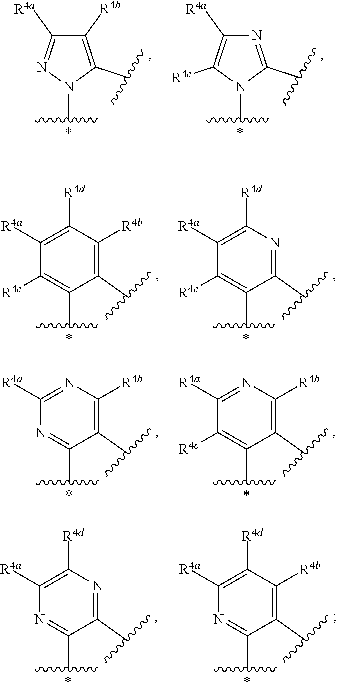

wherein * indicates the bonding site to R^{A};
R¹ is
(1) a C₁₋₆ alkyl group (e.g., methyl, ethyl),
(2) a C₁₋₆ alkoxy-C₁₋₆ alkyl group (e.g., methoxymethyl),
(3) a halo-C₁₋₆ alkyl group (e.g., fluoromethyl, difluoromethyl, fluoroethyl),
(4) a cyclopropyl group,
(5) a halo-cyclopropyl group (e.g., fluorocyclopropyl), or
(6) a mono- or di-C₁₋₆ alkylamino group (e.g., dimethylamino);
each R² is the same or different, and is the same or different, and is
(1) a C₁₋₆ alkyl group (e.g., methyl, ethyl),
(2) a halo-C₁₋₆ alkyl group (e.g., fluoromethyl, difluoromethyl),
(3) a C₁₋₆ alkoxy-C₁₋₆ alkyl group (e.g., methoxymethyl), or
(4) a hydroxy-C₁₋₆ alkyl group (e.g., hydroxymethyl);
each R³ is the same or different, and is the same or different, and is
(1) a C₁₋₆ alkyl group (e.g., methyl, ethyl),
(2) a C₁₋₆ alkoxy-C₁₋₆ alkyl group (e.g., methoxymethyl), or
(3) a halogen atom (e.g., fluorine);
R^{4a}, R^{4b}, R^{4c}, and R^{4d} are the same or different and each is
(1) a hydrogen atom,
(2) a C₁₋₆ alkyl group (e.g., methyl),
(3) a cyclopropyl group,
(4) a C₁₋₆ alkoxy group (e.g., methoxy),
(5) a halo-C₁₋₆ alkyl group (e.g., fluoromethyl, difluoromethyl, trifluoromethyl), or
(6) a halogen atom (e.g., fluorine, chlorine, bromine);
R^{A} has the following structure:

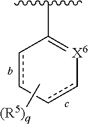

which is selected from the group consisting of:

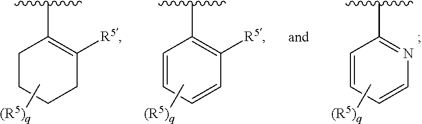

wherein
each R⁵ is the same or different, and is a halogen atom (e.g., fluorine, chlorine), or a C₁₋₆ alkoxy group (e.g., methoxy); R^{5′} is a hydrogen atom, or a halogen atom (e.g., fluorine); and q is an integer of 0 to 4 (e.g., 0, 1, 2, 3, 4);
L¹ is a bond, —NR⁶—, or —O—; wherein R⁶ is a hydrogen atom, or an optionally substituted C₁₋₆ alkyl group (e.g., methyl);
L² is a bond or —CH₂—;
X¹ is N or CR^{3′};
R^{3′} is
(1) a hydrogen atom,
(2) a C₁₋₆ alkyl group (e.g., methyl, ethyl),
(3) a C₁₋₆ alkoxy-C₁₋₆ alkyl group (e.g., methoxymethyl), or
(4) a halogen atom (e.g., fluorine);
m is an integer of 0 to 3 (i.e., 0, 1, 2, or 3);
n is an integer of 0 to 3 (i.e., 0, 1, 2, or 3); and
is an integer of 0 to 3 (i.e., 0, 1, 2, or 3);
   or a salt thereof.

In some embodiments of formula (I), the preferred compound (I) is a compound wherein
the moiety

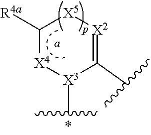

is selected from the group consisting of:

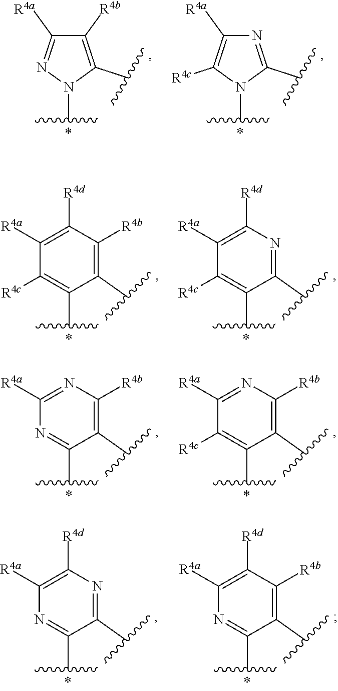

wherein * indicates the bonding site to R^{A};
R¹ is
(1) a C₁₋₆ alkyl group (e.g., methyl, ethyl),
(2) a C₁₋₆ alkoxy-C₁₋₆ alkyl group (e.g., methoxymethyl),
(3) a halo-C₁₋₆ alkyl group (e.g., fluoromethyl, difluoromethyl, fluoroethyl),
(4) a cyclopropyl group,
(5) a halo-cyclopropyl group (e.g., fluorocyclopropyl), or
(6) a mono- or di-C₁₋₆ alkylamino group (e.g., dimethylamino);
each R² is the same or different, and is
(1) a C₁₋₆ alkyl group (e.g., methyl, ethyl),
(2) a halo-C₁₋₆ alkyl group (e.g., fluoromethyl, difluoromethyl),
(3) a C₁₋₆ alkoxy-C₁₋₆ alkyl group (e.g., methoxymethyl), or
(4) a hydroxyl-C₁₋₆ alkyl group (e.g., hydroxymethyl);
each R³ is the same or different, and is
(1) a C₁₋₆ alkyl group (e.g., methyl, ethyl),
(2) a C₁₋₆ alkoxy-C₁₋₆ alkyl group (e.g., methoxymethyl), or
(3) a halogen atom (e.g., fluorine).
L¹ is a bond, —NR⁶—, or —O—; wherein R⁶ is a hydrogen atom, or a C₁₋₆ alkyl group (e.g., methyl);
L² is a bond or —CH₂—;
X¹ is N or CR^{3′}; wherein R^{3′} is a hydrogen atom;
R^{4a} is
(1) a hydrogen atom,
(2) a C₁₋₆ alkyl group (e.g., methyl),
(3) a cyclopropyl group,
(4) a C₁₋₆ alkoxy group (e.g., methoxy),
(5) a halo-C₁₋₆ alkyl group (e.g., fluoromethyl, difluoromethyl, trifluoromethyl), or
(6) a halogen atom (e.g., fluorine, chlorine, bromine);
X² is N or CR^{4b}; wherein R^{4b} is a hydrogen atom, or a halogen atom (e.g., fluorine);
X⁴ is N or CR^{4c}; wherein R^{4c} is a hydrogen atom, or a halogen atom (e.g., fluorine);
X⁵ is N or CR^{4d}; wherein R^{4d} is a hydrogen atom;
m is an integer of 0 to 3;
n is an integer of 0 to 3;
is an integer of 0 to 3; and
R^{A} has the following structure:

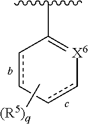

which is selected from the group consisting of:

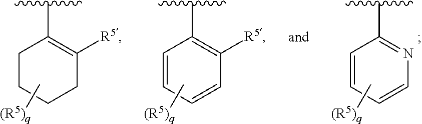

wherein
each R⁵ is the same or different, and is a halogen atom (e.g., fluorine, chlorine), or a C₁₋₆ alkoxy group (e.g., methoxy); R^{5′} is a hydrogen atom, or a halogen atom (e.g., fluorine); and q is an integer of 0 to 4 (e.g., 0, 1, 2, 3 or 4), or a salt thereof.

In some embodiments, the preferred compound (I) is a compound of formula (Ia):

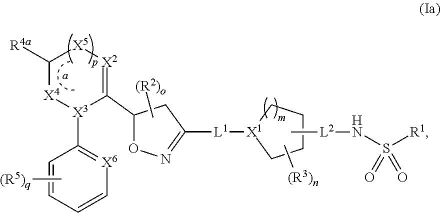

wherein
q is an integer of 0 to 4 (i.e., 0, 1, 2, 3, or 4); and the other symbols are as defined above;
   or a salt thereof.

In some embodiments, the preferred compound (I) is a compound of formula (Ib):

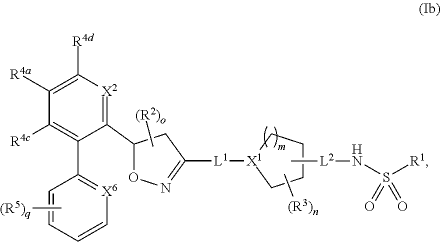

wherein
q is an integer of 0 to 4 (i.e., 0, 1, 2, 3, or 4); and
the other symbols are as defined above;
   or a salt thereof.

In some embodiments, the preferred compound (I) is a compound of formula (Ic):

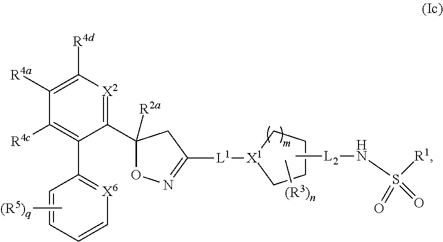

wherein
R^{2a} is a hydrogen atom, or an optionally substituted C₁₋₆ alkyl group;
q is an integer of 0 to 4 (i.e., 0, 1, 2, 3, or 4); and
the other symbols are as defined above;
   or a salt thereof.

In some embodiments, the preferred compound (I) is a compound of formula (Id):

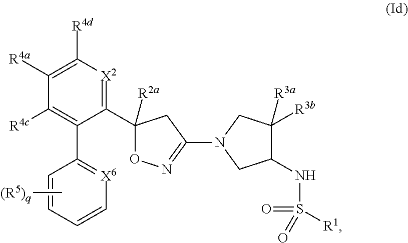

wherein
R^{2a} is a hydrogen atom, or an optionally substituted C₁₋₆ alkyl group;
R^{3a} and R^{3b} are the same or different and each is a hydrogen atom, an optionally substituted C₁₋₆ alkyl group, or a halogen atom;
q is an integer of 0 to 4; and
the other symbols are as defined in claim 2;
   or a salt thereof (hereinafter referred to as “compound (Id)”).

In some embodiments, the preferred compound (Id) is a compound wherein
R¹ is
(1) a C₁₋₆ alkyl group (e.g., methyl, ethyl),
(2) a C₁₋₆ alkoxy-C₁₋₆ alkyl group (e.g., methoxymethyl),
(3) a halo-C₁₋₆ alkyl group (e.g., fluoromethyl, difluoromethyl, fluoroethyl),
(4) a cyclopropyl group,
(5) a halo-cyclopropyl group (e.g., fluorocyclopropyl), or
(6) a mono- or di-C₁₋₆ alkylamino group (e.g., dimethylamino);
R^{2a} is
(1) a hydrogen atom,
(2) a C₁₋₆ alkyl group (e.g., methyl, ethyl),
(3) a halo-C₁₋₆ alkyl group (e.g., fluoromethyl, difluoromethyl),
(4) a C₁₋₆ alkoxy-C₁₋₆ alkyl group (e.g., methoxymethyl), or
(5) a hydroxyl-C₁₋₆ alkyl group (e.g., hydroxymethyl);
R^{3a} and R^{3b} are the same or different and each is
(1) a hydrogen atom,
(2) a C₁₋₆ alkyl group (e.g., methyl, ethyl),
(3) a C₁₋₆ alkoxy-C₁₋₆ alkyl group (e.g. methoxymethyl), or
(4) a halogen atom (e.g., fluorine);
X² is N or CR^{4b};
R^{4a}, R^{4b}, R^{4c}, and R^{4d} are the same or different and each is
(1) a hydrogen atom,
(2) a C₁₋₆ alkyl group (e.g., methyl),
(3) a cyclopropyl group,
(4) a C₁₋₆ alkoxy group (e.g., methoxy),
(5) a halo-C₁₋₆ alkyl group (e.g., fluoromethyl, difluoromethyl, trifluoromethyl), or
(6) a halogen atom (e.g., fluorine, chlorine, bromine);
X⁶ is N or CR^{5′};
each R⁵ is the same or different, and is a halogen atom (e.g., fluorine, chlorine);
R^{5′} is a hydrogen atom, or a halogen atom (e.g., fluorine); and
q is an integer of 0 to 4 (i.e., 0, 1, 2, 3, or 4).

In some embodiments, the preferred compound (I) is a compound of formula (Ie):

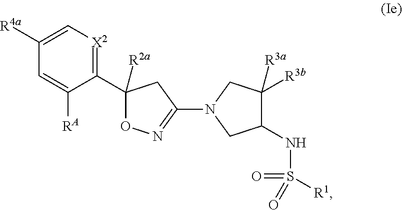

wherein
R¹ is
(1) a C₁₋₆ alkyl group (e.g., methyl, ethyl), or
(2) a halo-C₁₋₆ alkyl group (e.g., fluoromethyl);
R^{2a} is
(1) a hydrogen atom,
(2) a C₁₋₆ alkyl group (e.g., methyl), or
(3) a halo-C₁₋₆ alkyl group (e.g., fluoromethyl);
R^{3a} and R^{3b} are the same or different and each is
(1) a C₁₋₆ alkyl group (e.g., methyl), or
(2) a halogen atom (e.g., fluorine);
R^{4a} is
(1) a C₁₋₆ alkyl group (e.g., methyl), or
(2) a halogen atom (e.g., fluorine, chlorine);
X² is N or CR^{4b}; wherein R^{4b} is a halogen atom (e.g., fluorine); and
R^{A} is a phenyl group, or a pyridyl group, wherein each of the phenyl group and pyridyl group is optionally substituted by one to three halogen atoms (e.g., fluorine) (including 2,6-difluorophenyl, 2,4,6-trifluorophenyl, 4,6-difluoropyridin-2-yl), or salt thereof (hereinafter referred to as “compound (Ie)”).

In some embodiments, the preferred compound (Ie) is a compound wherein
R¹ is
(1) a C₁₋₆ alkyl group (e.g., methyl, ethyl), or
(2) a halo-C₁₋₆ alkyl group (e.g., fluoromethyl);
R^{2a} is
(1) a hydrogen atom,
(2) a C₁₋₆ alkyl group (e.g., methyl), or
(3) a halo-C₁₋₆ alkyl group (e.g., fluoromethyl);
R^{3a} and R^{3b} are each a halogen atom (e.g., fluorine);
R^{4a} is
(1) a C₁₋₆ alkyl group (e.g., methyl), or
(2) a halogen atom (e.g., fluorine, chlorine);
X² is N or CR^{4b}; wherein R^{4b} is a halogen atom (e.g., fluorine); and
R^{A} is a phenyl group, or a pyridyl group, wherein each of the phenyl group and pyridyl group is optionally substituted by one to three halogen atoms (e.g., fluorine) (including 2,6-difluorophenyl, 2,4,6-trifluorophenyl, 4,6-difluoropyridin-2-yl); or a salt thereof.

In some embodiments, the more preferred compound (Ie) is a compound wherein
R¹ is
(1) a C₁₋₆ alkyl group (e.g., methyl), or
(2) a halo-C₁₋₆ alkyl group (e.g., fluoromethyl);
R^{2a} is
(1) a C₁₋₆ alkyl group (e.g., methyl), or
(2) a halo-C₁₋₆ alkyl group (e.g., fluoromethyl);
R^{3a} and R^{3b} are each a halogen atom (e.g., fluorine);
R^{4a} is a halogen atom (e.g., fluorine); X² is N; and
R^{A} is a phenyl group optionally substituted by one to three halogen atoms (e.g., fluorine) (including 2,6-difluorophenyl);
   or a salt thereof.

In some embodiments, the further more preferred compound (Ie) is a compound wherein
R¹ is a C₁₋₆ alkyl group (e.g., methyl);
R^{2a} is a halo-C₁₋₆ alkyl group (e.g., fluoromethyl);
R^{3a} and R^{3b} are each a halogen atom (e.g., each fluorine);
R^{4a} is a halogen atom (e.g., fluorine); X² is N; and
R^{A} is a phenyl group optionally substituted by one to three halogen atoms (e.g., fluorine) (including 2,6-difluorophenyl); or a salt thereof.

Specific examples of compound (I) include the compounds of the below-mentioned Examples 1 to 93, and 96 to 178.

In one aspect, compound (I) is preferably
N-[(3R)-4,4-difluoro-1-{(5S)-5-[5-methyl-3-(2,4,6-trifluorophenyl)pyridin-2-yl]-4,5-dihydro-1,2-oxazol-3-yl}pyrrolidin-3-yl] methanesulfonamide or a salt thereof (Example 11);
N-[(3R)-1-{5-[5-chloro-3-(2,4,6-trifluorophenyl)pyridin-2-yl]-4,5-dihydro-1,2-oxazol-3-yl}-4,4-difluoropyrrolidin-3-yl]methanesulfonamide (optical isomer) or a salt thereof (Example 39);
N-[(3R)-1-{5-[5-chloro-3-(2,4,6-trifluorophenyl)pyridin-2-yl]-4,5-dihydro-1,2-oxazol-3-yl}-4,4-difluoropyrrolidin-3-yl]ethanesulfonamide (optical isomer) or a salt thereof (Example 42);
N-[(3R)-1-{5-[5-chloro-3-(2,6-difluorophenyl)pyridin-2-yl]-4,5-dihydro-1,2-oxazol-3-yl}-4,4-difluoropyrrolidin-3-yl]methanesulfonamide (optical isomer) or a salt thereof (Example 49);
N-[(3R)-1-{(5S)-5-[3-(2,6-difluorophenyl)-5-fluoropyridin-2-yl]-5-(fluoromethyl)-4,5-dihydro-1,2-oxazol-3-yl}-4,4-difluoropyrrolidin-3-yl]methanesulfonamide or a salt thereof (Example 109);
N-[(3R)-4,4-difluoro-1-{5-(fluoromethyl)-5-[5-fluoro-3-(2,4,6-trifluorophenyl)pyridin-2-yl]-4,5-dihydro-1,2-oxazol-3-yl}pyrrolidin-3-yl]methanesulfonamide (optical isomer) or a salt thereof (Example 147);

N-[(3R)-1-{(5S)-5-[3-(2,6-difluorophenyl)-5-fluoropyridin-2-yl]-5-(fluoromethyl)-4,5-dihydro-1,2-oxazol-3-yl}-4,4-difluoropyrrolidin-3-yl]-1-fluoromethanesulfonamide or a salt thereof (Example 149);
N-[(3R)-1-{5-[2-(3,5-difluoropyridin-2-yl)-6-fluoro-4-methylphenyl]-4,5-dihydro-1,2-oxazol-3-yl}-4,4-difluoropyrrolidin-3-yl]methanesulfonamide or a salt thereof (Example 154); and
optical isomer of N-{(3R,4S)-4-fluoro-4-methyl-1-[(5S)-5-(2′,3,5,6′-tetrafluoro[1,1′-biphenyl]-2-yl)-4,5-dihydro-1,2-oxazol-3-yl]pyrrolidin-3-yl}methanesulfonamide or N-{(3S,4R)-4-fluoro-4-methyl-1-[(5S)-5-(2′,3,5,6′-tetrafluoro[1,1′-biphenyl]-2-yl)-4,5-dihydro-1,2-oxazol-3-yl]pyrrolidin-3-yl}methanesulfonamide or N-{(3R,4S)-4-fluoro-4-methyl-1-[(5R)-5-(2′,3,5,6′-tetrafluoro[1,1′-biphenyl]-2-yl)-4,5-dihydro-1,2-oxazol-3-yl]pyrrolidin-3-yl}methanesulfonamide or N-{(3S,4R)-4-fluoro-4-methyl-1-[(5R)-5-(2′,3,5,6′-tetrafluoro[1,1′-biphenyl]-2-yl)-4,5-dihydro-1,2-oxazol-3-yl]pyrrolidin-3-yl}methanesulfonamide or a salt thereof (Example 164).

In one aspect, compound (I) is preferably
N-[(3R)-4,4-difluoro-1-{(5S)-5-[5-methyl-3-(2,4,6-trifluorophenyl)pyridin-2-yl]-4,5-dihydro-1,2-oxazol-3-yl}pyrrolidin-3-yl]methanesulfonamide or a salt thereof (Example 11);
N-[(3R)-1-{(5S)-5-[3-(2,6-difluorophenyl)-5-fluoropyridin-2-yl]-5-(fluoromethyl)-4,5-dihydro-1,2-oxazol-3-yl}-4,4-difluoropyrrolidin-3-yl]methanesulfonamide or a salt thereof (Example 109);
N-[(3R)-1-{(5S)-5-[3-(2,6-difluorophenyl)-5-fluoropyridin-2-yl]-5-(fluoromethyl)-4,5-dihydro-1,2-oxazol-3-yl}-4,4-difluoropyrrolidin-3-yl]-1-fluoromethanesulfonamide or a salt thereof (Example 149); and
N-[(3R)-1-{(5S)-5-[3-(2,6-difluorophenyl)-5-fluoropyridin-2-yl]-5-methyl-4,5-dihydro-1,2-oxazol-3-yl}-4,4-difluoropyrrolidin-3-yl]methanesulfonamide or a salt thereof (Example 165).

As a salt of a compound of formula (I), a pharmacologically acceptable salt is preferable, and examples of such salt include a salt with inorganic base, a salt with organic base, a salt with inorganic acid, a salt with organic acid, a salt with basic or acidic amino acid and the like.

Preferable examples of the salt with inorganic base include alkali metal salts such as sodium salt, potassium salt and the like, alkaline earth metal salts such as calcium salt, magnesium salt and the like, aluminum salt, ammonium salt and the like.

Preferable examples of the salt with organic base include salts with trimethylamine, triethylamine, pyridine, picoline, ethanolamine, diethanolamine, triethanolamine, tromethamine[tris(hydroxymethyl)methylamine], tert-butylamine, cyclohexylamine, benzylamine, dicyclohexylamine, N,N-dibenzylethylenediamine and the like.

Preferable examples of the salt with inorganic acid include salts with hydrochloric acid, hydrobromic acid, nitric acid, sulfuric acid, phosphoric acid and the like.

Preferable examples of the salt with organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Preferable examples of the salt with basic amino acid include salts with arginine, lysine, ornithine and the like. Preferable examples of the salt with acidic amino acid include salts with aspartic acid, glutamic acid and the like.

The production method of the compound of the present invention is explained below.

The raw material compound and reagent used and the compound obtained in each step in the following production method may be each in a form of a salt, and examples of such salt include those similar to the salts of the compound of formula (I), and the like.

When the compound obtained in each step is a free form, it can be converted to the objective salt according to a method known per se. When the compound obtained in each step is a salt, it can be converted to the objective free form or the other salt according to a method known per se.

The compound obtained in each step can be used directly as the reaction mixture or as a crude product for the next reaction. Alternatively, the compound obtained in each step can be isolated and purified from a reaction mixture according to a method known per se, for example, a separation means such as concentration, crystallization, recrystallization, distillation, solvent extraction, fractional distillation, column chromatography and the like.

When the raw material compound and reagent used in each step are commercially available, the commercially available product can also be used directly.

In the reaction in each step, while the reaction time varies depending on the kind of the reagent and solvent to be used, it is generally 1 min-48 hr, preferably 10 min-8 hr, unless otherwise specified.

In the reaction in each step, while the reaction temperature varies depending on the kind of the reagent and solvent to be used, it is generally −78° C.-300° C., preferably −78° C.-150° ° C., unless otherwise specified.

In the reaction in each step, while the pressure varies depending on the kind of the reagent and solvent to be used, it is generally 1 atm-20 atm, preferably 1 atm-3 atm, unless otherwise specified.

Microwave synthesizer such as Initiator manufactured by Biotage and the like may be used for the reaction in each step. While the reaction temperature varies depending on the kind of the reagent and solvent to be used, it is generally room temperature −300° C., preferably 50° C.-250° C., unless otherwise specified. While the reaction time varies depending on the kind of the reagent and solvent to be used, it is generally 1 min-48 hr, preferably 1 min-8 hr, unless otherwise specified.

In the reaction in each step, the reagent is used in an amount of 0.5-20 equivalents, preferably 0.8 equivalents-5 equivalents, relative to the substrate, unless otherwise specified. When the reagent is used as a catalyst, the reagent is used in an amount of 0.001 equivalent-1 equivalent, preferably 0.01 equivalent-0.2 equivalent, relative to the substrate. When the reagent is used as a reaction solvent, the reagent is used in a solvent amount.

Unless otherwise specified, the reaction in each step is carried out without solvent, or by dissolving or suspending the raw material compound in a suitable solvent. Examples of the solvent include those described in Examples and the following solvents.
alcohols: methanol, ethanol, tert-butyl alcohol, 2-methoxyethanol and the like;
ethers: diethyl ether, diphenyl ether, tetrahydrofuran, 1,2-dimethoxyethane and the like;
aromatic hydrocarbons: chlorobenzene, toluene, xylene and the like;
saturated hydrocarbons: cyclohexane, hexane and the like;
amides: N,N-dimethylformamide, N-methylpyrrolidone and the like;
halogenated hydrocarbons: dichloromethane, carbon tetrachloride and the like;
nitriles: acetonitrile and the like;
sulfoxides: dimethyl sulfoxide and the like;
aromatic organic bases: pyridine and the like;
anhydrides: acetic anhydride and the like;
organic acids: formic acid, acetic acid, trifluoroacetic acid and the like;
inorganic acids: hydrochloric acid, sulfuric acid and the like;
esters: ethyl acetate and the like;
ketones: acetone, methyl ethyl ketone and the like; water.

The above-mentioned solvent can be used in a mixture of two or more kinds thereof in an appropriate ratio.

When a base is used for the reaction in each step, examples thereof include those described in Examples and the following bases.
inorganic bases: sodium hydroxide, magnesium hydroxide, sodium carbonate, calcium carbonate, sodium hydrogen carbonate and the like;
organic bases: triethylamine, diethylamine, pyridine, 4-dimethylaminopyridine, N,N-dimethylaniline, 1,4-diazabicyclo[2.2.2]octane, 1,8-diazabicyclo[5.4.0]-7-undecene, imidazole, piperidine and the like;
metal alkoxides: sodium ethoxide, potassium tert-butoxide and the like;
alkali metal hydrides: sodium hydride and the like;
metal amides: sodium amide, lithium diisopropylamide, lithium hexamethyldisilazide and the like;
organic lithiums: n-butyllithium and the like.

When an acid or an acid catalyst is used for the reaction in each step, examples thereof include those described in Examples and the following acids and acid catalysts. inorganic acids: hydrochloric acid, sulfuric acid, nitric acid, hydrobromic acid, phosphoric acid and the like; organic acids: acetic acid, trifluoroacetic acid, citric acid, p-toluenesulfonic acid, 10-camphorsulfonic acid and the like; Lewis acid: boron trifluoride diethyl ether complex, zinc iodide, anhydrous aluminum chloride, anhydrous zinc chloride, anhydrous iron chloride and the like.

Unless otherwise specified, the reaction in each step is carried out according to a method known per se, for example, the method described in Jikken Kagaku Kouza, 5th Edition, vol. 13-19 (the Chemical Society of Japan ed.); Shin Jikken Kagaku Kouza, vol. 14-15 (the Chemical Society of Japan ed.); Fine Organic Chemistry, Revised 2nd Edition (L. F. Tietze, Th. Eicher, Nankodo); Organic Name Reactions, the Reaction Mechanism and Essence, Revised Edition (Hideo Togo, Kodansha); ORGANIC SYNTHESES Collective Volume I-VII (John Wiley & Sons Inc.); Modern Organic Synthesis in the Laboratory A Collection of Standard Experimental Procedures (Jie Jack Li, OXFORD UNIVERSITY); Comprehensive Heterocyclic Chemistry III, Vol. 1-Vol. 14 (Elsevier Japan); Strategic Applications of Named Reactions in Organic Synthesis (translated by Kiyoshi Tomioka, Kagakudojin); Comprehensive Organic Transformations (VCH Publishers Inc.), 1989, or the like, or the method described in Examples.

In each step, the protection or deprotection reaction of an functional group is carried out according to a method known per se, for example, the method described in “Protective Groups in Organic Synthesis, 4th Ed”, Wiley-Interscience, Inc., 2007 (Theodora W. Greene, Peter G. M. Wuts); “Protecting Groups 3rd Ed.” Thieme, 2004 (P. J. Kocienski), or the like, or the method described in Examples.

Examples of the protecting group for a hydroxy group of an alcohol and the like and a phenolic hydroxy group include ether-type protecting groups such as methoxymethyl ether, benzyl ether, tert-butyldimethylsilyl ether, tetrahydropyranyl ether and the like; carboxylate ester-type protecting groups such as acetate ester and the like; sulfonate ester-type protecting groups such as methanesulfonate ester and the like; carbonate ester-type protecting groups such as tert-butylcarbonate and the like, and the like.

Examples of the protecting group for a carbonyl group of an aldehyde include acetal-type protecting groups such as dimethylacetal and the like; cyclic acetal-type protecting groups such as 1,3-dioxane and the like, and the like.

Examples of the protecting group for a carbonyl group of a ketone include ketal-type protecting groups such as dimethylketal and the like; cyclic ketal-type protecting groups such as 1,3-dioxane and the like; oxime-type protecting groups such as O-methyloxime and the like; hydrazone-type protecting groups such as N,N-dimethylhydrazone and the like, and the like.

Examples of the protecting group for a carboxyl group include ester-type protecting groups such as methyl ester and the like; amide-type protecting groups such as N,N-dimethylamide and the like, and the like.

Examples of the protecting group for a thiol include ether-type protecting groups such as benzyl thioether and the like; ester-type protecting groups such as thioacetate ester, thiocarbonate, thiocarbamate and the like, and the like.

Examples of the protecting group for an amino group and an aromatic heterocycle such as imidazole, pyrrole, indole and the like include carbamate-type protecting groups such as benzyl carbamate and the like; amide-type protecting groups such as acetamide and the like; alkyl amine-type protecting groups such as N-triphenylmethylamine and the like; sulfonamide-type protecting groups such as methanesulfonamide and the like, and the like.

The protecting groups can be removed according to a method known per se, for example, by employing a method using acid, base, ultraviolet rays, hydrazine, phenylhydrazine, sodium N-methyldithiocarbamate, tetrabutylammonium fluoride, palladium acetate, trialkylsilyl halide (e.g., trimethylsilyl iodide, trimethylsilyl bromide) and the like, a reduction method, and the like.

When reduction reaction is carried out in each step, examples of the reducing agent to be used include metal hydrides such as lithium aluminum hydride, sodium triacetoxyborohydride, sodium cyanoborohydride, diisobutylaluminum hydride (DIBAL-H), sodium borohydride, tetramethylammonium triacetoxyborohydride and the like; boranes such as borane tetrahydrofuran complex and the like; Raney nickel; Raney cobalt; hydrogen; formic acid; triethylsilane and the like. When carbon-carbon double bond or triple bond is reduced, a method using a catalyst such as palladium-carbon, Lindlar's catalyst and the like may be employed.

When oxidation reaction is carried out in each step, examples of the oxidizing agent to be used include peroxides such as m-chloroperbenzoic acid (mCPBA), hydrogen peroxide, tert-butylhydroperoxide and the like; perchlorates such as tetrabutylammonium perchlorate and the like; chlorates such as sodium chlorate and the like; chlorites such as sodium chlorite and the like; periodates such as sodium periodate and the like; hypervalent iodine reagents such as iodosylbenzene and the like; reagents containing manganese such as manganese dioxide, potassium permanganate and the like; leads such as lead tetraacetate and the like; reagents containing chromium such as pyridinium chlorochromate (PCC), pyridinium dichromate (PDC), Jones reagent and the like; halogen compounds such as N-bromosuccinimide (NBS) and the like; oxygen; ozone; sulfur trioxide-pyridine complex; osmium tetroxide; selenium dioxide; 2,3-dichloro-5,6-dicyano-1,4-benzoquinone (DDQ) and the like.

When radical cyclization reaction is carried out in each step, examples of the radical initiator to be used include azo compounds such as azobisisobutyronitrile (AIBN) and the like; water-soluble radical initiators such as 4-4′-azobis-4-cyanopentanoic acid (ACPA) and the like; triethylboron in the presence of air or oxygen; benzoyl peroxide and the like. Examples of the radical reagent to be used include tributylstannane, tristrimethylsilylsilane, 1,1,2,2-tetraphenyldisilane, diphenylsilane, samarium iodide and the like.

When Wittig reaction is carried out in each step, examples of the Wittig reagent to be used include alkylidene phosphoranes and the like. The alkylidene phosphoranes can be prepared according to a method known per se, for example, by reacting a phosphonium salt with a strong base.

When Horner-Emmons reaction is carried out in each step, examples of the reagent to be used include phosphonoacetates such as methyl dimethylphosphonoacetate, ethyl diethylphosphonoacetate and the like; and bases such as alkali metal hydrides, organic lithiums and the like.

When Friedel-Crafts reaction is carried out in each step, a combination of a Lewis acid and an acid chloride or a combination of a Lewis acid and an alkylating agent (e.g., an alkyl halide, an alcohol, an olefin etc.) is used as a reagent. Alternatively, an organic acid or an inorganic acid can also be used instead of a Lewis acid, and an anhydride such as acetic anhydride and the like can also be used instead of an acid chloride.

When aromatic nucleophilic substitution reaction is carried out in each step, a nucleophile (e.g., an amine, imidazole etc.) and a base (e.g., an organic base etc.) are used as a reagent.

When nucleophilic addition reaction by a carbo anion, nucleophilic 1,4-addition reaction (Michael addition reaction) by a carbo anion or nucleophilic substitution reaction by a carbo anion is carried out in each step, and examples of the base to be used for generation of the carbo anion include organic lithiums, metal alkoxides, inorganic bases, organic bases and the like.

When Grignard reaction is carried out in each step, examples of the Grignard reagent to be used include arylmagnesium halides such as phenylmagnesium bromide and the like; and alkylmagnesium halides such as methylmagnesium bromide and the like. The Grignard reagent can be prepared according to a method known per se, for example, by reacting an alkyl halide or an aryl halide with a metal magnesium in an ether or tetrahydrofuran as a solvent.

When Knoevenagel condensation reaction is carried out in each step, a compound having an activated methylene group with two electron withdrawing groups (e.g., malonic acid, diethyl malonate, malononitrile etc.) and a base (e.g., an organic base, a metal alkoxide, an inorganic base) are used as a reagent.

When Vilsmeier-Haack reaction is carried out in each step, phosphoryl chloride and an amide derivative (e.g., N,N-dimethylformamide etc.) are used as a reagent.

When azidation reaction of an alcohol, an alkyl halide or a sulfonate is carried out in each step, examples of the azidating agent to be used include diphenylphosphorylazide (DPPA), trimethylsilylazide, sodium azide and the like. For example, for the azidation reaction of an alcohol, a method using diphenylphosphorylazide and 1,8-diazabicyclo[5.4.0]undec-7-ene (DBU), a method using trimethylsilylazide and a Lewis acid, and the like are employed.

When reductive amination reaction is carried out in each step, examples of the reducing agent to be used include sodium triacetoxyborohydride, sodium cyanoborohydride, hydrogen, formic acid and the like. When the substrate is an amine compound, examples of the carbonyl compound to be used include paraformaldehyde, aldehydes such as acetaldehyde and the like, and ketones such as cyclohexanone and the like. When the substrate is a carbonyl compound, examples of the amine to be used include ammonia, primary amines such as methylamine and the like; secondary amines such as dimethylamine and the like, and the like.

When Mitsunobu reaction is carried out in each step, an azodicarboxylate (e.g., diethyl azodicarboxylate (DEAD), diisopropyl azodicarboxylate (DIAD) etc.) and triphenylphosphine are used as a reagent.

When esterification reaction, amidation reaction or urea formation reaction is carried out in each step, examples of the reagent to be used include acyl halides such as acid chlorides, acid bromides and the like; activated carboxylic acids such as acid anhydrides, activated esters, sulfates and the like. Examples of the activating agent of the carboxylic acid include carbodiimide condensing agents such as 1-ethyl-3-(3-dimethylaminopropyl) carbodiimide hydrochloride (WSCD) and the like; triazine condensing agents such as 4-(4,6-dimethoxy-1,3,5-triazin-2-yl)-4-methylmorpholinium chloride n-hydrate (DMT-MM) and the like; carbonate condensing agents such as 1,1-carbonyldiimidazole (CDI) and the like; diphenylphosphoryl azide (DPPA); benzotriazol-1-yloxy-trisdimethylaminophosphonium salt (BOP reagent); 2-chloro-1-methyl-pyridinium iodide (Mukaiyama reagent); thionyl chloride; lower alkyl haloformates such as ethyl chloroformate and the like; 0-(7-azabenzotriazol-1-yl)-N,N,N′,N′-tetramethyluronium hexafluorophosphorate (HATU); sulfuric acid; combinations thereof and the like. When carbodiimide condensing agent is used, an additive such as 1-hydroxybenzotriazole (HOBt), N-hydroxysuccinimide (HOSu), dimethylaminopyridine (DMAP) and the like may be added to the reaction system.

When coupling reaction is carried out in each step, examples of the metal catalyst to be used include palladium compounds such as palladium(II) acetate, tetrakis(triphenylphosphine) palladium(0), dichlorobis(triphenylphosphine) palladium(II), dichlorobis(triethylphosphine) palladium(II), tris(dibenzylideneacetone) dipalladium(0), 1,1′-bis(diphenylphosphino) ferrocene palladium(II) chloride and the like; nickel compounds such as tetrakis(triphenylphosphine) nickel (0) and the like; rhodium compounds such as tris(triphenylphosphine) rhodium (III) chloride and the like; cobalt compounds; copper compounds such as copper oxide, copper (I) iodide and the like; platinum compounds and the like. In addition, a base can be added to the reaction system, and examples thereof include inorganic bases and the like.

When thiocarbonylation reaction is carried out in each step, phosphorus pentasulfide is typically used as the thiocarbonylating agent. Alternatively, a reagent having a 1,3,2,4-dithiadiphosphetane-2,4-disulfide structure (e.g., 2,4-bis(4-methoxyphenyl)-1,3,2,4-dithiadiphosphetane-2,4-disulfide (Lawesson reagent) etc.) can also be used instead of phosphorus pentasulfide.

When Wohl-Ziegler reaction is carried out in each step, examples of the halogenating agent to be used include N-iodosuccinimide, N-bromosuccinimide (NBS), N-chlorosuccinimide (NCS), bromine, sulfuryl chloride and the like. In addition, the reaction can be accelerated by subjecting a radical initiator such as heat, light, benzoyl peroxide, azobisisobutyronitrile and the like to the reaction system reaction.

When halogenation reaction of a hydroxy group is carried out in each step, examples of the halogenating agent to be used include hydrohalic acids and acid halides of inorganic acids, specifically, hydrochloric acid, thionyl chloride, phosphorus oxychloride and the like for chlorination, 48% hydrobromic acid and the like for bromination. In addition, a method of producing an alkyl halide by reacting an alcohol with triphenylphosphine and carbon tetrachloride or carbon tetrabromide or the like can be employed. Alternatively, a method of producing an alkyl halide via two steps comprising converting an alcohol to the corresponding sulfonate, and then reacting the sulfonate with lithium bromide, lithium chloride or sodium iodide can also be employed.

When Arbuzov reaction is carried out in each step, examples of the reagent to be used include alkyl halides such as ethyl bromoacetate and the like; and phosphites such as triethyl phosphite, tri (isopropyl)phosphite and the like.

When sulfonate esterification reaction is carried out in each step, examples of the sulfonating agent to be used include methanesulfonyl chloride, p-toluenesulfonyl chloride, methanesulfonic anhydride, p-toluenesulfonic anhydride and the like.

When hydrolysis reaction is carried out in each step, an acid or a base is used as a reagent. For acid hydrolysis reaction of tert-butyl ester, formic acid, triethylsilane and the like may be added to reductively-trap tert-butyl cation which is by-produced.

When dehydration reaction is carried out in each step, examples of the dehydrating agent to be used include sulfuric acid, diphosphorus pentaoxide, phosphorus oxychloride, N,N′-dicyclohexylcarbodiimide, alumina, polyphosphoric acid and the like.

Compound (I) can be produced from compound (1) according to the method shown in the following Scheme 1. In the formulas, X⁷ is a halogen atom, P¹ is a protecting group, LG¹ is a leaving group, and the other symbols are as defined above.

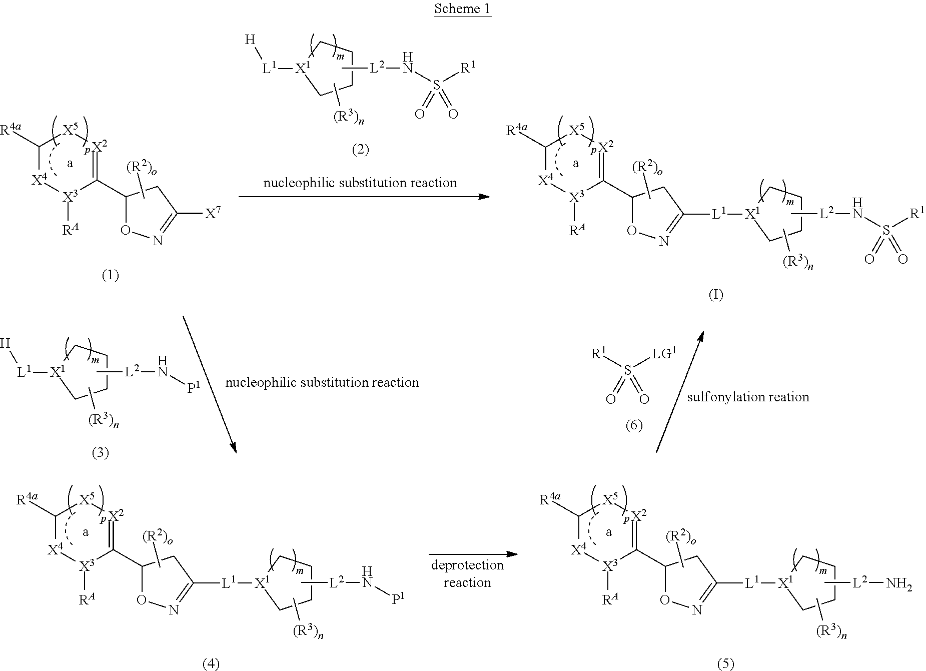

Examples of the “protecting group” for P¹ include those exemplified as the above-mentioned “protecting group for an amino group and an aromatic heterocycle such as imidazole, pyrrole, indole and the like”.

Examples of the “leaving group” for LG¹ include halogen atoms, optionally halogenated C₁₋₆ alkylsulfonyloxy (e.g., methanesulfonyloxy, ethanesulfonyloxy, trifluoromethanesulfonyloxy), C₆₋₁₄ arylsulfonyloxy optionally substituted by C₁₋₆ alkyl (e.g., benzenesulfonyloxy, toluenesulfonyloxy) and the like.

Compound (I) can be produced by subjecting compound (1) to a nucleophilic substitution reaction with compound (2) and base. Examples of the base to be used include inorganic bases, organic bases, alkali metal hydrides and the like.

Compound (4) can be produced by subjecting compound (1) to a nucleophilic substitution reaction with compound (3).

Compound (5) can be produced by subjecting compound (4) to a deprotection reaction.

Compound (I) can also be produced by subjecting compound (5) to a sulfonylation reaction with compound (6). Examples of compound (6) to be used include the corresponding sulfonyl chloride, the corresponding sulfamoyl chloride, and the like.

Compound (1) may be commercially easily available or can be produced according to a method shown in Scheme 3, 5 or 7, or a method known per se.

Compound (2) may be commercially easily available or can be produced according to a method shown in Scheme 4, or a method known per se.

Compounds (3) and (6) may be commercially easily available or can be produced according to a method known per se.

Compound (I)-1, which is compound (I) wherein p is 1, can be produced from compound (7) according to the method shown in the following Scheme 2. In the formulas, X⁸ is a halogen atom, and the other symbols are as defined above.

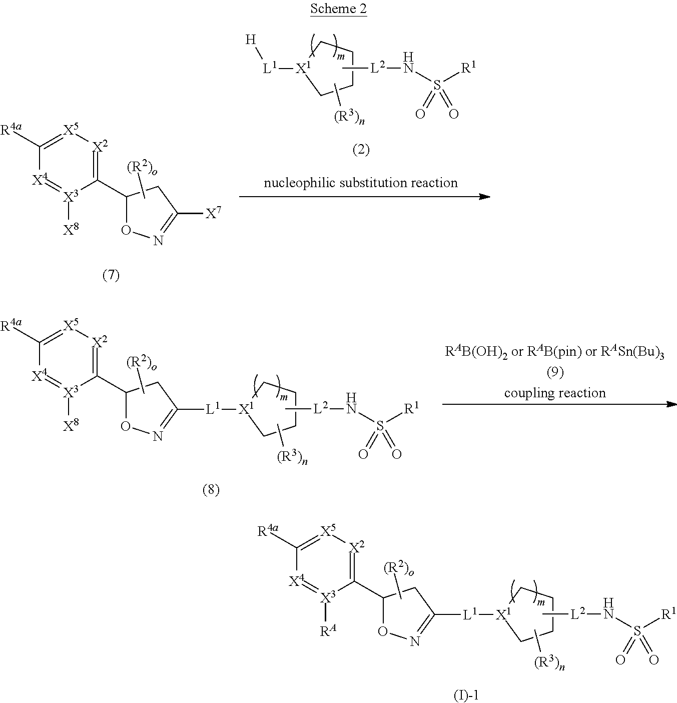

Compound (8) can be produced by subjecting compound (7) to a nucleophilic substitution reaction with compound (2) and base. Examples of the base to be used include inorganic bases, organic bases, alkali metal hydrides and the like.

Compound (I)-1 can be produced by subjecting compound (8) to a coupling reaction with compound (9).

Compound (2) may be commercially easily available or can be produced according to a method shown in Scheme 4, or a method known per se.

Compound (7) may be commercially easily available or can be produced according to a method shown in Scheme 8 or 9, or a method known per se.

Compound (9) may be commercially easily available or can be produced according to a method known per se.

Compound (1)-2, (1)-4, and (1)-5 can be produced from compound (1)-1 according to the method shown in the following Scheme 3. In the formulas, R⁷ is a C₁₋₆ alkyl group, and the other symbols are as defined above.

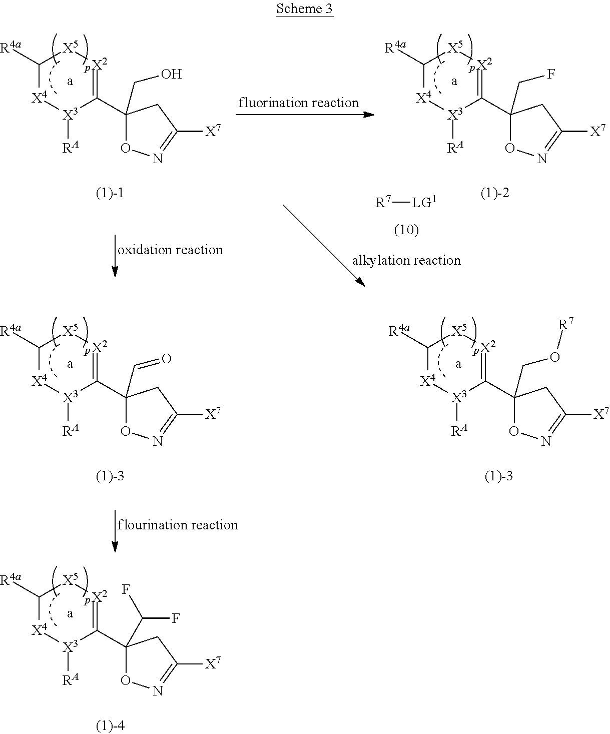

Compound (1)-2 can be produced by subjecting compound (1)-1 to a fluorination reaction. Examples of the fluorination reagent to be used include (diethylamino)sulfur trifluoride, bis(2-methoxyethyl)aminosulfur trifluoride and the like.

Compound (1)-3 can be produced by subjecting compound (1)-1 to an oxidation reaction.

Compound (1)-4 can be produced by subjecting compound (1)-3 to a fluorination reaction.

Compound (1)-5 can be produced by subjecting compound (1)-1 to an alkylation reaction with compound (10).

Compounds (1)-1 and (10) may be commercially easily available or can be produced according to a method known per se.

Compound (2)-1 can be produced from compound (11) or compound (13) according to the method shown in the following Scheme 4. In the formulas, P² is a protecting group, R⁸ is an optionally substituted C₁₋₆ alkyl group, or an optionally substituted C₆₋₁₄ aryl group, and the other symbols are as defined above.

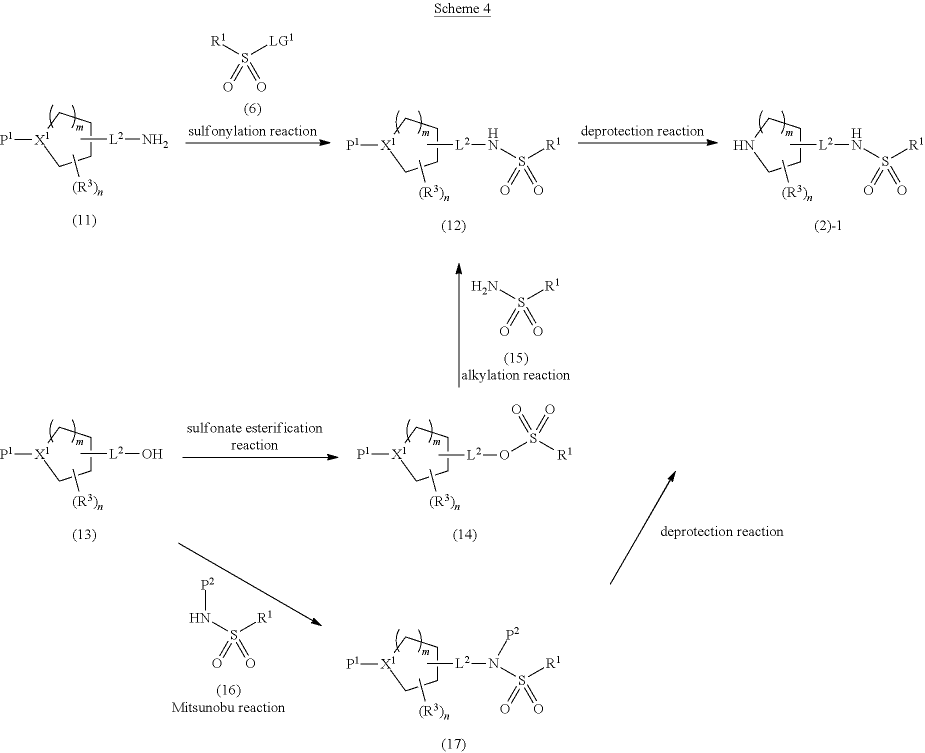

Compound (12) can be produced by subjecting compound (11) to a sulfonylation reaction with compound (6).

Compound (2)-1 can be produced by subjecting compound (12) to a deprotection reaction.

Compound (14) can be produced by subjecting compound (13) to a sulfonate esterification reaction. Examples of the sulfonating agent to be used include methanesulfonyl chloride, p-toluenesulfonyl chloride, methanesulfonic anhydride, p-toluenesulfonic anhydride, trifluoromethanesulfonic anhydride and the like.

Compound (12) can also be produced by subjecting compound (14) to an alkylation reaction with compound (15).

Compound (17) can be produced by subjecting compound (13) to Mitsunobu reaction with compound (16).

Compound (2)-1 can also be produced by subjecting compound (17) to a deprotection reaction.

Compounds (6), (11), (13), (15) and (16) may be commercially easily available or can be produced according to a method known per se.

Compound (1)-6 and compound (4)-1 can be produced from compound (18) according to the method shown in the following Scheme 5. In the formulas, R⁹ and R¹⁰ and R¹¹ are each a hydrogen atom, or an optionally substituted C₁₋₆ alkyl group, and the other symbols are as defined above.

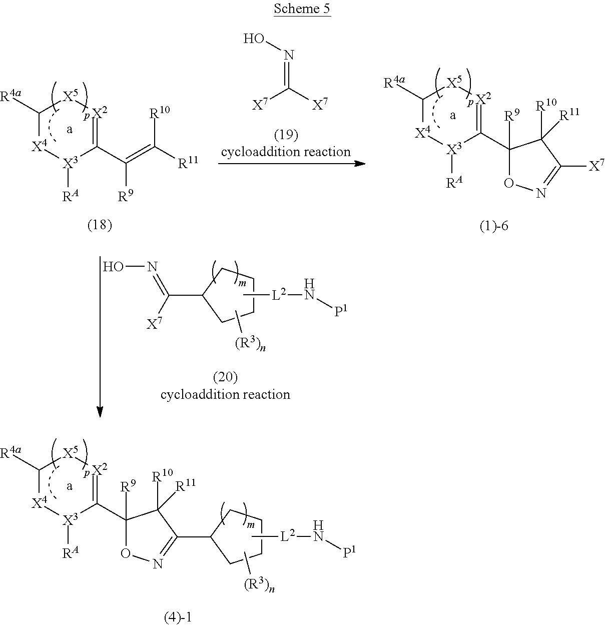

Compound (1)-6 can be produced by subjecting compound (18) to a cycloaddition reaction with compound (19) and base. Examples of the base to be used include inorganic bases, organic bases and the like.

Compound (4)-1 can be produced by subjecting compound (18) to a cycloaddition reaction with compound (20) and base. Examples of the base to be used include inorganic bases, organic bases and the like.

Compound (18) may be commercially easily available or can be produced according to a method shown in Scheme 6, or a method known per se.

Compounds (19) and (20) may be commercially easily available or can be produced according to a method known per se.

Compound (18)-1 can be produced from compound (21), compound (26), or compound (28) according to the method shown in the following Scheme 6. In the formulas, X⁹ and X¹⁰ are each a halogen atom, and the other symbols are as defined above.

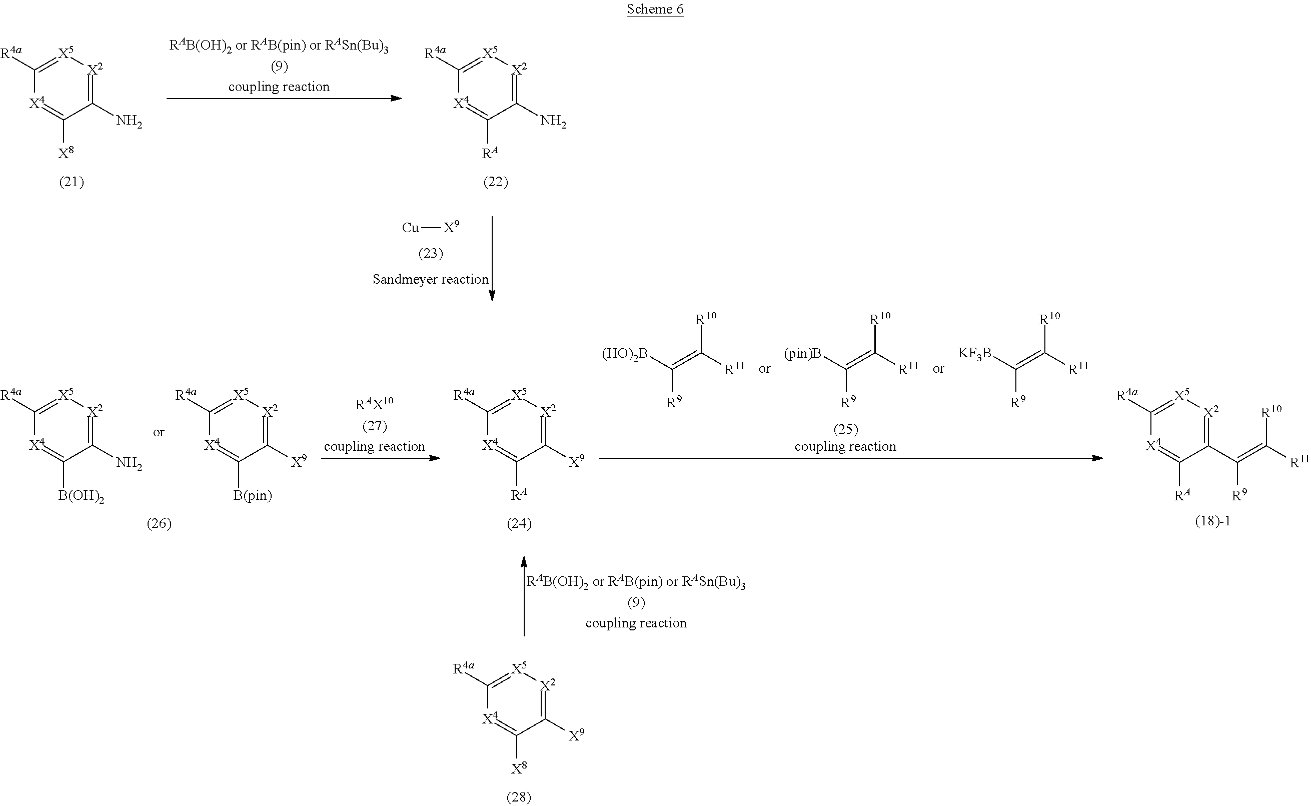

Compound (22) can be produced by subjecting compound (21) to a coupling reaction with compound (9).

Compound (24) can be produced by subjecting compound (22) to Sandmeyer reaction with compound (23). Sandmeyer reaction is carried out according to a method known per se, for example, the method described in Organic Name Reactions, the Reaction Mechanism and Essence, Revised Edition (Hideo Togo, Kodansha); Strategic Applications of Named Reactions in Organic Synthesis (translated by Kiyoshi Tomioka, Kagakudojin) or the like, or the method described in Examples.

Compound (24) can also be produced by subjecting compound (26) to a coupling reaction with compound (27).

Compound (24) can also be produced by subjecting compound (28) to a coupling reaction with compound (9).

Compound (18)-1 can be produced by subjecting compound (24) to a coupling reaction with compound (25).

Compounds (9), (21), (23) and (25)-(28) may be commercially easily available or can be produced according to a method known per se.

Compound (1)-7 can be produced from compound (29) according to the method shown in the following Scheme 7. In the formulas, X¹¹ is a halogen atom, and the other symbols are as defined above.

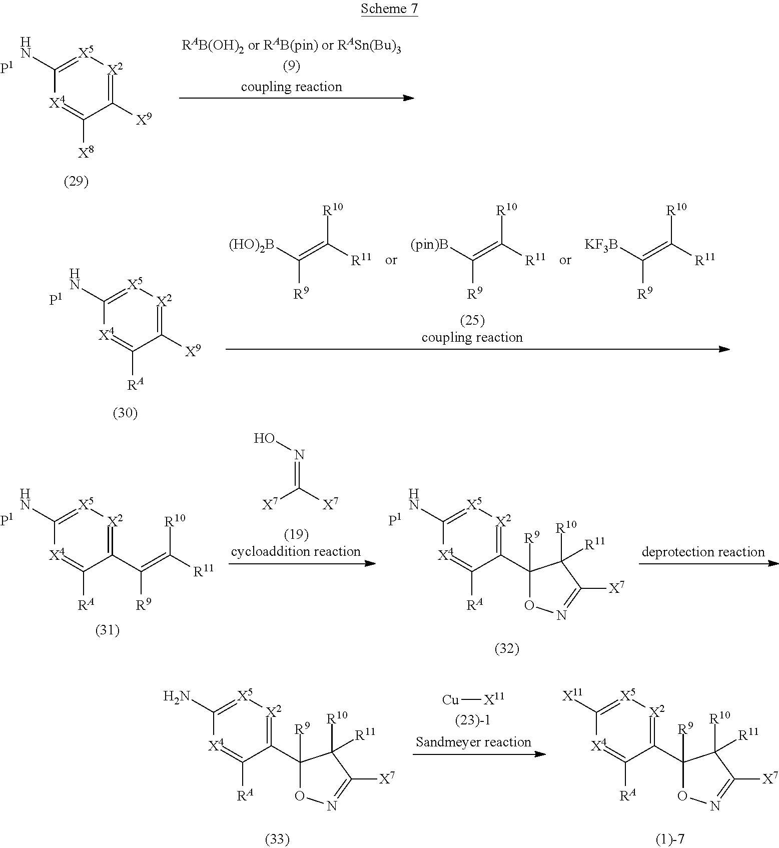

Compound (30) can be produced by subjecting compound (29) to a coupling reaction with compound (9).

Compound (31) can be produced by subjecting compound (30) to a coupling reaction with compound (25).

Compound (32) can be produced by subjecting compound (31) to a cycloaddition reaction with compound (19) and base. Examples of the base to be used include inorganic bases, organic bases and the like.

Compound (33) can be produced by subjecting compound (32) to a deprotection reaction.

Compound (1)-7 can be produced by subjecting compound (33) to Sandmeyer reaction with compound (23)-1.

Compounds (9), (19), (23)-1, (25) and (29) may be commercially easily available or can be produced according to a method known per se.

Compound (7)-2, (7)-4, and (7)-5 can be produced from compound (7)-1 according to the method shown in the following Scheme 8. In the formulas, each symbol is as defined above.

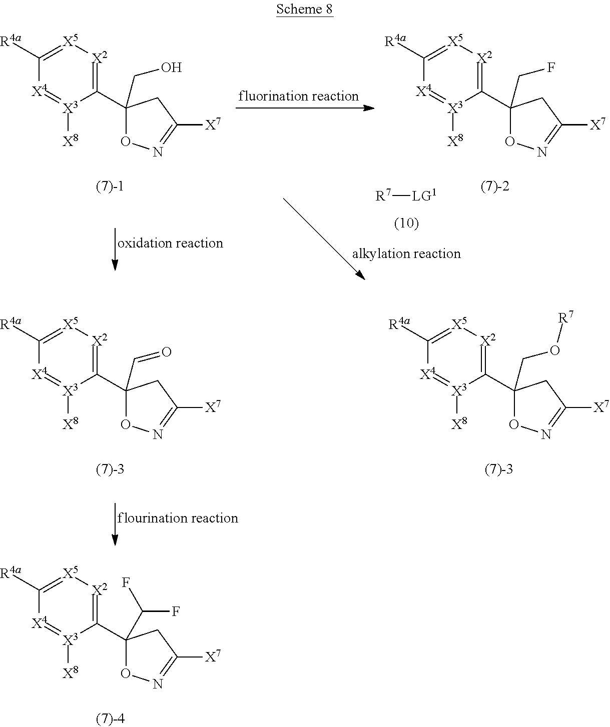

Compound (7)-2 can be produced by subjecting compound (7)-1 to a fluorination reaction.

Compound (7)-3 can be produced by subjecting compound (7)-1 to an oxidation reaction.

Compound (7)-4 can be produced by subjecting compound (7)-3 to a fluorination reaction.

Compound (7)-5 can be produced by subjecting compound (7)-1 to an alkylation reaction with compound (10).

Compounds (7)-1 and (10) may be commercially easily available or can be produced according to a method known per se.

Compound (7)-6 can be produced from compound (28) according to the method shown in the following Scheme 9. In the formulas, each symbol is as defined above.

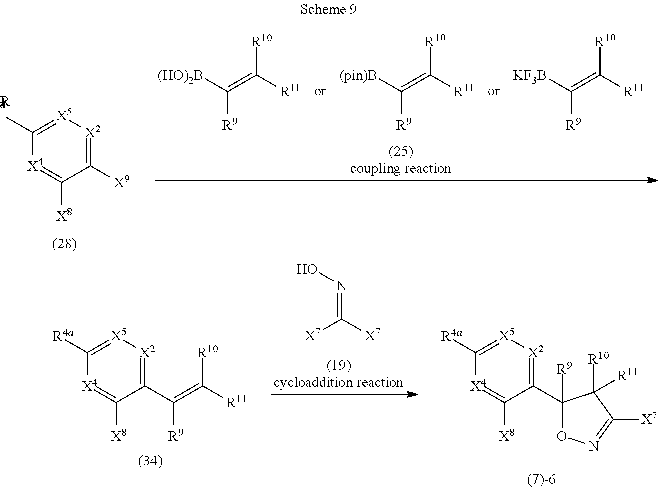

Compound (34) can be produced by subjecting compound (28) to a coupling reaction with compound (25).

Compound (7)-6 can be produced by subjecting compound (34) to a cycloaddition reaction with compound (19) and base. Examples of the base to be used include inorganic bases, organic bases and the like.

Compounds (19), (25) and (28) may be commercially easily available or can be produced according to a method known per se.

Compound (22) can be produced from compound (21)-1 according to the method shown in the following Scheme 10. In the formulas, each symbol is as defined above.

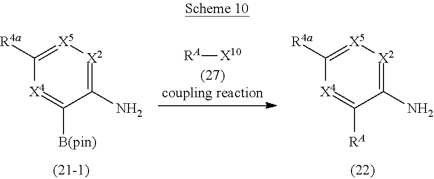

Compound (22) can be produced by subjecting compound (21-1) to a coupling reaction with compound (27).

Compounds (21)-1 and (27) may be commercially easily available or can be produced according to a method known per se.

In the thus-obtained compound (I), an intramolecular functional group can also be converted to an object functional group by a combination of chemical reactions known per se. Examples of the chemical reaction include oxidation reaction, reduction reaction, alkylation reaction, acylation reaction, ureation reaction, hydrolysis reaction, amination reaction, esterification reaction, aryl coupling reaction, deprotection reaction and the like.

In the above-mentioned production method, when a starting compound has an amino group, a carboxyl group, a hydroxy group, a carbonyl group or a mercapto group as a substituent, a protecting group generally used in the peptide chemistry may be introduced into these groups, and the object compound can be obtained by removing the protecting group as necessary after the reaction.

Compound (I) obtained by the above-mentioned production method can be isolated and purified by a known means, such as solvent extraction, liquid conversion, phase transfer, crystallization, recrystallization, chromatography and the like.

When compound (I) contains optical isomer, stereoisomer, regio isomer and rotamer, these compounds are also included in compound (I), and each can be obtained as a single product by a synthesis method or a separation method known per se. For example, when an optical isomer exists in compound (I), an optical isomer resolved from the compound is also encompassed in compound (I).

Here, an optical isomer can be produced by a method known per se.

Compound (I) may be a crystal.

A crystal of compound (I) (hereinafter sometimes to be abbreviated as the crystal of the present invention) can be produced by crystallizing compound (I), by applying a crystallization method known per se.

In the present specification, the melting point means a melting point measured, for example, by micro melting point apparatus (Yanako, MP-500D or Buchi, B-545), DSC (differential scanning calorimetry analysis) apparatus (METTLER TOLEDO, DSC1) and the like.

Generally, the melting point sometimes varies depending on the measurement device, measurement condition and the like. The crystal in the present specification may be a crystal showing a melting point different from the values described in the present specification as long as the difference is within a general error range.

The crystal of the present invention is superior in the physicochemical properties (e.g., melting point, solubility, stability) and biological properties (e.g., pharmacokinetics (absorbability, distribution, metabolism, excretion), efficacy expression), and is extremely useful as a medicament.

Compound (I) may be used as a prodrug. A prodrug of the compound (I) means a compound which is converted to the compound (I) of the present invention with a reaction due to an enzyme, an gastric acid, etc. under the physiological condition in the living body, that is, a compound which is converted to the compound (I) of the present invention with oxidation, reduction, hydrolysis, etc. according to an enzyme; a compound which is converted to the compound (I) of the present invention by hydrolysis etc. due to gastric acid, etc.

A prodrug of compound (I) may be a compound obtained by subjecting an amino group in compound (I) to an acylation, alkylation or phosphorylation (e.g., a compound obtained by subjecting an amino group in compound (I) to an eicosanoylation, alanylation, pentylaminocarbonylation, (5-methyl-2-oxo-1,3-dioxolen-4-yl) methoxycarbonylation, tetrahydrofuranylation, pyrrolidylmethylation, pivaloyloxymethylation and tert-butylation, etc.); a compound obtained by subjecting a hydroxy group in compound (I) to an acylation, alkylation, phosphorylation or boration (e.g., a compound obtained by subjecting an hydroxy group in compound (I) to an acetylation, palmitoylation, propanoylation, pivaloylation, succinylation, fumarylation, alanylation, dimethylaminomethylcarbonylation, etc.); a compound obtained by subjecting a carboxyl group in compound (I) to an esterification or amidation (e.g., a compound obtained by subjecting a carboxyl group in compound (I) to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification and methylamidation, etc.) and the like. Any of these compounds can be produced from compound (I) by a method known per se.

A prodrug for compound (I) may also be one which is converted into compound (I) under a physiological condition, such as those described in IYAKUHIN no KAIHATSU (Development of Pharmaceuticals), Vol. 7, Design of Molecules, p. 163-198, Published by HIROKAWA SHOTEN (1990).

In the present specification, a prodrug may form a salt, and as such salt, those exemplified as a salt of the compound represented by the above-mentioned formula (I) can be mentioned.

Compound (I) may be labeled with an isotope (e.g., ³H, ¹³C, ¹⁴C, ¹⁸F, ³⁵S, ¹²⁵I) and the like.

Compound (I) labeled with or substituted by an isotope can be used, for example, as a tracer used for Positron Emission Tomography (PET) (PET tracer), and is useful in the field of medical diagnosis and the like.

Furthermore, compound (I) may be a hydrate or a non-hydrate, or a non-solvate (e.g., anhydride), or a solvate (e.g., hydrate).

Compound (I) also encompasses a deuterium conversion form wherein ¹H is converted to —H (D).

Furthermore, compound (I) may be a pharmaceutically acceptable cocrystal or cocrystal salt. The cocrystal or cocrystal salt means a crystalline substance constituted with two or more special solids at room temperature, each having different physical properties (e.g., structure, melting point, melting heat, hygroscopicity, solubility and stability). The cocrystal or cocrystal salt can be produced by a cocrystallization method known per se.

Compound (I) or a prodrug thereof (hereinafter sometimes to be simply abbreviated as the compound of the present invention) can be used as it is or in the form of a pharmaceutical composition (also referred to as a medicament) by mixing with a pharmacologically acceptable carrier etc. to mammals (e.g., human, mouse, rat, rabbit, dog, cat, bovine, horse, swine, monkey) as an agent for the prophylaxis or treatment of various diseases mentioned below.

As pharmacologically acceptable carriers, various organic or inorganic carrier substances conventionally used as preparation materials can be used. These are incorporated as excipient, lubricant, binder and disintegrant for solid preparations; or solvent, solubilizing agent, suspending agent, isotonicity agent, buffer and soothing agent for liquid preparations; and the like; and preparation additives such as preservative, antioxidant, colorant, sweetening agent and the like can be added as necessary.

Preferable examples of the excipient include lactose, sucrose, D-mannitol, D-sorbitol, starch, gelatinated starch, dextrin, crystalline cellulose, low-substituted hydroxypropylcellulose, sodium carboxymethylcellulose, gum arabic, pullulan, light anhydrous silicic acid, synthetic aluminum silicate and magnesium alumino metasilicate.

Preferable examples of the lubricant include magnesium stearate, calcium stearate, talc and colloidal silica.

Preferable examples of the binder include gelatinated starch, sucrose, gelatin, gum arabic, methylcellulose, carboxymethylcellulose, sodium carboxymethylcellulose, crystalline cellulose, sucrose, D-mannitol, trehalose, dextrin, pullulan, hydroxypropylcellulose, hydroxypropylmethylcellulose and polyvinylpyrrolidone.

Preferable examples of the disintegrant include lactose, sucrose, starch, carboxymethylcellulose, calcium carboxymethylcellulose, croscarmellose sodium, sodium carboxymethyl starch, light anhydrous silicic acid and low-substituted hydroxypropylcellulose.

Preferable examples of the solvent include water for injection, physiological brine, Ringer's solution, alcohol, propylene glycol, polyethylene glycol, sesame oil, corn oil, olive oil and cottonseed oil.

Preferable examples of the solubilizing agent include polyethylene glycol, propylene glycol, D-mannitol, trehalose, benzyl benzoate, ethanol, trisaminomethane, cholesterol, triethanolamine, sodium carbonate, sodium citrate, sodium salicylate and sodium acetate.

Preferable examples of the suspending agent include surfactants such as stearyltriethanolamine, sodium lauryl sulfate, lauryl aminopropionate, lecithin, benzalkonium chloride, benzethonium chloride, glycerol monostearate and the like; hydrophilic polymers such as poly(vinyl alcohol), polyvinylpyrrolidone, carboxymethylcellulose sodium, methylcellulose, hydroxymethylcellulose, hydroxyethylcellulose, hydroxypropylcellulose and the like, polysorbates; and polyoxyethylene hydrogenated castor oil.

Preferable examples of the isotonicity agent include sodium chloride, glycerol, D-mannitol, D-sorbitol and glucose. Preferable examples of the buffer include buffers of phosphate, acetate, carbonate, citrate etc.

Preferable examples of the soothing agent include benzyl alcohol.

Preferable examples of the preservative include p-oxybenzoate esters, chlorobutanol, benzyl alcohol, phenethyl alcohol, dehydroacetic acid and sorbic acid.

Preferable examples of the antioxidant include sulfite salts and ascorbate salts.

Preferable examples of the colorant include aqueous food tar colors (e.g., food colors such as Food Color Red Nos. 2 and 3, Food Color Yellow Nos. 4 and 5, Food Color Blue Nos. 1 and 2 and the like food colors), water insoluble lake dyes (e.g., aluminum salt of the above-mentioned aqueous food tar color), natural dyes (e.g., β-carotene, chlorophyll, red iron oxide) and the like.

Preferable examples of the sweetening agent include saccharin sodium, dipotassium glycyrrhizinate, aspartame and stevia.

Examples of the dosage form of the above-mentioned pharmaceutical composition include oral preparations such as tablet (including sugar-coated tablet, film-coated tablet, sublingual tablet, orally disintegrating tablet, buccal tablet), capsule (including soft capsule, microcapsule), pill, granule, powder, troche, syrup, liquid, emulsion, suspension, aerosol, films (e.g., orally disintegrable films, oral mucosa-adhesive film) and the like; and parenteral agents such as injection (e.g., subcutaneous injection, intravenous injection, intramuscular injection, intraperitoneal injection, drip infusion), external preparation (e.g., transdermal absorption type preparation, ointment, lotion, adhesive preparation), suppository (e.g., rectal suppository, vaginal suppository), pellet, nasal preparation, pulmonary preparation (inhalant), eye drop and the like. The compound and medicament of the present invention can be respectively safely administered orally or parenterally (e.g., intrarectal, intravenous, intraarterial, intramuscular, subcutaneous, intraorgan, intranasal, intradermal, instillation, intracerebral, intravaginal, intraperitoneal, intratumoral, proximal tumor administrations, and administration to the lesion).

These preparations may be a release control preparation (e.g., sustained-release microcapsule) such as an immediate-release preparation, a sustained-release preparation and the like.

The pharmaceutical composition can be produced according to a method conventionally used in the field of pharmaceutical formulation, for example, the method described in the Japanese Pharmacopoeia, and the like.

While the content of the compound of the present invention in the pharmaceutical composition of the present invention varies depending on the dosage form, dose of the compound of the present invention and the like, it is, for example, about 0.1 to 100 wt %.

When an oral preparation is produced, coating may be applied where necessary for the purpose of taste masking, enteric solubility or sustainability.

Examples of the coating base used for coating include sugar coating base, water-soluble film coating base, enteric film coating base, and sustained-release film coating base.

As the sugar coating base, sucrose is used, and one or more kinds selected from talc, and the precipitated calcium carbonate, gelatin, gum arabic, pullulan, carnauba wax and the like may be further used in combination.

Examples of the water-soluble film coating base include cellulose polymers such as hydroxypropylcellulose, hydroxypropylmethylcellulose, hydroxyethylcellulose, methylhydroxyethylcellulose and the like; synthetic polymers such as polyvinyl acetal diethylaminoacetate, aminoalkylmethacrylate copolymer E [Eudragit E (trade name)], polyvinylpyrrolidone and the like; and polysaccharides such as pullulan and the like.

Examples of the enteric film coating base include cellulose polymers such as hydroxypropylmethylcellulose phthalate, hydroxypropylmethylcellulose acetate succinate, carboxymethylethylcellulose, cellulose acetate phthalate and the like; acrylic acid polymers such as methacrylic acid copolymer L [Eudragit L (trade name)], methacrylic acid copolymer LD [Eudragit L-30D-55 (trade name)], methacrylic acid copolymer S [Eudragit S (trade name)] and the like; and naturally-occurring substances such as shellac and the like.

Examples of the sustained-release film coating base include cellulose polymers such as ethylcellulose and the like; and acrylic acid polymers such as aminoalkylmethacrylate copolymer RS [Eudragit RS (trade name)], ethyl acrylate-methyl methacrylate copolymer suspension [Eudragit NE (trade name)] and the like.

Two or more kinds of the above-mentioned coating bases may be used in a mixture at an appropriate ratio. In addition, for example, light shielding agents such as titanium oxide, red ferric oxide and the like may also be used during coating.

Since the compound of the present invention shows low toxicity (e.g., acute toxicity, chronic toxicity, genetic toxicity, reproductive toxicity, cardiotoxicity, carcinogenicity) and less side effects, it can be used as a prophylactic or therapeutic agent, or diagnostic agent for various diseases in mammals (e.g., human, bovine, horse, dog, cat, monkey, mouse, rat).

Moreover, the compound of the present invention is expected to be superior in central migration.

The compound of the present invention has an excellent an orexin type 2 receptor agonist activity, and may treat, prevent or ameliorate the risk of various neurological and psychiatric diseases associated with an orexin type 2 receptor. The compound of the present invention is useful as an agent for the prophylaxis or treatment of various diseases such as narcolepsy, idiopathic hypersomnia, hypersomnia, sleep apnea syndrome, narcolepsy syndrome accompanied by narcolepsy-like symptoms, hypersomnia syndrome accompanied by daytime hypersomnia (e.g., Kleine Levin syndrome, major depression with hypersomnia, Lewy body dementia, Parkinson's disease, progressive supranuclear paralysis, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalitis, limbic encephalitis, Hashimoto's encephalopathy), coma, loss of consciousness, obesity (e.g., malignant mastocytosis, exogenous obesity, hyperinsulinar obesity, hyperplasmic obesity, hypop hyseal adiposity, hypoplasmic obesity, hypothyroid obesity, hypothalamic obesity, symptomatic obesity, infantile obesity, upper body obesity, alimentary obesity, hypogonadal obesity, systemic mastocytosis, simple obesity, central obesity), insulin resistance syndrome, Alzheimer's disease, disturbance of consciousness such as coma and the like, side effects and complications due to anesthesia, sleep disturbance, sleep problem, insomnia, Intermittent sleep, nocturnal myoclonus, REM sleep interruption, jet lag, jet lag syndrome, sleep disorder of alternating worker, sleep disorder, night terror, depression, major depression, sleepwalking disease, enuresis, sleep disorder, Alzheimer's dusk, diseases associated with circadian rhythm, fibromyalgia, condition arising from decline in the quality of sleep, overeating, obsessive compulsive eating disorder, obesity-related disease, hypertension, diabetes, elevated plasma insulin concentration and insulin resistance, hyperlipidemia, hyperlipemia, endometrial cancer, breast cancer, prostate cancer, colorectal cancer, cancer, osteoarthritis, obstructive sleep apnea, cholelithiasis, gallstones, cardiac disease, abnormal heartbeat, arrhythmia, myocardial infarction, congestive cardiac failure, cardiac failure, coronary heart disease, cardiovascular disorder, sudden death, polycysticovarian disease, craniopharingioma, Froelich's syndrome, growth hormone deficient, normal mutant short stature, Turner's syndrome, children suffering from acute lymphoblastic leukemia, syndrome X, reproductive hormone abnormality, declining fertility, infertility, male gonadal function decline, sexual and reproductive dysfunction such as female male hirsutism, fetal defects associated with pregnant women obesity, gastrointestinal motility disorders such as obesity-related gastroesophageal reflux, obesity hypoventilation syndrome (Pickwick syndrome), respiratory diseases such as dyspnea, inflammation such as systemic inflammation of the vascular system, arteriosclerosis, hypercholesterolemia, hyperuricemia, lower back pain, gall bladder disease, gout, kidney cancer, risk of secondary outcomes of obesity such as lowering the risk of left ventricular hypertrophy, migraine pain, headache, neuropathic pain, Parkinson's disease, psychosis, schizophrenia, facial flushing, night sweats, diseases of the genital/urinary system, diseases related to sexual function or fertility, dysthymic disorder, bipolar disorder, bipolar I disorder, bipolar II disorder, cyclothymic disorder, acute stress disorder, agoraphobia, generalized anxiety disorder, obsessive disorder, panic attack, panic disorder, posttraumatic stress disorder, separation anxiety disorder, social phobia, anxiety disorder, acute neurological and psychiatric disorders such as cardiac bypass surgery and post-transplant cerebral deficit, stroke, ischemic stroke, cerebral ischemia, spinal cord trauma, head trauma, perinatal hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorders associated with muscle spasticity, delirium, amnestic disorder, age-related cognitive decline, schizoaffective disorder, delusional disorder, drug addiction, dyskinesia, chronic fatigue syndrome, fatigue, medication-induced Parkinsonism syndrome, Jill-do La Tourette's syndrome, chorea, myoclonus, tic, restless legs syndrome, dystonia, dyskinesia, attention deficit hyperactivity disorder (ADHD), behavior disorder, urinary incontinence, withdrawal symptoms, trigeminal neuralgia, hearing loss, tinnitus, nerve damage, retinopathy, macular degeneration, vomiting, cerebral edema, pain, bone pain, arthralgia, toothache, cataplexy, and traumatic brain injury (TBI).

Particularly, the compound of the present invention is useful as an agent for the prophylaxis or treatment of narcolepsy, idiopathic hypersomnia, hypersomnia, sleep apnea syndrome, narcolepsy syndrome accompanied by narcolepsy-like symptoms, hypersomnia syndrome accompanied by daytime hypersomnia (e.g., Parkinson's disease, Guillain-Barre syndrome and Kleine Levin syndrome), Alzheimer's disease, obesity, insulin resistance syndrome, cardiac failure, diseases related to bone loss, sepsis, disturbance of consciousness such as coma and the like, side effects and complications due to anesthesia, and the like, or anesthetic antagonist.

In some aspects, the compound of the present invention is useful as an agent for the prophylaxis or treatment of narcolepsy, idiopathic hypersomnia, hypersomnia, sleep apnea syndrome, narcolepsy syndrome accompanied by narcolepsy-like symptoms, hypersomnia syndrome accompanied by daytime hypersomnia, Alzheimer's disease, obesity, insulin resistance syndrome, cardiac failure, diseases related to bone loss, sepsis, disturbance of consciousness, side effects and complications due to anesthesia.

In some aspects, the compound of the present invention is useful as an agent for the prophylaxis or treatment of narcolepsy, idiopathic hypersomnia, hypersomnia, or sleep apnea syndrome.

In some aspects, the compound of the present invention is useful as an agent for the prophylaxis or treatment of narcolepsy.

Central disorders of hypersomnolence (CDH) are characterized by excessive daytime sleepiness in the absence of other sleep disorders but with the setting of adequate and regular sleep habits. Central disorders of hypersomnolence (CDH) include narcolepsy type 1, narcolepsy type 2 and idiopathic hypersomnia. Kleine-Levin syndrome as well as insufficient sleep syndrome and hypersomnia caused by a medical condition, medication or substance, or psychiatric condition are also considered central orders of hypersomnolence. Evaluation of central disorders of hypersomnolence (CDH) includes sleep testing in the sleep lab (polysomnography, PSG) followed by a multiple sleep latency test (MSLT).

Symptoms that are associated with narcolepsy involve abnormal intrusions of REM sleep features into wake: for example, sleep-related hallucinations, sleep paralysis, or vivid dreams and dream-reality confusion. These symptoms can also occur in people without a sleep disorder.

Some patients with narcolepsy also experience cataplexy (narcolepsy, type 1) which is loss of muscle tone triggered by emotion, typically laughter or anticipation. Cataplexy can be generalized or partial and isn't associated with any loss of consciousness. This muscle weakness typically improves within seconds to minutes.

Patients with idiopathic hypersomnia (IDH) often describe excessive daytime sleepiness, prolonged sleep duration (more than 10-11 hours of sleep nightly), and severe difficulty waking up in the morning (sleep inertia). In contrast to narcolepsy, patients with IDH often describe long, unrefreshing daytime naps. Another common symptom of IDH is “brain fog,” a feeling of cognitive clouding during the day. Klein-Levin syndrome is a rare disorder of cyclic hypersomnia.

An aspect of the disclosure is a method for treating a subject having one or more central disorders of hypersomnolence (CDH), the method comprising administering to the subject a compound of the present invention.

Another aspect of the disclosure is the use of a compound of the present invention for the manufacture of an agent for treating one or more central disorders of hypersomnolence (CDH) in a subject.

Another aspect of the disclosure is a compound of the present invention for use in treating one or more central disorders of hypersomnolence (CDH) in a subject. Another aspect is a method or use of the previous aspects, wherein the subject is narcoleptic. In one aspect, the subject has been diagnosed with narcolepsy type 1. In another aspect, the subject has been diagnosed with narcolepsy type 2. In still another aspect, the subject has been diagnosed with idiopathic hypersomnia.

Another aspect is a method or use of the previous aspects, wherein administering the compound of the invention reduces daytime sleepiness, reduces instances of loss of muscle control, and/or reduces instances of interrupted sleep in the subject. Another aspect is a method or use wherein a compound of the present invention is administered in an amount effective to reduce excessive daytime sleepiness in adults with narcolepsy. Another aspect is a method or use wherein a compound of the present invention is administered in an amount effective to increase in mean sleep latency.

Another aspect is a method or use wherein a compound of the present invention is administered in an amount effective to reduce cataplexy events.

Another aspect is a method or use wherein a compound of the present invention is administered in an amount effective to decrease disrupted nocturnal sleep in the subject.

While the dose of the compound of the present invention varies depending on the subject of administration, administration route, target disease, symptom and the like, for example, when the compound of the present invention is administered orally or parenterally to an adult patient, its dose is for example, about 0.01 to 100 mg/kg body weight per dose, preferably 0.1 to 50 mg/kg body weight per dose and more preferably 0.5 to 20 mg/kg body weight per dose. This amount is desirably administered in one to 3 portions daily.

The compound of the present invention can be used in combination with other drugs (hereinafter to be abbreviated as concomitant drug).

By combining the compound of the present invention and a concomitant drug, a superior effect, for example,
(1) the dose can be reduced as compared to single administration of the compound of the present invention or a concomitant drug,
(2) the drug to be combined with the compound of the present invention can be selected according to the condition of patients (mild case, severe case and the like),
(3) the period of treatment can be set longer by selecting a concomitant drug having different action and mechanism from the compound of the present invention,
(4) a sustained treatment effect can be designed by selecting a concomitant drug having different action and mechanism from the compound of the present invention,
(5) a synergistic effect can be afforded by a combined use of the compound of the present invention and a concomitant drug, and the like, can be achieved.

In the present specification, the compound of the present invention and a concomitant drug used in combination are referred to as the “combination agent of the present invention”.

When using the combination agent of the present invention, the administration time of the compound of the present invention and the concomitant drug is not restricted, and the compound of the present invention or a pharmaceutical composition thereof, or the concomitant drug or a pharmaceutical composition thereof can be administered to an administration subject simultaneously, or may be administered at different times. The dosage of the concomitant drug may be determined according to the dose clinically used, and can be appropriately selected depending on an administration subject, administration route, disease, combination and the like.

The administration mode of the combination agent of the present invention and the concomitant drug is not particularly limited, and the compound of the present invention and the concomitant drug only need to be combined on administration. Examples of such administration mode include the following: (1) administration of a single preparation obtained by simultaneously processing the compound of the present invention and the concomitant drug, (2) simultaneous administration of two kinds of preparations of the compound of the present invention and the concomitant drug, which have been separately produced, by the same administration route, (3) administration of two kinds of preparations of the compound of the present invention and the concomitant drug, which have been separately produced, by the same administration route in a staggered manner, (4) simultaneous administration of two kinds of preparations of the compound of the present invention and the concomitant drug, which have been separately produced, by different administration routes, (5) administration of two kinds of preparations of the compound of the present invention and the concomitant drug, which have been separately produced, by different administration routes in a staggered manner (e.g., administration in the order of the compound of the present invention and the concomitant drug, or in the reverse order) and the like.

The dose of the concomitant drug can be appropriately determined based on the dose employed in clinical situations. The mixing ratio of the compound of the present invention and a concomitant drug can be appropriately determined depending on the administration subject, administration route, target disease, symptom, combination and the like.

For example, the content of the compound of the present invention in the combination agent of the present invention differs depending on the form of a preparation, and usually from about 0.01 to about 100 wt %, preferably from about 0.1 to about 50 wt %, further preferably from about 0.5 to about 20 wt, based on the whole preparation.

The content of the concomitant drug in the combination agent of the present invention differs depending on the form of a preparation, and usually from about 0.01 to about 100 wt %, preferably from about 0.1 to about 50 wt %, further preferably from about 0.5 to about 20 wt %, based on the whole preparation.

The content of additives such as a carrier and the like in the combination agent of the present invention differs depending on the form of a preparation, and usually from about 1 to about 99.99 wt %, preferably from about 10 to about 90 wt %, based on the preparation.

Similar contents may be employed even when the compound of the present invention and a concomitant drug are separately formulated into preparations.

Examples of the concomitant drug include the followings. A therapeutic drug for narcolepsy (e.g., methylphenidate, amphetamine, pemoline, phenelzine, protriptyline, sodium oxybate, modafinil, caffeine), antiobesity drug (amphetamine, benzfetamine, bromocriptine, bupropion, diethylpropion, exenatide, fenfluramine, liothyronine, liraglutide, mazindol, methamphetamine, octreotide, octreotide, orlistat, phendimetrazine, phendimetrazine, phenmetrazine, phentermine, Qnexa (registered trade mark), phenylpropanolamine, pramlintide, propylhexedrine, recombinant leptin, sibutramine, topiramate, zimelidine, zonisamide, Lorcaserin, metformin), acetylcholine esterase inhibitor (e.g., donepezil, rivastigmine, galanthamine, zanapezil, idebenone, tacrine), antidementia agent (e.g., memantine), inhibitor of β amyloid protein production, secretion, accumulation, aggregation and/or deposition, β secretase inhibitor (e.g., 6-(4-biphenylyl) methoxy-2-[2-(N,N-dimethylamino) ethyl]tetralin, 6-(4-biphenylyl) methoxy-2-(N,N-dimethylamino) methyltetralin, 6-(4-biphenylyl) methoxy-2-(N,N-dipropylamino) methyltetralin, 2-(N,N-dimethylamino) methyl-6-(4′-methoxybiphenyl-4-yl) methoxytetralin, 6-(4-biphenylyl) methoxy-2-[2-(N,N-diethylamino) ethyl]tetralin, 2-[2-(N,N-dimethylamino)ethyl]-6-(4′-methylbiphenyl-4-yl) methoxytetralin, 2-[2-(N,N-dimethylamino)ethyl]-6-(4′-methoxybiphenyl-4-yl) methoxytetralin, 6-(2′,4′-dimethoxybiphenyl-4-yl) methoxy-2-[2-(N,N-dimethylamino) ethyl]tetralin, 6-[4-(1,3-benzodioxol-5-yl) phenyl]methoxy-2-[2-(N,N-dimethylamino) ethyl]tetralin, 6-(3′,4′-dimethoxybiphenyl-4-yl) methoxy-2-[2-(N,N-dimethylamino) ethyl]tetralin, an optically active form thereof, a salt thereof and a hydrate thereof, OM99-2 (WO01/00663)), γ secretase inhibitor, β amyloid protein aggregation inhibitor (e.g., PTI-00703, ALZHEMED (NC-531), PPI-368 (National Publication of International Patent Application No. 11-514333), PPI-558 (National Publication of International Patent Application No. 2001-500852), SKF-74652 (Biochem. J. (1999), 340 (1), 283-289)), β amyloid vaccine, β amyloid-degrading enzyme and the like, brain function enhancer (e.g., aniracetam, nicergoline), therapeutic drug for Parkinson's disease [(e.g., dopamine receptor agonist (e.g., L-DOPA, bromocriptine, pergolide, talipexole, pramipexole, cabergoline, amantadine), monoamine oxidase enzyme (MAO) inhibitor (e.g., deprenyl, selegiline, remacemide, riluzole), anticholinergic agent (e.g., trihexyphenidyl, biperiden), COMT inhibitor (e.g., entacapone)], therapeutic drug for amyotrophic lateral sclerosis (e.g., riluzole etc., neurotrophic factor), therapeutic drug for abnormal behavior accompanying progress of dementia, wandering and the like (e.g., sedative, anti-anxiety drug), apoptosis inhibitor (e.g., CPI-1189, IDN-6556, CEP-1347), neuronal differentiation-regenerate promoter (e.g., leteprinim, xaliproden; SR-57746-A), SB-216763, Y-128, VX-853, prosaptide, 5,6-dimethoxy-2-[2,2,4,6,7-pentamethyl-3-(4-methylphenyl)-2,3-dihydro-1-benzofuran-5-yl]isoindoline, 5,6-dimethoxy-2-[3-(4-isopropylphenyl)-2,2,4,6,7-pentamethyl-2,3-dihydro-1-benzofuran-5-yl]isoindoline, 6-[3-(4-isopropylphenyl)-2,2,4,6,7-pentamethyl-2,3-dihydro-1-benzofuran-5-yl]-6,7-dihydro-5H-[1,3] dioxolo[4,5-f]isoindole and an optically active form, salt or hydrate thereof), non-steroidal antiinflammatory agents (meloxicam, tenoxicam, indomethacin, ibuprofen, celecoxib, rofecoxib, aspirin, indomethacin etc.), steroid drug (dexamethasone, hexestrol, cortisone acetate etc.), disease-modifying anti-rheumatic drug (DMARDs), anti-cytokine drug (e.g., TNF inhibitor, MAP kinase inhibitor), therapeutic agent for incontinence, frequent urination (e.g., flavoxate hydrochloride, oxybutynin hydrochloride, propiverine hydrochloride), phosphodiesterase inhibitor (e.g., sildenafil (citrate)), dopamine agonist (e.g., apomorphine), antiarrhythmic drugs (e.g., mexiletine), sex hormone or a derivative thereof (e.g., progesterone, estradiol, estradiol benzoate), therapeutic agent for osteoporosis (e.g., alfacalcidol, calcitriol, elcatonin, calcitonin salmon, estriol, ipriflavone, pamidronate disodium, alendronate sodium hydrate, incadronate disodium), parathyroid hormone (PTH), calcium receptor antagonists, therapeutic drug for insomnia (e.g., benzodiazepines medicament, non-benzodiazepines medicament, melatonin agonist, orexin receptor antagonists), therapeutic drug for schizophrenia (e.g., typical antipsychotic agents such as haloperidol and the like; atypical antipsychotic agents such as clozapine, olanzapine, risperidone, aripiprazole and the like; medicament acting on metabotropic glutamate receptor or ion channel conjugated-type glutamate receptor; phosphodiesterase inhibitor), benzodiazepines medicament (chlordiazepoxide, diazepam, potassium clorazepate, lorazepam, clonazepam, alprazolam etc.), L-type calcium channel inhibitor (pregabalin etc.), tricyclic or tetracyclic antidepressant (imipramine hydrochloride, amitriptyline hydrochloride, desipramine hydrochloride, clomipramine hydrochloride etc.), selective serotonin reuptake inhibitor (fluvoxamine maleate, fluoxetine hydrochloride, citalopram hydrobromide, sertraline hydrochloride, paroxetine hydrochloride, escitalopram oxalate etc.), serotonin-noradrenaline reuptake inhibitor (venlafaxine hydrochloride, duloxetine hydrochloride, desvenlafaxine hydrochloride etc.), noradrenaline reuptake inhibitor (reboxetine mesylate etc.), mirtazapine, trazodone hydrochloride, nefazodone hydrochloride, bupropion hydrochloride, setiptiline maleate, 5-HT_{1A} agonist, (buspirone hydrochloride, tandospirone citrate, osemozotan hydrochloride etc.), 5-HT_{2A} antagonist, 5-HT_{2A} inverse agonist, 5-HT₃ antagonist (cyamemazine etc.), heart non-selective β inhibitor (propranolol hydrochloride, oxprenolol hydrochloride etc.), histamine H₁ antagonist (hydroxyzine hydrochloride etc.), CRF antagonist, other antianxiety drug (meprobamate etc.), tachykinin antagonist (MK-869, saredutant etc.), medicament that acts on metabotropic glutamate receptor, CCK antagonist, β3 adrenaline antagonist (amibegron hydrochloride etc.), GAT-1 inhibitor (tiagabine hydrochloride etc.), N-type calcium channel inhibitor, carbonic anhydrase II inhibitor, NMDA glycine moiety agonist, NMDA antagonist (memantine etc.), peripheral benzodiazepine receptor agonist, vasopressin antagonist, vasopressin V1b antagonist, vasopressin V1a antagonist, phosphodiesterase inhibitor, opioid antagonist, opioid agonist, uridine, nicotinic acid receptor agonist, thyroid hormone (T3, T4), TSH, TRH, MAO inhibitor (phenelzine sulfate, tranylcypromine sulfate, moclobemide etc.), therapeutic drug for bipolar disorder (lithium carbonate, sodium valproate, lamotrigine, riluzole, felbamate etc.), cannabinoid CB1 antagonist (rimonabant etc.), FAAH inhibitor, sodium channel inhibitor, anti-ADHD drug (methylphenidate hydrochloride, methamphetamine hydrochloride etc.), therapeutic drug for alcoholism, therapeutic drug for autism, therapeutic drug for chronic fatigue syndrome, therapeutic drug for spasm, therapeutic drug for fibromyalgia syndrome, therapeutic drug for headache, therapeutic drug for quitting smoking, therapeutic drug for myasthenia gravis, therapeutic drug for cerebral infarction, therapeutic drug for mania, therapeutic drug for hypersomnia, therapeutic drug for pain, therapeutic drug for dysthymia, therapeutic drug for autonomic ataxia, therapeutic drug for male and female sexual dysfunction, therapeutic drug for migraine, therapeutic drug for pathological gambler, therapeutic drug for restless legs syndrome, therapeutic drug for substance addiction, therapeutic drug for alcohol-related syndrome, therapeutic drug for irritable bowel syndrome, therapeutic drug for lipid abnormality such as cholesterol-lowering drug (statin series (pravastatin sodium, atorvastatin, simvastatin, rosuvastatin etc.), fibrate (clofibrate etc.), squalene synthetase inhibitor), therapeutic drug for abnormal behavior or suppressant of dromomania due to dementia (sedatives, antianxiety drug etc.), therapeutic drug for diabetes, therapeutic agent for diabetic complications, therapeutic drug for hypertension, therapeutic drug for hypotension, diuretic, chemotherapeutic agent, immunotherapeutic agent, antithrombotic agent, anti-cancer agent and the like.

Two or more kinds of the above-mentioned concomitant drug may be used in a mixture at an appropriate ratio.

When the compound of the present invention is applied to each of the above-mentioned diseases, it can also be used in combination with biologics (e.g., antibody drug, nucleic acid or nucleic acid derivative, aptamer drug, vaccine preparation), or can be used in combination with a gene therapy method and the like, or can also be used in combination with a treatment in psychiatric field without using drugs.

Examples of the antibody drug and vaccine preparation include vaccine preparation against angiotensin II, vaccine preparation against CETP, CETP antibody, antibody against TNFα antibody and other cytokines, amyloid β vaccine preparation, vaccine for type 1 diabetes (e.g., DIAPEP-277 of Peptor), anti-HIV antibody and HIV vaccine preparation, as well as antibodies or vaccine preparations against cytokines, renin-angiotensin type enzymes and products thereof, antibodies or vaccine preparations against enzymes or proteins involved in blood lipid metabolism, antibodies or vaccines relating to enzymes and proteins involved in blood coagulation or fibrinolysis system, antibodies or vaccine preparations against proteins involved in sugar metabolism and insulin resistance, and the like. In addition, it can be used in combination with biologics relating to growth factors such as GH, IGF and the like.

Examples of the gene therapy method include a treatment method using gene relating to cytokine, renin-angiotensin type enzyme and product thereof, G protein, G protein conjugated receptor and phosphorylating enzyme thereof, a treatment method using a DNA decoy such as NFκB decoy and the like, a treatment method using antisense, a treatment method using a gene relating to an enzyme or protein involved in blood lipid metabolism (e.g., a gene relating to metabolism, excretion and absorption of cholesterol or triglyceride or HDL-cholesterol or blood phospholipid), a treatment method using a gene relating to an enzyme or protein involved in angiogenesis therapy for peripheral vascular obstruction and the like (e.g., growth factors such as HGF, VEGF etc.), a treatment method using a gene relating to a protein involved in glucose metabolism and insulin resistance, antisense against cytokines such as TNF etc., and the like.

Examples of the treatment method in the psychiatric field without using drug include modified electroconvulsive therapy, deep brain stimulation therapy, repetitive transcranial magnetic stimulation therapy, psychotherapy including cognitive behavioral therapy and the like.

The compound of the present invention can also be used in combination with various organ regeneration methods such as cardiac regeneration, renal regeneration, pancreatic regeneration, revascularization and the like, cell transplantation therapy utilizing bone marrow cells (bone marrow-derived mononuclear cell, myelogenic stem cell), or artificial organ utilizing tissue engineering (e.g., artificial blood vessel, cardiomyocyte sheet).

### EXAMPLES

The present invention is explained in detail in the following by referring to Examples, Experimental Examples and Formulation Examples. However, the examples do not limit the present invention and the examples can be modified within the scope of the present invention.

The “room temperature” in the following Examples is generally about 10° ° C. to about 35° C. The ratio for mixed solvent is, unless otherwise specified, a volume mixing ratio and % means wt % unless otherwise specified.

The elution by column chromatography in the Examples was performed under the observation by TLC (Thin Layer Chromatography) unless otherwise specified. In the observation by TLC, 60 F₂₅₄ manufactured by Merck was used as a TLC plate, the solvent used as an elution solvent in column chromatography was used as an eluent, and UV detector was used for the detection. In silica gel column chromatography, the indication of NH means use of aminopropylsilane-bonded silica gel and the indication of Diol means use of 3-(2,3-dihydroxypropoxy) propylsilane-bonded silica gel. In preparative HPLC (high performance liquid chromatography), the indication of C18 means use of octadecyl-bonded silica gel. The ratio for elution solvent is, unless otherwise specified, a volume mixing ratio.

For the analysis of ¹H NMR, ACD/SpecManager (trade name) software and the like were used. Peaks of a hydroxyl group, an amino group and the like, having very mild proton peak, are not sometimes described.

MS was measured by LC/MS. As the ionization method, ESI method, or APCI method was used. The data indicates actual measured value (found). While molecular ion peak is generally observed, a fragment ion is sometimes observed. In the case of a salt, a molecular ion peak or fragment ion peak of free form is generally observed.

The unit of sample concentration (c) for optical rotation ([α]D) is g/100 mL.

Elemental analysis value (Anal.) is described as calculated value (Calcd) and actual measured value (Found).

The retention time in the Examples was measured by liquid chromatograph method. The measurement conditions are as follows, unless otherwise specified.
column: YMC PackPro C18 2.0 mm i.d.×75 mm (3 μm) mobile phase: the solution prepared by adding 0.04 M Britton-Robinson buffer solution (pH 6.5) to methanol (5:2), and mixing them well, and adjusting the pH to 7.4 with 0.2 M sodium hydroxide solution.

Peaks by powder X-ray diffraction in the Examples mean peaks measured at room temperature by Ultima IV (Rigaku Corporation, Japan) using Cu Kα radiation as a radiation source.

The measurement conditions are as follows.
Electric pressure/Electric current: 40 kV/50 mA
Scan speed: 6 degrees/min
Scan range of 2 Theta: 2-35 degrees

The crystallinity by powder X-ray diffraction in the Examples was calculated by the Hermans method.

In the following Examples, the following abbreviations are used.
mp: melting point
MS: mass spectrum
M: mol concentration
N: normality
CDCl₃: deuterochloroform
DMSO-d₆: deuterodimethyl sulfoxide
¹H NMR: proton nuclear magnetic resonance
LC/MS: liquid chromatograph mass spectrometer
ESI: Electrospray Ionization
APCI: Atomospheric Pressure Chemical Ionization
Et₂O: diethyl ether
DIPEA: diisopropylethylamine
IPE: diisopropyl ether
NBS: N-bromosuccinimide
CPME: cyclopentyl methyl ether
DIAD: diisopropylethylamine
NMP: N-methyl-2-pyrrolidone
TFA: trifluoroacetic acid
THF: tetrahydrofuran
DMSO: dimethyl sulfoxide
DMA: N,N-dimethylacetamide
DME: 1,2-dimethoxyethane
DMF: N,N-dimethylformamide
Pd₂ (dba) 3: Tris(dibenzylideneacetone) dipalladium(0)
TEA: triethylamine
DPPA: diphenylphosphoryl azide
MeOH: methanol
EtOH: ethanol
IPA: 2-propanol
EtOAc: ethyl acetate
CH₂Cl₂: dichloromethane
t-BuOH: tert-Butyl alcohol
DAST: (diethylamino) sulfur trifluoride
NCS: N-chlorosuccinimide
Pd (Ph₃P)₄: tetrakis(triphenylphosphine) palladium(0)
Pd (OAc)₂: palladium(II) acetate
Pd(dppf) Cl₂: [1,1′-bis(diphenylphosphino) ferrocene]palladium(II) dichloride
Pd(dppf) Cl₂·CH₂Cl₂: [1,1′-bis(diphenylphosphino) ferrocene]palladium(II) dichloride dichloromethane adduct
SFC: supercritical fluid chromatography

### Example 8

### N-{(3S)-1-[5-(2′,6′-Difluoro-5-methyl[1,1′-biphenyl]-2-yl)-4,5-dihydro-1,2-oxazol-3-yl]pyrrolidin-3-yl}ethanesulfonamide

### A) tert-Butyl (3S)-3-[(ethanesulfonyl) amino]pyrrolidine-1-carboxylate

To a solution of tert-butyl (S)-3-aminopyrrolidine-1-carboxylate (1.00 g) and triethylamine (2.10 mL) in THF (22 mL) was added dropwise ethanesulfonyl chloride (0.712 mL) at room temperature. The mixture was stirred at room temperature overnight. The insoluble material was removed by filtration, and the filtrate was concentrated under reduced pressure. The residue was purified by silica gel column chromatography (EtOAc/hexane) to give the title compound (1.28 g). ¹H NMR (300 MHZ, DMSO-d₆) δ 1.18 (3H, t, J=7.4 Hz), 1.39 (9H, s), 1.70-1.84 (1H, m), 1.98-2.09 (1H, m), 3.00-3.25 (4H, m), 3.33-3.39 (1H, m), 3.46 (1H, dd, J=10.9, 6.5 Hz), 3.83 (1H, br s), 7.38 (1H, d, J=6.3 Hz).

### B) N-[(3S)-Pyrrolidin-3-yl]ethanesulfonamide hydrochloride

A mixture of tert-butyl (3S)-3-[(ethanesulfonyl) amino] pyrrolidine-1-carboxylate (2.84 g) and 4 M hydrogen chloride in EtOAc (12.8 mL) was stirred at room temperature overnight. To the resulting suspension was added heptane with stirring and then the precipitates were collected, washed with EtOAc, dried under reduced pressure at 50° C. to give the title compound (2.09 g).

¹H NMR (300 MHz, DMSO-d₆) δ 1.20 (3H, t, J=7.3 Hz), 1.87 (1H, dq, J=13.5, 6.7 Hz), 2.04-2.22 (1H, m), 2.92-3.35 (6H, m), 3.99 (1H, sxt, J=6.2 Hz), 7.56 (1H, d, J=6.4 Hz), 9.31 (2H, br s).

### C) 3-Bromo-5-(2-bromo-4-methylphenyl)-4,5-dihydro-1,2-oxazole

Sodium hydrogen carbonate (128 mg) was added to a mixture of 2-bromo-4-methyl-1-vinylbenzene (150 mg) and hydroxycarbonimidic dibromide (232 mg) in EtOAc (3.05 mL) at room temperature. The mixture was stirred at 70° C. for 7 hours. The precipitate was removed by filtration and the filtrate was concentrated under reduced pressure. The residue was purified by silica gel column chromatography (EtOAc/hexane) to give the title compound (216 mg).

¹H NMR (400 MHZ, CDCl₃) δ 2.34 (3H, s), 3.03 (1H, dd, J=17.4, 7.6 Hz), 3.80 (1H, dd, J=17.4, 11.0 Hz), 5.89 (1H, dd, J=11.0, 7.7 Hz), 7.16 (1H, d, J=7.8 Hz), 7.32-7.51 (2H, m).

### D) N-{(3S)-1-[5-(2-Bromo-4-methylphenyl)-4,5-dihydro-1,2-oxazol-3-yl]pyrrolidin-3-yl}ethanesulfonamide

A mixture of 3-bromo-5-(2-bromo-4-methylphenyl)-4,5-dihydro-1,2-oxazole (55 mg), N-[(3S)-pyrrolidin-3-yl]ethanesulfonamide hydrochloride (48.1 mg) and sodium carbonate (91 mg) was stirred at 130° C. 24 hours. The mixture was poured into water and the resulting mixture was extracted with EtOAc. The organic layer was separated, washed with brine, and concentrated under reduced pressure. The residue was purified by silica gel column chromatography (MeOH/EtOAc) to give the title compound (62 mg).

MS: [M+H]⁺ 416.0.

### E) N-{(3S)-1-[5-(2′,6′-Difluoro-5-methyl[1,1′-biphenyl]-2-yl)-4,5-dihydro-1,2-oxazol-3-yl]pyrrolidin-3-yl}ethanesulfonamide

Tri-tert-butylphosphonium tetrafluoroborate (4.32 mg) and Pd2(dba)3 (4.28 mg) were added to a mixture of N-{(3S)-1-[5-(2-bromo-4-methylphenyl)-4,5-dihydro-1,2-oxazol-3-yl]pyrrolidin-3-yl}ethanesulfonamide (62.0 mg), (2,6-difluorophenyl) boronic acid (47.0 mg), potassium fluoride (17.3 mg) in THF (1.24 mL) and water (0.248 mL). The mixture was stirred at 140° C. for 1 hour under microwave irradiation. The crude material was purified by preparative HPLC (C18, mobile phase: water (containing 10 mM ammonium bicarbonate)/acetonitrile). Then the desired fractions were concentrated to give the title compound (13.0 mg).

¹H NMR (400 MHz, DMSO-d₆) δ 1.18 (3H, td, J=7.3, 1.1 Hz), 1.74-1.86 (1H, m), 2.08-2.13 (1H, m), 2.33 (3H, s), 2.80-3.29 (7H, m), 3.41 (1H, ddd, J=12.4, 10.4, 6.5 Hz), 3.81-3.92 (1H, m), 5.03 (1H, t, J=9.3 Hz), 7.07 (1H, s), 7.23 (2H, td, J=8.6, 2.6 Hz), 7.32 (1H, d, J=7.8 Hz), 7.39 (1H, d, J=5.0 Hz), 7.47-7.60 (2H, m).

### Example 11

### N-[(3R)-4,4-Difluoro-1-{(5S)-5-[5-methyl-3-(2,4,6-trifluorophenyl)pyridin-2-yl]-4,5-dihydro-1,2-oxazol-3-yl}pyrrolidin-3-yl]methanesulfonamide

### A) 5-Methyl-3-(2,4,6-trifluorophenyl)pyridin-2-amine

Tri-tert-butylphosphonium tetrafluoroborate (1.63 g) and Pd2(dba)3 (1.61 g) were added to a mixture of 3-bromo-5-methylpyridin-2-amine (7.00 g), (2,4,6-trifluorophenyl) boronic acid (9.87 g) and potassium fluoride (4.35 g) in THF (104 mL) and water (20.8 mL) at room temperature. The mixture was stirred at 80° C. under nitrogen atmosphere overnight. The mixture was poured into brine and the resulting mixture was extracted with EtOAc. The organic layer was separated, washed with brine, dried over anhydrous sodium sulfate, passed through an NH silica gel pad and concentrated under reduced pressure. The solid was triturated with IPE to give the title compound (9.15 g).

MS: [M+H]⁺ 239.0.

### B) 2-Chloro-5-methyl-3-(2,4,6-trifluorophenyl)pyridine

A solution of sodium nitrite (7.95 g) in water (5 mL) was added dropwise to a solution of 5-methyl-3-(2,4,6-trifluorophenyl)pyridin-2-amine (9.15 g) in conc. hydrogen chloride aqueous solution (80 mL) at −10° C. The mixture was stirred at −10° C. for 10 minutes. Copper (I) chloride (22.8 g) was added portionwise to the mixture at −10° C. The mixture was stirred at room temperature overnight. The mixture was diluted with EtOAc and water, and the resulting mixture was extracted with EtOAc. The organic layer was separated, washed with brine and water and concentrated under reduced pressure. The residue was purified by silica gel column chromatography (EtOAc/hexane) to give the title compound (5.50 g).

MS: [M+H]⁺ 257.9.

### C) 2-Ethenyl-5-methyl-3-(2,4,6-trifluorophenyl)pyridine

Pd(dppf) Cl₂·CH₂C12 (190 mg) was added to a mixture of 2-chloro-5-methyl-3-(2,4,6-trifluorophenyl)pyridine (400 mg), potassium vinyltrifluoroborate (475 mg), potassium carbonate (429 mg) in DME (8.63 mL) and water (1.73 mL) at room temperature. The mixture was stirred at 110° C. under argon atmosphere for 2 hours under microwave irradiation. The organic layer was separated and concentrated under reduced pressure. The residue was purified by NH silica gel column chromatography (EtOAc/hexane) to give the title compound (358 mg).

MS: [M+H]⁺ 250.0.

### D) 2-(3-Bromo-4,5-dihydro-1,2-oxazol-5-yl)-5-methyl-3-(2,4,6-trifluorophenyl)pyridine

Sodium hydrogen carbonate (241 mg) was added to a mixture of 2-ethenyl-5-methyl-3-(2,4,6-trifluorophenyl)pyridine (358 mg) and hydroxycarbonimidic dibromide (437 mg) in EtOAc (5.75 mL) at room temperature. The mixture was stirred at 75° C. overnight. The precipitate was removed by filtration and the filtrate was concentrated under reduced pressure. The residue was purified by silica gel column chromatography (EtOAc/hexane) to give the title compound (293 mg).

MS: [M+H]⁺ 371.0.

### E) 2-[(5S)-3-Bromo-4,5-dihydro-1,2-oxazol-5-yl]-5-methyl-3-(2,4,6-trifluorophenyl)pyridine

2-(3-Bromo-4,5-dihydro-1,2-oxazol-5-yl)-5-methyl-3-(2,4,6-trifluorophenyl)pyridine (9.0 g) was optically resolved by SFC to give the title compound with shorter retention time (4.3 g).

### Chiral Separation Condition

Column: CHIRALPAK IC (trade name) (250 mm*50 mm, 10 μm) Mobile phase: CO2/IPA including 0.1% ammonium hydroxide=750/250 (v/v)

MS: [M+H]⁺ 371.0.

### F) N-[(3R)-4,4-Difluoro-1-{(5S)-5-[5-methyl-3-(2,4,6-trifluorophenyl)pyridin-2-yl]-4,5-dihydro-1,2-oxazol-3-yl}pyrrolidin-3-yl]methanesulfonamide

A mixture of 2-[(5S)-3-bromo-4,5-dihydro-1,2-oxazol-5-yl]-5-methyl-3-(2,4,6-trifluorophenyl)pyridine (1.10 g) and N-[(3R)-4,4-difluoropyrrolidin-3-yl]methanesulfonamide hydrochloride (1.40 g) in pyridine (7.41 mL) was stirred in a sealed tube at 135° C. for 15 hours. The mixture was purified by silica gel column chromatography (EtOAc/hexane) and by NH silica gel column chromatography (EtOAc/hexane). The residue was crystallized from EtOAc/hexane to give the title compound (667 mg).

¹H NMR (400 MHZ, CDCl₃) δ 2.40 (3H, s), 3.08 (3H, s), 3.15 (1H, dd, J=15.7, 9.9 Hz), 3.24-3.32 (1H, m), 3.66-4.03 (4H, m), 4.33 (1H, dquin, J=14.0, 8.9 Hz), 4.84 (1H, d, J=9.8 Hz), 5.49 (1H, t, J=9.1 Hz), 6.78 (2H, dtt, J=17.3, 8.8, 2.3 Hz), 7.40-7.42 (1H, m), 8.53 (1H, d, J=1.8 Hz).

### Example 12

### N-[(3R)-1-{5-[1-(2,6-Difluorophenyl)-4-methyl-1H-imidazol-2-yl]-5-methyl-4,5-dihydro-1,2-oxazol-3-yl}-4,4-difluoropyrrolidin-3-yl]methanesulfonamide

### A) 2-Bromo-1-(2,6-difluoro-4-nitrophenyl)-4-methyl-1H-imidazole

Potassium carbonate (1.72 g) was added to a solution of 2-bromo-4-methyl-1H-imidazole (1.00 g) and 1,2,3-trifluoro-5-nitrobenzene (1.65 g) in DMF (15 mL) at room temperature. The mixture was stirred at 60° C. overnight. To the mixture was added water and the mixture was extracted with EtOAc. The organic layer was separated, washed with brine, dried over anhydrous sodium sulfate and concentrated under reduced pressure to give the title compound (1.40 g) as a mixture with the regioisomer.

MS: [M+H]⁺ 317.9.

### B) 4-(2-Bromo-4-methyl-1H-imidazol-1-yl)-3,5-difluoroaniline

Iron (1.23 g) was added to a solution of 2-bromo-1-(2,6-difluoro-4-nitrophenyl)-4-methyl-1H-imidazole (1.40 g, including regioisomer) and 1 M hydrogen chloride aqueous solution (2 mL) in EtOH (15 mL) at room temperature. The mixture was stirred at 80° C. for 1 hour. The precipitate was removed through Celite (trade name). The filtrate was concentrated under reduced pressure. The residue was dissolved to EtOAc and sodium hydrogen carbonate aqueous solution The mixture was extracted with EtOAc.

The organic layer was separated, washed with brine and water and concentrated under reduced pressure. The residue was purified by silica gel column chromatography (EtOAc/hexane) to give the title compound (1.12 g).

MS: [M+H]⁺ 287.9.

### C) 2-Bromo-1-(2,6-difluorophenyl)-4-methyl-1H-imidazole

To a solution of n-pentyl nitrite (1.03 mL) in THF (38.9 mL) was added 4-(2-bromo-4-methyl-1H-imidazol-1-yl)-3,5-difluoroaniline (1.12 g) portionwise at 0° C. The mixture was stirred at 0° C. for 15 minutes. Then hypophosphoric acid (2.13 mL) was added to the solution and the mixture was stirred at 0° C. for 30 minutes. The mixture was neutralized with saturated sodium hydrogen carbonate aqueous solution and the resulting mixture was extracted with EtOAc. The organic layer was washed with brine, dried over anhydrous sodium sulfate and concentrated under reduced pressure. The residue was purified by silica gel column chromatography (EtOAc/hexane) to give the title compound (595 mg).

¹H NMR (400 MHZ, CDCl₃) δ 2.28 (3H, s), 6.80 (1H, s), 7.09 (2H, t, J=8.3 Hz), 7.46 (1H, quin, J=7.3 Hz).

### D) 1-(2,6-Difluorophenyl)-4-methyl-2-(prop-1-en-2-yl)-1H-imidazole

Pd(dppf) Cl₂·CH₂Cl₂ (93.0 mg) was added to a mixture of 2-bromo-1-(2,6-difluorophenyl)-4-methyl-1H-imidazole (205 mg), 4,4,5,5-tetramethyl-2-(prop-1-en-2-yl)-1,3,2-dioxaborolane (191 mg), potassium carbonate (235 mg) in DME (4 mL) and water (0.800 mL) at room temperature. The mixture was stirred at 110° C. under argon atmosphere for 2 hours under microwave irradiation. The organic layer was separated and concentrated under reduced pressure. The residue was purified by NH silica gel column chromatography (EtOAc/hexane) to give the title compound (125 mg).

MS: [M+H]⁺ 235.0.

### E) 3-Bromo-5-[1-(2,6-difluorophenyl)-4-methyl-1H-imidazol-2-yl]-5-methyl-4,5-dihydro-1,2-oxazole

Sodium hydrogen carbonate (134 mg) was added to a solution of 1-(2,6-difluorophenyl)-4-methyl-2-(prop-1-en-2-yl)-1H-imidazole (125 mg) and hydroxycarbonimidic dibromide (216 mg) in EtOAc (3 mL) at room temperature. The mixture was stirred at 70° C. for 5 hours. The precipitate was removed by filtration. The filtrate was concentrated under reduced pressure. The residue was purified by silica gel column chromatography (EtOAc/hexane) to give the title compound (71.0 mg).

MS: [M+H]⁺ 355.9.

### F) N-[(3R)-1-{5-[1-(2,6-Difluorophenyl)-4-methyl-1H-imidazol-2-yl]-5-methyl-4,5-dihydro-1,2-oxazol-3-yl}-4,4-difluoropyrrolidin-3-yl]methanesulfonamide

A mixture of 3-bromo-5-[1-(2,6-difluorophenyl)-4-methyl-1H-imidazol-2-yl]-5-methyl-4,5-dihydro-1,2-oxazole (71 mg), (N-[(3R)-4,4-difluoropyrrolidin-3-yl]methanesulfonamide hydrochloride (70.8 mg) in pyridine (0.498 mL) was stirred at 140° ° C. overnight. The residue was purified by silica gel column chromatography (EtOAc/hexane) and by preparative HPLC (C18, mobile phase: water (containing 10 mM ammonium bicarbonate)/acetonitrile). Then the desired fractions were concentrated to give the title compound (46.3 mg).

¹H NMR (300 MHZ, DMSO-d₆) δ 1.49 (3H, d, J=2.3 Hz), 2.12 (3H, d, J=0.8 Hz), 2.99 (3H, d, J=0.6 Hz), 3.03-3.26 (2H, m), 3.43-3.91 (4H, m), 4.07-4.48 (1H, m), 6.97 (1H, dd, J=1.7, 1.1 Hz), 7.18-7.37 (2H, m), 7.47-7.72 (1H, m), 7.95 (1H, dd, J=9.1, 4.1 Hz).

### Example 20

### N-[(1s,3s)-3-({5-[3-(2,6-Difluorophenyl)-5-methylpyridin-2-yl]-4,5-dihydro-1,2-oxazol-3-yl}oxy) cyclobutyl]methanesulfonamide

### A) 2-Chloro-3-(2,6-difluorophenyl)-5-methylpyridine

Tri-tert-butylphosphonium tetrafluoroborate (0.703 g) and Pd2(dba)3 (1.11 g) were added to a mixture of 3-bromo-2-chloro-5-methylpyridine (5.00 g), (2,6-difluorophenyl) boronic acid (4.97 g) and potassium fluoride (2.81 g) in THF (67.3 mL) and water (13.5 mL) at room temperature. The mixture was stirred at 80° C. under nitrogen atmosphere for 2 hours. The mixture was poured into brine and the resulting mixture was extracted with EtOAc. The organic layer was separated, washed with brine, dried over anhydrous sodium sulfate and concentrated under reduced pressure. The residue was purified by NH silica gel column chromatography (EtOAc/hexane) to give the title compound (5.55 g).

MS: [M+H]⁺ 239.9.

### B) 3-(2,6-Difluorophenyl)-2-ethenyl-5-methylpyridine

Pd(dppf) Cl₂·CH₂C12 (0.562 g) was added to a mixture of 2-chloro-3-(2,6-difluorophenyl)-5-methylpyridine (1.10 g), potassium vinyltrifluoroborate (1.35 g), potassium carbonate (1.27 g) in DME (17 mL) and water (3 mL) at room temperature. The mixture was stirred at 110° C. under argon atmosphere for 3.5 hours under microwave irradiation. The organic layer was separated and concentrated under reduced pressure. The residue was purified by NH silica gel column chromatography (EtOAc/hexane) to give the title compound (6.10 g).

MS: [M+H]⁺ 232.0.

### C) 2-(3-Bromo-4,5-dihydro-1,2-oxazol-5-yl)-3-(2,6-difluorophenyl)-5-methylpyridine

Sodium hydrogen carbonate (7.34 g) was added to a mixture of 3-(2,6-difluorophenyl)-2-ethenyl-5-methylpyridine (5.05 g) and hydroxycarbonimidic dibromide (8.86 g) in EtOAc (87 mL) at room temperature. The mixture was stirred at 70° C. overnight. The precipitate was removed by filtration and the filtrate was concentrated under reduced pressure. The residue was purified by silica gel column chromatography (EtOAc/hexane) to give the title compound (6.37 g).

¹H NMR (400 MHZ, CDCl₃) δ 2.41 (3H, s), 3.41 (1H, dd, J=17.1, 11.0 Hz), 3.89 (1H, dd, J=17.1, 9.0 Hz), 5.55-5.71 (1H, m), 6.95-7.09 (2H, m), 7.30-7.48 (2H, m), 8.57 (1H, d, J=1.8 Hz).

### D) tert-Butyl [(1s,3s)-3-({5-[3-(2,6-difluorophenyl)-5-methylpyridin-2-yl]-4,5-dihydro-1,2-oxazol-3-yl}oxy) cyclobutyl]carbamate

To a solution of tert-butyl ((1s,3s)-3-hydroxycyclobutyl) carbamate (31.8 mg) in THF (0.5 mL) was added sodium hydride (60% in oil, 6.80 mg) at room temperature. After being stirred at room temperature for 30 minutes, 2-(3-bromo-4,5-dihydro-1,2-oxazol-5-yl)-3-(2,6-difluorophenyl)-5-methylpyridine (40 mg) in THF (0.1 mL) was added to the reaction mixture. The mixture was refluxed overnight. The mixture was quenched with saturated ammonium chloride aqueous solution at room temperature and the resulting mixture was extracted with EtOAc. The organic layer was separated, washed with water and brine, dried over anhydrous sodium sulfate and concentrated under reduced pressure. The residue was purified by silica gel column chromatography (EtOAc/hexane) to give the title compound (29.4 mg).

MS: [M+H]⁺ 460.1.

### E) (1s,3s)-3-({5-[3-(2,6-Difluorophenyl)-5-methylpyridin-2-yl]-4,5-dihydro-1,2-oxazol-3-yl}oxy) cyclobutan-1-amine trifluoroacetic acid

To a solution tert-butyl [(1s,3s)-3-({5-[3-(2,6-difluorophenyl)-5-methylpyridin-2-yl]-4,5-dihydro-1,2-oxazol-3-yl}oxy)cyclobutyl]carbamate (29.4 mg) in toluene (1 mL) was added TFA (1 mL). The mixture was stirred at room temperature for 30 minutes. The mixture was concentrated under reduced pressure to give the title compound (30.3 mg).

MS: [M+H-(TFA)]+360.1.

### F) N-[(1s,3s)-3-({5-[3-(2,6-Difluorophenyl)-5-methylpyridin-2-yl]-4,5-dihydro-1,2-oxazol-3-yl}oxy) cyclobutyl]methanesulfonamide

To a cooled (0° C.) mixture of (1s,3s)-3-({5-[3-(2,6-Difluorophenyl)-5-methylpyridin-2-yl]-4,5-dihydro-1,2-oxazol-3-yl}oxy) cyclobutan-1-amine trifluoroacetic acid (30.3 mg) and TEA (0.089 mL) in THF (1 mL) was added methanesulfonic anhydride (14.5 mg) and the mixture was stirred at room temperature for 1 hour. After concentration of the resulting mixture, the residue was purified by silica gel column chromatography (EtOAc/hexane) to give the title compound (23.5 mg).

¹H NMR (400 MHZ, CDCl₃) δ 2.06-2.15 (2H, m), 2.40 (3H, s), 2.93 (3H, s), 2.94-3.12 (3H, m), 3.56-3.76 (2H, m), 4.44 (1H, d, J=8.8 Hz), 4.60 (1H, t, J=7.0 Hz), 5.52 (1H, t, J=9.7 Hz), 6.95-7.05 (2H, m), 7.34-7.44 (2H, m), 8.56 (1H, d, J=1.8 Hz).

### Example 22

### N-[(3R)-1-{(5S)-5-[3-(2,6-Difluorophenyl)-5-methylpyridin-2-yl]-4,5-dihydro-1,2-oxazol-3-yl}-4,4-difluoropyrrolidin-3-yl]ethanesulfonamide

### A) 2-[(5S)-3-Bromo-4,5-dihydro-1,2-oxazol-5-yl]-3-(2,6-difluorophenyl)-5-methylpyridine

2-(3-Bromo-4,5-dihydro-1,2-oxazol-5-yl)-3-(2,6-difluorophenyl)-5-methylpyridine (253 mg) was optically resolved by HPLC. The fraction with shorter retention time was concentrated under reduced pressure to give the title compound (111 mg).

### Chiral Separation Condition

Column: CHIRALPAK IH (trade name) (20 mmID*250 mmL, 5 μm)

Mobile phase: Hexane/IPA=800/200 (v/v)

MS (found): 353.1, 355.1.

### B) N-[(3R)-1-{(5S)-5-[3-(2,6-Difluorophenyl)-5-methylpyridin-2-yl]-4,5-dihydro-1,2-oxazol-3-yl}-4,4-difluoropyrrolidin-3-yl]ethanesulfonamide

A mixture of 2-[(5S)-3-bromo-4,5-dihydro-1,2-oxazol-5-yl]-3-(2,6-difluorophenyl)-5-methylpyridine (50.0 mg), N-[(3R)-4,4-difluoropyrrolidin-3-yl]ethanesulfonamide hydrochloride (71.0 mg) in pyridine (0.354 mL) was stirred in a sealed tube at 140° C. for 15 hours. The residue was purified by silica gel column chromatography (EtOAc/hexane). The desired fraction was concentrated under reduced pressure. The residue was triturated with IPE to give the title compound (22.1 mg).

¹H NMR (400 MHZ, CDCl₃) δ 1.42 (3H, t, J=7.3 Hz), 2.40 (3H, s), 3.09-3.21 (3H, m), 3.27 (1H, t, J=9.6 Hz), 3.69-3.86 (3H, m), 3.91 (1H, dd, J=9.9, 7.9 Hz), 4.11-4.41 (1H, m), 4.66 (1H, d, J=9.5 Hz), 5.51 (1H, t, J=9.0 Hz), 6.76-7.11 (2H, m), 7.29-7.47 (2H, m), 8.54 (1H, d, J=2.0 Hz).

### Example 23

### N-[(1s,3s)-3-{5-[5-Methyl-3-(2,4,6-trifluorophenyl)pyridin-2-yl]-4,5-dihydro-1,2-oxazol-3-yl}cyclobutyl]methanesulfonamide

### A) tert-Butyl [(1s,3s)-3-{5-[5-Methyl-3-(2,4,6-trifluorophenyl)pyridin-2-yl]-4,5-dihydro-1,2-oxazol-3-yl}cyclobutyl]carbamate

A mixture of tert-butyl ((1s,3s)-3-formylcyclobutyl) carbamate (1.24 g), hydroxylamine hydrochloride (0.388 mL) and pyridine (1.01 mL) in EtOH (10 mL) and water (3 mL) was stirred at room temperature for 2 hours. The mixture was extracted with EtOAc. The organic layer was washed with brine, dried over anhydrous magnesium sulfate and concentrated under reduced pressure. A mixture of the residue and NCS (0.831 g) in DMF (15 mL) was stirred at room temperature for 1 hour. The mixture was diluted with water and the resulting mixture was extracted with EtOAc. The organic layer was washed with brine three times, dried over anhydrous magnesium sulfate and concentrated under reduced pressure to give the residue. TEA (0.380 mL) was added to a mixture of 5-methyl-3-(2,4,6-trifluorophenyl)-2-vinylpyridine (200 mg) and 339 mg of the residue in THF (2 mL) at room temperature. The mixture was stirred at 50° C. for 1 hour. To the mixture was added water and the mixture was extracted with EtOAc. The organic layer was separated and concentrated under reduced pressure. The residue was purified by silica gel column chromatography (EtOAc/hexane) to give the title compound (355 mg).

MS: [M+H]⁺ 462.1.

### B) N-[(1s,3s)-3-{5-[5-Methyl-3-(2,4,6-trifluorophenyl)pyridin-2-yl]-4,5-dihydro-1,2-oxazol-3-yl}cyclobutyl]methanesulfonamide

A mixture of tert-Butyl [(1s,3s)-3-{5-[5-Methyl-3-(2,4,6-trifluorophenyl)pyridin-2-yl]-4,5-dihydro-1,2-oxazol-3-yl}cyclobutyl]carbamate (355 mg) in TFA (1 mL) was stirred at room temperature for 20 minutes. The mixture was concentrated under reduced pressure. To a solution of the residue and TEA (0.537 mL) in THF (2 mL) was added methanesulfonyl chloride (132 mg). The mixture was stirred at 0° C. for 10 minutes. Then TEA (0.3 mL) and methanesulfonyl chloride (30.0 mg) were added to the mixture at room temperature and the reaction mixture was stirred for 10 minutes at room temperature. The mixture was purified by silica gel column chromatography (EtOAc/hexane) to give the title compound (265 mg).

¹H NMR (300 MHZ, CDCl₃) δ 2.14-2.28 (2H, m), 2.39 (3H, s), 2.63-2.78 (2H, m), 2.81-2.96 (4H, m), 3.10 (1H, dd, J=16.8, 10.7 Hz), 3.53 (1H, dd, J=16.9, 7.9 Hz), 3.96 (1H, sxt, J=8.2 Hz), 4.84-5.04 (1H, m), 5.45 (1H, dd, J=10.2, 8.7 Hz), 6.64-6.90 (2H, m), 7.34-7.45 (1H, m), 8.52 (1H, d, J=2.3 Hz).

### Example 30

### N-[(3S)-1-{(5S)-5-[3-(2,6-Difluorophenyl)-5-methylpyridin-2-yl]-4,5-dihydro-1,2-oxazol-3-yl}pyrrolidin-3-yl]-1-fluorocyclopropane-1-sulfonamide

### A) tert-Butyl (3S)-3-[(tert-butoxycarbonyl) (1-fluorocyclopropane-1-sulfonyl)amino]pyrrolidine-1-carboxylate

To a solution of tert-butyl ((1-fluorocyclopropyl) sulfonyl) carbamate (479 mg), tert-butyl (R)-3-hydroxypyrrolidine-1-carboxylate (374 mg) and triphenylphosphine (1.05 g) in toluene (20 mL) was added 1.9 M DIAD in toluene (2.63 mL) at 0° C. The mixture was stirred at room temperature overnight. The mixture was diluted with EtOAc and poured into water at room temperature. The organic layer was separated, washed with water and brine, dried over anhydrous sodium sulfate, and concentrated under reduced pressure. The residue was purified by silica gel column chromatography (EtOAc/hexane) to give the title compound (587 mg).

¹H NMR (300 MHZ, DMSO-d₆) δ 1.39 (9H, s), 1.48 (9H, s), 1.61-1.83 (4H, m), 2.07-2.19 (1H, m), 2.20-2.33 (1H, m), 3.12-3.28 (1H, m), 3.36-3.59 (3H, m), 4.77-4.96 (1H, m).

### B) 1-Fluoro-N-[(3S)-pyrrolidin-3-yl]cyclopropane-1-sulfonamide hydrochloride

tert-Butyl (3S)-3-[(tert-butoxycarbonyl) (1-fluorocyclopropane-1-sulfonyl)amino] pyrrolidine-1-carboxylate (587 mg) was dissolved in 4 M hydrogen chloride in CPME (6 mL). The mixture was stirred at room temperature under a dry atmosphere overnight. The mixture was concentrated. The residue was triturated with EtOAc and filtered to give the title compound (364 mg).

¹H NMR (300 MHz, DMSO-d₆) δ 1.34-1.62 (4H, m), 1.89 (1H, td, J=13.6, 7.2 Hz), 2.18 (1H, dq, J=13.6, 7.0 Hz), 3.01 (1H, dd, J=12.0, 6.0 Hz), 3.13-3.30 (2H, m), 3.35-3.46 (1H, m), 4.15 (1H, quin, J=6.4 Hz), 8.26-8.72 (1H, m), 9.10 (2H, br s).

### C) N-[(3S)-1-{(5S)-5-[3-(2,6-Difluorophenyl)-5-methylpyridin-2-yl]-4,5-dihydro-1,2-oxazol-3-yl}pyrrolidin-3-yl]-1-fluorocyclopropane-1-sulfonamide

To a mixture of 2-[(5S)-3-bromo-4,5-dihydro-1,2-oxazol-5-yl]-3-(2,6-difluorophenyl)-5-methylpyridine (43.3 mg) and DIPEA (0.143 mL) in NMP (0.50 mL) was added 1-fluoro-N-[(3S)-pyrrolidin-3-yl]cyclopropane-1-sulfonamide hydrochloride (20 mg). The mixture was stirred at 120° C. for 3 hours. The mixture was quenched with water and the resulting mixture was extracted with EtOAc. The organic layer was separated, washed with water and brine, dried over anhydrous sodium sulfate and concentrated under reduced pressure. The residue was purified by silica gel column chromatography (EtOAc/hexane and NH, EtOAc/hexane) to give the title compound (18 mg).

¹H NMR (300 MHz, DMSO-d₆) § 1.40-1.58 (4H, m), 1.82-1.93 (1H, m), 2.09-2.19 (1H, m), 2.36 (3H, s), 3.09 (1H, dd, J=10.4, 5.5 Hz), 3.17-3.28 (3H, m), 3.45 (1H, dd, J=10.4, 6.6 Hz), 3.70 (1H, dd, J=15.6, 8.8 Hz), 4.03-4.09 (1H, m), 5.21 (1H, t, J=9.2 Hz), 7.19-7.29 (2H, m), 7.49-7.60 (1H, m), 7.62 (1H, d, J=1.9 Hz), 8.33 (1H, d, J=7.9 Hz), 8.54 (1H, d, J=1.9 Hz).

### Example 39

### N-[(3R)-1-{5-[5-Chloro-3-(2,4,6-trifluorophenyl)pyridin-2-yl]-4,5-dihydro-1,2-oxazol-3-yl}-4,4-difluoropyrrolidin-3-yl]methanesulfonamide (optical isomer)

### A) tert-Butyl [6-chloro-5-(2,4,6-trifluorophenyl)pyridin-3-yl]carbamate

Tri-tert-butylphosphonium tetrafluoroborate (0.755 g) and Pd2(dba)3 (0.794 g) were added to a mixture of tert-butyl (5-bromo-6-chloropyridin-3-yl) carbamate (5.34 g), (2,4,6-trifluorophenyl) boronic acid (3.66 g) and potassium fluoride (2.02 g) in THF (55 mL) and water (11 mL) at room temperature. The mixture was stirred at 80° C. under nitrogen atmosphere for 2 hours. The mixture was poured into water and the resulting mixture was extracted with EtOAc. The organic layer was separated, washed with brine, dried over anhydrous sodium sulfate, and concentrated under reduced pressure. The residue was purified by NH silica gel column chromatography (EtOAc/hexane) to give the title compound (5.58 g).

¹H NMR (300 MHZ, CDCl₃) δ 1.52 (9H, s), 6.59 (1H, br s), 6.78 (2H, dd, J=8.7, 7.3 Hz), 8.09 (1H, br s), 8.27 (1H, d, J=2.8 Hz).

### B) tert-Butyl [6-Ethenyl-5-(2,4,6-trifluorophenyl)pyridin-3-yl]carbamate

Pd(dppf) Cl₂·CH₂C12 (221 mg) was added to a mixture of tert-butyl [6-chloro-5-(2,4,6-trifluorophenyl)pyridin-3-yl]carbamate (648 mg), potassium vinyltrifluoroborate (532 mg), potassium carbonate (749 mg) in DME (10 mL) and water (2.00 mL) at room temperature. The mixture was stirred at 110° C. under argon atmosphere for 3.5 hours under microwave irradiation. The organic layer was separated and concentrated under reduced pressure. The residue was purified by NH silica gel column chromatography (EtOAc/hexane) to give the title compound (510 mg).

MS: [M+H]⁺ 351.0.

### C) tert-Butyl [6-(3-bromo-4,5-dihydro-1,2-oxazol-5-yl)-5-(2,4,6-trifluorophenyl)pyridin-3-yl]carbamate

Sodium hydrogen carbonate (489 mg) was added to a mixture of tert-butyl [6-ethenyl-5-(2,4,6-trifluorophenyl)pyridin-3-yl]carbamate (510 mg) and hydroxycarbonimidic dibromide (591 mg) in EtOAc (6.0 mL) at room temperature. The mixture was stirred at 70° C. overnight. The precipitate was removed by filtration and the filtrate was concentrated under reduced pressure. The residue was purified by silica gel column chromatography (EtOAc/hexane) to give the title compound (577 mg).

MS: [M+H]⁺ 472.0.

### D) 2-(3-Bromo-4,5-dihydro-1,2-oxazol-5-yl)-5-chloro-3-(2,4,6-trifluorophenyl)pyridine

TFA (3 mL) was added to tert-Butyl [6-(3-bromo-4,5-dihydro-1,2-oxazol-5-yl)-5-(2,4,6-trifluorophenyl)pyridin-3-yl]carbamate (577 mg) in a flask at 0° C. The mixture was stirred at room temperature for 30 minutes. After evaporation of the solvent under reduced pressure, the residue was dissolved to conc. hydrochloric acid (5 mL). To the mixture was added sodium nitrite (253 mg) in water (1 mL) dropwise at 0° C. The mixture was stirred at 0° C. for 10 minutes. Copper (I) chloride (726 mg) was added portionwise. The mixture was stirred at 0° C. for 1 hour. To the mixture was added EtOAca and water, and the mixture was extracted with EtOAc. The organic layer was separated, washed with brine, dried over anhydrous sodium sulfate and concentrated under reduced pressure. The residue was purified by silica gel column chromatography (EtOAc/hexane) to give the title compound (270 mg).

MS: [M+H]⁺ 390.9.

### E) N-[(3R)-1-{5-[5-Chloro-3-(2,4,6-trifluorophenyl)pyridin-2-yl]-4,5-dihydro-1,2-oxazol-3-yl}-4,4-difluoropyrrolidin-3-yl]methanesulfonamide

A mixture of 2-(3-bromo-4,5-dihydro-1,2-oxazol-5-yl)-5-chloro-3-(2,4,6-trifluorophenyl)pyridine (100 mg), N-[(3R)-4,4-difluoropyrrolidin-3-yl]methanesulfonamide hydrochloride (121 mg) in pyridine (0.638 mL) was stirred in a sealed tube at 140° C. for 15 hours. The mixture was purified by silica gel column chromatography (EtOAc/hexane) to give the title compound (95 mg).

¹H NMR (400 MHZ, CDCl₃) δ 3.09 (3H, s), 3.12-3.41 (2H, m), 3.69-3.95 (4H, m), 4.34 (1H, dtd, J=13.3, 8.9, 4.3 Hz), 4.80-4.95 (1H, m), 5.49 (1H, t, J=8.8 Hz), 6.63-7.05 (2H, m), 7.62 (1H, d, J=2.3 Hz), 8.53-8.79 (1H, m).

### F) N-[(3R)-1-{5-[5-Chloro-3-(2,4,6-trifluorophenyl)pyridin-2-yl]-4,5-dihydro-1,2-oxazol-3-yl}-4,4-difluoropyrrolidin-3-yl]methanesulfonamide (optical isomer)

N-[(3R)-1-{5-[5-Chloro-3-(2,4,6-trifluorophenyl)pyridin-2-yl]-4,5-dihydro-1,2-oxazol-3-yl}-4,4-difluoropyrrolidin-3-yl]methanesulfonamide (77.5 mg) was optically resolved by HPLC. The fraction with longer retention time was concentrated under reduced pressure to give the title compound (40.2 mg).

### Chiral Separation Condition

Column: CHIRALPAK IB-N5 (trade name) (20 mmID*250 mmL, 5 μm)

Mobile Phase: Hexane/EtOH=600/400 (v/v)

¹H NMR (300 MHz, CDCl₃) δ 3.09 (3H, s), 3.14-3.39 (2H, m), 3.64-3.98 (4H, m), 4.34 (1H, dt, J=13.8, 8.9 Hz), 4.75 (1H, d, J=9.8 Hz), 5.50 (1H, t, J=8.8 Hz), 6.72-6.87 (2H, m), 7.59-7.64 (1H, m), 8.65 (1H, d, J=2.3 Hz).

### Example 42

### N-[(3R)-1-{5-[5-Chloro-3-(2,4,6-trifluorophenyl)pyridin-2-yl]-4,5-dihydro-1,2-oxazol-3-yl}-4,4-difluoropyrrolidin-3-yl]ethanesulfonamide (optical isomer)

### A) tert-Butyl (4R)-4-[(ethanesulfonyl) amino]-3,3-difluoropyrrolidine-1-carboxylate

To a solution of tert-butyl (R)-4-amino-3,3-difluoropyrrolidine-1-carboxylate (1.13 g) and TEA (3.53 mL) in DME (20 mL) was added ethanesulfonyl chloride (0.675 mL) at 5° C. The mixture was stirred at room temperature for 3 hours. The mixture was diluted with EtOAc and poured into saturated sodium hydrogen carbonate aqueous solution at 0° C. The organic layer was separated, washed with water and brine, dried over anhydrous sodium sulfate, and concentrated under reduced pressure. The residue was purified by silica gel column chromatography (EtOAc/hexane) to give the title compound (0.708 g).

¹H NMR (300 MHz, CDCl₃) δ 1.42 (3H, t, J=7.3 Hz), 1.46 (9H, s), 3.06-3.26 (3H, m), 3.62-3.90 (2H, m), 3.91-4.09 (1H, m), 4.12-4.36 (1H, m), 4.57 (1H, d, J=9.8 Hz).

### B) N-[(3R)-4,4-Difluoropyrrolidin-3-yl]ethanesulfonamide hydrochloride

To a solution of tert-butyl (4R)-4-[(ethanesulfonyl)amino]-3,3-difluoropyrrolidine-1-carboxylate (708 mg) in EtOAc (5.0 mL) was added 4 M hydrogen chloride in EtOAc (10 mL) at room temperature. The mixture was stirred at room temperature over weekend. Precipitated solid was collected by filtration, washed with EtOAc, and dried under reduced pressure to give the title compound (513 mg).

¹H NMR (300 MHz, DMSO-d₆) δ 1.15-1.30 (3H, m), 2.98-3.16 (3H, m), 3.58-3.88 (3H, m), 4.23-4.52 (1H, m), 8.07 (1H, d, J=9.0 Hz), 9.15-9.77 (2H, m).

### C) N-[(3R)-1-{5-[5-Chloro-3-(2,4,6-trifluorophenyl)pyridin-2-yl]-4,5-dihydro-1,2-oxazol-3-yl}-4,4-difluoropyrrolidin-3-yl]ethanesulfonamide

A mixture of 2-(3-bromo-4,5-dihydro-1,2-oxazol-5-yl)-5-chloro-3-(2,4,6-trifluorophenyl)pyridine (100 mg), N-[(3R)-4,4-difluoropyrrolidin-3-yl]ethanesulfonamide hydrochloride (128 mg) in pyridine (0.638 mL) was stirred in a sealed tube at 140° ° C. for 15 hours. The mixture was purified by silica gel column chromatography (EtOAc/hexane) to give the title compound (115 mg).

¹H NMR (400 MHZ, CDCl₃) δ 1.42 (3H, t, J=7.3 Hz), 2.97-3.44 (4H, m), 3.63-3.99 (4H, m), 4.22-4.44 (1H, m), 4.70 (1H, t, J=10.6 Hz), 5.49 (1H, t, J=8.7 Hz), 6.57-6.93 (2H, m), 7.62 (1H, d, J=2.2 Hz), 8.64 (1H, dd, J=2.2, 1.5 Hz).

### D) N-[(3R)-1-{5-[5-Chloro-3-(2,4,6-trifluorophenyl)pyridin-2-yl]-4,5-dihydro-1,2-oxazol-3-yl}-4,4-difluoropyrrolidin-3-yl]ethanesulfonamide (optical isomer)

N-[(3R)-1-{5-[5-Chloro-3-(2,4,6-trifluorophenyl)pyridin-2-yl]-4,5-dihydro-1,2-oxazol-3-yl}-4,4-difluoropyrrolidin-3-yl]ethanesulfonamide (101 mg) was optically resolved by HPLC.

The fraction with longer retention time was concentrated under reduced pressure. The residue was washed with IPE to give the title compound (38.7 mg).

### Chiral Separation Condition

Column: CHIRALPAK IB-N5 (trade name) (20 mmID*250 mmL, 5 μm)

Mobile Phase: Hexane/Ethanol=700/300 (v/v)

¹H NMR (300 MHz, CDCl₃) δ 1.42 (3H, t, J=7.3 Hz), 3.04-3.35 (4H, m), 3.64-4.00 (4H, m), 4.21-4.42 (1H, m), 4.63 (1H, d, J=10.2 Hz), 5.49 (1H, t, J=8.7 Hz), 6.70-6.88 (2H, m), 7.58-7.64
(1H, m), 8.65 (1H, d, J=2.3 Hz).

### Example 49

### N-[(3R)-1-{5-[5-Chloro-3-(2,6-difluorophenyl)pyridin-2-yl]-4,5-dihydro-1,2-oxazol-3-yl}-4,4-difluoropyrrolidin-3-yl]methanesulfonamide (optical isomer)

### A) 5-Chloro-3-(2,6-difluorophenyl)pyridin-2-amine

A mixture of 3-bromo-5-chloro-pyridin-2-amine (15.0 g), (2,6-difluorophenyl) boronic acid (14.8 g), Pd2(dba)3 (3.31 g), tri-tert-butylphosphonium tetrafluoroborate (3.15 g) and potassium fluoride (8.40 g) in THF (150 mL) and water (30 mL) was stirred at 80° C. for 1 hour under nitrogen atmosphere. The reaction mixture was diluted with water and the resulting mixture was extracted with EtOAc. The combined organic layer was washed with brine, dried over anhydrous sodium sulfate and concentrated under reduced pressure. The residue was purified by silica gel column chromatography (EtOAc/petroleum ether) to give the title compound (17.0 g).

¹H NMR (400 MHz, DMSO-d₆) δ 5.98 (2H, s), 7.16-7.24 (2H, m), 7.46-7.57 (2H, m), 8.03 (1H, d, J=2.4 Hz).

### B) 5-Chloro-3-(2,6-difluorophenyl)-2-iodopyridine

To a mixture of 5-chloro-3-(2,6-difluorophenyl)pyridin-2-amine (8.50 g), diiodomethane (37.8 g) and copper (I) iodide (10.1 g) in THE (90 mL) was added n-pentyl nitrite (8.28 g) at room temperature. The mixture was stirred at 70° C. for 5 hours. The solid was removed by filtration, and the filtrate was concentrated in vacuo. The residue was purified by silica gel column chromatography (EtOAc/petroleum ether) to give the title compound (6.70 g).

¹H NMR (400 MHZ, DMSO-d₆) δ 7.26-7.32 (2H, m), 7.60-7.69 (1H, m), 8.10 (1H, d, J=2.4 Hz), 8.58 (1H, d, J=2.4 Hz).

### C) 5-Chloro-3-(2,6-difluorophenyl)-2-ethenylpyridine

To a mixture of 5-chloro-3-(2,6-difluorophenyl)-2-iodopyridine (6.70 g), potassium vinyltrifluoroborate (5.11 g) and potassium carbonate (5.27 g) in DME (100 mL) and water (20 mL) was added Pd(dppf) Cl₂·CH₂Cl₂ (2.33 g). The mixture was stirred at 100° C. for 14 hours under nitrogen atmosphere. The reaction mixture was diluted with water and the resulting mixture was extracted with EtOAc. The combined organic layer was washed with brine, dried over anhydrous sodium sulfate and concentrated under reduced pressure. The residue was purified by silica gel column chromatography (EtOAc/petroleum ether) to give the title compound (3.20 g).

¹H NMR (400 MHZ, DMSO-d₆) δ 5.46-5.51 (1H, m), 6.35-6.40 (1H, m), 6.41-6.48 (1H, m), 7.27-7.32 (2H, m), 7.57-7.66 (1H, m), 8.05 (1H, d, J=2.4 Hz), 8.73 (1H, d, J=2.4 Hz).

### D) 2-(3-Bromo-4,5-dihydro-1,2-oxazol-5-yl)-5-chloro-3-(2,6-difluorophenyl)pyridine

To a solution of hydroxycarbonimidic dibromide (11.1 g) and 5-chloro-3-(2,6-difluorophenyl)-2-ethenylpyridine (6.90 g) in EtOAc (70 mL) was added sodium hydrogen carbonate (9.21 g). The mixture was stirred at 60° ° C. for 14 hours. The reaction mixture was concentrated under reduced pressure. The residue was purified by silica gel column chromatography (EtOAc/petroleum ether) to give the title compound (8.50 g).

¹H NMR (400 MHZ, DMSO-d₆) δ 3.62-3.75 (2H, m), 5.48-5.55 (1H, m), 7.30 (2H, q, J=9.2 Hz), 7.58-7.67 (1H, m), 8.17 (1H, d, J=2.4 Hz), 8.84 (1H, d, J=2.4 Hz).

### E) 2-(3-Bromo-4,5-dihydro-1,2-oxazol-5-yl)-5-chloro-3-(2,6-difluorophenyl)pyridine (optical isomer)

2-(3-Bromo-4,5-dihydro-1,2-oxazol-5-yl)-5-chloro-3-(2,6-difluorophenyl)pyridine (8.50 g) was optically resolved by SFC. The fraction with longer retention time was concentrated under reduced pressure to give the title compound (4.02 g).

### Chiral Separation Condition

Column: CHIRALPAK AY (trade name) (250 mm*50 mm, 10 μm)

Mobile Phase: CO2/EtOH including 0.1% ammonium hydroxide=850/150 (v/v)

¹H NMR (400 MHZ, DMSO-d₆) δ 3.66-3.72 (2H, m), 5.48-5.54 (1H, m), 7.30 (2H, q, J=9.2 Hz), 7.58-7.67 (1H, m), 8.17 (1H, d, J=2.4 Hz), 8.84 (1H, d, J=2.4 Hz).

### F) N-[(3R)-1-{5-[5-Chloro-3-(2,6-difluorophenyl)pyridin-2-yl]-4,5-dihydro-1,2-oxazol-3-yl}-4,4-difluoropyrrolidin-3-yl]methanesulfonamide (optical isomer)

A mixture of 2-(3-bromo-4,5-dihydro-1,2-oxazol-5-yl)-5-chloro-3-(2,6-difluorophenyl)pyridine (optical isomer) (1.00 g), N-[(3R)-4,4-difluoropyrrolidin-3-yl]methanesulfonamide hydrochloride (1.27 g) in pyridine (9 mL) was stirred in a sealed tube at 140° C. overnight. The mixture was quenched with water and the resulting mixture was extracted with EtOAc. The organic layer was separated, washed with 1 M hydrogen chloride aqueous solution and brine, dried over anhydrous magnesium sulfate and concentrated under reduced pressure. The residue was purified by column chromatography (EtOAc/hexane) to give the title compound (671 mg).

¹H NMR (300 MHZ, DMSO-d₆) δ 2.99 (3H, s), 3.12-3.23 (1H, m), 3.27-3.37 (1H, m), 3.56-3.87 (4H, m), 4.23-4.42 (1H, m), 5.29 (1H, t, J=9.0 Hz), 7.23-7.32 (2H, m), 7.55-7.66 (1H, m), 7.98 (1H, s), 8.12 (1H, d, J=2.1 Hz), 8.80 (1H, d, J=2.4 Hz).

### Example 50

### N-[(3S)-1-{5-[2-(3,5-Difluoropyridin-2-yl)-4-methylphenyl]-4,5-dihydro-1,2-oxazol-3-yl}pyrrolidin-3-yl]methanesulfonamide

### A) 2-(2-Chloro-5-methylphenyl)-3,5-difluoropyridine

A mixture of (2-chloro-5-methylphenyl) boronic acid (527 mg), 2-bromo-3,5-difluoropyridine (500 mg), Pd(Ph3P) 4 (149 mg) and sodium carbonate (546 mg) in DME (10 mL) and water (2.50 mL) was stirred at 80° C. under argon atmosphere for 3.5 hours. Water was added to the mixture and the mixture was extracted with EtOAc. The organic layer was washed with water and brine, dried over anhydrous sodium sulfate, and concentrated under reduced pressure. The residue was purified by silica gel column chromatography (EtOAc/hexane) to give the title compound (544 mg).

MS: [M+H]⁺ 239.9.

### B) 2-(2-Ethenyl-5-methylphenyl)-3,5-difluoropyridine

A mixture of 2-(2-chloro-5-methylphenyl)-3,5-difluoropyridine (540 mg), 4,4,5,5-tetramethyl-2-vinyl-1,3,2-dioxaborolane (0.956 mL), Pd2(dba)3 (206 mg), 2-dicyclohexylphosphino-2′,6′-dimethoxy-1, l′-biphenyl (278 mg) and sodium carbonate (478 mg) in toluene (10 mL) and water (2.3 mL) was heated at 110° C. for 8 hours under microwave irradiation. Water was added to the reaction mixture and the mixture was extracted with EtOAc. The organic layer was washed with water and brine, dried over anhydrous sodium sulfate, and concentrated under reduced pressure. The residue was purified by silica gel column chromatography (EtOAc/hexane) to give the title compound (504 mg).

MS: [M+H]⁺ 231.9.

### C) 2-[2-(3-Bromo-4,5-dihydro-1,2-oxazol-5-yl)-5-methylphenyl]-3,5-difluoropyridine

A mixture of 2-(2-ethenyl-5-methylphenyl)-3,5-difluoropyridine (500 mg), hydroxycarbonimidic dibromide (877 mg) and sodium hydrogen carbonate (727 mg) in EtOAc (7.5 mL) was stirred at 65° C. under nitrogen atmosphere for 7 hours. The solid was removed by filtration and the filtrate was concentrated under reduced pressure. The residue was purified by silica gel column chromatography (EtOAc/hexane) to give the title compound (617 mg).

MS: [M+H]⁺ 352.9.

### D) N-[(3S)-1-{5-[2-(3,5-Difluoropyridin-2-yl)-4-methylphenyl]-4,5-dihydro-1,2-oxazol-3-yl}pyrrolidin-3-yl]methanesulfonamide

A mixture of 2-[2-(3-bromo-4,5-dihydro-1,2-oxazol-5-yl)-5-methylphenyl]-3,5-difluoropyridine (20.0 mg), N-[(3S)-pyrrolidin-3-yl]methanesulfonamide hydrochloride (28.4 mg) and DIPEA (0.099 mL) in NMP (0.35 mL) was stirred in a sealed tube at 120° C. for 6 hours. The mixture was purified by preparative HPLC (C18, mobile phase: water (containing 10 mM ammonium bicarbonate)/acetonitrile). The desired fraction was collected and extracted with EtOAc, washed with water and brine, dried over anhydrous sodium sulfate and concentrated under reduced pressure to give the title compound (3.2 mg).

¹H NMR (300 MHz, DMSO-d₆) δ 1.73-1.90 (1H, m), 2.03-2.17 (1H, m), 2.34 (3H, s), 2.85-2.92 (1H, m), 2.94 (3H, s), 2.99-3.08 (1H, m), 3.13-3.28 (3H, m), 3.38-3.49 (1H, m), 3.85-3.98 (1H, m), 5.15-5.25 (1H, m), 7.16 (1H, s), 7.31-7.39 (2H, m), 7.54 (1H, d, J=7.9 Hz), 8.08 (1H, td, J=9.4, 2.5 Hz), 8.61 (1H, d, J=2.4 Hz).

### Example 59

### N-[(3R)-1-{5-(Difluoromethyl)-5-[3-(2,6-difluorophenyl)-5-methylpyridin-2-yl]-4,5-dihydro-1,2-oxazol-3-yl}-4,4-difluoropyrrolidin-3-yl]methanesulfonamide (optical isomer)

### A) 2-[3-(2,6-Difluorophenyl)-5-methylpyridin-2-yl]prop-2-en-1-ol

Pd(dppf) Cl₂·CH₂C12 (348 mg) was added to a mixture of 2-(4,4,5,5-tetramethyl-1,3,2-dioxaborolan-2-yl) prop-2-en-1-ol (783 mg), 2-chloro-3-(2,6-difluorophenyl)-5-methylpyridine (510 mg), potassium carbonate (588 mg) in DME (12 mL) and water (2.4 mL) at room temperature. The mixture was stirred at 110° C. under argon atmosphere for 3 hours under microwave irradiation. The organic layer was separated and concentrated under reduced pressure. The residue was purified by NH silica gel column chromatography (EtOAc/hexane) to give the title compound (616 mg).

MS: [M+H]⁺ 262.0.

### B) {3-Bromo-5-[3-(2,6-difluorophenyl)-5-methylpyridin-2-yl]-4,5-dihydro-1,2-oxazol-5-yl}methanol

Sodium hydrogen carbonate (3.01 g) was added to a mixture of 2-[3-(2,6-difluorophenyl)-5-methylpyridin-2-yl]prop-2-en-1-ol (2.34 g) and hydroxycarbonimidic dibromide (3.63 g) in EtOAc (35.8 mL) at room temperature. The mixture was stirred at 70° C. overnight. The precipitate was removed by filtration and the filtrate was concentrated under reduced pressure. The residue was purified by silica gel column chromatography (EtOAc/hexane) to give the title compound (2.14 g).

MS: [M+H]⁺ 382.9.

### C) 2-[3-Bromo-5-(difluoromethyl)-4,5-dihydro-1,2-oxazol-5-yl]-3-(2,6-difluorophenyl)-5-methylpyridine

Oxalyl chloride (0.110 mL) was added to a solution of DMSO (0.119 mL) in THF (4.18 mL) at −78° C. The mixture was stirred at −78° C. under nitrogen atmosphere for 10 minutes. {3-Bromo-5-[3-(2,6-difluorophenyl)-5-methylpyridin-2-yl]-4,5-dihydro-1,2-oxazol-5-yl}methanol (160 mg) in THF (5 mL) was added. The mixture was stirred at −78° C. under nitrogen atmosphere for 30 minutes. TEA (0.291 mL) was added. The mixture was stirred at −78° C. to room temperature for 30 minutes. The mixture was quenched with saturated sodium hydrogen carbonate aqueous solution and the resulting mixture was extracted with EtOAc. The organic layer was separated, washed with brine, dried over anhydrous sodium sulfate and concentrated under reduced pressure to give the residue. DAST (0.089 mL) was added to a solution of the residue in CH₂Cl₂ (4 mL) at 0° C. The mixture was gradually warmed up to room temperature and stirred at room temperature under nitrogen atmosphere over weekend. The mixture was quenched with water at room temperature and the resulting mixture was extracted with EtOAc. The organic layer was separated, washed with brine, dried over anhydrous sodium sulfate and concentrated under reduced pressure. The residue was purified by silica gel column chromatography (EtOAc/hexane) to give the title compound (76 mg).

MS: [M+H]⁺ 402.9.

### D) N-[(3R)-1-{5-(Difluoromethyl)-5-[3-(2,6-difluorophenyl)-5-methylpyridin-2-yl]-4,5-dihydro-1,2-oxazol-3-yl}-4,4-difluoropyrrolidin-3-yl]methanesulfonamide

A mixture of 2-[3-bromo-5-(difluoromethyl)-4,5-dihydro-1,2-oxazol-5-yl]-3-(2,6-difluorophenyl)-5-methylpyridine (76 mg), N-[(3R)-4,4-difluoropyrrolidin-3-yl]methanesulfonamide hydrochloride (89 mg) in pyridine (0.47 mL) was stirred in a sealed tube at 140° C. for 15 hours. The mixture was purified by silica gel column chromatography (EtOAc/hexane) to give the title compound (41 mg).

¹H NMR (300 MHZ, CDCl₃) δ 2.39 (3H, s), 3.07 (3H, s), 3.13-3.29 (1H, m), 3.49-3.99 (5H, m), 4.30 (1H, dt, J=14.1, 8.8 Hz), 4.76 (1H, dd, J=9.8, 3.4 Hz), 5.68-6.32 (1H, m), 6.84-7.11 (2H, m), 7.30-7.43 (2H, m), 8.48 (1H, s).

### E) N-[(3R)-1-{5-(Difluoromethyl)-5-[3-(2,6-difluorophenyl)-5-methylpyridin-2-yl]-4,5-dihydro-1,2-oxazol-3-yl}-4,4-difluoropyrrolidin-3-yl]methanesulfonamide (optical isomer)

N-[(3R)-1-{5-(Difluoromethyl)-5-[3-(2,6-difluorophenyl)-5-methylpyridin-2-yl]-4,5-dihydro-1,2-oxazol-3-yl}-4,4-difluoropyrrolidin-3-yl]methanesulfonamide (40.2 mg) was optically resolved by HPLC. The fraction with shorter retention time was concentrated under reduced pressure to give the title compound (17.8 mg).

### Chiral Separation Condition

Column: CHIRALPAK IG (trade name) (20 mmID*250 mmL, 5 μm)

Mobile Phase: Hexane/EtOH=500/500 (v/v)

¹H NMR (300 MHZ, CDCl₃) δ 2.39 (3H, s), 3.07 (3H, s), 3.20 (1H, t, J=9.4 Hz), 3.52 (1H, d, J=16.9 Hz), 3.64 (2H, dd, J=19.0, 8.5 Hz), 3.82 (1H, dd, J=10.7, 7.7 Hz), 3.90-4.02 (1H, m), 4.25-4.42 (1H, m), 4.72 (1H, d, J=10.5 Hz), 5.87-6.31 (1H, m), 6.91-7.06 (2H, m), 7.34-7.42 (2H, m), 8.46-8.52 (1H, m).

### Example 64

### N-(4,4-Difluoro-3-methyl-1-{(5S)-5-[5-methyl-3-(2,4,6-trifluorophenyl)pyridin-2-yl]-4,5-dihydro-1,2-oxazol-3-yl}pyrrolidin-3-yl) methanesulfonamide

### A) Benzyl 3-[(tert-butoxycarbonyl)amino]-4,4-difluoro-3-methylpyrrolidine-1-carboxylate

To a solution of 1-((benzyloxy) carbonyl)-4,4-difluoro-3-methylpyrrolidine-3-carboxylic acid (161 mg) in t-BuOH (2 mL) were added TEA (0.150 mL) and DPPA (0.175 mL) at room temperature. The mixture was stirred at 100° C. overnight. The mixture was purified by silica gel column chromatography (EtOAc/hexane) to give the title compound (130 mg).

MS: [M+Na]+393.1.

### B) Benzyl 3,3-difluoro-4-[(methanesulfonyl) amino]-4-methylpyrrolidine-1-carboxylate

A mixture of benzyl 3-[(tert-butoxycarbonyl) amino]-4,4-difluoro-3-methylpyrrolidine-1-carboxylate (130 mg) and 4 M hydrogen chloride in CPME (1 mL) was stirred at room temperature for 1 hour. The mixture was concentrated under reduced pressure. The residue was combined with THE (2 mL), and then methanesulfonyl chloride (0.136 mL) and TEA (0.391 mL) were added. The mixture was stirred at room temperature overnight. The mixture was quenched with water and the resulting mixture was extracted with EtOAc. The organic layer was separated, washed with brine, dried over anhydrous magnesium sulfate and concentrated under reduced pressure. The residue was purified by silica gel column chromatography (EtOAc/hexane) to give the title compound (37.2 mg).

MS: [M+Na]+371.0.

### C) N-(4,4-Difluoro-3-methylpyrrolidin-3-yl) methanesulfonamide

Under hydrogen atmosphere, a mixture of benzyl 3,3-difluoro-4-[(methanesulfonyl)amino]-4-methylpyrrolidine-1-carboxylate (45.6 mg), 10% palladium on carbon (10 mg) and MeOH (1 mL) was stirred at room temperature overnight. The catalyst was filtered off, and the filtrate was concentrated under reduced pressure. The residue was combined with 20% palladium hydroxide on carbon (10 mg) and MeOH (1 mL). The mixture was stirred at room temperature under hydrogen atmosphere for 1 hour. The catalyst was filtered off, and the filtrate was concentrated under reduced pressure to give the title compound (26.5 mg).

¹H NMR (300 MHZ, CDCl₃) δ 1.56 (3H, d, J=3.0 Hz), 3.12 (3H, s), 3.15-3.51 (4H, m).

### D) N-(4,4-Difluoro-3-methyl-1-{(5S)-5-[5-methyl-3-(2,4,6-trifluorophenyl)pyridin-2-yl]-4,5-dihydro-1,2-oxazol-3-yl}pyrrolidin-3-yl) methanesulfonamide

A mixture of 2-[(5S)-3-bromo-4,5-dihydro-1,2-oxazol-5-yl]-5-methyl-3-(2,4,6-trifluorophenyl)pyridine (45.9 mg), N-(4,4-difluoro-3-methylpyrrolidin-3-yl)methanesulfonamide (26.5 mg) and pyridine (0.3 mL) was stirred in a sealed tube at 140° C. overnight. The mixture was purified by preparative HPLC (C18, mobile phase: water (containing 10 mM ammonium bicarbonate)/acetonitrile). Then the desired fractions were concentrated to give the title compound (6.8 mg).

¹H NMR (300 MHZ, DMSO-d₆) δ 1.51 (3H, s), 2.36 (3H, s), 3.03 (3H, d, J=1.9 Hz), 3.22-3.31 (1H, m), 3.41-3.51 (1H, m), 3.59-3.84 (4H, m), 5.27 (1H, t, J=9.6 Hz), 7.28-7.44 (2H, m), 7.54-8.02 (2H, m), 8.54 (1H, d, J=1.8 Hz).

### Example 72

### N-[(3R)-4,4-Difluoro-1-{5-[6-methoxy-4-(2,4,6-trifluorophenyl)pyridin-3-yl]-4,5-dihydro-1,2-oxazol-3-yl}pyrrolidin-3-yl]methanesulfonamide

### A) 2-Methoxy-4-(2,4,6-trifluorophenyl)pyridine

To a solution of 4-bromo-2-methoxypyridine (1.59 g) and (2,4,6-trifluorophenyl) boronic acid (2.97 g) in DME (20 mL) and water (10.0 mL) was added potassium fluoride (1.47 g), Pd2(dba)3 (0.581 g) and tri-t-butylphosphonium tetrafluoroborate (0.491 g) and the reaction mixture was stirred for 1 hour at 100° C. under nitrogen atmosphere. After cooling to room temperature, the mixture was quenched with water and the resulting mixture was extracted with EtOAc. The organic layer was separated, washed with brine, dried over anhydrous magnesium sulfate and concentrated under reduced pressure. The residue was purified by silica gel column chromatography (EtOAc/hexane) to give the title compound (1.67 g).

MS: [M+H]⁺ 240.0.

### B) 5-Bromo-2-methoxy-4-(2,4,6-trifluorophenyl)pyridine

To a solution of 2-methoxy-4-(2,4,6-trifluorophenyl)pyridine (900 mg) in DMF (20 mL) was added NBS (1.01 g) and the reaction mixture was stirred for 3 hours at 100° C. After cooling to room temperature, the mixture was quenched with water and the resulting mixture was extracted with EtOAc. The organic layer was separated, washed with brine, dried over anhydrous magnesium sulfate and concentrated under reduced pressure. The residue was purified by silica gel column chromatography (EtOAc/hexane) to give the title compound (300 mg).

MS: [M+H]⁺ 317.9.

### C) 5-Ethenyl-2-methoxy-4-(2,4,6-trifluorophenyl)pyridine

The mixture of 5-bromo-2-methoxy-4-(2,4,6-trifluorophenyl)pyridine (300 mg), potassium vinyltrifluoroborate (253 mg), potassium carbonate (261 mg) and Pd(dppf) Cl₂·CH₂Cl₂ (116 mg) in DME (4 mL) and water (1.00 mL) was heated at 110° C. for 2 hours under microwave irradiation. After cooling to room temperature, to the mixture were added potassium carbonate (130 mg), potassium vinyltrifluoroborate (126 mg) and bis(di-tert-butyl (4-dimethylaminophenyl)phosphine)dichloropalladium(II) (95 mg), and the mixture was heated at 120° C. for 2 hours under microwave irradiation. After cooling to room temperature, the reaction mixture was concentrated. The residue was purified by silica gel column chromatography (EtOAc/hexane) to give the title compound (223 mg).

MS: [M+H]⁺ 265.9.

### D) 5-(3-Bromo-4,5-dihydro-1,2-oxazol-5-yl)-2-methoxy-4-(2,4,6-trifluorophenyl)pyridine

To a solution of 5-ethenyl-2-methoxy-4-(2,4,6-trifluorophenyl)pyridine (220 mg) and hydroxycarbonimidic dibromide (336 mg) in EtOAc (10 mL) was added sodium hydrogen carbonate (279 mg) and the reaction mixture was stirred for 10 hours at 70° C. After cooling to room temperature, the reaction mixture was concentrated. The residue was purified by silica gel column chromatography (EtOAc/hexane) to give the title compound (226 mg).

¹H NMR (300 MHZ, DMSO-d₆) δ 3.24 (1H, dd, J=17.5, 9.6 Hz), 3.59 (1H, dd, J=17.5, 11.5 Hz), 3.91 (3H, s), 5.49 (1H, t, J=10.4 Hz), 6.77-7.01 (1H, m), 7.27-7.49 (2H, m), 8.38 (1H, s).

### E) N-[(3R)-4,4-Difluoro-1-{5-[6-methoxy-4-(2,4,6-trifluorophenyl)pyridin-3-yl]-4,5-dihydro-1,2-oxazol-3-yl}pyrrolidin-3-yl]methanesulfonamide

To a solution of 5-(3-bromo-4,5-dihydro-1,2-oxazol-5-yl)-2-methoxy-4-(2,4,6-trifluorophenyl)pyridine (50 mg) and N-[(3R)-4,4-difluoropyrrolidin-3-yl]methanesulfonamide hydrochloride (45.8 mg) in NMP (0.5 mL) was added DIPEA (0.226 mL) and the reaction mixture was stirred for 24 hours at 130° C. in a sealed tube. Then The crude material was purified by preparative HPLC (C18, mobile phase: water (containing 10 mM ammonium bicarbonate)/acetonitrile) and the desired fractions were concentrated, and the residue was purified by silica gel column chromatography (EtOAc/hexane) to give the title compound (11 mg).

¹H NMR (300 MHZ, CDCl₃) δ 2.78-2.98 (1H, m), 3.07 (3H, s), 3.10-3.30 (2H, m), 3.62-3.87 (3H, m), 3.95-4.01 (3H, m), 4.22-4.43 (1H, m), 5.03-5.18 (1H, m), 5.25-5.41 (1H, m), 6.62 (1H, s), 6.75-6.90 (2H, m), 8.45 (1H, s).

### Example 73

### N-[1-{(5S)-5-[3-(2,6-Difluorophenyl)-5-methylpyridin-2-yl]-4,5-dihydro-1,2-oxazol-3-yl}-3-(methoxymethyl) pyrrolidin-3-yl]methanesulfonamide (optical isomer)

### A) Benzyl 3-[(methanesulfonyl)amino]-3-(methoxymethyl) pyrrolidine-1-carboxylate

A mixture of benzyl 3-((tert-butoxycarbonyl)amino)-3-(methoxymethyl) pyrrolidine-1-carboxylate (2.00 g) and 4 M hydrogen chloride in CPME (30 mL) was stirred at room temperature for 2 hours. After evaporation, to a mixture of the residue and TEA (1.53 mL) in THE (20 mL) was added methanesulfonyl chloride (0.637 mL) at 0° C. The mixture was stirred at room temperature under argon atmosphere for 30 minutes. The mixture was neutralized with saturated sodium hydrogen carbonate aqueous solution at 0° C. and the resulting mixture was extracted with EtOAc. The organic layer was separated, washed with water and brine, dried over anhydrous magnesium sulfate and concentrated under reduced pressure. The residue was purified by silica gel column chromatography (EtOAc/hexane) to give the title compound (1.61 g).

MS: [M+H]⁺ 365.0.

### B) N-[1-{(5S)-5-[3-(2,6-Difluorophenyl)-5-methylpyridin-2-yl]-4,5-dihydro-1,2-oxazol-3-yl}-3-(methoxymethyl) pyrrolidin-3-yl]methanesulfonamide

Under hydrogen atmosphere, a mixture of benzyl 3-[(methanesulfonyl)amino]-3-(methoxymethyl) pyrrolidine-1-carboxylate (1.61 g) and 10% palladium on carbon (1 g) in MeOH (15 mL) was stirred at room temperature for 2 hours. The catalyst was removed by filtration and the filtrate was concentrated under reduced pressure. A mixture of 83.0 mg of the residue and 2-[(5S)-3-bromo-4,5-dihydro-1,2-oxazol-5-yl]-3-(2,6-difluorophenyl)-5-methylpyridine (70.0 mg) in NMP (0.6 mL) was stirred in a sealed tube at 120° C. for 6 hours. The mixture was purified by silica gel column chromatography (EtOAc/hexane and MeOH/EtOAc) to give the title compound (75.7 mg).

MS: [M+H]⁺ 481.1

### C) N-[1-{(5S)-5-[3-(2,6-Difluorophenyl)-5-methylpyridin-2-yl]-4,5-dihydro-1,2-oxazol-3-yl}-3-(methoxymethyl) pyrrolidin-3-yl]methanesulfonamide (optical isomer)

N-[1-{(5S)-5-[3-(2,6-Difluorophenyl)-5-methylpyridin-2-yl]-4,5-dihydro-1,2-oxazol-3-yl}-3-(methoxymethyl) pyrrolidin-3-yl]methanesulfonamide (100 mg) was optically resolved by HPLC. The fraction with longer retention time was concentrated under reduced pressure to give the title compound (48.2 mg).

### Chiral Separation Condition

Column: CHIRALPAK IC (trade name) (20 mmID*250 mmL, 5 μm)

Mobile Phase: Hexane/EtOH=250/750 (v/v)

¹H NMR (300 MHz, DMSO-d₆) δ 1.90-2.04 (1H, m), 2.06-2.17 (1H, m), 2.36 (3H, s), 2.94 (3H, s), 3.13-3.37 (7H, m), 3.44 (1H, d, J=11.3 Hz), 3.49 (2H, d, J=1.1 Hz), 3.66 (1H, dd, J=15.4, 8.7 Hz), 5.21 (1H, t, J=9.0 Hz), 7.24 (2H, q, J=8.9 Hz), 7.33 (1H, s), 7.48-7.59 (1H, m), 7.62 (1H, d, J=1.9 Hz), 8.53
(1H, d, J=1.5 Hz)

### Example 82

### 1-Fluoro-N-[(3R,4S)-4-fluoro-1-{(5S)-5-[5-methyl-3-(2,4,6-trifluorophenyl)pyridin-2-yl]-4,5-dihydro-1,2-oxazol-3-yl}pyrrolidin-3-yl]methanesulfonamide

### A) Benzyl [(3R,4S)-4-fluoro-1-{(5S)-5-[5-methyl-3-(2,4,6-trifluorophenyl)pyridin-2-yl]-4,5-dihydro-1,2-oxazol-3-yl}pyrrolidin-3-yl]carbamate

A mixture of 2-[(5S)-3-bromo-4,5-dihydro-1,2-oxazol-5-yl]-5-methyl-3-(2,4,6-trifluorophenyl)pyridine (250 mg), DIPEA (1.18 mL), benzyl ((3R,4S)-4-fluoropyrrolidin-3-yl) carbamate hydrochloride (278 mg) in NMP (2.0 mL) was stirred in a sealed tube at 140° C. overnight. The mixture was purified by silica gel column chromatography (EtOAc/hexane) to give the title compound (138 mg).

MS: [M+H]⁺ 529.1.

### B) (3R,4S)-4-Fluoro-1-{(5S)-5-[5-methyl-3-(2,4,6-trifluorophenyl)pyridin-2-yl]-4,5-dihydro-1,2-oxazol-3-yl}pyrrolidin-3-amine

To a mixture of benzyl [(3R,4S)-4-fluoro-1-{(5S)-5-[5-methyl-3-(2,4,6-trifluorophenyl)pyridin-2-yl]-4,5-dihydro-1,2-oxazol-3-yl}pyrrolidin-3-yl]carbamate (137 mg) in EtOH (2 mL) was added 10% palladium on carbon (20 mg). The mixture was stirred at room temperature under hydrogen atmosphere for 2 hours. The catalyst was removed by filtration, and the filtrate was concentrated under reduced pressure to give the title compound (98 mg).

MS: [M+H]⁺ 395.0.

### C) 1-Fluoro-N-[(3R,4S)-4-fluoro-1-{(5S)-5-[5-methyl-3-(2,4,6-trifluorophenyl)pyridin-2-yl]-4,5-dihydro-1,2-oxazol-3-yl}pyrrolidin-3-yl]methanesulfonamide

To a stirred solution of (3R,4S)-4-fluoro-1-{(5S)-5-[5-methyl-3-(2,4,6-trifluorophenyl)pyridin-2-yl]-4,5-dihydro-1,2-oxazol-3-yl}pyrrolidin-3-amine (30 mg) and DIPEA (0.027 mL) in THF (2 mL) was added fluoromethanesulfonyl chloride (12.1 mg). The mixture was stirred at room temperature for 2 hours. The mixture was quenched with water and the resulting mixture was extracted with EtOAc. The organic layer was separated and dried over anhydrous magnesium sulfate and concentrated under reduced pressure. The residue was purified by preparative HPLC (C18, mobile phase: water (containing 10 mM ammonium bicarbonate)/acetonitrile) and by silica gel column chromatography (EtOAc/hexane) to give the title compound (18.5 mg).

¹H NMR (300 MHZ, CDCl₃) δ 2.40 (3H, s), 3.11-3.24 (1H, m), 3.26-3.37 (1H, m), 3.58-3.77 (2H, m), 3.78-3.93 (2H, m), 4.18-4.36 (1H, m), 5.02-5.16 (2H, m), 5.17-5.27 (2H, m), 5.49 (1H, t, J=9.3 Hz), 6.70-6.85 (2H, m), 7.39-7.46 (1H, m), 8.50-8.57 (1H, m).

### Example 99

### N-[(3S)-1-{(5S)-5-[3-(2,6-Difluorophenyl)-5-methylpyridin-2-yl]-4,5-dihydro-1,2-oxazol-3-yl}pyrrolidin-3-yl]-1-fluoroethane-1-sulfonamide

### A) tert-Butyl (3S)-3-{(Ethanesulfonyl) [(4-methoxyphenyl)methyl]amino}pyrrolidine-1-carboxylate

4-Methoxybenzyl chloride (3.90 mL) was added to a suspension of tert-butyl (3S)-3-[(ethanesulfonyl)amino] pyrrolidine-1-carboxylate (6.67 g) and potassium carbonate (9.93 g) in DMF (50 mL) at room temperature. The mixture was stirred at room temperature over weekend. The mixture was poured into water and the resulting mixture was extracted with EtOAc. The organic layer was separated, washed with water and brine, dried over anhydrous magnesium sulfate and concentrated under reduced pressure. The residue was purified by NH silica gel column chromatography (EtOAc/hexane) to obtain the title compound (9.55 g).

MS: [M+Na]+421.1.

### B) tert-Butyl (3S)-3-{(1-fluoroethanesulfonyl) [(4-methoxyphenyl)methyl]amino}pyrrolidine-1-carboxylate

n-Butyllithium (1.6 M in hexane, 17.3 mL) was added dropwise to a solution of tert-butyl (3S)-3-{(ethanesulfonyl) [(4-methoxyphenyl)methyl]amino}pyrrolidine-1-carboxylate (5.0 g) in THE (50 mL) at −78° C. The mixture was stirred at −78° C. under nitrogen atmosphere for 30 minutes. A solution of N-fluorobis(phenylsulfonyl)amine (8.70 g) in THF (10 mL) was added to the mixture and the mixture was stirred at room temperature under nitrogen atmosphere overnight. The mixture was quenched with saturated ammonium chloride aqueous solution at 0° C. and the resulting mixture was extracted with EtOAc. The organic layer was separated, washed with water and brine, dried over anhydrous magnesium sulfate and concentrated under reduced pressure. The residue was purified by silica gel column chromatography (EtOAc/hexane) and by preparative HPLC (C18, mobile phase: water (containing 10 mM ammonium bicarbonate)/acetonitrile). Then the desired fractions were concentrated to give the title compound (1.60 g).

¹H NMR (300 MHZ, DMSO-d₆) δ 1.35 (9H, br s), 1.53-1.70 (3H, m), 1.84-3.70 (6H, m), 3.74 (3H, s), 4.31-4.50 (3H, m), 5.67-5.96 (1H, m), 6.88-6.96 (2H, m), 7.28 (2H, d, J=8.3 Hz).

### C) 1-Fluoro-N-[(3S)-pyrrolidin-3-yl]ethane-1-sulfonamide

A mixture of tert-Butyl (3S)-3-{(1-fluoroethanesulfonyl) [(4-methoxyphenyl)methyl]amino}pyrrolidine-1-carboxylate (1.58 g), TFA (20 mL), and water (1.00 mL) was stirred at 60° ° C. over weekend. The residue was purified by NH silica gel column chromatography (EtOAc/hexane) to give the title compound (340 mg).

¹H NMR (400 MHZ, DMSO-d₆) δ 1.51-1.65 (3H, m), 1.71-1.83 (1H, m), 2.03-2.16 (1H, m), 2.85 (1H, td, J=12.3, 5.7 Hz), 2.97-3.17 (2H, m), 3.24 (1H, ddd, J=11.6, 7.0, 4.2 Hz), 3.94-4.02 (1H, m), 4.89-6.48 (2H, m), 5.51-5.72 (1H, m).

### D) N-[(3S)-1-{(5S)-5-[3-(2,6-Difluorophenyl)-5-methylpyridin-2-yl]-4,5-dihydro-1,2-oxazol-3-yl}pyrrolidin-3-yl]-1-fluoroethane-1-sulfonamide

A mixture of 2-[(5S)-3-bromo-4,5-dihydro-1,2-oxazol-5-yl]-3-(2,6-difluorophenyl)-5-methylpyridine (50 mg), 1-fluoro-N-[(3S)-pyrrolidin-3-yl]ethane-1-sulfonamide (50.0 mg) and DIPEA (0.247 mL) in NMP (0.4 mL) was stirred in a sealed tube at 120° C. for 6 hours. The mixture was purified by silica gel column chromatography (EtOAc/hexane) to give the title compound (18.0 mg).

¹H NMR (300 MHZ, CDCl₃) δ 1.62-1.75 (3H, m), 1.97-2.12 (2H, m), 2.14-2.27 (1H, m), 2.38 (3H, s), 3.16 (1H, dd, J=15.6, 9.6 Hz), 3.27-3.40 (2H, m), 3.42-3.59 (2H, m), 3.74 (1H, ddd, J=15.7, 8.9, 3.2 Hz), 4.12-4.23 (1H, m), 5.11-6.38 (2H, m), 6.93-7.07 (2H, m), 7.30-7.44 (2H, m), 8.50 (1H, dd, J=4.1, 1.9 Hz).

### Example 107

### Optical isomer of 1-fluoro-N-[(3R,4S)-4-fluoro-1-{(5S)-5-[5-methyl-3-(2,4,6-trifluorophenyl)pyridin-2-yl]-4,5-dihydro-1,2-oxazol-3-yl}pyrrolidin-3-yl]cyclopropane-1-sulfonamide or 1-fluoro-N-[(3S,4R)-4-fluoro-1-{(5S)-5-[5-methyl-3-(2,4,6-trifluorophenyl)pyridin-2-yl]-4,5-dihydro-1,2-oxazol-3-yl}pyrrolidin-3-yl]cyclopropane-1-sulfonamide

### A) rac-tert-Butyl (3S,4S)-3-fluoro-4-[(trifluoromethanesulfonyl)oxy]pyrrolidine-1-carboxylate

To a mixture of rac-tert-butyl (3S,4S)-3-fluoro-4-hydroxypyrrolidine-1-carboxylate (528 mg), pyridine (0.6 mL) and acetonitrile (10 mL) was added dropwise trifluoromethanesulfonic anhydride (0.6 mL) at −10° C., and the mixture was stirred at −10° C. for 1 hour. The mixture was quenched with aqueous ammonium chloride solution and the resulting mixture was extracted with EtOAc. The organic layer was separated, dried over anhydrous magnesium sulfate and concentrated under reduced pressure. The residue was purified by silica gel column chromatography (EtOAc/hexane) to give the title compound (833 mg).

¹H NMR (300 MHz, DMSO-d₆) δ 1.41 (9H, s), 3.46-3.89 (4H, m), 5.39-5.64 (1H, m), 5.70-5.86 (1H, m).

### B) 1-Fluorocyclopropane-1-sulfonamide

A mixture of tert-butyl ((1-fluorocyclopropyl) sulfonyl) carbamate (893 mg) and TFA (3 mL) was stirred at room temperature for 2.5 hours. The mixture was concentrated under reduced pressure to give the title compound (580 mg).

¹H NMR (300 MHZ, DMSO-d₆) δ 1.30-1.56 (4H, m), 7.51 (2H, s).

### C) rac-tert-Butyl (3S,4R)-3-fluoro-4-[(1-fluorocyclopropane-1-sulfonyl)amino]pyrrolidine-1-carboxylate

To a solution of 1-fluorocyclopropane-1-sulfonamide (575 mg) and DMF (9 mL) was added sodium hydride (60% in oil, 179 mg) at 0° C., and the mixture was stirred at 0° C. for 10 minutes. A solution of rac-tert-butyl (35,4S)-3-fluoro-4-[(trifluoromethanesulfonyl)oxy]pyrrolidine-1-carboxylate (1.23 g) in DMF (3 mL) was added dropwise at 0° C., and the mixture was warmed up to room temperature and stirred under nitrogen atmosphere overnight. The mixture was quenched with aqueous ammonium chloride solution and the resulting mixture was extracted with EtOAc. The organic layer was separated, dried over anhydrous magnesium sulfate and concentrated under reduced pressure. The residue was purified by silica gel column chromatography (EtOAc/hexane) to give the title compound (0.575 g).

¹H NMR (300 MHz, DMSO-d₆) δ 1.32-1.63 (13H, m), 3.13 (1H, td, J=10.1, 3.6 Hz), 3.41-3.49 (1H, m), 3.53-3.69 (2H, m), 4.08-4.34 (1H, m), 4.84-5.16 (1H, m), 8.49 (1H, d, J=8.7 Hz).

### D) rac-1-Fluoro-N-((3R,4S)-4-fluoropyrrolidin-3-yl) cyclopropane-1-sulfonamide hydrochloride

A mixture of rac-tert-butyl (3S,4R)-3-fluoro-4-[(1-fluorocyclopropane-1-sulfonyl)amino]pyrrolidine-1-carboxylate (575 mg), 4 M hydrogen chloride in CPME (4.5 mL) was stirred at room temperature for 1.5 hours. The mixture was concentrated under reduced pressure, and the solid was collected by filtration and washed with IPE/EtOAc to give the title compound (411 mg).

¹H NMR (300 MHz, DMSO-d₆) δ 1.39-1.66 (4H, m), 3.04 (1H, t, J=11.3 Hz), 3.43-3.67 (3H, m), 4.16-4.43 (1H, m), 4.99-5.26 (1H, m), 8.69 (1H, br s), 9.43 (2H, br s).

### E) Optical isomer of 1-fluoro-N-[(3R,4S)-4-fluoro-1-{(5S)-5-[5-methyl-3-(2,4,6-trifluorophenyl)pyridin-2-yl]-4,5-dihydro-1,2-oxazol-3-yl}pyrrolidin-3-yl]cyclopropane-1-sulfonamide or 1-fluoro-N-[(3S,4R)-4-fluoro-1-{(5S)-5-[5-methyl-3-(2,4,6-trifluorophenyl)pyridin-2-yl]-4,5-dihydro-1,2-oxazol-3-yl}pyrrolidin-3-yl]cyclopropane-1-sulfonamide

A mixture of 2-[(5S)-3-bromo-4,5-dihydro-1,2-oxazol-5-yl]-5-methyl-3-(2,4,6-trifluorophenyl)pyridine (59.2 mg), rac-1-fluoro-N-((3R,4S)-4-fluoropyrrolidin-3-yl) cyclopropane-1-sulfonamide hydrochloride (62.2 mg), cesium carbonate (144 mg) and t-BuOH (0.8 mL) was stirred at 140° C. for 5 hours in a sealed tube. The mixture was quenched with aqueous ammonium chloride solution and the resulting mixture was extracted with EtOAc. The organic layer was separated, dried over anhydrous magnesium sulfate and concentrated under reduced pressure. The residue was purified by silica gel column chromatography (EtOAc/hexane). The desired fraction was concentrated under reduced pressure. The residue was solidified by IPE to give the title compound (19.1 mg).

¹H NMR (300 MHz, DMSO-d₆) δ 1.40-1.63 (4H, m), 2.36 (3H, s), 3.13-3.27 (2H, m), 3.42-3.79 (4H, m), 4.10-4.35 (1H, m), 4.94-5.19 (1H, m), 5.25 (1H, t, J=9.4 Hz), 7.27-7.42 (2H, m), 7.63 (1H, d, J=1.9 Hz), 8.49-8.61 (2H, m).

### Example 109

### N-[(3R)-1-{(5S)-5-[3-(2,6-Difluorophenyl)-5-fluoropyridin-2-yl]-5-(fluoromethyl)-4,5-dihydro-1,2-oxazol-3-yl}-4,4-difluoropyrrolidin-3-yl]methanesulfonamide

### A) 2-Chloro-3-(2,6-difluorophenyl)-5-fluoropyridine

Tri-tert-butylphosphonium tetrafluoroborate (0.414 g) and Pd2(dba)3 (0.653 g) were added to a mixture of 3-bromo-2-chloro-5-fluoropyridine (3.00 g), (2,6-difluorophenyl) boronic acid (2.93 g) and potassium fluoride (1.66 g) in THF (39.6 mL) and water (7.92 mL) at room temperature. The mixture was stirred at 80° C. under nitrogen atmosphere for 2 hours. After cooling to room temperature, the mixture was poured into brine and the resulting mixture was extracted with EtOAc. The organic layer was separated, washed with brine, dried over anhydrous sodium sulfate and concentrated under reduced pressure. The residue was purified by NH silica gel column chromatography (EtOAc/hexane) to give the title compound (3.11 g).

MS: [M+H]⁺ 243.9.

### B) 2-[3-(2,6-Difluorophenyl)-5-fluoropyridin-2-yl]prop-2-en-1-ol

Pd(dppf) Cl₂·CH₂C12 (0.654 g) was added to a mixture of 2-(4,4,5,5-tetramethyl-1,3,2-dioxaborolan-2-yl) prop-2-en-1-ol (1.96 g), 2-chloro-3-(2,6-difluorophenyl)-5-fluoropyridine (1.30 g), potassium carbonate (2.21 g) in DME (15 mL) and water (3.0 mL) at room temperature. The mixture was stirred at 120° C. under argon atmosphere for 10 hours under microwave irradiation. The mixture was diluted with EtOAc and water, and insoluble material was removed through Celite (trade name). The organic layer was separated, dried over anhydrous sodium sulfate, passed through an NH silica gel pad and concentrated under reduced pressure. The residue was purified by silica gel column chromatography (EtOAc/hexane) to give the title compound (0.850 g).

MS: [M+H]⁺ 266.0.

### C) {3-Bromo-5-[3-(2,6-difluorophenyl)-5-fluoropyridin-2-yl]-4,5-dihydro-1,2-oxazol-5-yl}methanol

Sodium hydrogen carbonate (1.77 g) was added to a mixture of 2-[3-(2,6-difluorophenyl)-5-fluoropyridin-2-yl]prop-2-en-1-ol (1.40 g) and hydroxycarbonimidic dibromide (2.14 g) in EtOAc (21.1 mL) at room temperature. The mixture was stirred at 70° C. overnight. The precipitate was removed by filtration and the filtrate was concentrated under reduced pressure. The residue was purified by silica gel column chromatography (EtOAc/hexane) to give the title compound (0.871 g).

¹H NMR (300 MHz, CDCl₃) δ 3.10 (1H, dd, J=7.7, 7.0 Hz), 3.37-3.61 (1H, m), 3.66-3.86 (2H, m), 4.14-4.16 (1H, m), 6.72-7.12 (2H, m), 7.30-7.48 (2H, m), 8.48 (1H, d, J=2.6 Hz).

### D) 2-[3-Bromo-5-(fluoromethyl)-4,5-dihydro-1,2-oxazol-5-yl]-3-(2,6-difluorophenyl)-5-fluoropyridine

DAST (0.743 mL) was added to a solution of {3-bromo-5-[3-(2,6-difluorophenyl)-5-fluoropyridin-2-yl]-4,5-dihydro-1,2-oxazol-5-yl}methanol (871 mg) in CH₂Cl₂ (15 mL) at 0° C. The mixture was gradually warmed up to room temperature and stirred at room temperature under nitrogen atmosphere for 6 hours. DAST (0.149 mL) was added to the mixture at room temperature. The mixture was stirred at room temperature under nitrogen atmosphere overnight. The mixture was quenched with saturated sodium hydrogen carbonate aqueous solution at room temperature and the resulting mixture was extracted with EtOAc. The organic layer was separated, washed with brine, dried over anhydrous sodium sulfate and concentrated under reduced pressure. The residue was purified by silica gel column chromatography (EtOAc/hexane) to give the title compound (580 mg).

¹H NMR (300 MHz, CDCl₃) δ 3.52-3.67 (1H, m), 3.71-3.95 (1H, m), 4.58-5.11 (2H, m), 6.80-7.09 (2H, m), 7.30-7.54 (2H, m), 8.49 (1H, d, J=2.6 Hz).

### E) 2-[(5S)-3-Bromo-5-(fluoromethyl)-4,5-dihydro-1,2-oxazol-5-yl]-3-(2,6-difluorophenyl)-5-fluoropyridine

2-[3-Bromo-5-(fluoromethyl)-4,5-dihydro-1,2-oxazol-5-yl]-3-(2,6-difluorophenyl)-5-fluoropyridine (6.00 g) was optically resolved by SFC. The fraction with longer retention time was concentrated under reduced pressure to give the title compound (2.78 g).

### Chiral Separation Condition

Column: CHIRALCEL OJ (trade name) (250 mm*50 mm, 10 μm)

Mobile phase: CO2/EtOH including 0.1% ammonium hydroxide=850/150 (v/v)

¹H NMR (400 MHZ, DMSO-d₆) δ 3.51-3.57 (1H, m), 3.64-3.71 (1H, m), 4.76-4.86 (1H, m), 4.87-4.97 (1H, m), 7.14-7.22 (2H, m), 7.50-7.59 (1H, m), 7.96 (1H, dd, J=9.2, 2.8 Hz), 8.75 (1H, d, J=2.8 Hz).

### F) tert-Butyl (4R)-3,3-difluoro-4-[(methanesulfonyl)amino]pyrrolidine-1-carboxylate

To a mixture of tert-butyl (R)-4-amino-3,3-difluoropyrrolidine-1-carboxylate (1.65 g), TEA (3.10 mL) and THF (35 mL) was added a solution of methanesulfonic anhydride (1.94 g) in THF (5 mL) at 0° C. The mixture was stirred at room temperature under argon atmosphere overnight. To the mixture was added saturated sodium hydrogen carbonate aqueous solution at 0° C. and the resulting mixture was extracted with EtOAc. The organic layer was separated, washed with water and brine, dried over anhydrous sodium sulfate, and evaporated to dryness. The residue was purified by silica gel column chromatography (EtOAc/hexane) to give the title compound (1.63 g).

¹H NMR (300 MHZ, DMSO-d₆) δ 1.41 (9H, s), 2.99 (3H, s), 3.08-3.19 (1H, m), 3.60-3.76 (2H, m), 3.76-3.85 (1H, m), 4.23-4.44 (1H, m), 7.93 (1H, d, J=9.0 Hz).

### G) N-[(3R)-4,4-Difluoropyrrolidin-3-yl]methanesulfonamide hydrochloride

A mixture of tert-butyl (4R)-3,3-difluoro-4-[(methanesulfonyl)amino] pyrrolidine-1-carboxylate (1.62 g) and 4 M hydrogen chloride in EtOAc (30 mL) was stirred at room temperature for 1 hour. Then the solvent was removed under reduced pressure. To the residue was added a mixture of EtOAc/hexane (1/1). The resulting solid was collected by filtration, washed with EtOAc/hexane (1/1) to give the title compound (1.07 g).

¹H NMR (300 MHZ, DMSO-d₆) δ 3.02 (3H, s), 3.06-3.18 (1H, m), 3.68-3.82 (3H, m), 4.35-4.49 (1H, m), 8.11 (1H, d, J=9.2 Hz), 9.81 (2H, br s).

### H) N-[(3R)-1-{(5S)-5-[3-(2,6-Difluorophenyl)-5-fluoropyridin-2-yl]-5-(fluoromethyl)-4,5-dihydro-1,2-oxazol-3-yl}-4,4-difluoropyrrolidin-3-yl]methanesulfonamide

A mixture of 2-[(5S)-3-bromo-5-(fluoromethyl)-4,5-dihydro-1,2-oxazol-5-yl]-3-(2,6-difluorophenyl)-5-fluoropyridine (1.70 g), N-[(3R)-4,4-difluoropyrrolidin-3-yl]methanesulfonamide hydrochloride (2.07 g) and pyridine (10.9 mL) was stirred in a sealed tube at 135° C. overnight. The mixture was purified by silica gel column chromatography (EtOAc/hexane) and by NH silica gel column chromatography (EtOAc/hexane). The residue was crystallized from EtOAc/hexane to give the title compound (924 mg).

¹H NMR (400 MHZ, CDCl₃) δ 3.07 (3H, s), 3.12-3.19 (1H, m), 3.46 (1H, dd, J=16.4, 0.9 Hz), 3.54-3.71 (3H, m), 3.77 (1H, dd, J=9.9, 8.3 Hz), 4.15-4.39 (1H, m), 4.58-4.89 (3H, m), 6.91-7.06 (2H, m), 7.28-7.38 (2H, m), 8.49 (1H, d, J=2.8 Hz).

### Example 131

### N-[(3R)-1-{5-[3-(2,6-Difluorophenyl)-5-fluoropyridin-2-yl]-5-(methoxymethyl)-4,5-dihydro-1,2-oxazol-3-yl}-4,4-difluoropyrrolidin-3-yl]methanesulfonamide

### A) 2-[3-Bromo-5-(methoxymethyl)-4,5-dihydro-1,2-oxazol-5-yl]-3-(2,6-difluorophenyl)-5-fluoropyridine

To a solution of {3-bromo-5-[3-(2,6-difluorophenyl)-5-fluoropyridin-2-yl]-4,5-dihydro-1,2-oxazol-5-yl}methanol (150 mg) and N,N,N′,N′-tetramethyl-1,8-naphthalenediamine (415 mg) in EtOAc (5 mL) was added trimethyloxoniumtetrafluoroborate (229 mg) at 0° C. The mixture was stirred at room temperature for 2 hours under nitrogen atmosphere. The mixture was filtered, quenched with saturated ammonium chloride aqueous solution and the resulting mixture was extracted with EtOAc. The organic layer was separated, washed with water and brine, dried over anhydrous magnesium sulfate and concentrated under reduced pressure. The residue was purified by NH silica gel column chromatography (EtOAc/hexane) to give the title compound (134 mg).

MS: [M+H]⁺ 400.9.

### B) N-[(3R)-1-{5-[3-(2,6-Difluorophenyl)-5-fluoropyridin-2-yl]-5-(methoxymethyl)-4,5-dihydro-1,2-oxazol-3-yl}-4,4-difluoropyrrolidin-3-yl]methanesulfonamide

A mixture of 2-[3-bromo-5-(methoxymethyl)-4,5-dihydro-1,2-oxazol-5-yl]-3-(2,6-difluorophenyl)-5-fluoropyridine (30.0 mg), N-[(3R)-4,4-difluoropyrrolidin-3-yl]methanesulfonamide hydrochloride (35.4 mg) in pyridine (0.25 mL) was stirred in a sealed tube at 140° C. for 6 hours. The mixture was purified by preparative HPLC (C18, mobile phase: water (containing 10 mM ammonium bicarbonate)/acetonitrile). Then the desired fractions were concentrated to give the title compound (23.6 mg).

¹H NMR (300 MHz, DMSO-d₆) δ 2.98 (3H, s), 3.00-3.10 (1H, m), 3.18-3.23 (3H, m), 3.36-3.74 (7H, m), 4.20-4.37 (1H, m), 7.08-7.19 (2H, m), 7.42-7.53 (1H, m), 7.76-7.82 (1H, m), 7.93 (1H, br s), 8.69 (1H, dd, J=2.8, 1.5 Hz).

### Example 147

### N-[(3R)-4,4-Difluoro-1-{5-(fluoromethyl)-5-[5-fluoro-3-(2,4,6-trifluorophenyl)pyridin-2-yl]-4,5-dihydro-1,2-oxazol-3-yl}pyrrolidin-3-yl]methanesulfonamide (optical isomer)

### A) 5-Fluoro-3-(2,4,6-trifluorophenyl)pyridin-2-amine

Tri-tert-butylphosphonium tetrafluoroborate (0.304 g) and Pd2(dba)3 (0.301 g) were added to a mixture of 3-bromo-5-fluoropyridin-2-amine (2.00 g), (2,4,6-trifluorophenyl) boronic acid (3.32 g) and potassium fluoride (1.22 g) in THF (29.1 mL) and water (5.82 mL) at room temperature. The mixture was stirred at 80° C. under nitrogen atmosphere for 2 hours. The mixture was poured into brine and the resulting mixture was extracted with EtOAc. The organic layer was separated, washed with brine, dried over anhydrous sodium sulfate, passed through an NH silica gel pad and concentrated under reduced pressure. The solid was triturated with IPE to give the title compound (1.81 g).

MS: [M+H]⁺ 242.9.

### B) 5-Fluoro-2-iodo-3-(2,4,6-trifluorophenyl)pyridine

To a stirred mixture of 5-fluoro-3-(2,4,6-trifluorophenyl)pyridin-2-amine (1.70 g), diiodomethane (4.52 mL), copper (I) iodide (2.01 g) in THF (35 mL) was added n-pentyl nitrite (3.73 mL) at room temperature. The mixture was stirred at 70° C. for 5 hours. The solid was removed by filtration, and the filtrate was concentrated under reduced pressure. The residue was purified by silica gel column chromatography (EtOAc/hexane) to give the title compound (1.58 g).

¹H NMR (300 MHZ, CDCl₃) δ 6.76-6.87 (2H, m), 7.29 (1H, dd, J=8.2, 3.0 Hz), 8.37 (1H, d, J=3.0 Hz).

### C) 2-[5-Fluoro-3-(2,4,6-trifluorophenyl)pyridin-2-yl]prop-2-en-1-ol

A mixture of 5-fluoro-2-iodo-3-(2,4,6-trifluorophenyl)pyridine (900 mg), 2-(4,4,5,5-tetramethyl-1,3,2-dioxaborolan-2-yl) prop-2-en-1-ol (704 mg), potassium carbonate (1.06 g), PdCl2 (dppf) (187 mg), DME (15 mL) and water (3.00 mL) was heated at 110° C. for 1 hour under microwave irradiation. The mixture was quenched with water and the resulting mixture was extracted with EtOAc. The organic layer was separated and dried over anhydrous magnesium sulfate and concentrated under reduced pressure. The residue was purified by silica gel column chromatography (EtOAc/hexane) to give the title compound (546 mg).

MS: [M+H]⁺ 283.9.

### D) {3-Bromo-5-[5-fluoro-3-(2,4,6-trifluorophenyl)pyridin-2-yl]-4,5-dihydro-1,2-oxazol-5-yl}methanol

Sodium hydrogen carbonate (485 mg) was added to a mixture of 2-[5-fluoro-3-(2,4,6-trifluorophenyl)pyridin-2-yl]prop-2-en-1-ol (545 mg) and hydroxycarbonimidic dibromide (781 mg) in EtOAc (10 mL) at room temperature. The mixture was stirred at 75° C. overnight. The precipitate was removed by filtration and the filtrate was concentrated under reduced pressure. The residue was purified by column chromatography (EtOAc/hexane) to give the title compound (586 mg).

MS: [M+H]⁺ 404.9.

### E) 2-[3-Bromo-5-(fluoromethyl)-4,5-dihydro-1,2-oxazol-5-yl]-5-fluoro-3-(2,4,6-trifluorophenyl)pyridine

DAST (0.477 mL) was added to a solution of {3-bromo-5-[5-fluoro-3-(2,4,6-trifluorophenyl)pyridin-2-yl]-4,5-dihydro-1,2-oxazol-5-yl}methanol (585 mg) in CH₂Cl₂: (10 mL) at 0° C. The mixture was gradually warmed up to room temperature and stirred at room temperature under nitrogen atmosphere for 6 hours. The mixture was quenched with saturated sodium hydrogen carbonate aqueous solution at room temperature and the resulting mixture was extracted with EtOAc. The organic layer was separated, washed with brine, dried over anhydrous sodium sulfate and concentrated under reduced pressure. The residue was purified by silica gel column chromatography (EtOAc/hexane) to give the title compound (375 mg).

¹H NMR (300 MHz, CDCl₃) δ 3.57-3.83 (2H, m), 4.62-4.94 (2H, m), 6.73-6.85 (2H, m), 7.33 (1H, dd, J=8.3, 2.8 Hz), 8.51 (1H, d, J=2.8 Hz).

### F) 2-[3-Bromo-5-(fluoromethyl)-4,5-dihydro-1,2-oxazol-5-yl]-5-fluoro-3-(2,4,6-trifluorophenyl)pyridine (optical isomer)

2-[3-Bromo-5-(fluoromethyl)-4,5-dihydro-1,2-oxazol-5-yl]-5-fluoro-3-(2,4,6-trifluorophenyl)pyridine (4.00 g) was optically resolved by SFC. The desired fraction with shorter retention time was concentrated under vacuum to give the title compound (1.74 g).

### Chiral Separation Condition

Column: CHIRALPAK IG (trade name) (250 mm*50 mm, 10 μm)

Mobile phase: CO2/MeOH including 0.1% ammonium hydroxide=900/100 (v/v)

¹H NMR (400 MHZ, DMSO-d₆) δ 3.47-3.55 (1H, m), 3.66-3.74 (1H, m), 4.76-4.86 (1H, m), 4.88-4.98 (1H, m), 7.28-7.36 (2H, m), 7.99 (1H, dd, J=8.8, 2.8 Hz), 8.76 (1H, d, J=2.8 Hz).

### G) N-[(3R)-4,4-Difluoro-1-{5-(fluoromethyl)-5-[5-fluoro-3-(2,4,6-trifluorophenyl)pyridin-2-yl]-4,5-dihydro-1,2-oxazol-3-yl}pyrrolidin-3-yl]methanesulfonamide (optical isomer)

A mixture of 2-[3-bromo-5-(fluoromethyl)-4,5-dihydro-1,2-oxazol-5-yl]-5-fluoro-3-(2,4,6-trifluorophenyl)pyridine (optical isomer) (90.0 mg), N-[(3R)-4,4-difluoropyrrolidin-3-yl]methanesulfonamide hydrochloride (105 mg) and pyridine (0.553 mL) was stirred in a sealed tube at 135° C. overnight. The mixture was purified by silica gel column chromatography (EtOAc/hexane). The residue was triturated with IPE/EtOAc to give the title compound (38.4 mg).

¹H NMR (400 MHZ, CDCl₃) δ 3.07 (3H, s), 3.13-3.23 (1H, m), 3.47 (1H, dd, J=16.4, 0.9 Hz), 3.58-3.71 (3H, m), 3.80 (1H, dd, J=9.9, 8.2 Hz), 4.19-4.36 (1H, m), 4.56-4.68 (1H, m), 4.69-4.77 (2H, m), 6.66-6.82 (2H, m), 7.28 (1H, dd, J=8.4, 2.8 Hz), 8.51 (1H, d, J=2.7 Hz).

### Example 149

### N-[(3R)-1-{(5S)-5-[3-(2,6-Difluorophenyl)-5-fluoropyridin-2-yl]-5-(fluoromethyl)-4,5-dihydro-1,2-oxazol-3-yl}-4,4-difluoropyrrolidin-3-yl]-1-fluoromethanesulfonamide

### A) tert-Butyl (4R)-3,3-difluoro-4-[(fluoromethanesulfonyl)amino]pyrrolidine-1-carboxylate

To a solution of tert-butyl (R)-4-amino-3,3-difluoropyrrolidine-1-carboxylate (250 mg) in pyridine (4 mL) was added dropwise fluoromethanesulfonyl chloride (179 mg) at 0° C.. The mixture was stirred at room temperature for 3 hours. The mixture was quenched with water and the resulting mixture was extracted with EtOAc. The organic layer was separated, washed with 1 M hydrogen chloride aqueous solution and brine, dried over anhydrous magnesium sulfate and concentrated under reduced pressure. The residue was purified by silica gel column chromatography (EtOAc/hexane) to give the title compound (321 mg).

¹H NMR (300 MHz, CDCl₃) δ 1.47 (9H, s), 3.20-3.33 (1H, m), 3.60-3.91 (2H, m), 3.92-4.07 (1H, m), 4.19-4.41 (1H, m), 5.08 (1H, s), 5.23 (1H, s), 5.31 (1H, br s).

### B) N-[(3R)-4,4-Difluoropyrrolidin-3-yl]-1-fluoromethanesulfonamide hydrochloride

To a solution of tert-butyl (4R)-3,3-difluoro-4-[(fluoromethanesulfonyl)amino]pyrrolidine-1-carboxylate (321 mg) in EtOAc (5 mL) was added 4 M hydrogen chloride in EtOAc (5 mL). The mixture was stirred at room temperature overnight. The resulting precipitate was collected by filtration and washed with EtOAc to give the title compound (217 mg).

¹H NMR (300 MHz, DMSO-d₆) δ 3.05-3.23 (1H, m), 3.61-3.89 (3H, m), 4.29-4.58 (1H, m), 5.17-5.70 (2H, m), 8.45-9.13 (1H, m), 9.95 (2H, br s).

### C) N-[(3R)-1-{(5S)-5-[3-(2,6-Difluorophenyl)-5-fluoropyridin-2-yl]-5-(fluoromethyl)-4,5-dihydro-1,2-oxazol-3-yl}-4,4-difluoropyrrolidin-3-yl]-1-fluoromethanesulfonamide

A mixture of 2-[(5S)-3-bromo-5-(fluoromethyl)-4,5-dihydro-1,2-oxazol-5-yl]-3-(2,6-difluorophenyl)-5-fluoropyridine (50.0 mg), N-[(3R)-4,4-difluoropyrrolidin-3-yl]-1-fluoromethanesulfonamide hydrochloride (49.1 mg) in pyridine (0.40 mL) was stirred in a sealed tube at 140° C. overnight. The mixture was purified by NH silica gel column chromatography (MeOH/EtOAc) and by silica gel column chromatography (EtOAc/hexane) to give the title compound (23.0 mg).

¹H NMR (300 MHz, CDCl₃) δ 3.15-3.27 (1H, m), 3.41-3.78 (5H, m), 4.18-4.38 (1H, m), 4.59-4.73 (1H, m), 4.76-4.89 (1H, m), 5.06 (1H, s), 5.21 (1H, s), 5.37-5.54 (1H, m), 6.91-7.04 (2H, m), 7.29-7.44 (2H, m), 8.49 (1H, d, J=2.7 Hz).

### Example 154

### N-[(3R)-1-{5-[2-(3,5-Difluoropyridin-2-yl)-6-fluoro-4-methylphenyl]-4,5-dihydro-1,2-oxazol-3-yl}-4,4-difluoropyrrolidin-3-yl]methanesulfonamide

### A) 3-Bromo-5-(2-bromo-6-fluoro-4-methylphenyl)-4,5-dihydro-1,2-oxazole

Hydroxycarbonimidic dibromide (943 mg) was added to a mixture of 1-bromo-3-fluoro-5-methyl-2-vinylbenzene (500 mg), sodium hydrogen carbonate (1.56 g) and EtOAc (10 mL) at room temperature. The mixture was stirred at 70° C. for 1.5 hours. The precipitate was removed by filtration, and the filtrate was concentrated under reduced pressure. The residue was purified by silica gel column chromatography (EtOAc/hexane) to give the title compound (793 mg).

¹H NMR (300 MHZ, DMSO-d₆) δ 2.32 (3H, s), 3.40 (1H, td, J=8.8, 1.2 Hz), 3.80 (1H, ddd, J=17.6, 12.2, 2.3 Hz), 6.02-6.10 (1H, m), 7.19 (1H, dt, J=11.9, 0.7 Hz), 7.40 (1H, s).

### B) N-{(3R)-1-[5-(2-Bromo-6-fluoro-4-methylphenyl)-4,5-dihydro-1,2-oxazol-3-yl]-4,4-difluoropyrrolidin-3-yl}methanesulfonamide

N-[(3R)-4,4-Difluoropyrrolidin-3-yl]methanesulfonamide hydrochloride (492 mg) was added to a solution of 3-bromo-5-(2-bromo-6-fluoro-4-methylphenyl)-4,5-dihydro-1,2-oxazole (500 mg) in pyridine (3.71 mL). The mixture was stirred in a sealed tube at 140° C. overnight. The mixture was poured into saturated sodium hydrogen carbonate aqueous solution at room temperature and the resulting mixture was extracted with EtOAc. The organic layer was separated, washed with brine, dried over anhydrous magnesium sulfate and concentrated under reduced pressure. The residue was purified by silica gel column chromatography (EtOAc/hexane) to give the title compound (309 mg).

¹H NMR (300 MHZ, DMSO-d₆) δ 2.31 (3H, s), 3.00 (3H, s), 3.15-3.29 (2H, m), 3.39-3.56 (1H, m), 3.68-3.90 (3H, m), 4.25-4.46
(1H, m), 5.79 (1H, t, J=10.3 Hz), 7.14 (1H, d, J=11.6 Hz), 7.37 (1H, s), 7.99 (1H, dd, J=9.1, 4.2 Hz).

### C) N-[(3R)-1-{5-[2-(3,5-Difluoropyridin-2-yl)-6-fluoro-4-methylphenyl]-4,5-dihydro-1,2-oxazol-3-yl}-4,4-difluoropyrrolidin-3-yl]methanesulfonamide

Hexa-n-butylditin (0.391 mL) was added to a mixture of 2-bromo-3,5-difluoropyridine (100 mg), tricyclohexylphosphine (14.5 mg) and Pd2(dba)3 (47.2 mg) in DME (1.5 mL) at room temperature. The mixture was heated to 80° C. over 16 hours under nitrogen atmosphere. The reaction mixture was cooled to room temperature and filtered directly through Celite (trade name) with EtOAc. The resultant filtrate was concentrated under reduced pressure. The residue was suspended in DMF (1.5 mL) and added to a flask containing N-{(3R)-1-[5-(2-bromo-6-fluoro-4-methylphenyl)-4,5-dihydro-1,2-oxazol-3-yl]-4,4-difluoropyrrolidin-3-yl}methanesulfonamide (25.0 mg) and Pd(Ph3P) 4 (12.7 mg) at room temperature. The mixture was heated to 110° C. for 6 hours under argon atmosphere. The mixture was filtered through Celite (trade name) and the filtrate was diluted with water and the resulting mixture was extracted with EtOAc. The combined organic layers were subsequently washed with brine, dried over anhydrous magnesium sulfate and concentrated under reduced pressure. The residue was purified by preparative HPLC (C18, mobile phase: water (containing 10 mM ammonium bicarbonate)/acetonitrile) and by NH silica gel column chromatography (EtOAc/hexane) to give the title compound (5.0 mg).

¹H NMR (300 MHz, DMSO-d₆) δ 2.36 (3H, s), 2.99 (3H, s), 3.08-3.27 (2H, m), 3.35-3.46 (1H, m), 3.53-3.83 (3H, m), 4.24-4.41 (1H, m), 5.18-5.32 (1H, m), 7.02 (1H, s), 7.23 (1H, d, J=12.4 Hz), 7.98 (1H, s), 8.07 (1H, td, J=9.3, 1.7 Hz), 8.58 (1H, t, J=2.4 Hz).

### Example 164

### Optical isomer of N-{(3R,4S)-4-fluoro-4-methyl-1-[(5S)-5-(2′,3,5,6′-tetrafluoro[1,1′-biphenyl]-2-yl)-4,5-dihydro-1,2-oxazol-3-yl]pyrrolidin-3-yl}methanesulfonamide or N-{(3,4R)-4-fluoro-4-methyl-1-[(5S)-5-(2′,3,5,6′-tetrafluoro[1,1′-biphenyl]-2-yl)-4,5-dihydro-1,2-oxazol-3-yl]pyrrolidin-3-yl}methanesulfonamide or N-{(3R,4S)-4-fluoro-4-methyl-1-[(5R)-5-(2′,3,5,6′-tetrafluoro[1,1′-biphenyl]-2-yl)-4,5-dihydro-1,2-oxazol-3-yl]pyrrolidin-3-yl}methanesulfonamide or N-{(3S,4R)-4-fluoro-4-methyl-1-[(5R)-5-(2′,3,5,6′-tetrafluoro[1,1′-biphenyl]-2-yl)-4,5-dihydro-1,2-oxazol-3-yl]pyrrolidin-3-yl}methanesulfonamide

### A) Ethyl 4-{(tert-butoxycarbonyl) [(4-methoxyphenyl)methyl]amino}-2-fluoro-3-hydroxy-2-methylbutanoate

Under argon atmosphere, to a solution of diisopropylamine (15.8 g) in THF (150 mL) was added n-butyllithium (1.6 M in hexane, 97 mL) dropwise at 0° C., after which the mixture was stirred for 15 minutes. The mixture was then cooled to −78° C., after which a solution of ethyl 2-fluoropropanoate (15.6 g) in THF (50 mL) was added dropwise at the same temperature. The mixture was stirred at −78° C. for 30 minutes. A solution of tert-butyl (4-methoxybenzyl) (2-oxoethyl) carbamate (14.5 g) in THF (50 mL) was added dropwise, and the mixture was stirred at −78° C. for 1 hour, after which it was slowly warmed to room temperature and stirred at room temperature overnight. The mixture was quenched with saturated ammonium chloride aqueous solution at 0° C. and the resulting mixture was extracted with EtOAc. The organic layer was separated, washed with brine, dried over anhydrous magnesium sulfate and concentrated under reduced pressure. The residue was purified by silica gel column chromatography (EtOAc/hexane) to give the title compound (13.3 g).

MS: [M+Na]+422.2.

### B) rac-(3R,4S)-3-Fluoro-4-hydroxy-1-[(4-methoxyphenyl)methyl]-3-methylpyrrolidin-2-one

A mixture of ethyl 4-{(tert-butoxycarbonyl) [(4-methoxyphenyl)methyl]amino}-2-fluoro-3-hydroxy-2-methylbutanoate (13.3 g) and 4 M hydrogen chloride in EtOAc (130 mL) was stirred at room temperature for 1 hour. The mixture was concentrated under reduced pressure. A mixture of the residue, DIPEA (17.5 mL) and EtOH (400 mL) was stirred at 80° C. for 4 hours. The mixture was concentrated under reduced pressure. The residue was purified by silica gel column chromatography (EtOAc/hexane) to give the title compound (2.22 g).

MS: [M+H]⁺ 254.1.

### C) rac-(3R,4R)-4-Azido-3-fluoro-1-[(4-methoxyphenyl)methyl]-3-methylpyrrolidin-2-one

Under argon atmosphere, to a solution of rac-(3R,4S)-3-fluoro-4-hydroxy-1-[(4-methoxyphenyl)methyl]-3-methylpyrrolidin-2-one (8.73 g) and pyridine (13.9 mL) in acetonitrile (150 mL) was added trifluoromethanesulfonic anhydride (14.6 mL) dropwise, and the mixture was stirred at 0° C. for 2 hours. The mixture was quenched with water and the resulting mixture was extracted with EtOAc. The organic layer was separated, washed with brine, dried over anhydrous magnesium sulfate and concentrated under reduced pressure. A mixture of the residue, tetra-n-butylammonium azide (30.6 g) and acetonitrile (150 mL) was stirred at 80° C. for 2 hours. After cooling to room temperature, the mixture was quenched with water at room temperature and the resulting mixture was extracted with EtOAc. The organic layer was separated, washed with brine, dried over anhydrous magnesium sulfate and concentrated under reduced pressure. The residue was purified by silica gel column chromatography (EtOAc/hexane) to give the title compound (8.80 g).

¹H NMR (300 MHz, DMSO-d₆) δ 1.35-1.61 (3H, m), 3.08 (1H, ddd, J=10.7, 4.0, 1.1 Hz), 3.50 (1H, dd, J=10.9, 6.0 Hz), 3.74 (3H, s), 4.20-4.37 (2H, m), 4.39-4.61 (1H, m), 6.85-6.99 (2H, m), 7.08-7.21 (2H, m).

### D) rac-(3R,4R)-4-Amino-3-fluoro-1-[(4-methoxyphenyl)methyl]-3-methylpyrrolidin-2-one

Under hydrogen atmosphere, a mixture of rac-(3R,4R)-4-azido-3-fluoro-1-[(4-methoxyphenyl)methyl]-3-methylpyrrolidin-2-one (8.80 g) and 10% palladium on carbon (880 mg) in MeOH (90 mL) was stirred at room temperature for 3 hours. The catalyst was removed by filtration and the filtrate was concentrated under reduced pressure to give the title compound (7.80 g).

MS: [M+H]⁺ 253.1.

### E) rac-N-{(3R,4R)-4-Fluoro-1-[(4-methoxyphenyl)methyl]-4-methyl-5-oxopyrrolidin-3-yl}methanesulfonamide

To a solution of rac-(3R,4R)-4-amino-3-fluoro-1-[(4-methoxyphenyl)methyl]-3-methylpyrrolidin-2-one (7.80 g) in THE (80 mL) were added methanesulfonic anhydride (8.08 g) and TEA (8.62 mL) at 0° C. After being stirred at room temperature for 1 hour, the mixture was concentrated under reduced pressure. The residue was purified by silica gel column chromatography (EtOAc/hexane) to give the title compound (8.60 g).

MS: [M+H]⁺ 331.1.

### F) rel-N-{(3R,4R)-4-Fluoro-1-[(4-methoxyphenyl)methyl]-4-methyl-5-oxopyrrolidin-3-yl}methanesulfonamide (optical isomer)

rac-N-{(3R,4R)-4-Fluoro-1-[(4-methoxyphenyl)methyl]-4-methyl-5-oxopyrrolidin-3-yl}methanesulfonamide (10 g) was optically resolved by SFC. The desired fraction with longer retention time was concentrated under vacuum to give the title compound (4.77 g).

### Chiral Separation Condition

Column: CHIRALPAK AY (trade name) (250×30 mm I.D., 10 μm)

Mobile phase: CO2/EtOH=750/250 (v/v)

MS: [M+H]⁺ 331.1.

### G) rel-N-{(3R,4S)-4-Fluoro-1-[(4-methoxyphenyl)methyl]-4-methylpyrrolidin-3-yl}methanesulfonamide (optical isomer)

To a solution of rel-N-{(3R,4R)-4-fluoro-1-[(4-methoxyphenyl)methyl]-4-methyl-5-oxopyrrolidin-3-yl}methanesulfonamide (optical isomer) (1.00 g) in THF (10 mL) was added borane dimethyl sulfide complex in THE (1.9 M, 7.97 mL) dropwise at room temperature. The mixture was stirred at 50° C. for 5 hours. The reaction mixture was cooled to room temperature after which MeOH (10 mL) and 1 M sodium hydroxide aqueous solution (10 mL) were added dropwise. The mixture was stirred at 65° C. for 3 hours. After cooling to room temperature, water and brine were added, and the mixture was extracted with EtOAc. The organic layer were combined washed with brine, dried over anhydrous magnesium sulfate and concentrated under reduced pressure. The residue was washed with EtOAc/hexane (1/1) to give the title compound (653 mg).

MS: [M+H]⁺ 317.0.

### H) rel-N-[(3R,4S)-4-Fluoro-4-methylpyrrolidin-3-yl]methanesulfonamide (optical isomer)

Under hydrogen atmosphere, a mixture of rel-N-{(3R,4S)-4-fluoro-1-[(4-methoxyphenyl)methyl]-4-methylpyrrolidin-3-yl}methanesulfonamide (optical isomer) (653 mg) and 10% palladium on carbon (439 mg) in THF (40 mL) and MeOH (40 mL) was stirred at room temperature for 14 hours. The catalyst was removed by filtration and the filtrate was concentrated under reduced pressure. The residue was washed with hexane/EtOAc (1/4) to give the title compound (350 mg).

¹H NMR (300 MHz, DMSO-d₆) δ 1.28-1.44 (3H, m), 2.59-2.70 (1H, m), 2.87-2.96 (4H, m), 2.98-3.02 (1H, m), 3.07-3.18 (1H, m), 3.23-3.60 (2H, m), 7.03-7.39 (1H, m).

### I) 2-Ethenyl-2′,3,5,6′-tetrafluoro-1,1′-biphenyl

A mixture of 1-bromo-3,5-difluoro-2-vinylbenzene (0.816 mL), 2-(2,6-difluorophenyl)-4,4,5,5-tetramethyl-1,3,2-dioxaborolane (2.52 g), cesium carbonate (6.84 g), copper (I) chloride (0.520 g), Pd (OAc) 2 (0.059 g), and 2-dicyclohexylphosphino-2′,6′-dimethoxy-1,1′-biphenyl (0.431 g) in DMF (27 mL) was stirred at 100° C. for 10 hours. The reaction mixture was diluted with EtOAc, washed with water twice, and concentrated under reduced pressure. The residue was purified by NH silica gel column chromatography (EtOAc/hexane) to give the title compound (0.500 g).

¹H NMR (400 MHZ, DMSO-d₆) δ 5.42 (1H, dt, J=11.8, 1.6 Hz), 5.55 (1H, dt, J=17.9, 1.3 Hz), 6.25 (1H, dd, J=17.9, 11.7 Hz), 7.17-7.69 (5H, m).

### J) 3-Bromo-5-(2′,3,5,6′-tetrafluoro[1,1′-biphenyl]-2-yl)-4,5-dihydro-1,2-oxazole

Sodium hydrogen carbonate (1.67 g) was added to a mixture of 2-ethenyl-2′,3,5,6′-tetrafluoro-1,1′-biphenyl (500 mg) and hydroxycarbonimidic dibromide (804 mg) in EtOAc (7.93 mL) at room temperature. The mixture was stirred at 70° C. for 2 hours. The precipitate was removed by filtration and the filtrate was concentrated under reduced pressure. The residue was purified by silica gel column chromatography (EtOAc/hexane) to give the title compound (540 mg).

¹H NMR (400 MHZ, DMSO-d₆) δ 3.37 (1H, ddd, J=17.6, 9.2, 1.0 Hz), 3.70 (1H, ddd, J=17.5, 12.2, 2.2 Hz), 5.42 (1H, dd, J=11.5, 9.9 Hz), 7.21-7.33 (3H, m), 7.49-7.65 (2H, m).

### K) 3-Bromo-5-(2′,3,5,6′-tetrafluoro[1,1′-biphenyl]-2-yl)-4,5-dihydro-1,2-oxazole (optical isomer)

3-Bromo-5-(2′,3,5,6′-tetrafluoro[1,1′-biphenyl]-2-yl)-4,5-dihydro-1,2-oxazole (1.50 g) was optically resolved by SFC to give the title compound with shorter retention time (601 mg).

### Chiral Separation Condition

Column: CHIRALCEL OJ (trade name) (250 mm*50 mm, 10 μm)

Mobile phase: CO2/MeOH including 0.1% ammonium hydroxide=900/100 (v/v)

¹H NMR (400 MHZ, DMSO-d₆) δ 3.35-3.47 (1H, m), 3.63-3.75 (1H, m), 5.37-5.46 (1H, m), 7.21-7.32 (3H, m), 7.47-7.64 (2H, m)

### L) Optical isomer of N-{(3R,4S)-4-fluoro-4-methyl-1-[(5S)-5-(2′,3,5,6′-tetrafluoro[1,1′-biphenyl]-2-yl)-4,5-dihydro-1,2-oxazol-3-yl]pyrrolidin-3-yl}methanesulfonamide or N-{(3S,4R)-4-fluoro-4-methyl-1-[(5S)-5-(2′,3,5,6′-tetrafluoro[1,1′-biphenyl]-2-yl)-4,5-dihydro-1,2-oxazol-3-yl]pyrrolidin-3-yl}methanesulfonamide or N-{(3R,4S)-4-fluoro-4-methyl-1-[(5R)-5-(2′,3,5,6′-tetrafluoro[1,1′-biphenyl]-2-yl)-4,5-dihydro-1,2-oxazol-3-yl]pyrrolidin-3-yl}methanesulfonamide or N-{(3S,4R)-4-fluoro-4-methyl-1-[(5R]-5-(2′,3,5,6′-tetrafluoro[1,1′-biphenyl]-2-yl)-4,5-dihydro-1,2-oxazol-3-yl]pyrrolidin-3-yl}methanesulfonamide

The mixture of 3-bromo-5-(2′,3,5,6′-tetrafluoro[1,1′-biphenyl]-2-yl)-4,5-dihydro-1,2-oxazole (optical isomer) (37.4 mg), rel-N-[(3R,4S)-4-Fluoro-4-methylpyrrolidin-3-yl]methanesulfonamide (optical isomer) (39.2 mg) and DIPEA (0.052 mL) in t-BuOH (0.3 mL) was heated at 140° ° C. for 14 hours under microwave irradiation. After cooling to room temperature, the mixture was purified by silica gel column chromatography (EtOAc/hexane) and by NH silica gel column chromatography (EtOAc/hexane) to give the title compound (26.4 mg).

¹H NMR (300 MHZ, DMSO-d₆) δ 1.35-1.52 (3H, m), 2.97 (3H, s), 3.10-3.22 (1H, m), 3.26-3.30 (2H, m), 3.36-3.41 (1H, m), 3.48 (1H, s), 3.60-3.71 (1H, m), 3.73-3.96 (1H, m), 5.07 (1H, t, J=10.4 Hz), 7.09-7.36 (3H, m), 7.38-7.69 (3H, m).

### Example 165

### N-[(3R)-1-{(5S)-5-[3-(2,6-Difluorophenyl)-5-fluoropyridin-2-yl]-5-methyl-4,5-dihydro-1,2-oxazol-3-yl}-4,4-difluoropyrrolidin-3-yl]methanesulfonamide

### A) 2-(3-Bromo-5-methyl-4,5-dihydro-1,2-oxazol-5-yl)-3-(2,6-difluorophenyl)-5-fluoropyridine

To a mixture of 2-chloro-3-(2,6-difluorophenyl)-5-fluoropyridine (78.0 g), potassium isopropenyltrifluoroborate (142 g), potassium carbonate (133 g), and copper (I) iodide (3.05 g) in DME (780 mL) and water (156 mL) was added Pd(dppf) C₂·CH₂Cl₂: (39.2 g) at 20° C. The mixture was stirred at 110° C. for 4 hours under nitrogen atmosphere. The reaction mixture was diluted with water and extracted with EtOAc. The combined organic layers were washed with brine, dried over anhydrous sodium sulfate, filtered and concentrated under reduced pressure to give a residue. The residue was purified by silica gel column (EtOAc/petroleum ether) to give 3-(2,6-difluorophenyl)-5-fluoro-2-(prop-1-en-2-yl)pyridine (73 g, crude). To a mixture of 3-(2,6-difluorophenyl)-5-fluoro-2-(prop-1-en-2-yl)pyridine (73.0 g, crude), and hydroxycarbonimidic dibromide (42.8 g) in EtOAc (800 mL) was added sodium hydrogen carbonate (29.5 g). The mixture was stirred at 60° C. under nitrogen atmosphere for 12 hours. After being cooled to room temperature, the mixture was filtered and the filtrate was concentrated under reduced pressure to give a residue. The residue was purified by silica gel column chromatography (EtOAc/petroleum ether) to give the crude title compound, which was dissolved with EtOAc. The combined organic layers were washed with brine and water, dried over anhydrous sodium sulfate, filtered and concentrated under reduced pressure to give the title compound (36.5 g).

¹H NMR (400 MHZ, DMSO-d₆) δ 1.63 (3H, s), 3.46-3.50 (1H, m, overlapped with water's signal), 3.78-3.83 (1H, m), 7.08-7.24 (2H, m), 7.44-7.59 (1H, m), 7.85-7.88 (1H, m), 8.72 (1H, d, J=2.8 Hz).

### B) 2-[(5S)-3-Bromo-5-methyl-4,5-dihydro-1,2-oxazol-5-yl]-3-(2,6-difluorophenyl)-5-fluoropyridine

2-(3-Bromo-5-methyl-4,5-dihydro-1,2-oxazol-5-yl)-3-(2,6-difluorophenyl)-5-fluoropyridine (37.8 g) was optically resolved by SFC. The desired fraction with longer retention time was concentrated under vacuum. The residue was optically resolved again by SFC. The desired fraction with longer retention time was concentrated. The residue was diluted with water and extracted with EtOAc. The combined organic layers were washed with brine, dried over anhydrous sodium sulfate and concentrated under reduced pressure to give the title compound (9.33 g).

### Chiral Separation Condition

Column: CHIRALCEL OJ (trade name) (250 mm*50 mm, 10 μm)

Mobile phase: CO2/IPA including 0.1% ammonium hydroxide=800/200 (v/v)

¹H NMR (400 MHz, DMSO-d₆) δ 1.62 (3H, s), 3.45-3.50 (1H, m), 3.78-3.82 (1H, m), 7.14-7.20 (2H, m), 7.48-7.56 (1H, m), 7.84-7.87 (1H, m), 8.71 (1H, d, J=2.8 Hz).

### C) N-[(3R)-1-{(5S)-5-[3-(2,6-Difluorophenyl)-5-fluoropyridin-2-yl]-5-methyl-4,5-dihydro-1,2-oxazol-3-yl}-4,4-difluoropyrrolidin-3-yl]methanesulfonamide

To a mixture of 2-[(5S)-3-bromo-5-methyl-4,5-dihydro-1,2-oxazol-5-yl]-3-(2,6-difluorophenyl)-5-fluoropyridine (50.0 mg) in pyridine (1 mL) was added N-[(3R)-4,4-difluoropyrrolidin-3-yl]methanesulfonamide hydrochloride (63.8 mg). The mixture was stirred at 140° C. for 4 hours under microwave irradiation. The mixture was concentrated under reduced pressure. The crude material was purified by preparative HPLC (C18, mobile phase: water (containing 10 mM ammonium bicarbonate)/acetonitrile). Then the desired fractions were concentrated to give the title compound (23.6 mg).

¹H NMR (300 MHZ, DMSO-d₆) δ 1.53 (3H, s), 2.98 (3H, s), 3.06 (1H, t, J=9.3 Hz), 3.17 (1H, d, J=16.4 Hz), 3.50-3.65 (2H, m), 3.65-3.78 (2H, m), 4.16-4.36 (1H, m), 7.07-7.18 (2H, m), 7.40-7.53 (1H, m), 7.73-7.78 (1H, m), 7.93 (1H, br s), 8.68 (1H, d, J=2.8 Hz).

### Example 167

### N-[(3R)-1-{5-[2-(3,5-Difluoropyridin-2-yl)-6-fluoro-4-methylphenyl]-4,5-dihydro-1,2-oxazol-3-yl}-4,4-difluoropyrrolidin-3-yl]methanesulfonamide (optical isomer)

N-[(3R)-1-{5-[2-(3,5-Difluoropyridin-2-yl)-6-fluoro-4-methylphenyl]-4,5-dihydro-1,2-oxazol-3-yl}-4,4-difluoropyrrolidin-3-yl]methanesulfonamide (110 mg) was optically resolved by SFC to give the title compound with longer retention time (36 mg).

### Chiral Separation Condition

### First Separation

Column: CHIRALPAK AD (trade name) (250*30 mm I.D., 10 μm)

Mobile phase: CO2/IPA=750/250 (v/v)

### Second Separation

Column: CHIRALPAK AS (trade name) (250*30 mm I.D., 10 μm)

Mobile phase: CO2/EtOH=850/150 (v/v)

¹H NMR (300 MHZ, DMSO-d₆) δ 2.36 (3H, s), 2.99 (3H, s), 3.08-3.30 (2H, m), 3.36-3.48 (1H, m), 3.77 (3H, s), 4.25-4.43 (1H, m), 5.24 (1H, t, J=10.7 Hz), 7.03 (1H, s), 7.23 (1H, d, J=12.1 Hz), 7.99 (1H, d, J=9.2 Hz), 8.03-8.11 (1H, m), 8.58 (1H, d, J=2.4 Hz).

### Example 171

### N-[(3R)-1-{5-[4-Chloro-2-(3,5-difluoropyridin-2-yl)-6-fluorophenyl]-4,5-dihydro-1,2-oxazol-3-yl}-4,4-difluoropyrrolidin-3-yl]methanesulfonamide

### A) 4-Chloro-2-(3,5-difluoropyridin-2-yl)-6-fluoroaniline

A mixture of 4-chloro-2-fluoro-6-(4,4,5,5-tetramethyl-1,3,2-dioxaborolan-2-yl) aniline (233 mg), 2-bromo-3,5-difluoropyridine (183 mg), Pd2(dba)3 (157 mg), cesium fluoride (261 mg), and tri-tert-butylphosphonium tetrafluoroborate (124 mg) in THE (4 mL) was stirred at at 60° C. for 4 hours under argon atmosphere. The reaction mixture was directly filtered through celite (trade name), and the filtrate was concentrated under reduced pressure. The residue was purified by silica gel column chromatography (EtOAc/hexane) and by NH silica gel column chromatography (EtOAc/hexane) to give the title compound (117 mg).

MS: [M+H]⁺ 258.8.

### B) 2-(5-Chloro-3-fluoro-2-iodophenyl)-3,5-difluoropyridine

To a stirred solution of 4-chloro-2-(3,5-difluoropyridin-2-yl)-6-fluoroaniline (113 mg) in THF (4 mL) were added copper (I) iodide (125 mg), diiodomethane (0.282 mL) and n-pentyl nitrite (0.232 mL). The mixture was stirred at 70° C. for 6 hours under argon atmosphere. After cooling to room temperature, the mixture was dissolved in EtOAc and washed with 10% sodium thiosulfate aqueous solution and saturated sodium hydrogen carbonate aqueous solution. The organic layer was washed with brine, dried over anhydrous sodium sulfate and concentrated under reduced pressure. The residue was purified by silica gel column chromatography (EtOAc/hexane) to give the title compound (28.0 mg).

MS: [M+H]⁺ 369.9.

### C) 2-[2-(3-Bromo-4,5-dihydro-1,2-oxazol-5-yl)-5-chloro-3-fluorophenyl]-3,5-difluoropyridine

The mixture of 2-(5-chloro-3-fluoro-2-iodophenyl)-3,5-difluoropyridine (28 mg), 4,4,5,5-tetramethyl-2-vinyl-1,3,2-dioxaborolane (0.014 mL), potassium carbonate (31.4 mg), PdCl2 (dppf) (5.54 mg), and DME (1 mL)/water (0.20 mL) was heated at 90° C. for 1.5 hours under microwave irradiation. The mixture was filtered and the resultant residue was purified by silica gel column chromatography (EtOAc/hexane) to give 2-(5-chloro-2-ethenyl-3-fluorophenyl)-3,5-difluoropyridine (19 mg, crude). To this crude product was added EtOAc (1 mL), then sodium hydrogen carbonate (63.7 mg) and hydroxycarbonimidic dibromide (30.7 mg) were added at room temperature. The mixture was stirred at 70° C. for 1.5 hours. The mixture was filtered and the resultant residue was purified by silica gel column chromatography (EtOAc/hexane) to give the title compound (19 mg).

¹H NMR (300 MHz, CD₃OD) δ 3.42-3.80 (2H, m), 5.61-5.78 (1H, m), 7.28-7.34 (1H, m), 7.46 (1H, dd, J=10.8, 2.1 Hz), 7.75 (1H, ddd, J=9.4, 8.5, 2.4 Hz), 8.49 (1H, d, J=2.4 Hz).

### D) N-[(3R)-1-{5-[4-Chloro-2-(3,5-difluoropyridin-2-yl)-6-fluorophenyl]-4,5-dihydro-1,2-oxazol-3-yl}-4,4-difluoropyrrolidin-3-yl]methanesulfonamide

N-[(3R)-4,4-Difluoropyrrolidin-3-yl]methanesulfonamide hydrochloride (17.2 mg) was added to a mixture of pyridine (0.3 mL) and 2-[2-(3-bromo-4,5-dihydro-1,2-oxazol-5-yl)-5-chloro-3-fluorophenyl]-3,5-difluoropyridine (19 mg). The mixture was heated in a sealed tube at 140° ° C. overnight. The crude material was purified by preparative HPLC (C18, mobile phase: water (containing 10 mM ammonium bicarbonate)/acetonitrile) to give the title compound (2.3 mg).

¹H NMR (300 MHZ, CD₃OD) δ 3.05 (3H, s), 3.27 (1H, d, J=9.9 Hz), 3.43-3.91 (5H, m), 4.45 (1H, dd, J=14.4, 8.1 Hz), 5.57 (1H, td, J=10.6, 5.4 Hz), 7.28 (1H, dd, J=1.9, 1.0 Hz), 7.45 (1H, dd, J=10.7, 2.1 Hz), 7.74 (1H, td, J=8.9, 2.4 Hz), 8.41-8.50 (1H, m).

The compounds of the Examples are shown in the following tables. In these tables, MS represents an observed molecular ion (found). The compounds described in Examples 1-7, 9, 10, 13-19, 21, 24-29, 31-38, 40, 41, 43-48, 51-58, 60-63, 65-71, 74-81, 83-93, 96-98, 100-106, 108, 110-130, 132-146, 148, 150-153, 155-163, 166, 168-170, and 172-178 were synthesized in the same manner as in the reaction and purification described in the above-mentioned Examples.

TABLE 1-1

| Ex. | | | |
|---|---|---|---|
| No. | IUPAC Name | Structure | MS |
| 1 | N-{(3S)-1-[5-(2′,6′- difluoro[1,1′-biphenyl]-2-yl)- 4,5-dihydro-1,2-oxazol-3- yl]pyrrolidin-3- yl}methanesulfonamide | 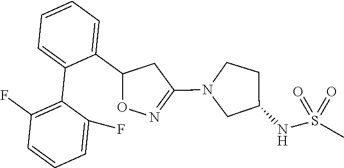 [SMILES: CS(=O)(=O)N[C@H]1CCN(C2=NOC(c3ccccc3-c3c(F)cccc3F)C2)C1] | 422.2 |
| 2 | N-{(3S)-1-[5-(2′,3′,4′,5′- tetrahydro[1,1′-biphenyl]-2-yl)- 4,5-dihydro-1,2-oxazol-3- yl]pyrrolidin-3- yl}methanesulfonamide | 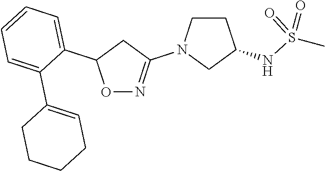 [SMILES: CS(=O)(=O)N[C@H]1CCN(C2=NOC(c3ccccc3C3=CCCCC3)C2)C1] | 390.2 |
| 3 | N-{(3S)-1-[5-(2′,6′- difluoro[1,1′-biphenyl]-2-yl)- 4,5-dihydro-1,2-oxazol-3- yl]pyrrolidin-3- yl}ethanesulfonamide | 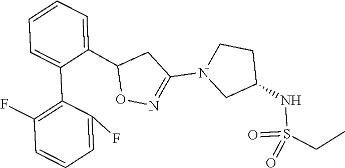 [SMILES: CCS(=O)(=O)N[C@H]1CCN(C2=NOC(c3ccccc3-c3c(F)cccc3F)C2)C1] | 436.2 |
| 4 | N-{(3R)-1-[5-(2′,6′- difluoro[1,1′-biphenyl]-2-yl)- 4,5-dihydro-1,2-oxazol-3-yl]-4,4- difluoropyrrolidin-3- yl}methanesulfonamide | 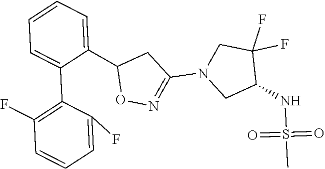 [SMILES: CS(=O)(=O)N[C@@H]1CN(C2=NOC(c3ccccc3-c3c(F)cccc3F)C2)CC1(F)F] | 458.1 |
| 5 | N-{(4S)-1-[5-(2′,6′- difluoro[1,1′-biphenyl]-2-yl)- 4,5-dihydro-1,2-oxazol-3- yl]azepan-4-yl}methanesulfonamide | 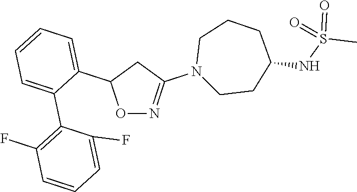 [SMILES: CS(=O)(=O)N[C@H]1CCCN(C2=NOC(c3ccccc3-c3c(F)cccc3F)C2)CC1] | 450.1 |
| 6 | N-{(3S)-1-[5-(2′,6′- difluoro[1,1′-biphenyl]-2-yl)- 4,5-dihydro-1,2-oxazol-3- yl]piperidin-3- yl}methanesulfonamide | 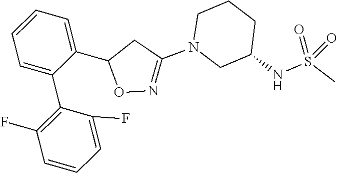 [SMILES: CS(=O)(=O)N[C@H]1CCCN(C2=NOC(c3ccccc3-c3c(F)cccc3F)C2)C1] | 436.1 |

TABLE 1-2

| Ex. | | | |
|---|---|---|---|
| No. | IUPAC Name | Structure | MS |
| 7 | N-{1-[5-(2′,6′-difluoro [1,1′- biphenyl]-2-yl)-4,5-dihydro-1,2- oxazol-3-yl]piperidin-4- yl}methanesulfonamide | 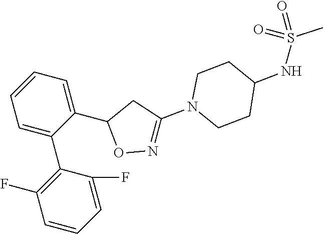 [SMILES: CS(=O)(=O)NC1CCN(C2=NOC(c3ccccc3-c3c(F)cccc3F)C2)CC1] | 436.1 |
| 8 | N-{(3S)-1-[5-(2′,6′-difluoro-5- methyl[1,1′-biphenyl]-2-yl)-4,5- dihydro-1,2-oxazol-3- yl]pyrrolidin-3- yl}ethanesulfonamide | 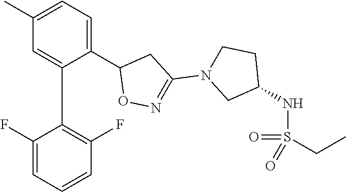 [SMILES: CCS(=O)(=O)N[C@H]1CCN(C2=NOC(c3ccc(C)cc3-c3c(F)cccc3F)C2)C1] | 450.2 |
| 9 | N-[(3S)-1-{5-[1-(2,6- difluorophenyl)-3- (trifluoromethyl)-1H-pyrazol-5- yl]-4,5-dihydro-1,2-oxazol-3- yl}pyrrolidin-3- yl]ethanesulfonamide | 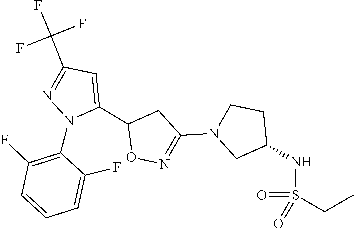 [SMILES: CCS(=O)(=O)N[C@H]1CCN(C2=NOC(c3cc(C(F)(F)F)nn3-c3c(F)cccc3F)C2)C1] | 494.2 |
| 10 | N-[(3R)-4,4-difluoro-1-{5-methyl- 5-[5-methyl-3-(2,4,6- trifluorophenyl)pyridin-2-yl]- 4,5-dihydro-1,2-oxazol-3- yl}pyrrolidin-3- yl]methanesulfonamide(optical isomer) | 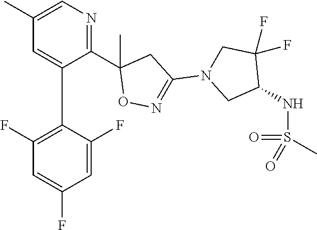 | 505.2 |
| 11 | N-[(3R)-4,4-difluoro-1-{(5S)-5- [5-methyl-3-(2,4,6- trifluorophenyl)pyridin-2-yl]- 4,5-dihydro-1,2-oxazol-3- yl}pyrrolidin-3- yl]methanesulfonamide | 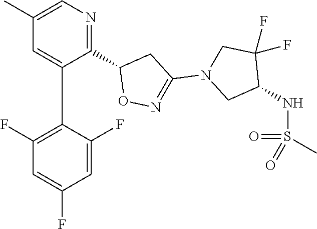 | 491.2 |
| 12 | N-[(3R)-1-{5-[1-(2,6- difluorophenyl)-4-methyl-1H- imidazol-2-yl]-5-methyl-4,5- dihydro-1,2-oxazol-3-yl}-4,4- difluoropyrrolidin-3- yl]methanesulfonamide | 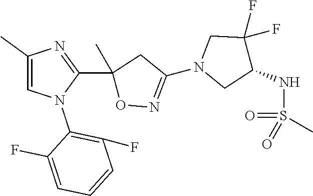 | 476.2 |

TABLE 1-3

| Ex. | | | |
|---|---|---|---|
| No. | IUPAC Name | Structure | MS |
| 13 | N-[(3R)-1-{5-(difluoromethyl)-5- [5-methyl-3-(2,4,6- trifluorophenyl)pyridin-2-yl]- 4,5-dihydro-1,2-oxazol-3-yl}-4,4- difluoropyrrolidin-3- yl]ethanesulfonamide(optical isomer) | 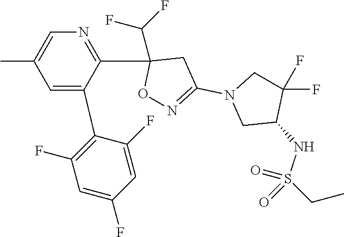 [SMILES: CCS(=O)(=O)N[C@@H]1CN(C2=NOC(c3ncc(C)cc3-c3c(F)cc(F)cc3F)(C(F)F)C2)CC1(F)F] | 555.1 |
| 14 | N-[(3R)-1-{5-(difluoromethyl)-5- [5-methyl-3-(2,4,6- trifluorophenyl)pyridin-2-yl]- 4,5-dihydro-1,2-oxazol-3-yl}-4,4- difluoropyrrolidin-3- yl]methanesulfonamide(optical isomer) | 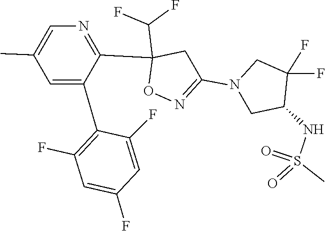 | 540.9 |
| 15 | N-[(3R)-4,4-difluoro-1-{5-[5- fluoro-3-(2,4,6- trifluorophenyl)pyridin-2-yl]-5- (hydroxymethyl)-4,5-dihydro-1,2- oxazol-3-yl}pyrrolidin-3- yl]methanesulfonamide | 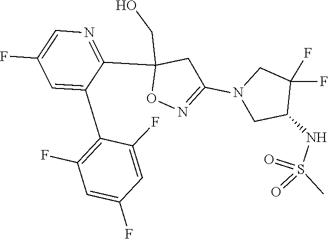 [SMILES: CS(=O)(=O)N[C@@H]1CN(C2=NOC(CO)(c3ncc(F)cc3-c3c(F)cc(F)cc3F)C2)CC1(F)F] | 525.1 |
| 16 | N-[(3R)-4,4-difluoro-1-{5- (hydroxymethyl)-5-[5-methyl-3- (2,4,6-trifluorophenyl)pyridin-2- yl]-4,5-dihydro-1,2-oxazol-3- yl}pyrrolidin-3- yl]methanesulfonamide(optical isomer) | 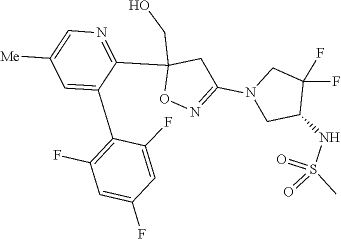 [SMILES: Cc1cnc(C2(CO)CC(N3C[C@@H](NS(C)(=O)=O)C(F)(F)C3)=NO2)c(-c2c(F)cc(F)cc2F)c1] | 521.2 |
| 17 | N-[(3,3-difluoro-1-{5-[5-methyl- 3-(2,4,6-trifluorophenyl)pyridin- 2-yl]-4,5-dihydro-1,2-oxazol-3- yl}azetidin-2- yl)methyl]methanesulfonamide | 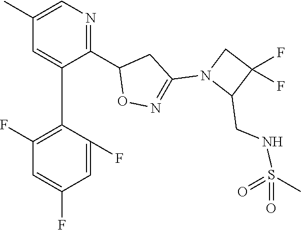 | 491.2 |
| 18 | N-[(3R)-1-{5-[5-cyclopropyl-3- (2,4,6-trifluorophenyl)pyridin-2- yl]-4,5-dihydro-1,2-oxazol-3-yl}- 4,4-difluoropyrrolidin-3- yl]methanesulfonamide(optical isomer) | 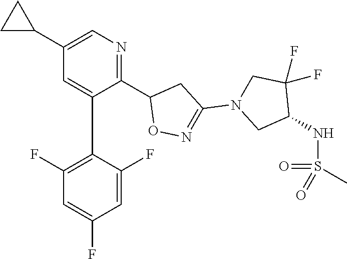 [SMILES: CS(=O)(=O)N[C@@H]1CN(C2=NOC(c3ncc(C4CC4)cc3-c3c(F)cc(F)cc3F)C2)CC1(F)F] | 517.1 |

TABLE 1-4

| Ex. | | | |
|---|---|---|---|
| No. | IUPAC Name | Structure | MS |
| 19 | N-[(1S,3R)-3-{5-[5-methyl-3- (2,4,6-trifluorophenyl)pyridin-2- yl]-4,5-dihydro-1,2-oxazol-3- yl}cyclopentyl]methanesulfonamide (optical isomer) | 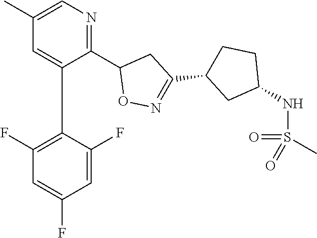 | 454.2 |
| 20 | N-[(1s,3s)-3-( {5-[3-(2,6- difluorophenyl)-5-methylpyridin- 2-yl]-4,5-dihydro-1,2-oxazol-3- yl}oxy)cyclobutyl] methanesulfonamide | 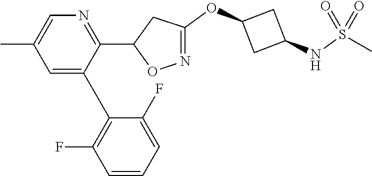 | 438.1 |
| 21 | N-[(3R)-1-{5-[3-(2,6- difluorophenyl)-5-methylpyridin- 2-yl]-4,5-dihydro-1,2-oxazol-3- yl}-4,4-difluoropyrrolidin-3- yl]methanesulfonamide(optical isomer) | 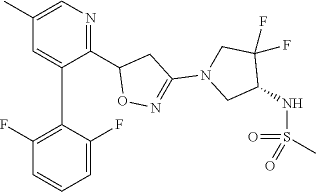 | 473.1 |
| 22 | N-[(3R)-1-{(5S)-5-[3-(2,6- difluorophenyl)-5-methylpyridin- 2-yl]-4,5-dihydro-1,2-oxazol-3- yl}-4,4-difluoropyrrolidin-3- yl]ethanesulfonamide | 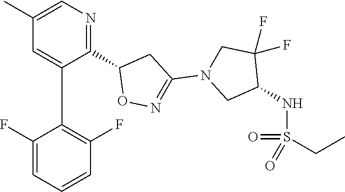 [SMILES: CCS(=O)(=O)N[C@@H]1CN(C2=NO[C@H](c3ncc(C)cc3-c3c(F)cccc3F)C2)CC1(F)F] | 487.1 |
| 23 | N-[(1s,3s)-3-{5-[5-methyl-3- (2,4,6-trifluorophenyl)pyridin-2- yl]-4,5-dihydro-1,2-oxazol-3- yl}cyclobutyl] methanesulfonamide | 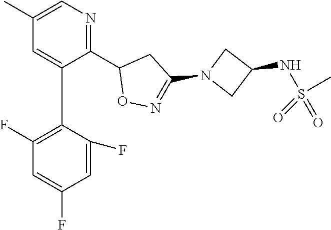 | 440.2 |
| 24 | N-[(1s,4s)-4-{5-[5-methyl-3- (2,4,6-trifluorophenyl)pyridin-2- yl]-4,5-dihydro-1,2-oxazol-3- yl}cyclohexyl] methanesulfonamide | 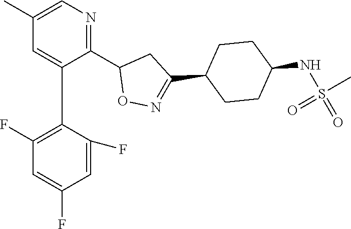 | 468.3 |

TABLE 1-5

| Ex. | | | MS |
|---|---|---|---|
| No. | IUPAC Name | Structure | |
| 25 | N-[(1S,3S)-3-{5-[5-methyl-3- (2,4,6-trifluorophenyl)pyridin-2- yl]-4,5-dihydro-1,2-oxazol-3- yl}cyclopentyl]methanesulfonamide | 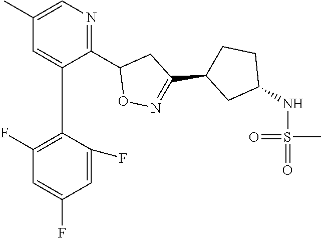 | 454.2 |
| 26 | N-{[(2S)-1-{5-[3-(2,6- difluorophenyl)-5-methylpyridin- 2-yl]-4,5-dihydro-1,2-oxazol-3- yl}pyrrolidin-2- yl]methyl}ethanesulfonamide | 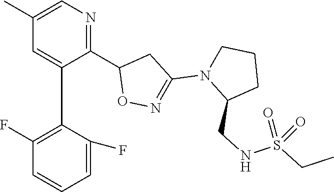 [SMILES: CCS(=O)(=O)NC[C@@H]1CCCN1C1=NOC(c2ncc(C)cc2-c2c(F)cccc2F)C1] | 465.3 |
| 27 | N-{(1s,3s)-3-[{5-[3-(2,6- difluorophenyl)-5-methylpyridin- 2-yl]-4,5-dihydro-1,2-oxazol-3- yl}(methyl)amino]cyclobutyl} methanesulfonamide | 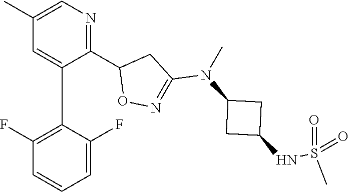 [SMILES: Cc1cnc(C2CC(N(C)[C@H]3C[C@@H](NS(C)(=O)=O)C3)=NO2)c(-c2c(F)cccc2F)c1] | 451.3 |
| 28 | N-[(4R)-1-{5-[3-(2,6- difluorophenyl)-5-methylpyridin- 2-yl]-4,5-dihydro-1,2-oxazol-3- yl}-5,5-difluoroazepan-4- yl]methanesulfonamide(optical isomer) | 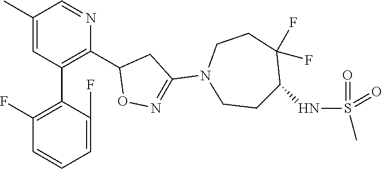 [SMILES: Cc1cnc(C2CC(N3CC[C@@H](NS(C)(=O)=O)C(F)(F)CC3)=NO2)c(-c2c(F)cccc2F)c1] | 501.2 |
| 29 | N-[(3S)-1-{(5S)-5-[3-(2,6- difluorophenyl)-5-methylpyridin- 2-yl]-4,5-dihydro-1,2-oxazol-3- yl}pyrrolidin-3- yl]methanesulfonamide | 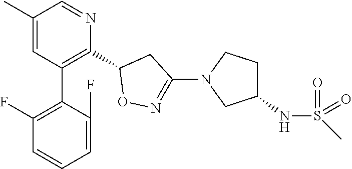 [SMILES: Cc1cnc([C@@H]2CC(N3CC[C@H](NS(C)(=O)=O)C3)=NO2)c(-c2c(F)cccc2F)c1] | 437.3 |
| 30 | N-[(3S)-1-{(5S)-5-[3-(2,6- difluorophenyl)-5-methylpyridin- 2-yl]-4,5-dihydro-1,2-oxazol-3- yl}pyrrolidin-3-yl]-1- fluorocyclopropane-1-sulfonamide | 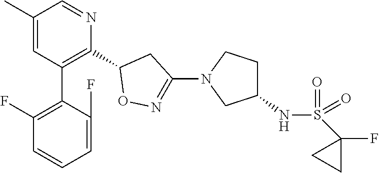 [SMILES: Cc1cnc([C@@H]2CC(N3CC[C@H](NS(=O)(=O)C4(F)CC4)C3)=NO2)c(-c2c(F)cccc2F)c1] | 481.3 |

TABLE 1-6

| Ex. | | | |
|---|---|---|---|
| No. | IUPAC Name | Structure | MS |
| 31 | N-[(3R)-1-{(5S)-5-[3-(2,6- difluorophenyl)-5-methylpyridin- 2-yl]-4,5-dihydro-1,2-oxazol-3- yl}-4,4-difluoropyrrolidin-3-yl]- 1,1-difluoromethanesulfonamide | 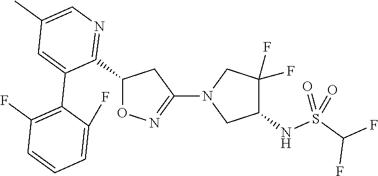 [SMILES: Cc1cnc([C@@H]2CC(N3C[C@@H](NS(=O)(=O)C(F)F)C(F)(F)C3)=NO2)c(-c2c(F)cccc2F)c1] | 509.2 |
| 32 | N-[(3R,4S)-1-{(5S)-5-[3-(2,6- difluorophenyl)-5-methylpyridin- 2-yl]-4,5-dihydro-1,2-oxazol-3- yl}-4-fluoropyrrolidin-3- yl]methanesulfonamide | 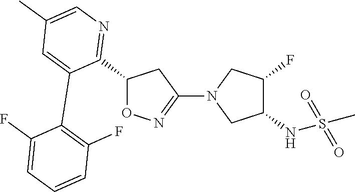 [SMILES: Cc1cnc([C@@H]2CC(N3C[C@H](F)[C@H](NS(C)(=O)=O)C3)=NO2)c(-c2c(F)cccc2F)c1] | 455.2 |
| 33 | N-[(3R)-1-{(5S)-5-[3-(2,6- difluorophenyl)-5-methylpyridin- 2-yl]-4,5-dihydro-1,2-oxazol-3- yl}-4,4-difluoropyrrolidin-3- yl]cyclopropanesulfonamide | 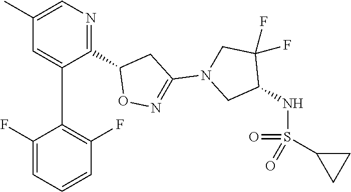 [SMILES: Cc1cnc([C@@H]2CC(N3C[C@@H](NS(=O)(=O)C4CC4)C(F)(F)C3)=NO2)c(-c2c(F)cccc2F)c1] | 499.3 |
| 34 | N-[(3R)-4,4-difluoro-1-{5-[5- fluoro-3-(2,4,6- trifluorophenyl)pyridin-2-yl]- 4,5-dihydro-1,2-oxazol-3- yl}pyrrolidin-3- yl]ethanesulfonamide | 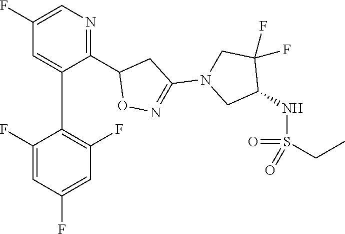 [SMILES: CCS(=O)(=O)N[C@@H]1CN(C2=NOC(c3ncc(F)cc3-c3c(F)cc(F)cc3F)C2)CC1(F)F] | 509.2 |
| 35 | N,N-dimethyl-N′-[(3S)-1-{5-[3- (2,4,6-trifluorophenyl)pyridin-2- yl]-4,5-dihydro-1,2-oxazol-3- yl}pyrrolidin-3-yl] sulfuric diamide | 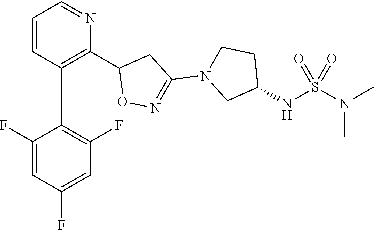 [SMILES: CN(C)S(=O)(=O)N[C@H]1CCN(C2=NOC(c3ncccc3-c3c(F)cc(F)cc3F)C2)C1] | 470.3 |
| 36 | 1-fluoro-N-[(3S)-1-{5-[5-fluoro- 3-(2,4,6-trifluorophenyl)pyridin- 2-yl]-4,5-dihydro-1,2-oxazol-3- yl}pyrrolidin-3- yl]methanesulfonamide | 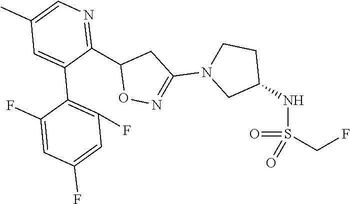 [SMILES: Cc1cnc(C2CC(N3CC[C@H](NS(=O)(=O)CF)C3)=NO2)c(-c2c(F)cc(F)cc2F)c1] | 477.2 |

TABLE 1-7

| Ex. | | | |
|---|---|---|---|
| No. | IUPAC Name | Structure | MS |
| 37 | N-[(3S)-1-{(5S)-5-[3-(2,6- difluorophenyl)-5-methylpyridin- 2-yl]-4,5-dihydro-1,2-oxazol-3- yl}pyrrolidin-3-yl]-1,1- difluoromethanesulfonamide | 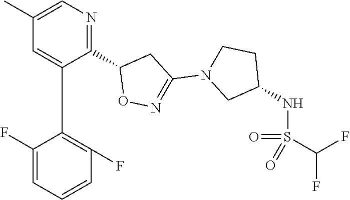 [SMILES: Cc1cnc([C@@H]2CC(N3CC[C@H](NS(=O)(=O)C(F)F)C3)=NO2)c(-c2c(F)cccc2F)c1] | 473.2 |
| 38 | N-[(3S)-1-{(5S)-5-[3-(2,6- difluorophenyl)-5-methylpyridin- 2-yl]-4,5-dihydro-1,2-oxazol-3- yl}pyrrolidin-3-yl]-1- fluoromethanesulfonamide | 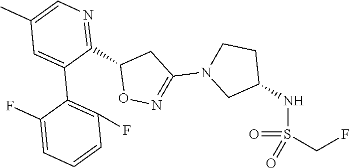 [SMILES: Cc1cnc([C@@H]2CC(N3CC[C@H](NS(=O)(=O)CF)C3)=NO2)c(-c2c(F)cccc2F)c1] | 455.3 |
| 39 | N-[(3R)-1-{5-[5-chloro-3-(2,4,6- trifluorophenyl)pyridin-2-yl]- 4,5-dihydro-1,2-oxazol-3-yl}-4,4- difluoropyrrolidin-3- yl]methanesulfonamide(optical isomer) | 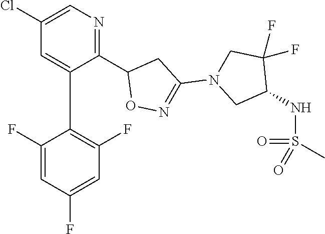 [SMILES: CS(=O)(=O)N[C@@H]1CN(C2=NOC(c3ncc(Cl)cc3-c3c(F)cc(F)cc3F)C2)CC1(F)F] | 511.1 |
| 40 | N-[(3R)-1-{5-[5-bromo-3-(2,4,6- trifluorophenyl)pyridin-2-yl]- 4,5-dihydro-1,2-oxazol-3-yl}-4,4- difluoropyrrolidin-3- yl]methanesulfonamide(optical isomer) | 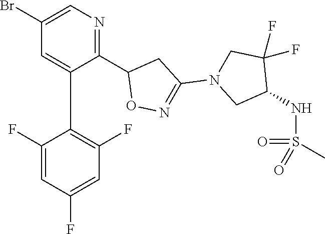 [SMILES: CS(=O)(=O)N[C@@H]1CN(C2=NOC(c3ncc(Br)cc3-c3c(F)cc(F)cc3F)C2)CC1(F)F] | 555.1 |
| 41 | N-[(3R)-1-{5-[5-(difluoromethyl)- 3-(2,4,6-trifluorophenyl)pyridin- 2-yl]-4,5-dihydro-1,2-oxazol-3- yl}-4,4-difluoropyrrolidin-3- yl]methanesulfonamide | 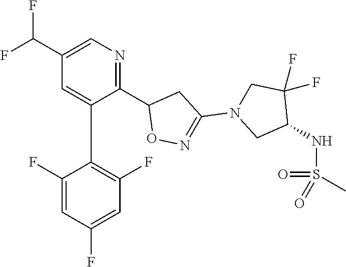 [SMILES: CS(=O)(=O)N[C@@H]1CN(C2=NOC(c3ncc(C(F)F)cc3-c3c(F)cc(F)cc3F)C2)CC1(F)F] | 527.1 |
| 42 | N-[(3R)-1-{5-[5-chloro-3-(2,4,6- trifluorophenyl)pyridin-2-yl]- 4,5-dihydro-1,2-oxazol-3-yl}-4,4- difluoropyrrolidin-3- yl]ethanesulfonamide(optical isomer) | 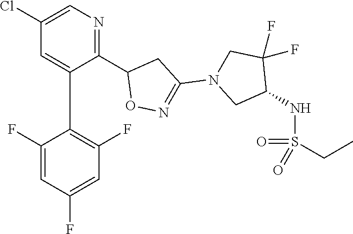 [SMILES: CCS(=O)(=O)N[C@@H]1CN(C2=NOC(c3ncc(Cl)cc3-c3c(F)cc(F)cc3F)C2)CC1(F)F] | 525.1 |

TABLE 1-8

| Ex. | | | |
|---|---|---|---|
| No. | IUPAC Name | Structure | MS |
| 43 | N-[(3R)-1-{5-[5-bromo-3-(2,4,6- trifluorophenyl)pyridin-2-yl]- 4,5-dihydro-1,2-oxazol-3-yl}-4,4- difluoropyrrolidin-3- yl]ethanesulfonamide(optical isomer) | 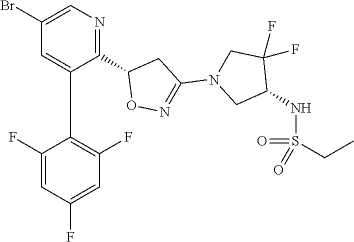 [SMILES: CCS(=O)(=O)N[C@@H]1CN(C2=NO[C@H](c3ncc(Br)cc3-c3c(F)cc(F)cc3F)C2)CC1(F)F] | 569.0 |
| 44 | N-[(3R,4R)-1-{(5S)-5-[3-(2,6- difluorophenyl)-5-methylpyridin- 2-yl]-4,5-dihydro-1,2-oxazol-3- yl}-4-fluoropyrrolidin-3- yl]methanesulfonamide | 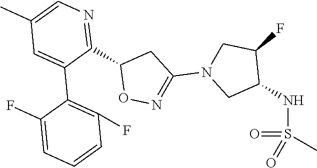 | 455.2 |
| 45 | N-[(3S)-1-{(5S)-5-[5-methyl-3- (2,4,6-trifluorophenyl)pyridin-2- yl]-4,5-dihydro-1,2-oxazol-3- yl}pyrrolidin-3- yl]methanesulfonamide | 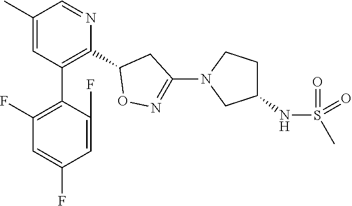 [SMILES: Cc1cnc([C@@H]2CC(N3CC[C@H](NS(C)(=O)=O)C3)=NO2)c(-c2c(F)cc(F)cc2F)c1] | 455.2 |
| 46 | 1-fluoro-N-[(3S)-1-{(5S)-5-[5- methyl-3-(2,4,6- trifluorophenyl)pyridin-2-yl]- 4,5-dihydro-1,2-oxazol-3- yl}pyrrolidin-3-yl]cyclopropane- 1-sulfonamide | 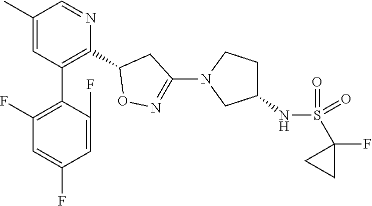 [SMILES: Cc1cnc([C@@H]2CC(N3CC[C@H](NS(=O)(=O)C4(F)CC4)C3)=NO2)c(-c2c(F)cc(F)cc2F)c1] | 499.3 |
| 47 | N-[(3R)-4,4-difluoro-1-{(5S)-5- [5-methyl-3-(2,4,6- trifluorophenyl)pyridin-2-yl]- 4,5-dihydro-1,2-oxazol-3- yl}pyrrolidin-3- yl]ethanesulfonamide | 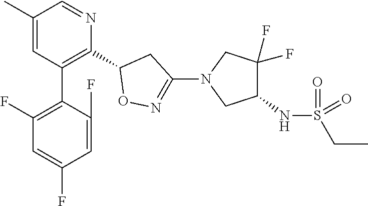 [SMILES: CCS(=O)(=O)N[C@@H]1CN(C2=NO[C@H](c3ncc(C)cc3-c3c(F)cc(F)cc3F)C2)CC1(F)F] | 505.2 |
| 48 | N-[(3R,4R)-4-fluoro-1-{(5S)-5-[5- methyl-3-(2,4,6- trifluorophenyl)pyridin-2-yl]- 4,5-dihydro-1,2-oxazol-3- yl}pyrrolidin-3- yl]methanesulfonamide | 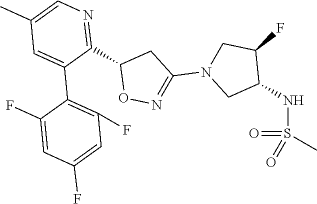 | 473.2 |

TABLE 1-9

| Ex. | | | |
|---|---|---|---|
| No. | IUPAC Name | Structure | MS |
| 49 | N-[(3R)-1-{5-[5-chloro-3-(2,6- difluorophenyl)pyridin-2-yl]-4,5- dihydro-1,2-oxazol-3-yl}-4,4- difluoropyrrolidin-3- yl]methanesulfonamide(optical isomer) | 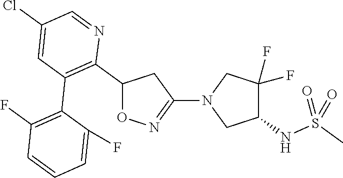 [SMILES: CS(=O)(=O)N[C@@H]1CN(C2=NOC(c3ncc(Cl)cc3-c3c(F)cccc3F)C2)CC1(F)F] | 493.2 |
| 50 | N-[(3S)-1-{5-[2-(3,5- difluoropyridin-2-yl)-4- methylphenyl]-4,5-dihydro-1,2- oxazol-3-yl}pyrrolidin-3- yl]methanesulfonamide | 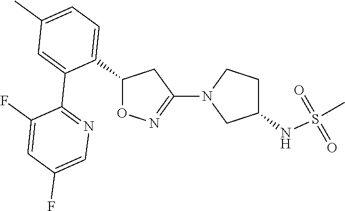 [SMILES: Cc1ccc([C@@H]2CC(N3CC[C@H](NS(C)(=O)=O)C3)=NO2)c(-c2ncc(F)cc2F)c1] | 437.3 |
| 51 | mixture of N-[(3R,4S)-1-{(5S)-5- [3-(2,6-difluorophenyl)-5- methylpyridin-2-yl]-4,5-dihydro- 1,2-oxazol-3-yl}-4-fluoro-4- methylpyrrolidin-3- yl]methanesulfonamide and N- [(3S,4R)-1-{(5S)-5-[3-(2,6- difluorophenyl)-5-methylpyridin- 2-yl]-4,5-dihydro-1,2-oxazol-3- yl}-4-fluoro-4-methylpyrrolidin- | 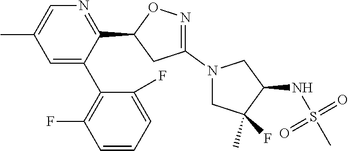 [SMILES: Cc1cnc([C@@H]2CC(N3C[C@@H](NS(C)(=O)=O)[C@@](C)(F)C3)=NO2)c(-c2c(F)cccc2F)c1] | 469.3 |
| | 3-yl]methanesulfonamide | | |
| 52 | N-[(3R)-1-{5-[5-bromo-3-(2,6- difluorophenyl)pyridin-2-yl]-4,5- dihydro-1,2-oxazol-3-yl}-4,4- difluoropyrrolidin-3- yl]methanesulfonamide | 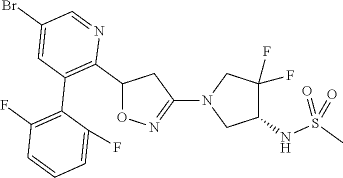 [SMILES: CS(=O)(=O)N[C@@H]1CN(C2=NOC(c3ncc(Br)cc3-c3c(F)cccc3F)C2)CC1(F)F] | 537.1 |
| 53 | N-[(3R)-1-{5-[3-(4-chloro-2,6- difluorophenyl)-5-methylpyridin- 2-yl]-4,5-dihydro-1,2-oxazol-3- yl}-4,4-difluoropyrrolidin-3- yl]methanesulfonamide | 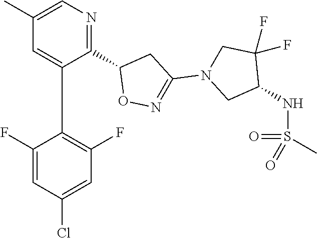 | 507.2 |

TABLE 1-10

| Ex. | | | |
|---|---|---|---|
| No. | IUPAC Name | Structure | MS |
| 54 | N-[(3R)-4,4-difluoro-1-{5-[5- methyl-3-(2,3,6- trifluorophenyl)pyridin-2-yl]- 4,5-dihydro-1,2-oxazol-3- yl}pyrrolidin-3- yl]methanesulfonamide | 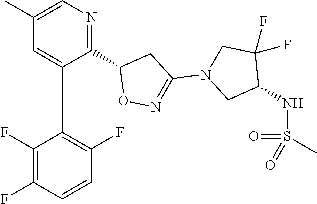 | 491.2 |
| 55 | N-[(3R)-4,4-difluoro-1-{(5S)-5- [5-methyl-3-(2,4,6- trifluorophenyl)pyridin-2-yl]- 4,5-dihydro-1,2-oxazol-3- yl}pyrrolidin-3- yl]cyclopropanesulfonamide | 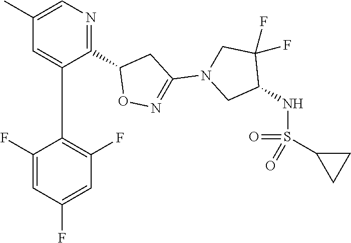 [SMILES: Cc1cnc([C@@H]2CC(N3C[C@@H](NS(=O)(=O)C4CC4)C(F)(F)C3)=NO2)c(-c2c(F)cc(F)cc2F)c1] | 517.2 |
| 56 | 1-fluoro-N-[(3S)-1-{(5S)-5-[5- methyl-3-(2,4,6- trifluorophenyl)pyridin-2-yl]- 4,5-dihydro-1,2-oxazol-3- yl}pyrrolidin-3- yl]methanesulfonamide | 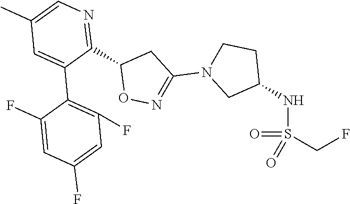 [SMILES: Cc1cnc([C@@H]2CC(N3CC[C@H](NS(=O)(=O)CF)C3)=NO2)c(-c2c(F)cc(F)cc2F)c1] | 473.3 |
| 57 | N-[(3R,4S)-4-fluoro-1-{(5S)-5-[5- methyl-3-(2,4,6- trifluorophenyl)pyridin-2-yl]- 4,5-dihydro-1,2-oxazol-3- yl}pyrrolidin-3- yl]methanesulfonamide | 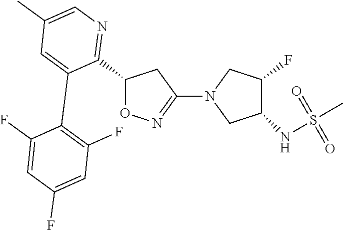 [SMILES: Cc1cnc([C@@H]2CC(N3C[C@H](F)[C@H](NS(C)(=O)=O)C3)=NO2)c(-c2c(F)cc(F)cc2F)c1] | 473.3 |
| 58 | N-[(3R)-4,4-difluoro-1-{(5S)-5- [5-methyl-3-(2,4,6- trifluorophenyl)pyridin-2-yl]- 4,5-dihydro-1,2-oxazol-3- yl}pyrrolidin-3-yl]-1- fluoromethanesulfonamide | 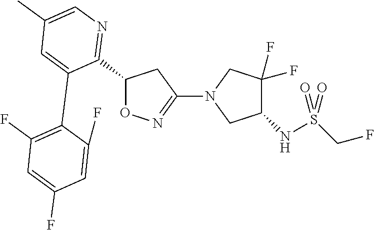 [SMILES: Cc1cnc([C@@H]2CC(N3C[C@@H](NS(=O)(=O)CF)C(F)(F)C3)=NO2)c(-c2c(F)cc(F)cc2F)c1] | 509.2 |
| 59 | N-[(3R)-1-{5-(difluoromethyl)-5- [3-(2,6-difluorophenyl)-5- methylpyridin-2-yl]-4,5-dihydro- 1,2-oxazol-3-yl}-4,4- difluoropyrrolidin-3- yl]methanesulfonamide(optical isomer) | 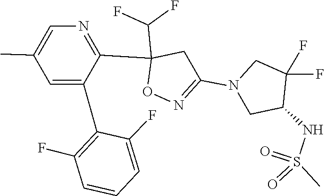 | 523.2 |

TABLE 1-11

| Ex. | | | |
|---|---|---|---|
| No. | IUPAC Name | Structure | MS |
| 60 | N-[(3R)-4,4-difluoro-1-{(5S)-5- [5-methyl-3-(2,4,6- trifluorophenyl)pyridin-2-yl]- 4,5-dihydro-1,2-oxazol-3- yl}pyrrolidin-3-yl]-1,1- difluoromethanesulfonamide | 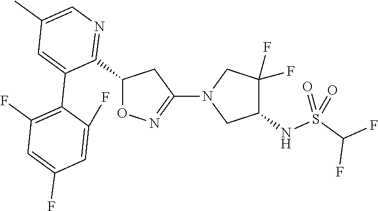 [SMILES: Cc1cnc([C@@H]2CC(N3C[C@@H](NS(=O)(=O)C(F)F)C(F)(F)C3)=NO2)c(-c2c(F)cc(F)cc2F)c1] | 527.1 |
| 61 | N-[(3R)-1-{5-[3-(2-chloro-6- fluorophenyl)-5-methylpyridin-2- yl]-4,5-dihydro-1,2-oxazol-3-yl}- 4,4-difluoropyrrolidin-3- yl]methanesulfonamide | 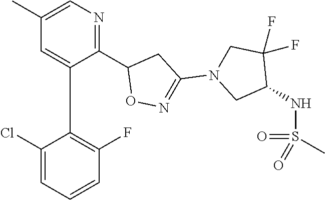 | 489.2 |
| 62 | mixture of N-[(1R,3S)-3-( {(5S)-5- [5-methyl-3-(2,4,6- trifluorophenyl)pyridin-2-yl]- 4,5-dihydro-1,2-oxazol-3- yl}oxy)cyclopentyl] methanesulfonamide and N-[(1S,3R)-3-( {(5S)-5- [5-methyl-3-(2,4,6- trifluorophenyl)pyridin-2-yl]- 4,5-dihydro-1,2-oxazol-3- yl}oxy)cyclopentyl] methanesulfonamide | 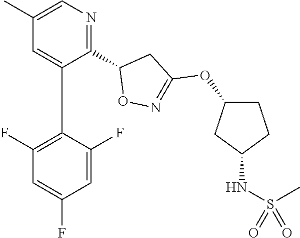 | 470.3 |
| 63 | N-[(3S,4R)-4-methyl-1-{(5S)-5-[5- methyl-3-(2,4,6- trifluorophenyl)pyridin-2-yl]- 4,5-dihydro-1,2-oxazol-3- yl}pyrrolidin-3- yl]methanesulfonamide | 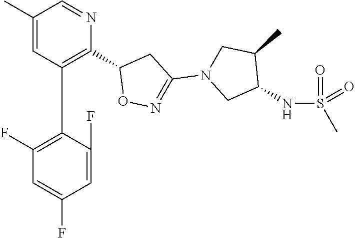 [SMILES: Cc1cnc([C@@H]2CC(N3C[C@@H](C)[C@H](NS(C)(=O)=O)C3)=NO2)c(-c2c(F)cc(F)cc2F)c1] | 469.3 |
| 64 | N-(4,4-difluoro-3-methyl-1-{(5S)- 5-[5-methyl-3-(2,4,6- trifluorophenyl)pyridin-2-yl]- 4,5-dihydro-1,2-oxazol-3- yl}pyrrolidin-3- yl)methanesulfonamide | 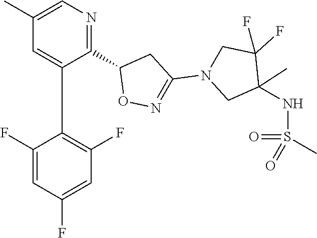 | 505.2 |

TABLE 1-12

| Ex. | | | |
|---|---|---|---|
| No. | IUPAC Name | Structure | MS |
| 65 | N-[(3R,4R)-1-{(5S)-5-[3-(2,6- difluorophenyl)-5-methylpyridin- 2-yl]-4,5-dihydro-1,2-oxazol-3- yl}-4-fluoropyrrolidin-3-yl]-1- fluoromethanesulfonamide | 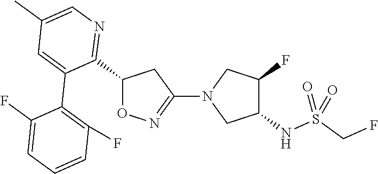 [SMILES: Cc1cnc([C@@H]2CC(N3C[C@@H](F)[C@H](NS(=O)(=O)CF)C3)=NO2)c(-c2c(F)cccc2F)c1] | 473.2 |
| 66 | 1,1-difluoro-N-[(3S)-1-{(5S)-5- [5-methyl-3-(2,4,6- trifluorophenyl)pyridin-2-yl]- 4,5-dihydro-1,2-oxazol-3- yl}pyrrolidin-3- yl]methanesulfonamide | 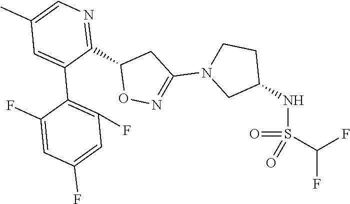 [SMILES: Cc1cnc([C@@H]2CC(N3CC[C@H](NS(=O)(=O)C(F)F)C3)=NO2)c(-c2c(F)cc(F)cc2F)c1] | 491.2 |
| 67 | N-[(3R)-1-{5-[3-(2,6- difluorophenyl)-5-fluoropyridin- 2-yl]-4,5-dihydro-1,2-oxazol-3- yl}-4,4-difluoropyrrolidin-3- yl]cyclopropanesulfonamide | 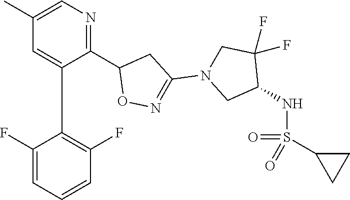 [SMILES: Cc1cnc(C2CC(N3C[C@@H](NS(=O)(=O)C4CC4)C(F)(F)C3)=NO2)c(-c2c(F)cccc2F)c1] | 503.2 |
| 68 | N-[(3R,4S)-1-{(5S)-5-[3-(2,6- difluorophenyl)-5-methylpyridin- 2-yl]-4,5-dihydro-1,2-oxazol-3- yl}-4-fluoropyrrolidin-3-yl]-1- fluoromethanesulfonamide | 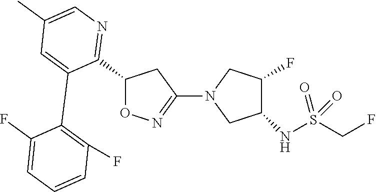 [SMILES: Cc1cnc([C@@H]2CC(N3C[C@H](F)[C@H](NS(=O)(=O)CF)C3)=NO2)c(-c2c(F)cccc2F)c1] | 473.3 |
| 69 | N-[(3R)-1-{5-[3-(2,6- difluorophenyl)-5-fluoropyridin- 2-yl]-4,5-dihydro-1,2-oxazol-3- yl}-4,4-difluoropyrrolidin-3- yl]methanesulfonamide | 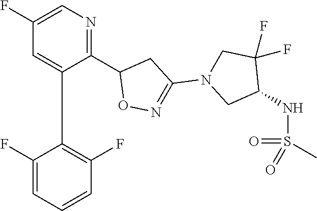 [SMILES: CS(=O)(=O)N[C@@H]1CN(C2=NOC(c3ncc(F)cc3-c3c(F)cccc3F)C2)CC1(F)F] | 477.2 |
| 70 | N-[(3R)-4,4-difluoro-1-{5-[5- fluoro-3-(2,4,6- trifluorophenyl)pyridin-2-yl]- 4,5-dihydro-1,2-oxazol-3- yl}pyrrolidin-3- yl]methanesulfonamide(optical isomer) | 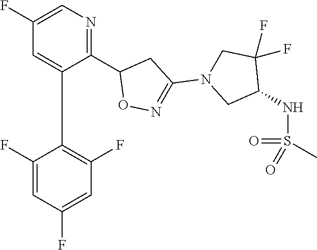 [SMILES: CS(=O)(=O)N[C@@H]1CN(C2=NOC(c3ncc(F)cc3-c3c(F)cc(F)cc3F)C2)CC1(F)F] | 495.2 |

TABLE 1-13

| Ex. | | | |
|---|---|---|---|
| No. | IUPAC Name | Structure | MS |
| 71 | optical isomer of N-[(3S,4R)-1- {(5S)-5-[3-(2,6-difluorophenyl)- 5-methylpyridin-2-yl]-4,5- dihydro-1,2-oxazol-3-yl}-4- methylpyrrolidin-3- yl]methanesulfonamide or N- [(3R,4S)-1-{(5S)-5-[3-(2,6- difluorophenyl)-5-methylpyridin- 2-yl]-4,5-dihydro-1,2-oxazol-3- yl}-4-methylpyrrolidin-3- yl]methanesulfonamide | 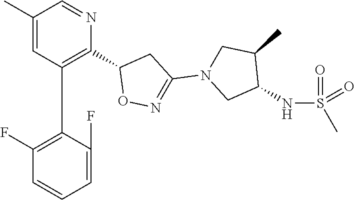 [SMILES: Cc1cnc([C@@H]2CC(N3C[C@@H](C)[C@H](NS(C)(=O)=O)C3)=NO2)c(-c2c(F)cccc2F)c1] | 451.2 |
| 72 | N-[(3R)-4,4-difluoro-1-{5-[6- methoxy-4-(2,4,6- trifluorophenyl)pyridin-3-yl]- 4,5-dihydro-1,2-oxazol-3- yl}pyrrolidin-3- yl]methanesulfonamide | 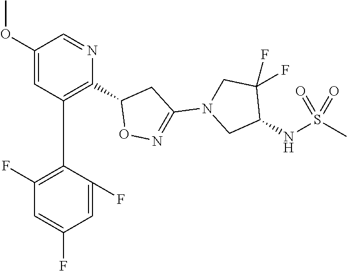 [SMILES: COc1cnc([C@@H]2CC(N3C[C@@H](NS(C)(=O)=O)C(F)(F)C3)=NO2)c(-c2c(F)cc(F)cc2F)c1] | 507.2 |
| 73 | N-[1-{(5S)-5-[3-(2,6- difluorophenyl)-5-methylpyridin- 2-yl]-4,5-dihydro-1,2-oxazol-3- yl}-3-(methoxymethyl)pyrrolidin- 3-yl]methanesulfonamide(optical isomer) | 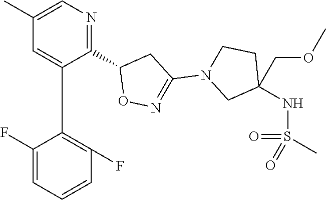 | 481.3 |
| 74 | N-[(1s,3r)-3-( {(5S)-5-[5-methyl- 3-(2,4,6-trifluorophenyl)pyridin- 2-yl]-4,5-dihydro-1,2-oxazol-3- yl}oxy)cyclobutyl] methanesulfonamide | 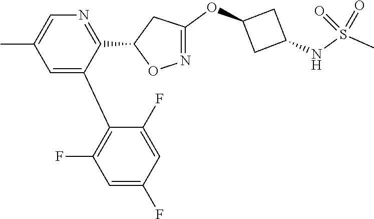 | 456.2 |

TABLE 1-14

| Ex. | | | |
|---|---|---|---|
| No. | IUPAC Name | Structure | MS |
| 75 | optical isomer of N-[(3R,4S)-4- fluoro-4-methyl-1-{(5S)-5-[5- methyl-3-(2,4,6- trifluorophenyl)pyridin-2-yl]- 4,5-dihydro-1,2-oxazol-3- yl}pyrrolidin-3- yl]methanesulfonamide or N- [(3S,4R)-4-fluoro-4-methyl-1- {(5S)-5-[5-methyl-3-(2,4,6- trifluorophenyl)pyridin-2-yl]- 4,5-dihydro-1,2-oxazol-3- yl}pyrrolidin-3- yl]methanesulfonamide | 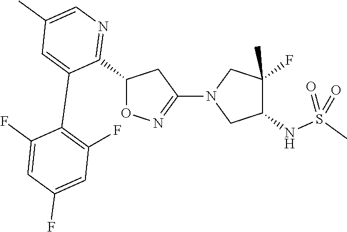 [SMILES: Cc1cnc([C@@H]2CC(N3C[C@@H](NS(C)(=O)=O)[C@@](C)(F)C3)=NO2)c(-c2c(F)cc(F)cc2F)c1] | 487.2 |
| 76 | N-[(3R)-1-{5-[3-(2,6- difluorophenyl)-5-methylpyridin- 2-yl]-5-methyl-4,5-dihydro-1,2- oxazol-3-yl}-4,4- difluoropyrrolidin-3- yl]methanesulfonamide | 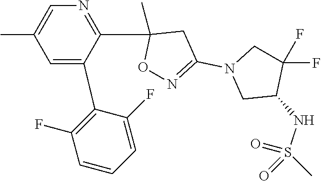 [SMILES: Cc1cnc(C2(C)CC(N3C[C@@H](NS(C)(=O)=O)C(F)(F)C3)=NO2)c(-c2c(F)cccc2F)c1] | 487.2 |
| 77 | N′-[(3R,4R)-4-fluoro-1-{(5S)-5- [5-methyl-3-(2,4,6- trifluorophenyl)pyridin-2-yl]- 4,5-dihydro-1,2-oxazol-3- yl}pyrrolidin-3-yl]-N,N- dimethylsulfuric diamide | 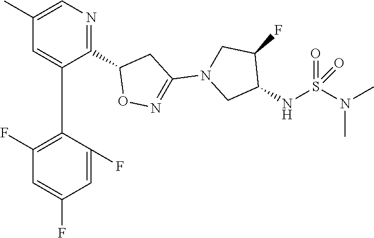 | 502.3 |
| 78 | N′-[(3R,4R)-1-{(5S)-5-[3-(2,6- difluorophenyl)-5-methylpyridin- 2-yl]-4,5-dihydro-1,2-oxazol-3- yl}-4-fluoropyrrolidin-3-yl]-N,N- dimethylsulfuric diamide | 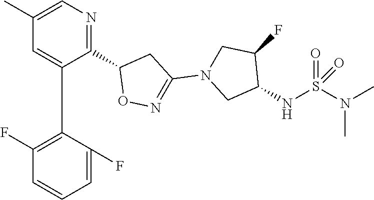 | 484.3 |
| 79 | N′-[(3R,4S)-1-{(5S)-5-[3-(2,6- difluorophenyl)-5-methylpyridin- 2-yl]-4,5-dihydro-1,2-oxazol-3- yl}-4-fluoropyrrolidin-3-yl]-N,N- dimethylsulfuric diamide | 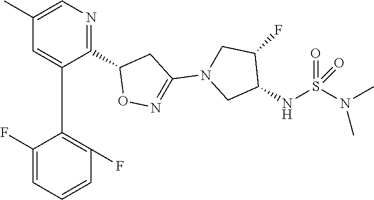 | 484.3 |

TABLE 1-15

| Ex. | | | |
|---|---|---|---|
| No. | IUPAC Name | Structure | MS |
| 80 | N-[(3R,4S)-1-{5-[5-chloro-3-(2,6- difluorophenyl)pyridin-2-yl]-4,5- dihydro-1,2-oxazol-3-yl}-4- fluoropyrrolidin-3- yl]methanesulfonamide | 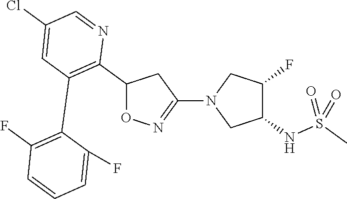 [SMILES: CS(=O)(=O)N[C@@H]1CN(C2=NOC(c3ncc(Cl)cc3-c3c(F)cccc3F)C2)C[C@@H]1F] | 475.2 |
| 81 | N-[(3R,4S)-1-{(5S)-5-[3-(2,6- difluorophenyl)-5-methylpyridin- 2-yl]-4,5-dihydro-1,2-oxazol-3- yl}-4-fluoropyrrolidin-3-yl]-1,1- difluoromethanesulfonamide | 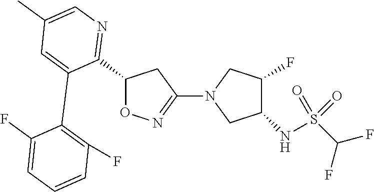 [SMILES: Cc1cnc([C@@H]2CC(N3C[C@H](F)[C@H](NS(=O)(=O)C(F)F)C3)=NO2)c(-c2c(F)cccc2F)c1] | 491.2 |
| 82 | 1-fluoro-N-[(3R,4S)-4-fluoro-1- {(5S)-5-[5-methyl-3-(2,4,6- trifluorophenyl)pyridin-2-yl]- 4,5-dihydro-1,2-oxazol-3- yl}pyrrolidin-3- yl]methanesulfonamide | 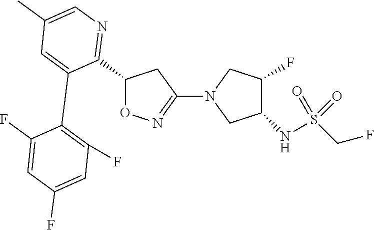 [SMILES: Cc1cnc([C@@H]2CC(N3C[C@H](F)[C@H](NS(=O)(=O)CF)C3)=NO2)c(-c2c(F)cc(F)cc2F)c1] | 491.2 |
| 83 | N′-[(3R,4S)-4-fluoro-1-{(5S)-5- [5-methyl-3-(2,4,6- trifluorophenyl)pyridin-2-yl]- 4,5-dihydro-1,2-oxazol-3- yl}pyrrolidin-3-yl]-N,N- dimethylsulfuric diamide | 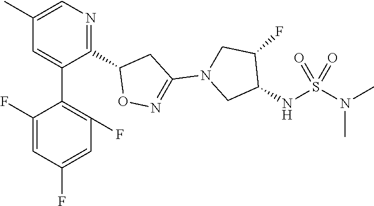 | 502.2 |
| 84 | N-[(3R,4S)-1-{5-[5-chloro-3- (2,4,6-trifluorophenyl)pyridin-2- yl]-4,5-dihydro-1,2-oxazol-3-yl}- 4-fluoropyrrolidin-3- yl]methanesulfonamide | 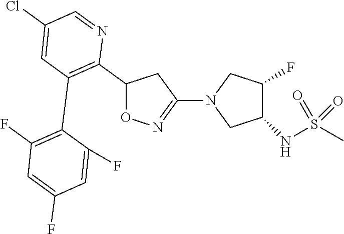 [SMILES: CS(=O)(=O)N[C@@H]1CN(C2=NOC(c3ncc(Cl)cc3-c3c(F)cc(F)cc3F)C2)C[C@@H]1F] | 493.3 |
| 85 | N-[(3R,4S)-1-{5-[5-bromo-3- (2,4,6-trifluorophenyl)pyridin-2- yl]-4,5-dihydro-1,2-oxazol-3-yl}- 4-fluoropyrrolidin-3- yl]methanesulfonamide | 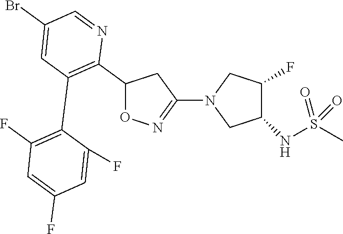 [SMILES: CS(=O)(=O)N[C@@H]1CN(C2=NOC(c3ncc(Br)cc3-c3c(F)cc(F)cc3F)C2)C[C@@H]1F] | 537.0 |

TABLE 1-16

| Ex. | | | |
|---|---|---|---|
| No. | IUPAC Name | Structure | MS |
| 86 | N-[(3R,4S)-1-{5-[3-(2,6- difluorophenyl)-5-methylpyridin- 2-yl]-5-(fluoromethyl)-4,5- dihydro-1,2-oxazol-3-yl}-4- fluoropyrrolidin-3- yl]methanesulfonamide(optical isomer) | 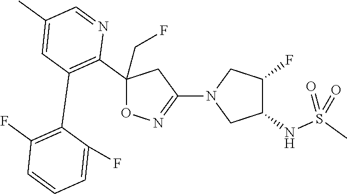 [SMILES: Cc1cnc(C2(CF)CC(N3C[C@H](F)[C@H](NS(C)(=O)=O)C3)=NO2)c(-c2c(F)cccc2F)c1] | 487.2 |
| 87 | N-[(3S)-1-{5-[3-(2,6- difluorophenyl)-5-fluoropyridin- 2-yl]-5-(fluoromethyl)-4,5- dihydro-1,2-oxazol-3- yl}pyrrolidin-3- yl]methanesulfonamide | 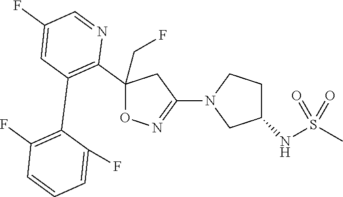 [SMILES: CS(=O)(=O)N[C@H]1CCN(C2=NOC(CF)(c3ncc(F)cc3-c3c(F)cccc3F)C2)C1] | 473.3 |
| 88 | N-[(3S)-1-{5-[3-(2,6- difluorophenyl)-5-fluoropyridin- 2-yl]-5-(fluoromethyl)-4,5- dihydro-1,2-oxazol-3- yl}pyrrolidin-3- yl]ethanesulfonamide | 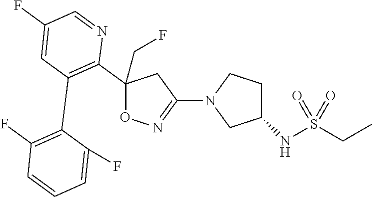 [SMILES: CCS(=O)(=O)N[C@H]1CCN(C2=NOC(CF)(c3ncc(F)cc3-c3c(F)cccc3F)C2)C1] | 487.3 |
| 89 | N-[(3S)-1-{5-[3-(2,6- difluorophenyl)-5-fluoropyridin- 2-yl]-5-(fluoromethyl)-4,5- dihydro-1,2-oxazol-3- yl}pyrrolidin-3-yl]-1- fluorocyclopropane-1-sulfonamide | 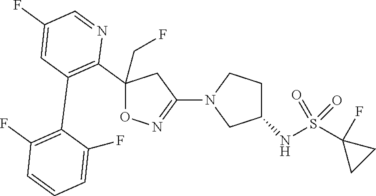 [SMILES: O=S(=O)(N[C@H]1CCN(C2=NOC(CF)(c3ncc(F)cc3-c3c(F)cccc3F)C2)C1)C1(F)CC1] | 517.3 |
| 90 | N-[(3R,4S)-4-fluoro-1-{5-[5- fluoro-3-(2,4,6- trifluorophenyl)pyridin-2-yl]- 4,5-dihydro-1,2-oxazol-3- yl}pyrrolidin-3- yl]methanesulfonamide(optical isomer) | 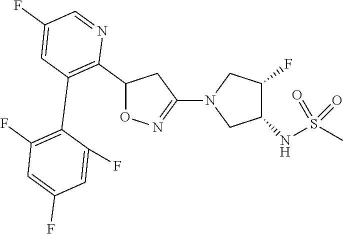 [SMILES: CS(=O)(=O)N[C@@H]1CN(C2=NOC(c3ncc(F)cc3-c3c(F)cc(F)cc3F)C2)C[C@@H]1F] | 477.2 |
| 91 | N-[(3R,4S)-4-fluoro-1-{(5S)-5-[5- methyl-3-(2,4,6- trifluorophenyl)pyridin-2-yl]- 4,5-dihydro-1,2-oxazol-3- yl}pyrrolidin-3-yl]-1- methoxymethanesulfonamide | 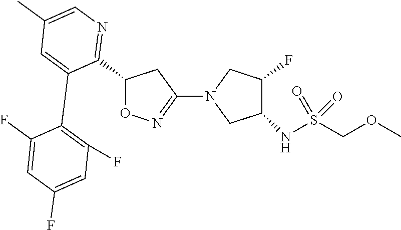 [SMILES: COCS(=O)(=O)N[C@@H]1CN(C2=NO[C@H](c3ncc(C)cc3-c3c(F)cc(F)cc3F)C2)C[C@@H]1F] | 503.3 |

TABLE 1-17

| Ex. | | | |
|---|---|---|---|
| No. | IUPAC Name | Structure | MS |
| 92 | N-[(3R,4S)-4-fluoro-1-{(5S)-5-[5- methyl-3-(2,4,6- trifluorophenyl)pyridin-2-yl]- 4,5-dihydro-1,2-oxazol-3- yl}pyrrolidin-3- yl]ethanesulfonamide | 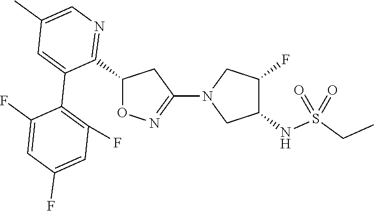 [SMILES: CCS(=O)(=O)N[C@@H]1CN(C2=NO[C@H](c3ncc(C)cc3-c3c(F)cc(F)cc3F)C2)C[C@@H]1F] | 487.2 |
| 93 | N-[(3R)-4,4-difluoro-1-{5- (fluoromethyl)-5-[5-methyl-3- (2,4,6-trifluorophenyl)pyridin-2- yl]-4,5-dihydro-1,2-oxazol-3- yl}pyrrolidin-3- yl]methanesulfonamide (optical isomer) | 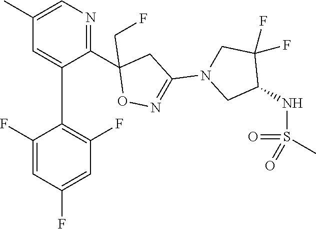 | 523.2 |
| 96 | N-[(3R,4S)-1-{5-[3-(2,6- difluorophenyl)-5-methylpyridin- 2-yl]-5-methyl-4,5-dihydro-1,2- oxazol-3-yl}-4-fluoropyrrolidin- 3-yl]methanesulfonamide | 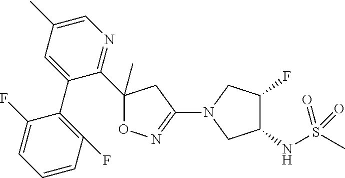 [SMILES: Cc1cnc(C2(C)CC(N3C[C@H](F)[C@H](NS(C)(=O)=O)C3)=NO2)c(-c2c(F)cccc2F)c1] | 469.3 |
| 97 | N-[(3R,4S)-4-fluoro-1-{(5S)-5-[5- methyl-3-(2,4,6- trifluorophenyl)pyridin-2-yl]- 4,5-dihydro-1,2-oxazol-3- yl}pyrrolidin-3- yl]cyclopropanesulfonamide | 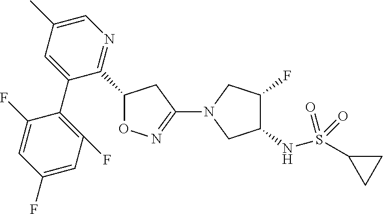 [SMILES: Cc1cnc([C@@H]2CC(N3C[C@H](F)[C@H](NS(=O)(=O)C4CC4)C3)=NO2)c(-c2c(F)cc(F)cc2F)c1] | 499.2 |
| 98 | N-[(3R,4S)-1-{5-[5-chloro-3-(2,6- difluorophenyl)pyridin-2-yl]-4,5- dihydro-1,2-oxazol-3-yl}-4- fluoropyrrolidin-3- yl]cyclopropanesulfonamide | 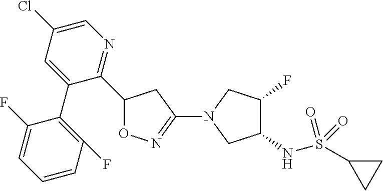 [SMILES: O=S(=O)(N[C@@H]1CN(C2=NOC(c3ncc(Cl)cc3-c3c(F)cccc3F)C2)C[C@@H]1F)C1CC1] | 501.2 |
| 99 | N-[(3S)-1-{(5S)-5-[3-(2,6- difluorophenyl)-5-methylpyridin- 2-yl]-4,5-dihydro-1,2-oxazol-3- yl}pyrrolidin-3-yl]-1- fluoroethane-1-sulfonamide | 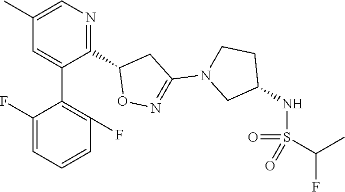 [SMILES: Cc1cnc([C@@H]2CC(N3CC[C@H](NS(=O)(=O)C(C)F)C3)=NO2)c(-c2c(F)cccc2F)c1] | 469.3 |

TABLE 1-18

| Ex. | | | |
|---|---|---|---|
| No. | IUPAC Name | Structure | MS |
| 100 | 1-fluoro-N-[(3S)-1-{(5S)-5-[5- methyl-3-(2,4,6- trifluorophenyl)pyridin-2-yl]- 4,5-dihydro-1,2-oxazol-3- yl}pyrrolidin-3-yl]ethane-1- sulfonamide | 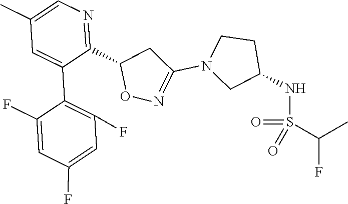 [SMILES: Cc1cnc([C@@H]2CC(N3CC[C@H](NS(=O)(=O)C(C)F)C3)=NO2)c(-c2c(F)cc(F)cc2F)c1] | 487.2 |
| 101 | N-[(3S)-1-{5-[5-chloro-3-(2,6- difluorophenyl)pyridin-2-yl]-4,5- dihydro-1,2-oxazol-3- yl}pyrrolidin-3-yl]-1- fluoromethanesulfonamide | 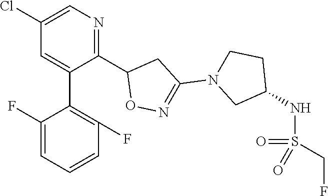 [SMILES: O=S(=O)(CF)N[C@H]1CCN(C2=NOC(c3ncc(Cl)cc3-c3c(F)cccc3F)C2)C1] | 475.2 |
| 102 | N-[(3S)-1-{5-[5-bromo-3-(2,6- difluorophenyl)pyridin-2-yl]-4,5- dihydro-1,2-oxazol-3- yl}pyrrolidin-3-yl]-1- fluoromethanesulfonamide | 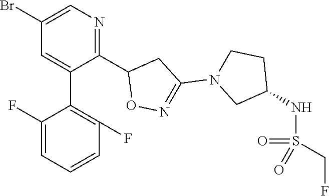 [SMILES: O=S(=O)(CF)N[C@H]1CCN(C2=NOC(c3ncc(Br)cc3-c3c(F)cccc3F)C2)C1] | 519.1 |
| 103 | N-[(3R,4S)-1-{5-[5-chloro-3-(2,6- difluorophenyl)pyridin-2-yl]-4,5- dihydro-1,2-oxazol-3-yl}-4- fluoropyrrolidin-3- yl]methanesulfonamide (optical isomer) | 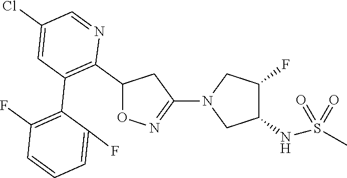 [SMILES: CS(=O)(=O)N[C@@H]1CN(C2=NOC(c3ncc(Cl)cc3-c3c(F)cccc3F)C2)C[C@@H]1F] | 475.2 |
| 104 | N-[(3R)-1-{5-[3-(2,6- difluorophenyl)-5-methylpyrazin- 2-yl]-5-(fluoromethyl)-4,5- dihydro-1,2-oxazol-3-yl}-4,4- difluoropyrrolidin-3- yl]methanesulfonamide | 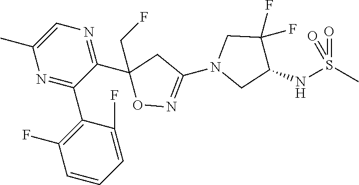 | 506.2 |
| 105 | N-[(3R,4S)-1-{5-[3-(2,6- difluorophenyl)-5-methylpyrazin- 2-yl]-5-(fluoromethyl)-4,5- dihydro-1,2-oxazol-3-yl}-4- fluoropyrrolidin-3- yl]methanesulfonamide | 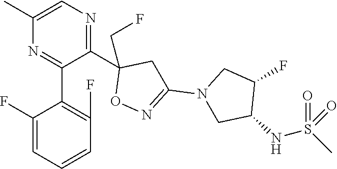 [SMILES: Cc1cnc(C2(CF)CC(N3C[C@H](F)[C@H](NS(C)(=O)=O)C3)=NO2)c(-c2c(F)cccc2F)n1] | 488.3 |

TABLE 1-19

| Ex. | | | |
|---|---|---|---|
| No. | IUPAC Name | Structure | MS |
| 106 | N-[(3R)-1-{5-[2-(3,5- difluoropyridin-2-yl)-4- methylphenyl]-4,5-dihydro-1,2- oxazol-3-yl}-4,4- difluoropyrrolidin-3- yl]methanesulfonamide | 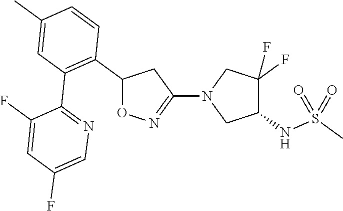 [SMILES: Cc1ccc(C2CC(N3C[C@@H](NS(C)(=O)=O)C(F)(F)C3)=NO2)c(-c2ncc(F)cc2F)c1] | 473.2 |
| 107 | optical isomer of 1-fluoro-N- [(3R,4S)-4-fluoro-1-{(5S)-5-[5- methyl-3-(2,4,6- trifluorophenyl)pyridin-2-yl]- 4,5-dihydro-1,2-oxazol-3- yl}pyrrolidin-3-yl]cyclopropane- 1-sulfonamide or 1-fluoro-N- [(3S,4R)-4-fluoro-1-{(5S)-5-[5- methyl-3-(2,4,6- trifluorophenyl)pyridin-2-yl]- 4,5-dihydro-1,2-oxazol-3- yl}pyrrolidin-3-yl]cyclopropane- 1-sulfonamide | 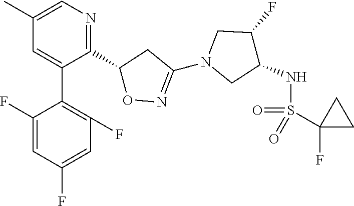 [SMILES: Cc1cnc([C@@H]2CC(N3C[C@H](F)[C@H](NS(=O)(=O)C4(F)CC4)C3)=NO2)c(-c2c(F)cc(F)cc2F)c1] | 517.2 |
| 108 | N-[(3R)-1-{5-[3-(2,6- difluorophenyl)-5-methylpyridin- 2-yl]-5-(fluoromethyl)-4,5- dihydro-1,2-oxazol-3-yl}-4,4- difluoropyrrolidin-3- yl]methanesulfonamide(optical isomer) | 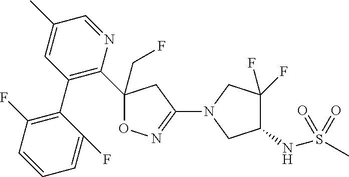 [SMILES: Cc1cnc(C2(CF)CC(N3C[C@@H](NS(C)(=O)=O)C(F)(F)C3)=NO2)c(-c2c(F)cccc2F)c1] | 505.3 |
| 109 | N-[(3R)-1-{(5S)-5-[3-(2,6- difluorophenyl)-5-fluoropyridin- 2-yl]-5-(fluoromethyl)-4,5- dihydro-1,2-oxazol-3-yl}-4,4- difluoropyrrolidin-3- yl]methanesulfonamide | 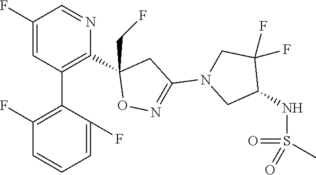 [SMILES: CS(=O)(=O)N[C@@H]1CN(C2=NO[C@](CF)(c3ncc(F)cc3-c3c(F)cccc3F)C2)CC1(F)F] | 509.2 |
| 110 | N-[(3R,4S)-4-fluoro-1-{5- (fluoromethyl)-5-[5-methyl-3- (2,4,6-trifluorophenyl)pyridin-2- yl]-4,5-dihydro-1,2-oxazol-3- yl}pyrrolidin-3- yl]methanesulfonamide (optical isomer) | 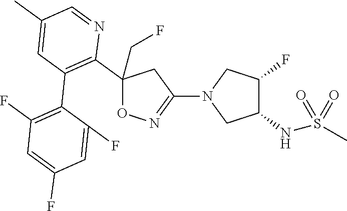 [SMILES: Cc1cnc(C2(CF)CC(N3C[C@H](F)[C@H](NS(C)(=O)=O)C3)=NO2)c(-c2c(F)cc(F)cc2F)c1] | 505.2 |

TABLE 1-20

| Ex. | | | |
|---|---|---|---|
| No. | IUPAC Name | Structure | MS |
| 111 | N-{(3R)-4,4-difluoro-1-[5- (2′,5,6′-trifluoro[1,1′- biphenyl]-2-yl)-4,5-dihydro-1,2- oxazol-3-yl]pyrrolidin-3- yl}methanesulfonamide | 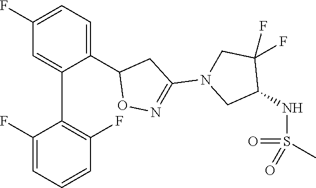 [SMILES: CS(=O)(=O)N[C@@H]1CN(C2=NOC(c3ccc(F)cc3-c3c(F)cccc3F)C2)CC1(F)F] | 476.3 |
| 112 | N-[(3R,4S)-1-{5-[5-chloro-3- (2,4,6-trifluorophenyl)pyridin-2- yl]-4,5-dihydro-1,2-oxazol-3-yl}- 4-fluoropyrrolidin-3- yl]methanesulfonamide (optical isomer) | 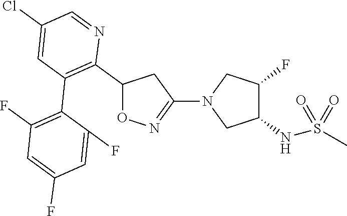 [SMILES: CS(=O)(=O)N[C@@H]1CN(C2=NOC(c3ncc(Cl)cc3-c3c(F)cc(F)cc3F)C2)C[C@@H]1F] | 493.2 |
| 113 | N-[(3R,4S)-4-fluoro-1-{5- (fluoromethyl)-5-[5-fluoro-3- (2,4,6-trifluorophenyl)pyridin-2- yl]-4,5-dihydro-1,2-oxazol-3- yl}pyrrolidin-3- yl]methanesulfonamide (optical isomer) | 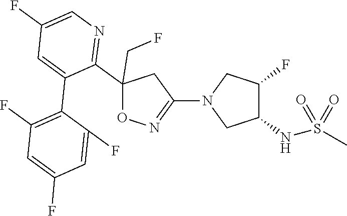 [SMILES: CS(=O)(=O)N[C@@H]1CN(C2=NOC(CF)(c3ncc(F)cc3-c3c(F)cc(F)cc3F)C2)C[C@@H]1F] | 509.2 |
| 114 | N-[(3R)-1-{5-[2-(2,6- difluorophenyl)-6-methylpyridin- 3-yl]-4,5-dihydro-1,2-oxazol-3- yl}-4,4-difluoropyrrolidin-3- yl]methanesulfonamide | 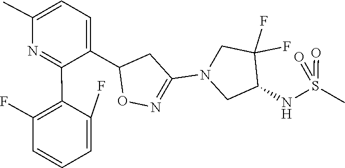 | 473.3 |
| 115 | N-[(3R,4S)-1-{5-[5-chloro-3-(2,6- difluorophenyl)pyridin-2-yl]-5- (fluoromethyl)-4,5-dihydro-1,2- oxazol-3-yl}-4-fluoropyrrolidin- 3-yl]ethanesulfonamide | 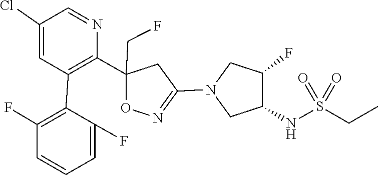 [SMILES: CCS(=O)(=O)N[C@@H]1CN(C2=NOC(CF)(c3ncc(Cl)cc3-c3c(F)cccc3F)C2)C[C@@H]1F] | 521.1 |
| 116 | N-[(3R,4S)-1-{5-[3-(2,6- difluorophenyl)-5-fluoropyridin- 2-yl]-5-(fluoromethyl)-4,5- dihydro-1,2-oxazol-3-yl}-4- fluoropyrrolidin-3- yl]methanesulfonamide (optical isomer) | 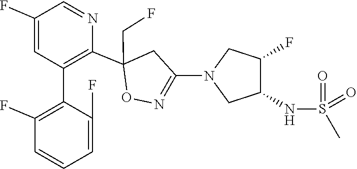 [SMILES: CS(=O)(=O)N[C@@H]1CN(C2=NOC(CF)(c3ncc(F)cc3-c3c(F)cccc3F)C2)C[C@@H]1F] | 491.3 |

TABLE 1-21

| Ex. | | | |
|---|---|---|---|
| No. | IUPAC Name | Structure | MS |
| 117 | optical isomer of N-[(3R,4S)-1- {(5S)-5-[3-(2,6-difluorophenyl)- 5-methylpyridin-2-yl]-4,5- dihydro-1,2-oxazol-3-yl}-4- fluoropyrrolidin-3-yl]-1- fluorocyclopropane-1-sulfonamide or N-[(3S,4R)-1-{(5S)-5-[3-(2,6- difluorophenyl)-5-methylpyridin- 2-yl]-4,5-dihydro-1,2-oxazol-3- yl}-4-fluoropyrrolidin-3-yl]-1- fluorocyclopropane-1-sulfonamide | 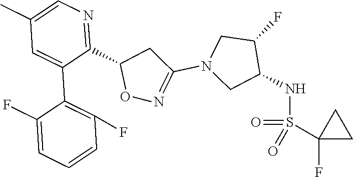 [SMILES: Cc1cnc([C@@H]2CC(N3C[C@H](F)[C@H](NS(=O)(=O)C4(F)CC4)C3)=NO2)c(-c2c(F)cccc2F)c1] | 499.2 |
| 118 | N-[(3R,4S)-1-{5-[3-(2,6- difluorophenyl)-5-fluoropyridin- 2-yl]-5-methyl-4,5-dihydro-1,2- oxazol-3-yl}-4-fluoropyrrolidin- 3-yl]methanesulfonamide | 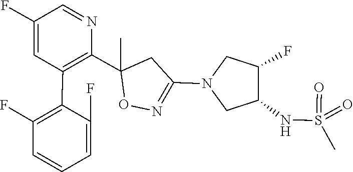 [SMILES: CC1(c2ncc(F)cc2-c2c(F)cccc2F)CC(N2C[C@H](F)[C@H](NS(C)(=O)=O)C2)=NO1] | 473.3 |
| 119 | N-{(3R,4S)-4-fluoro-1-[5- (2′,5,6′-trifluoro[1,1′- biphenyl]-2-yl)-4,5-dihydro-1,2- oxazol-3-yl]pyrrolidin-3- yl}methanesulfonamide | 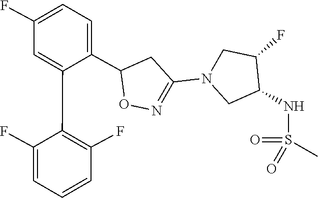 [SMILES: CS(=O)(=O)N[C@@H]1CN(C2=NOC(c3ccc(F)cc3-c3c(F)cccc3F)C2)C[C@@H]1F] | 458.2 |
| 120 | N′-[(3R,4S)-4-fluoro-1-{5-[5- fluoro-3-(2,4,6- trifluorophenyl)pyridin-2-yl]- 4,5-dihydro-1,2-oxazol-3- yl}pyrrolidin-3-yl]-N,N- dimethylsulfuric diamide (optical isomer) | 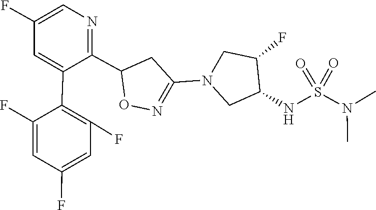 [SMILES: CN(C)S(=O)(=O)N[C@@H]1CN(C2=NOC(c3ncc(F)cc3-c3c(F)cc(F)cc3F)C2)C[C@@H]1F] | 506.2 |
| 121 | N′-[(3R,4S)-1-{5-[5-chloro-3- (2,6-difluorophenyl)pyridin-2- yl]-4,5-dihydro-1,2-oxazol-3-yl}- 4-fluoropyrrolidin-3-yl]-N,N- dimethylsulfuric diamide | 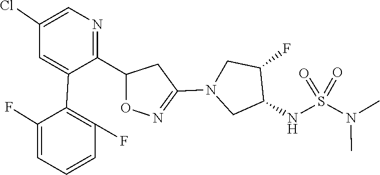 [SMILES: CN(C)S(=O)(=O)N[C@@H]1CN(C2=NOC(c3ncc(Cl)cc3-c3c(F)cccc3F)C2)C[C@@H]1F] | 504.2 |

TABLE 1-22

| Ex. | | | |
|---|---|---|---|
| No. | IUPAC Name | Structure | MS |
| 122 | N-[(3S)-1-{5-[3-(2,6- difluorophenyl)-5-fluoropyridin- 2-yl]-5-methyl-4,5-dihydro-1,2- oxazol-3-yl}pyrrolidin-3- yl]methanesulfonamide | 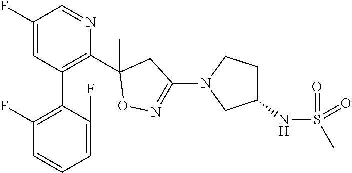 [SMILES: CC1(c2ncc(F)cc2-c2c(F)cccc2F)CC(N2CC[C@H](NS(C)(=O)=O)C2)=NO1] | 455.3 |
| 123 | N′-[(3R,4S)-1-{5-[4-chloro-2- (3,5-difluoropyridin-2- yl)phenyl]-4,5-dihydro-1,2- oxazol-3-yl}-4-fluoropyrrolidin- 3-yl]-N,N-dimethylsulfuric diamide | 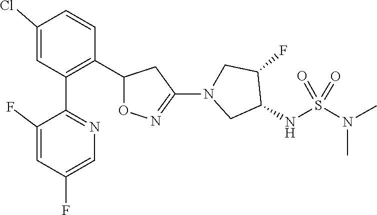 [SMILES: CN(C)S(=O)(=O)N[C@@H]1CN(C2=NOC(c3ccc(Cl)cc3-c3ncc(F)cc3F)C2)C[C@@H]1F] | 504.2 |
| 124 | N-[(3R,4S)-1-{5-[2-(3,5- difluoropyridin-2-yl)-4- methylphenyl]-4,5-dihydro-1,2- oxazol-3-yl}-4-fluoropyrrolidin- 3-yl]methanesulfonamide | 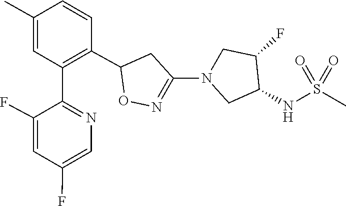 [SMILES: Cc1ccc(C2CC(N3C[C@H](F)[C@H](NS(C)(=O)=O)C3)=NO2)c(-c2ncc(F)cc2F)c1] | 455.3 |
| 125 | N′-[(3R,4S)-1-{5-[2-(3,5- difluoropyridin-2-yl)-4- methylphenyl]-4,5-dihydro-1,2- oxazol-3-yl}-4-fluoropyrrolidin- 3-yl]-N,N-dimethylsulfuric diamide | 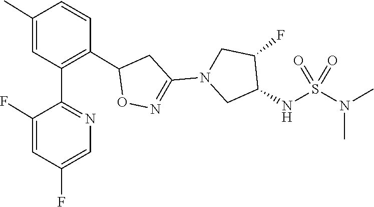 [SMILES: Cc1ccc(C2CC(N3C[C@H](F)[C@H](NS(=O)(=O)N(C)C)C3)=NO2)c(-c2ncc(F)cc2F)c1] | 484.3 |
| 126 | N-[(3R)-1-{5-(difluoromethyl)-5- [3-(2,6-difluorophenyl)-5- fluoropyridin-2-yl]-4,5-dihydro- 1,2-oxazol-3-yl}-4,4- difluoropyrrolidin-3- yl]methanesulfonamide | 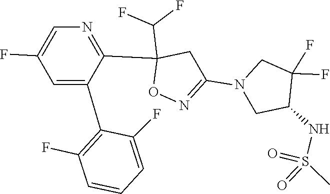 [SMILES: CS(=O)(=O)N[C@@H]1CN(C2=NOC(c3ncc(F)cc3-c3c(F)cccc3F)(C(F)F)C2)CC1(F)F] | 527.1 |
| 127 | N-[(3R)-1-{5-[4-(2,6- difluorophenyl)-6-methylpyridin- 3-yl]-4,5-dihydro-1,2-oxazol-3- yl}-4,4-difluoropyrrolidin-3- yl]methanesulfonamide | 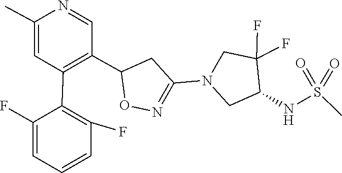 [SMILES: Cc1cc(-c2c(F)cccc2F)c(C2CC(N3C[C@@H](NS(C)(=O)=O)C(F)(F)C3)=NO2)cn1] | 473.0 |

TABLE 1-23

| Ex. | | | |
|---|---|---|---|
| No. | IUPAC Name | Structure | MS |
| 128 | N-[(3R)-4,4-difluoro-1-{(5S)-5- [5-fluoro-3-(2,4,6- trifluorophenyl)pyridin-2-yl]- 4,5-dihydro-1,2-oxazol-3- yl}pyrrolidin-3-yl]-1- fluoromethanesulfonamide (optical isomer) | 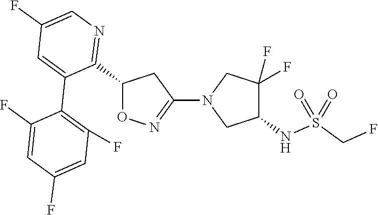 [SMILES: O=S(=O)(CF)N[C@@H]1CN(C2=NO[C@H](c3ncc(F)cc3-c3c(F)cc(F)cc3F)C2)CC1(F)F] | 513.2 |
| 129 | N-[(3R,4S)-1-{5-[5-chloro-3-(2,6- difluorophenyl)pyridin-2-yl]-5- (fluoromethyl)-4,5-dihydro-1,2- oxazol-3-yl}-4-fluoropyrrolidin- 3-yl]methanesulfonamide (optical isomer) | 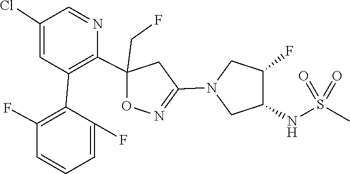 [SMILES: CS(=O)(=O)N[C@@H]1CN(C2=NOC(CF)(c3ncc(Cl)cc3-c3c(F)cccc3F)C2)C[C@@H]1F] | 507.2 |
| 130 | N-[(3R)-1-{5-[3-(2,6- difluorophenyl)-5-fluoropyridin- 2-yl]-5-ethyl-4,5-dihydro-1,2- oxazol-3-yl}-4,4- difluoropyrrolidin-3- yl]methanesulfonamide | 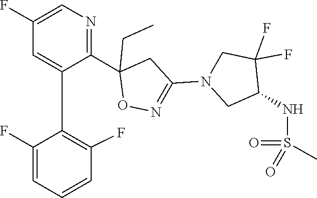 [SMILES: CCC1(c2ncc(F)cc2-c2c(F)cccc2F)CC(N2C[C@@H](NS(C)(=O)=O)C(F)(F)C2)=NO1] | 505.3 |
| 131 | N-[(3R)-1-{5-[3-(2,6- difluorophenyl)-5-fluoropyridin- 2-yl]-5-(methoxymethyl)-4,5- dihydro-1,2-oxazol-3-yl}-4,4- difluoropyrrolidin-3- yl]methanesulfonamide | 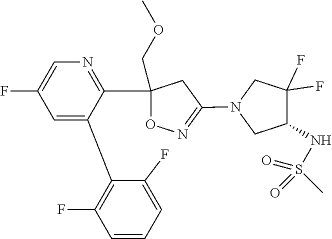 [SMILES: COCC1(c2ncc(F)cc2-c2c(F)cccc2F)CC(N2C[C@@H](NS(C)(=O)=O)C(F)(F)C2)=NO1] | 521.2 |
| 132 | N-[(3R,4S)-1-{5-[3-(2,6- difluorophenyl)-5-fluoropyridin- 2-yl]-5-(methoxymethyl)-4,5- dihydro-1,2-oxazol-3-yl}-4- fluoropyrrolidin-3- yl]ethanesulfonamide | 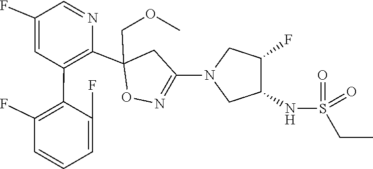 [SMILES: CCS(=O)(=O)N[C@@H]1CN(C2=NOC(COC)(c3ncc(F)cc3-c3c(F)cccc3F)C2)C[C@@H]1F] | 517.2 |
| 133 | N-[(3R,4S)-1-{5-[5-chloro-3-(2,6- difluorophenyl)pyridin-2-yl]-4,5- dihydro-1,2-oxazol-3-yl}-4- fluoropyrrolidin-3- yl]ethanesulfonamide (optical isomer) | 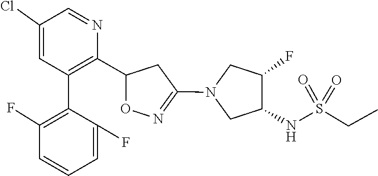 [SMILES: CCS(=O)(=O)N[C@@H]1CN(C2=NOC(c3ncc(Cl)cc3-c3c(F)cccc3F)C2)C[C@@H]1F] | 489.2 |

TABLE 1-24

| Ex. | | | |
|---|---|---|---|
| No. | IUPAC Name | Structure | MS |
| 134 | N-[(3R)-1-{5-[4-(2,6- difluorophenyl)-2- methylpyrimidin-5-yl]-4,5- dihydro-1,2-oxazol-3-yl}-4,4- difluoropyrrolidin-3- yl]methanesulfonamide | 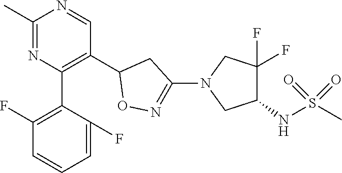 | 474.2 |
| 135 | N-[(3R)-1-{5-[5-(difluoromethyl)- 3-(2,6-difluorophenyl)pyridin-2- yl]-5-(hydroxymethyl)-4,5- dihydro-1,2-oxazol-3-yl}-4,4- difluoropyrrolidin-3- yl]methanesulfonamide | 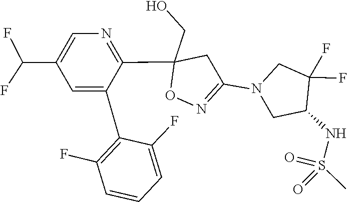 [SMILES: CS(=O)(=O)N[C@@H]1CN(C2=NOC(CO)(c3ncc(C(F)F)cc3-c3c(F)cccc3F)C2)CC1(F)F] | 539.2 |
| 136 | N-{(3R)-4,4-difluoro-1-[5- (2′,3,6,6′-tetrafluoro[1,1'- biphenyl]-2-yl)-4,5-dihydro-1,2- oxazol-3-yl]pyrrolidin-3- yl}methanesulfonamide | 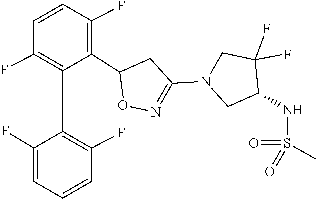 [SMILES: CS(=O)(=O)N[C@@H]1CN(C2=NOC(c3c(F)ccc(F)c3-c3c(F)cccc3F)C2)CC1(F)F] | 494.2 |
| 137 | N-{(3R,4S)-4-fluoro-1-[5- (2′,5,6,6′-tetrafluoro[1,1′- biphenyl]-2-yl)-4,5-dihydro-1,2- oxazol-3-yl]pyrrolidin-3- yl}methanesulfonamide | 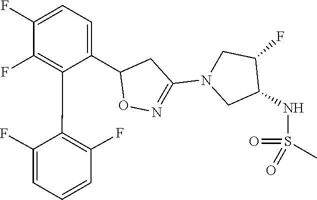 [SMILES: CS(=O)(=O)N[C@@H]1CN(C2=NOC(c3ccc(F)c(F)c3-c3c(F)cccc3F)C2)C[C@@H]1F] | 476.2 |
| 138 | N-[(3R)-1-{5-[2-(3,5- difluoropyridin-2-yl)-4,6- difluorophenyl]-4,5-dihydro-1,2- oxazol-3-yl}-4,4- difluoropyrrolidin-3- yl]methanesulfonamide | 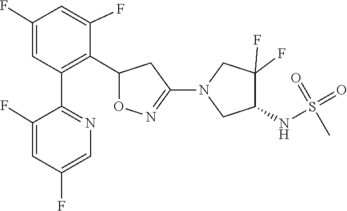 [SMILES: CS(=O)(=O)N[C@@H]1CN(C2=NOC(c3c(F)cc(F)cc3-c3ncc(F)cc3F)C2)CC1(F)F] | 495.2 |
| 139 | N-[(3R,4S)-1-{5-[2-(3,5- difluoropyridin-2-yl)-4,6- difluorophenyl]-4,5-dihydro-1,2- oxazol-3-yl}-4-fluoropyrrolidin- 3-yl]methanesulfonamide | 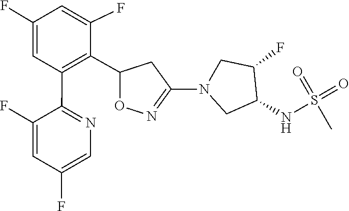 [SMILES: CS(=O)(=O)N[C@@H]1CN(C2=NOC(c3c(F)cc(F)cc3-c3ncc(F)cc3F)C2)C[C@@H]1F] | 477.2 |

TABLE 1-25

| Ex. | | | |
|---|---|---|---|
| No. | IUPAC Name | Structure | MS |
| 140 | N-{(3R)-4,4-difluoro-1-[5- (2′,5,6,6′-tetrafluoro[1,1′- biphenyl]-2-yl)-4,5-dihydro-1,2- oxazol-3-yl]pyrrolidin-3- yl}methanesulfonamide | 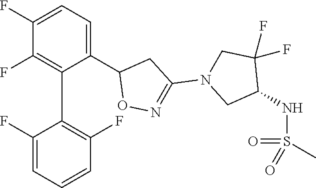 [SMILES: CS(=O)(=O)N[C@@H]1CN(C2=NOC(c3ccc(F)c(F)c3-c3c(F)cccc3F)C2)CC1(F)F] | 494.2 |
| 141 | N-{(3R,4S)-4-fluoro-1-[5- (2′,3,6′-trifluoro[1,1′- biphenyl]-2-yl)-4,5-dihydro-1,2- oxazol-3-yl]pyrrolidin-3- yl}methanesulfonamide | 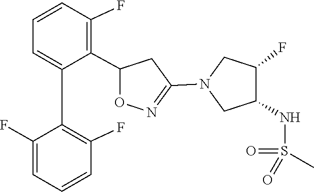 [SMILES: CS(=O)(=O)N[C@@H]1CN(C2=NOC(c3c(F)cccc3-c3c(F)cccc3F)C2)C[C@@H]1F] | 458.2 |
| 142 | N-[(3R)-1-{5-[5-chloro-3-(2,6- difluorophenyl)pyridin-2-yl]-5- (hydroxymethyl)-4,5-dihydro-1,2- oxazol-3-yl}-4,4- difluoropyrrolidin-3- yl]methanesulfonamide | 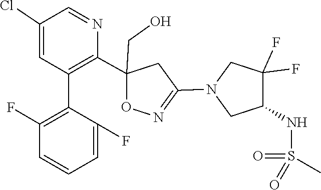 [SMILES: CS(=O)(=O)N[C@@H]1CN(C2=NOC(CO)(c3ncc(Cl)cc3-c3c(F)cccc3F)C2)CC1(F)F] | 523.1 |
| 143 | N-[(3R)-1-{5-[5-chloro-3-(2,6- difluorophenyl)pyridin-2-yl]-4,5- dihydro-1,2-oxazol-3-yl}-4,4- difluoropyrrolidin-3- yl]ethanesulfonamide (optical isomer) | 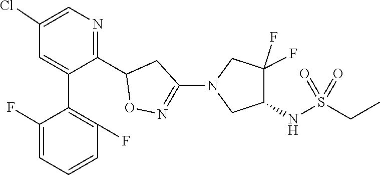 [SMILES: CCS(=O)(=O)N[C@@H]1CN(C2=NOC(c3ncc(Cl)cc3-c3c(F)cccc3F)C2)CC1(F)F] | 507.3 |
| 144 | N-{(3R,4S)-4-fluoro-1-[5- (2′,3,5,6′-tetrafluoro[1,1′- biphenyl]-2-yl)-4,5-dihydro-1,2- oxazol-3-yl]pyrrolidin-3- yl}methanesulfonamide (optical isomer) | 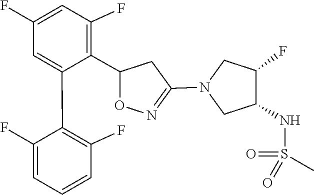 [SMILES: CS(=O)(=O)N[C@@H]1CN(C2=NOC(c3c(F)cc(F)cc3-c3c(F)cccc3F)C2)C[C@@H]1F] | 476.3 |
| 145 | N-{(3R)-4,4-difluoro-1-[5- (2′,3,4′,6′-tetrafluoro-5- methyl[1,1′-biphenyl]-2-yl)-4,5- dihydro-1,2-oxazol-3- yl]pyrrolidin-3- yl}methanesulfonamide | 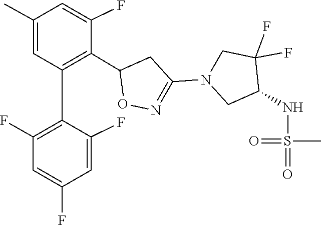 [SMILES: Cc1cc(F)c(C2CC(N3C[C@@H](NS(C)(=O)=O)C(F)(F)C3)=NO2)c(-c2c(F)cc(F)cc2F)c1] | 508.2 |

TABLE 1-26

| Ex. | | | |
|---|---|---|---|
| No. | IUPAC Name | Structure | MS |
| 146 | N-{(3R)-4,4-difluoro-1-[5- (2′,3,6′-trifluoro-5-methyl[1,1′- biphenyl]-2-yl)-4,5-dihydro-1,2- oxazol-3-yl]pyrrolidin-3- yl}methanesulfonamide | 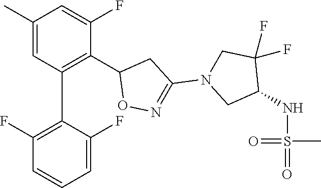 [SMILES: Cc1cc(F)c(C2CC(N3C[C@@H](NS(C)(=O)=O)C(F)(F)C3)=NO2)c(-c2c(F)cccc2F)c1] | 490.2 |
| 147 | N-[(3R)-4,4-difluoro-1-{5- (fluoromethyl)-5-[5-fluoro-3- (2,4,6-trifluorophenyl)pyridin-2- yl]-4,5-dihydro-1,2-oxazol-3- yl}pyrrolidin-3- yl]methanesulfonamide (optical isomer) | 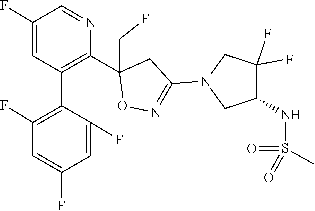 [SMILES: CS(=O)(=O)N[C@@H]1CN(C2=NOC(CF)(c3ncc(F)cc3-c3c(F)cc(F)cc3F)C2)CC1(F)F] | 527.2 |
| 148 | N-[(3R)-1-{5-[5-chloro-3-(2,6- difluorophenyl)pyridin-2-yl]-4,5- dihydro-1,2-oxazol-3-yl}-4,4- difluoropyrrolidin-3-yl]-1- fluoromethanesulfonamide | 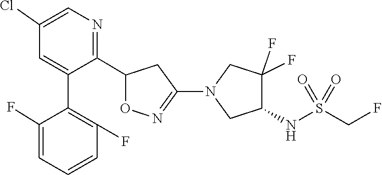 [SMILES: O=S(=O)(CF)N[C@@H]1CN(C2=NOC(c3ncc(Cl)cc3-c3c(F)cccc3F)C2)CC1(F)F] | 511.1 |
| 149 | N-[(3R)-1-{(5S)-5-[3-(2,6- difluorophenyl)-5-fluoropyridin- 2-yl]-5-(fluoromethyl)-4,5- dihydro-1,2-oxazol-3-yl}-4,4- difluoropyrrolidin-3-yl]-1- fluoromethanesulfonamide | 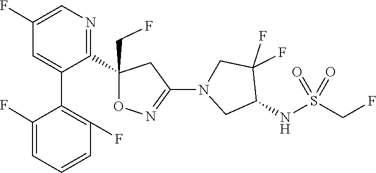 [SMILES: O=S(=O)(CF)N[C@@H]1CN(C2=NO[C@](CF)(c3ncc(F)cc3-c3c(F)cccc3F)C2)CC1(F)F] | 527.2 |
| 150 | N-[(3R)-1-{5-[3-(2,6- difluorophenyl)pyridin-2-yl]-5- (fluoromethyl)-4,5-dihydro-1,2- oxazol-3-yl}-4,4- difluoropyrrolidin-3- yl]methanesulfonamide (optical isomer) | 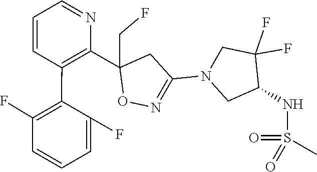 [SMILES: CS(=O)(=O)N[C@@H]1CN(C2=NOC(CF)(c3ncccc3-c3c(F)cccc3F)C2)CC1(F)F] | 491.3 |
| 151 | N-[(3R)-1-{5-[3-(2,6- difluorophenyl)pyridin-2-yl]-5- (fluoromethyl)-4,5-dihydro-1,2- oxazol-3-yl}-4,4- difluoropyrrolidin-3- yl]ethanesulfonamide (optical isomer) | 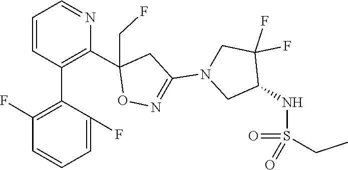 [SMILES: CCS(=O)(=O)N[C@@H]1CN(C2=NOC(CF)(c3ncccc3-c3c(F)cccc3F)C2)CC1(F)F] | 505.2 |

TABLE 1-27

| Ex. | | | |
|---|---|---|---|
| No. | IUPAC Name | Structure | MS |
| 152 | N-{(3R)-1-[5-(5′-chloro-3,5- difluoro[2,3′-bipyridin]-2′-yl)- 5-methyl-4,5-dihydro-1,2-oxazol- 3-yl]-4,4-difluoropyrrolidin-3- yl}methanesulfonamide | 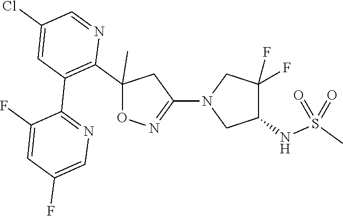 [SMILES: CC1(c2ncc(Cl)cc2-c2ncc(F)cc2F)CC(N2C[C@@H](NS(C)(=O)=O)C(F)(F)C2)=NO1] | 508.2 |
| 153 | N-[(3R)-1-{5-[5-(difluoromethyl)- 3-(2,6-difluorophenyl)pyridin-2- yl]-5-(fluoromethyl)-4,5-dihydro- 1,2-oxazol-3-yl}-4,4- difluoropyrrolidin-3- yl]methanesulfonamide (optical isomer) | 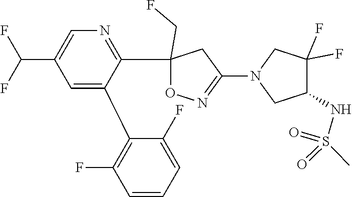 [SMILES: CS(=O)(=O)N[C@@H]1CN(C2=NOC(CF)(c3ncc(C(F)F)cc3-c3c(F)cccc3F)C2)CC1(F)F] | 541.2 |
| 154 | N-[(3R)-1-{5-[2-(3,5- difluoropyridin-2-yl)-6-fluoro-4- methylphenyl]-4, 5-dihydro-1, 2- oxazol-3-yl}-4, 4- difluoropyrrolidin-3- yl]methanesulfonamide | 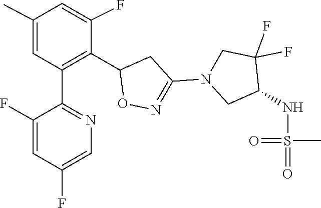 | 491.2 |
| 155 | N-{(3R,4S)-4-fluoro-4-methyl-1- [5-(2′,3,6′-trifluoro[1,1′- biphenyl]-2-yl)-4,5-dihydro-1,2- oxazol-3-yl]pyrrolidin-3- yl}methanesulfonamide or N- {(3S,4R)-4-fluoro-4-methyl-1-[5- (2′,3,6′-trifluoro[1,1′- biphenyl]-2-yl)-4,5-dihydro-1,2- oxazol-3-yl]pyrrolidin-3- yl)methanesulfonamide | 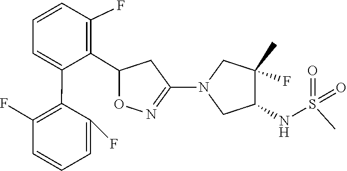 [SMILES: C[C@]1(F)CN(C2=NOC(c3c(F)cccc3-c3c(F)cccc3F)C2)C[C@H]1NS(C)(=O)=O] | 472.3 |
| 156 | N-{(3R)-4,4-difluoro-1-[5- (2′,3,5,6′-tetrafluoro[1,1′- biphenyl]-2-yl)-4,5-dihydro-1,2- oxazol-3-yl]pyrrolidin-3- yl}methanesulfonamide (optical isomer) | 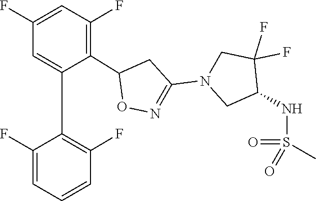 [SMILES: CS(=O)(=O)N[C@@H]1CN(C2=NOC(c3c(F)cc(F)cc3-c3c(F)cccc3F)C2)CC1(F)F] | 494.1 |

TABLE 1-28

| Ex. | | | |
|---|---|---|---|
| No. | IUPAC Name | Structure | MS |
| 157 | N-[(3R)-4,4-difluoro-1-{5- (fluoromethyl)-5-[3-(2,4,6- trifluorophenyl)pyridin-2-yl]- 4,5-dihydro-1,2-oxazol-3- yl}pyrrolidin-3- yl]methanesulfonamide (optical isomer) | 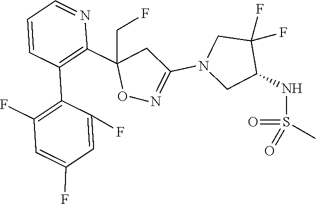 [SMILES: CS(=O)(=O)N[C@@H]1CN(C2=NOC(CF)(c3ncccc3-c3c(F)cc(F)cc3F)C2)CC1(F)F] | 509.2 |
| 158 | N-[(3R)-1-{5-[3-(2,6- difluorophenyl)-5-fluoropyridin- 2-yl]-5-methyl-4,5-dihydro-1,2- oxazol-3-yl}-4,4- difluoropyrrolidin-3- yl]methanesulfonamide (optical isomer) | 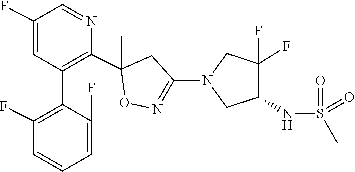 [SMILES: CC1(c2ncc(F)cc2-c2c(F)cccc2F)CC(N2C[C@@H](NS(C)(=O)=O)C(F)(F)C2)=NO1] | 491.1 |
| 159 | N-{(3R,4S)-4-fluoro-1-[5- (2′,3,6,6′-tetrafluoro[1,1′- biphenyl]-2-yl)-4,5-dihydro-1,2- oxazol-3-yl]pyrrolidin-3- yl}methanesulfonamide (optical isomer) | 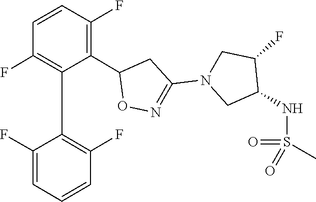 | 476.2 |
| 160 | N-{(3R)-4,4-difluoro-1-[5- (2′,3,6′-trifluoro[1,1′- biphenyl]-2-yl)-4,5-dihydro-1,2- oxazol-3-yl]pyrrolidin-3- yl}methanesulfonamide (optical isomer) | 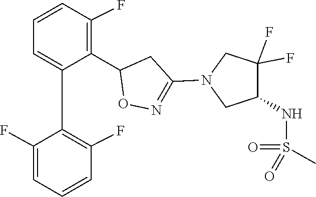 [SMILES: CS(=O)(=O)N[C@@H]1CN(C2=NOC(c3c(F)cccc3-c3c(F)cccc3F)C2)CC1(F)F] | 476.3 |
| 161 | N-[(3R)-1-{5-[2-(3,5- difluoropyridin-2-yl)-4- methylphenyl]-5-methyl-4,5- dihydro-1,2-oxazol-3-yl}-4,4- difluoropyrrolidin-3- yl]methanesulfonamide | 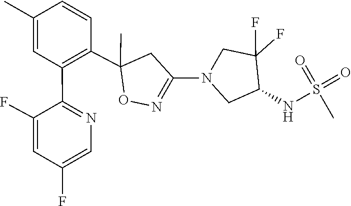 [SMILES: Cc1ccc(C2(C)CC(N3C[C@@H](NS(C)(=O)=O)C(F)(F)C3)=NO2)c(-c2ncc(F)cc2F)c1] | 487.2 |
| 162 | N-[(3R,4S)-1-{(5S)-5-[3-(2,6- difluorophenyl)-5-fluoropyridin- 2-yl]-5-(fluoromethyl)-4,5- dihydro-1,2-oxazol-3-yl}-4- fluoropyrrolidin-3-yl]-1- fluoromethanesulfonamide | 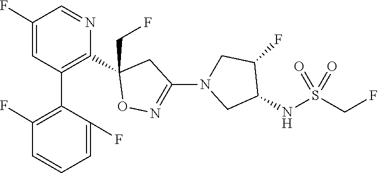 [SMILES: O=S(=O)(CF)N[C@@H]1CN(C2=NO[C@](CF)(c3ncc(F)cc3-c3c(F)cccc3F)C2)C[C@@H]1F] | 509.2 |

TABLE 1-29

| Ex. | | | |
|---|---|---|---|
| No. | IUPAC Name | Structure | MS |
| 163 | 1-fluoro-N-[(3R,4S)-4-fluoro-1- {5-(fluoromethyl)-5-[5-fluoro-3- (2,4,6-trifluorophenyl)pyridin-2- yl]-4,5-dihydro-1,2-oxazol-3- yl}pyrrolidin-3- yl]methanesulfonamide (optical isomer) | 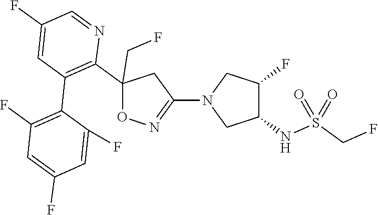 [SMILES: O=S(=O)(CF)N[C@@H]1CN(C2=NOC(CF)(c3ncc(F)cc3-c3c(F)cc(F)cc3F)C2)C[C@@H]1F] | 527.1 |
| 164 | optical isomer of N-{(3R,4S)-4- fluoro-4-methyl-1-[(5S)-5- (2′,3,5,6′-tetrafluoro[1,1′- biphenyl]-2-yl)-4,5-dihydro-1,2- oxazol-3-yl]pyrrolidin-3- yl}methanesulfonamide or N- {(3S,4R) -4-fluoro-4-methyl-1- [(5S)-5-(2′,3,5,6′- tetrafluoro[1,1′-biphenyl]-2-yl)- 4,5-dihydro-1,2-oxazol-3- yl]pyrrolidin-3- | 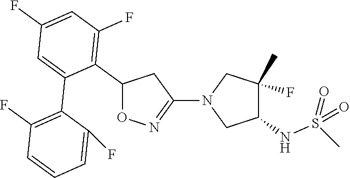 [SMILES: C[C@]1(F)CN(C2=NOC(c3c(F)cc(F)cc3-c3c(F)cccc3F)C2)C[C@H]1NS(C)(=O)=O] | 490.2 |
| | yl}methanesulfonamide or N- | | |
| | {(3R,4S)-4-fluoro-4-methyl-1- | | |
| | [(5R)-5-(2′,3,5,6′- | | |
| | tetrafluoro[1,1′-biphenyl]-2-yl)- | | |
| | 4,5-dihydro-1,2-oxazol-3- | | |
| | yl]pyrrolidin-3- | | |
| | yl}methanesulfonamide or N- | | |
| | {(3S,4R)-4-fluoro-4-methyl-1- | | |
| | [(5R)-5-(2′,3,5,6′- | | |
| | tetrafluoro[1,1′-biphenyl]-2-yl)- | | |
| | 4,5-dihydro-1,2-oxazol-3- | | |
| | yl]pyrrolidin-3- | | |
| | yl}methanesulfonamide | | |

TABLE 1-30

| Ex. No. | IUPAC Name | Structure | MS |
|---|---|---|---|
| 165 | N-[(3R)-1-{(5S)-5-[3-(2,6-difluorophenyl)-5- fluoropyridin-2-yl]-5-methyl-4,5-dihydro-1,2- oxazol-3-yl}-4,4-difluoropyrrolidin-3- yl]methanesulfonamide | 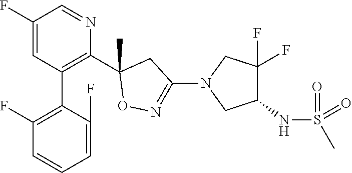 [SMILES: C[C@@]1(c2ncc(F)cc2-c2c(F)cccc2F)CC(N2C[C@@H](NS(C)(=O)=O)C(F)(F)C2)=NO1] | 491.1 |
| 166 | N-{(3R,4S)-4-fluoro-1-[5-(2′,3,5,6,6′- pentafluoro[1,1′-biphenyl]-2-yl)- 4,5-dihydro-1,2-oxazol-3-yl]pyrrolidin-3- yl}methanesulfonamide (optical isomer) | 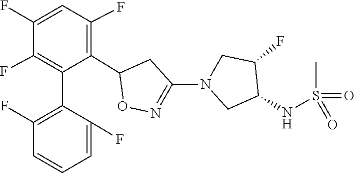 [SMILES: CS(=O)(=O)N[C@@H]1CN(C2=NOC(c3c(F)cc(F)c(F)c3-c3c(F)cccc3F)C2)C[C@@H]1F] | 494.0 |
| 167 | N-[(3R)-1-{5-[2-(3,5-difluoropyridin-2- yl)-6-fluoro-4-methylphenyl]-4,5- dihydro-1,2-oxazol-3-yl}-4,4- difluoropyrrolidin-3- yl]methanesulfonamide (optical isomer) | 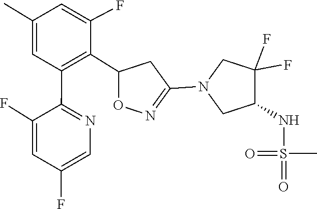 | 491.0 |
| 168 | optical isomer of N-{(3R,4S)-4-fluoro- 4-methyl-1-[(5S)-5-(2′,3,6,6′- tetrafluoro[1,1′-biphenyl]-2-yl)-4,5- dihydro-1,2-oxazol-3-yl]pyrrolidin-3- yl} methanesulfonamide or N-{(3S,4R)- 4-fluoro-4-methyl-1-[(5S)-5-(2′,3,6,6′- tetrafluoro[1,1′-biphenyl]-2-yl)-4,5- dihydro-1,2-oxazol-3-yl]pyrrolidin-3- yl}methanesulfonamide or N-{(3R,4S)- 4-fluoro-4-methyl-1-[(5R)-5-(2′,3,6,6′- tetrafluoro[1,1'-biphenyl]-2-yl)-4,5- | 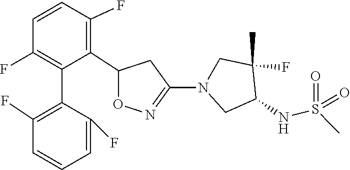 [SMILES: C[C@]1(F)CN(C2=NOC(c3c(F)ccc(F)c3-c3c(F)cccc3F)C2)C[C@H]1NS(C)(=O)=O] | 490.2 |
| | dihydro-1,2-oxazol-3-yl] pyrrolidin-3- | | |
| | yl}methanesulfonamide or N-{(3S,4R)- | | |
| | 4-fluoro-4-methyl-1-[(5R)-5-(2′,3,6,6′- | | |
| | tetrafluoro[1,1′-biphenyl]-2-yl)-4,5- | | |
| | dihydro-1,2-oxazol-3-yl]pyrrolidin- | | |
| | 3-yl}methanesulfonamide | | |
| 169 | N-{(3R)-4,4-difluoro-1-[5-(2′,3,5,6′- tetrafluoro[1,1′-biphenyl]-2-yl)-4,5- dihydro-1,2-oxazol-3-yl]pyrrolidin- 3-yl}-1-fluoromethanesulfonamide (optical isomer) | 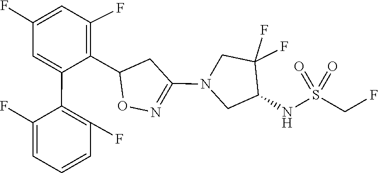 [SMILES: O=S(=O)(CF)N[C@@H]1CN(C2=NOC(c3c(F)cc(F)cc3-c3c(F)cccc3F)C2)CC1(F)F] | 512.2 |
| 170 | N-{(3R)-4,4-difluoro-1-[5-(2′,3,6′- trifluoro[1,1′-biphenyl]-2-yl)-4,5- dihydro-1,2-oxazol-3-yl]pyrrolidin- 3-yl}-1-fluoromethanesulfonamide | 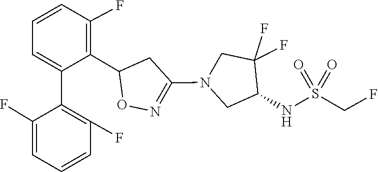 [SMILES: O=S(=O)(CF)N[C@@H]1CN(C2=NOC(c3c(F)cccc3-c3c(F)cccc3F)C2)CC1(F)F] | 494.2 |
| 171 | N-[(3R)-1-{5-[4-chloro-2-(3,5- difluoropyridin-2-yl)-6- fluorophenyl]-4,5-dihydro- 1,2-oxazol-3-yl}-4,4- difluoropyrrolidin-3- yl]methanesulfonamide | 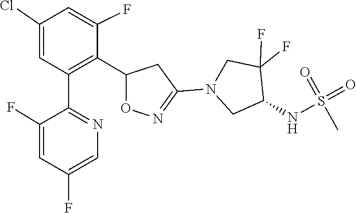 [SMILES: CS(=O)(=O)N[C@@H]1CN(C2=NOC(c3c(F)cc(Cl)cc3-c3ncc(F)cc3F)C2)CC1(F)F] | 511.2 |

TABLE 1-31

| Ex. No. | IUPAC Name | Structure | MS |
|---|---|---|---|
| 172 | N-{(3R)-4,4-difluoro-1-[5- (2′,3,5,6,6′-pentafluoro[1,1′- biphenyl]-2-yl)-4,5-dihydro- 1,2-oxazol-3-yl]pyrrolidin- 3-yl}methanesulfonamide (optical isomer) | 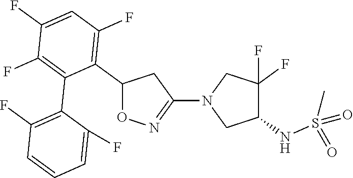 [SMILES: CS(=O)(=O)N[C@@H]1CN(C2=NOC(c3c(F)cc(F)c(F)c3-c3c(F)cccc3F)C2)CC1(F)F] | 511.9 |
| 173 | N-{(3R)-4,4-difluoro-1-[5- (2′,3,6,6′-tetrafluoro[1,1′- biphenyl]-2-yl)-4,5- dihydro-1,2-oxazol-3-yl] pyrrolidin-3-yl}-1- fluoromethanesulfonamide (optical isomer) | 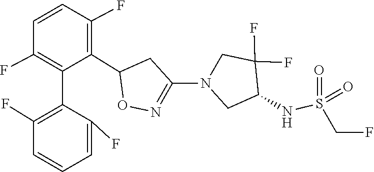 [SMILES: O=S(=O)(CF)N[C@@H]1CN(C2=NOC(c3c(F)ccc(F)c3-c3c(F)cccc3F)C2)CC1(F)F] | 512.0 |
| 174 | N-{(3R)-4,4-difluoro-1- [5-(2′,3,5,6,6′-pentafluoro [1,1′-biphenyl]-2-yl)- 4,5-dihydro-1,2-oxazol- 3-yl]pyrrolidin-3-yl}-1- fluoromethanesulfonamide (optical isomer) | 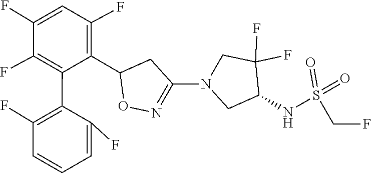 [SMILES: O=S(=O)(CF)N[C@@H]1CN(C2=NOC(c3c(F)cc(F)c(F)c3-c3c(F)cccc3F)C2)CC1(F)F] | 530.1 |
| 175 | N-{(3R)-1-[5-(2′,3-difluoro- 6′-methoxy-5-methyl[1,1′- biphenyl]-2-yl)-4,5-dihydro- 1,2-oxazol-3-yl]-4,4- difluoropyrrolidin-3- yl}methanesulfonamide | 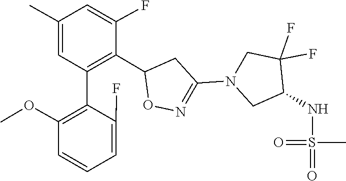 | 502.3 |
| 176 | N-[(3R)-4,4-difluoro-1-{5- [2-fluoro-6-(3-fluoropyridin- 2-yl)-4-methylphenyl]-4,5- dihydro-1,2-oxazol-3-yl} pyrrolidin-3- yl]methanesulfonamide | 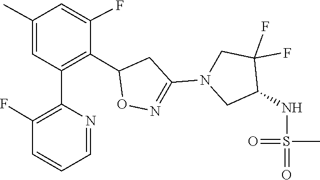 | 473.2 |
| 177 | N-[(3R)-1-{5-[2-(3,5- difluoropyridin-2-yl)-6- fluoro-4-methylphenyl]- 4,5-dihydro-1,2-oxazol- 3-yl}-4,4- difluoropyrrolidin-3-yl]-1- fluoromethanesulfonamide | 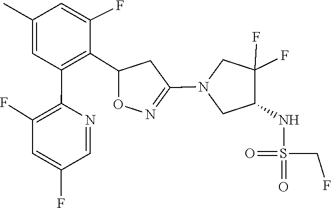 [SMILES: Cc1cc(F)c(C2CC(N3C[C@@H](NS(=O)(=O)CF)C(F)(F)C3)=NO2)c(-c2ncc(F)cc2F)c1] | 509.2 |
| 178 | N-[(3R,4S)-1-{5-[2-(3,5- difluoropyridin-2-yl)-6- fluoro-4-methylphenyl]- 4,5-dihydro-1,2-oxazol- 3-yl}-4- fluoropyrrolidin-3- yl]methanesulfonamide | 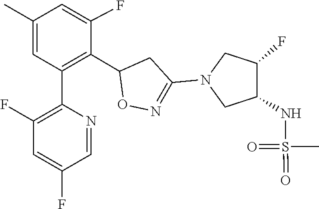 | 473.2 |

### Experimental Example 1: Acquisition of Cells Stably Expressing Human Orexin Type 2 Receptor (hOX2R)

In order to obtain cell clones that stably express human orexin type 2 receptor, human orexin type 2 (residues 1-444, NCBI Reference Sequence: AK314279) was constructed in a pcDNA3.1 (+) vector (Invitrogen) to express in mammalian cells. This plasmid DNA was transfected into CHO-K1 cells by electroporation, and stable clones were obtained by limiting dilution using resistance to G418 as a selection marker.

### Experimental Example 2: Measurement of Orexin Type 2 Receptor Agonist Activity

CHO cells in which human OX2 receptor was forcibly expressed were seeded at 10,000 cells/well into a 384-well black transparent bottom plate (BD falcon), and were incubated at 37° C., 5% CO2 for 1 day after incubation at room temperature for 30 minutes. After removing the cell plate medium, assay buffer containing calcium indicator A (HBSS (Thermo Fisher Scientific), 20 mM HEPES (Thermo Fisher Scientific), 0.1% BSA (Sigma-Aldrich), 2.5 μg/mL Fluo-4 AM (Dojin Kagaku), 0.08% Pluronic F127 (Dojin Kagaku), 1.25 mM probenecid (Dojin Kagaku)) was added at 30 μL/well. After incubation in a 5% CO2 incubator at 37° C. for 30 minutes, it was incubated at room temperature for another 30 minutes. Add 10 μL/well of the test compound diluted with assay buffer B (HBSS, 20 mM HEPES, 0.1% BSA), and use FDSS uCELL (Hamamatsu Photonics) to measure the fluorescence value every 1 second for 1 minute, and then every 2 seconds for 1 minute. It was measured for 1 minute and 40 seconds. The amount of fluorescence value when DMSO is added instead of the test compound is defined as 0%, and the amount of fluorescence value when orexin A (human) (Peptide Institute) with a final concentration of 10 nM is added is defined as 100%. The activity (%) of the test compound was calculated. Table 2 shows the activity of each compound at a concentration of 3 μM. As is clear from this result, the compound of the present invention was shown to have orexin type 2 receptor activating activity.

TABLE 2

| Test compound | OX2R agonist activity (3 μM, %) |
|---|---|
| 1 | 103 |
| 2 | 77 |
| 3 | 95 |
| 4 | 104 |
| 5 | 106 |
| 6 | 98 |
| 7 | 53 |
| 8 | 104 |
| 9 | 13 |
| 10 | 102 |
| 11 | 96 |
| 12 | 71 |
| 13 | 107 |
| 14 | 106 |
| 15 | 92 |
| 16 | 99 |
| 17 | 80 |
| 18 | 103 |
| 19 | 92 |
| 20 | 65 |
| 21 | 100 |
| 22 | 102 |
| 23 | 42 |
| 24 | 20 |
| 25 | 88 |
| 26 | 42 |
| 27 | 77 |
| 28 | 93 |
| 29 | 96 |
| 30 | 104 |
| 31 | 104 |
| 32 | 98 |
| 33 | 102 |
| 34 | 105 |
| 35 | 92 |
| 36 | 103 |
| 37 | 103 |
| 38 | 104 |
| 39 | 97 |
| 40 | 103 |
| 41 | 107 |
| 42 | 100 |
| 43 | 103 |
| 44 | 95 |
| 45 | 101 |
| 46 | 99 |
| 47 | 93 |
| 48 | 94 |
| 49 | 95 |
| 50 | 99 |
| 51 | 99 |
| 52 | 93 |
| 53 | 98 |
| 54 | 92 |
| 55 | 104 |
| 56 | 92 |
| 57 | 105 |
| 58 | 103 |
| 59 | 101 |
| 60 | 105 |
| 61 | 98 |
| 62 | 17 |
| 63 | 101 |
| 64 | 100 |
| 65 | 97 |
| 66 | 99 |
| 67 | 98 |
| 68 | 104 |
| 69 | 95 |
| 70 | 94 |
| 71 | 108 |
| 72 | 105 |
| 73 | 102 |
| 74 | 58 |
| 75 | 105 |
| 76 | 108 |
| 77 | 102 |
| 78 | 102 |
| 79 | 100 |
| 80 | 97 |
| 81 | 100 |
| 82 | 98 |
| 83 | 103 |
| 84 | 97 |
| 85 | 99 |
| 86 | 95 |
| 87 | 104 |
| 88 | 112 |
| 89 | 93 |
| 90 | 98 |
| 91 | 98 |
| 92 | 92 |
| 93 | 99 |
| 96 | 99 |
| 97 | 103 |
| 98 | 108 |
| 99 | 101 |
| 100 | 99 |
| 101 | 98 |
| 102 | 95 |
| 103 | 96 |
| 104 | 102 |
| 105 | 97 |
| 106 | 108 |
| 107 | 98 |
| 108 | 105 |
| 109 | 101 |
| 110 | 100 |
| 111 | 102 |
| 112 | 98 |
| 113 | 100 |
| 114 | 87 |
| 115 | 100 |
| 116 | 100 |
| 117 | 105 |
| 118 | 100 |
| 119 | 101 |
| 120 | 102 |
| 121 | 98 |
| 122 | 102 |
| 123 | 100 |
| 124 | 97 |
| 125 | 104 |
| 126 | 98 |
| 127 | 100 |
| 128 | 105 |
| 129 | 102 |
| 130 | 104 |
| 131 | 112 |
| 132 | 110 |
| 133 | 107 |
| 134 | 103 |
| 135 | 115 |
| 136 | 114 |
| 137 | 102 |
| 138 | 105 |
| 139 | 94 |
| 140 | 89 |
| 141 | 97 |
| 142 | 102 |
| 143 | 109 |
| 144 | 98 |
| 145 | 105 |
| 146 | 102 |
| 147 | 98 |
| 148 | 98 |
| 149 | 96 |
| 150 | 95 |
| 151 | 107 |
| 152 | 107 |
| 153 | 96 |
| 154 | 102 |
| 155 | 99 |
| 156 | 94 |
| 157 | 92 |
| 158 | 100 |
| 159 | 99 |
| 160 | 104 |
| 161 | 103 |
| 162 | 103 |
| 163 | 98 |
| 164 | 97 |
| 165 | 100 |
| 166 | 102 |
| 167 | 102 |
| 168 | 103 |
| 169 | 98 |
| 170 | 102 |
| 171 | 109 |
| 172 | 99 |
| 173 | 100 |
| 174 | 107 |
| 175 | 112 |
| 176 | 109 |
| 177 | 106 |
| 178 | 111 |

### Experimental Example 3: Evaluation of Wake-Promoting Effects in Cynomolgus Monkeys

The wake-promoting effects were evaluated by measuring the electroencephalogram (EEG), electromyogram (EMG) and locomotor activity in cynomolgus monkeys. Under isoflurane anesthesia (0.5-5%, Pfizer Japan Inc., Tokyo, Japan), male cynomolgus monkeys (2-3 years old, Hamri Co., Ltd., Ibaraki, Japan) were surgically implanted with radio-telemetry transmitters (L03-F3, Data Sciences International Inc., MN, USA). The two EEG electrodes were stereotaxically positioned at the parietal area and secured to the cranium with stainless-steel screws in contact with the dura. Bilateral EMG electrodes were implanted into the back cervical muscles.

After at least a 1-month recovery period in home cages, the monkeys were habituated to the recording chamber (an acrylic cage 60 W×55 D×75 H (cm)) located in a soundproof and electrically shielded room by the time animals sleep sufficiently in the experimental room. Cortical EEG, EMG and locomotor activity were recorded using the telemetry system (PhysioTel Digital telemetry platform, Data Sciences International Inc.). The signals were semi-automatically scored in 20-second epochs by a sleep scoring system (SleepSign, Kissei Comtec Co., Ltd., Nagano, Japan).

Test compound (3 mg/kg) suspended in 0.5% methylcellulose aqueous solution or vehicle (i.e., 0.5% methylcellulose aqueous solution) was administered orally (p.o.) to monkeys at zeitgeber time 12 in a volume of 5 mL/kg body weight with a pretest-posttest design (n=2), and then EEG, EMG and locomotor activity were recorded. The time spent in wakefulness for 4 hours after administration (% of vehicle treatment) was calculated by using SleepSign. The results are shown in Table 3.

TABLE 3

| Example | Dose | Wakefulness time |
|---|---|---|
| No | (mg/kg, p.o.) | (% of vehicle treatment) (Mean, n = 2) |
| 11 | 3 | 638.80 |
| 109 | 3 | 558.37 |
| 149 | 3 | 282.21 |
| 165 | 3 | 716.25 |

As is clear from Table 3, the test compounds of the present invention increased the wakefulness time compared to the vehicle treatment group in cynomolgus monkeys. That is, these compounds were suggested to be potential therapeutics for narcolepsy.

### Formulation Example 1 (Production of Capsule)

| | |
|---|---|
| 1) compound of Example 1 | 30 mg |
| 2) crystalline cellulose | 10 mg |
| 3) lactose | 19 mg |
| 4) magnesium stearate | 1 mg |
| total | 60 mg |

1), 2), 3) and 4) are mixed and filled in a gelatin capsule.

### Formulation Example 2 (Production of Tablet)

| | |
|---|---|
| 1) compound of Example 1 | 30 g |
| 2) lactose | 50 g |
| 3) cornstarch | 15 g |
| 4) calcium carboxymethylcellulose | 44 g |
| 5) magnesium stearate | 1 g |
| 1000 tablets | 140 g in total |

The total amount of 1), 2), 3) and 30 g of 4) are kneaded with water, vacuum dried and sieved. The sieved powder is mixed with 14 g of 4) and 1 g of 5), and the mixture is punched by a tableting machine. In this way, 1000 tablets containing 30 mg of the compound of Example 1 per tablet are obtained.

### INDUSTRIAL APPLICABILITY

The compound of the present invention has an orexin type 2 receptor agonist activity, and is useful as an agent for the prophylaxis or treatment of narcolepsy.

This application is based on patent application No. 63/385,598 filed on Nov. 30, 2022 in United States, the contents of which are encompassed in full herein.

1. A compound of formula (I) 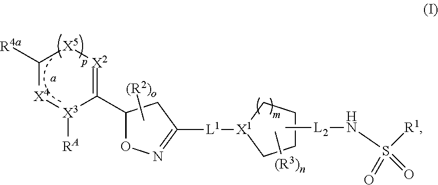
wherein
R¹ is a C₁₋₆ alkyl group, a C₃₋₇ cycloalkyl group, or a mono- or di-C₁₋₆ alkylamino group, wherein each of the C₁₋₆ alkyl group, C₃₋₇ cycloalkyl group, and mono- or di-C₁₋₆ alkylamino group is optionally substituted;
each R² is the same or different, and is an optionally substituted C₁₋₆ alkyl group;
each R³ is the same or different, and is an optionally substituted C₁₋₆ alkyl group, or a halogen atom;
R^{A} is an optionally substituted 3- to 8-membered non-aromatic heterocyclic group, an optionally substituted 5- to 6-membered aromatic heterocyclic group, an optionally substituted C₆₋₁₄ aryl group, or an optionally substituted C₃₋₁₀ cycloalkyl group;
L¹ is a bond, —NR⁶—, or —O—; wherein R⁶ is a hydrogen atom, or an optionally substituted C₁₋₆ alkyl group;
L² is a bond or —CH₂—;
X¹ is N or CR^{3′};
X² is N or CR^{4b};
X³ is N or C;
X⁴ is N or CR^{4c};
X⁵ is N or CR^{4d};
provided that no more than two of X², X³, X⁴, and X⁵ are N;
R^{3′} is a hydrogen atom, an optionally substituted C₁₋₆ alkyl group, or a halogen atom;
R^{4a}, R^{4b}, R^{4c}, and R^{4d} are the same or different and each is a hydrogen atom, an optionally substituted C₁₋₆ alkyl group, an optionally substituted C₃₋₇ cycloalkyl group, a halogen atom, or an optionally substituted C₁₋₆ alkoxy group;
m is an integer of 0 to 3;
n is an integer of 0 to 3;
o is an integer of 0 to 3;
p is an integer of 0 or 1; and [symbol]
comprises one single bond and one double bond when p is 0 or one single bond and two double bonds when p is 1 to provide an aromatic ring structure;
or a salt thereof.

2. The compound according to claim 1, wherein R^{A} has the following structure: 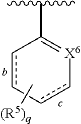
wherein
X⁶ is N or CR^{5′};
each R⁵ is the same or different, and is a halogen atom, or a C₁₋₆ alkoxy group;
R^{5′} is a hydrogen atom, or a halogen atom;
q is an integer of 0 to 7; and [symbol]
and [symbol] are the same and are both single bonds or both double bonds;
or a salt thereof.

3. The compound according to claim 1, wherein X¹ is CR^{3′}, m is an integer of 0 to 2, and n is 0; or a salt thereof.

4. The compound according to claim 1, wherein X¹ is N; or a salt thereof.

5. The compound according to claim 2, wherein
R¹ is a C₁₋₆ alkyl group, a C₁₋₆ alkoxy-C₁₋₆ alkyl group, a halo-C₁₋₆ alkyl group, a cyclopropyl group, a halo-cyclopropyl group, or a mono- or di-C₁₋₆ alkylamino group;
each R² is the same or different, and is a C₁₋₆ alkyl group, a halo-C₁₋₆ alkyl group, a C₁₋₆ alkoxy-C₁₋₆ alkyl group, or a hydroxy-C₁₋₆ alkyl group;
each R³ is the same or different, and is a C₁₋₆ alkyl group, a C₁₋₆ alkoxy-C₁₋₆ alkyl group, or a halogen atom;
R^{4a} is a hydrogen atom, a C₁₋₆ alkyl group, a cyclopropyl group, a C₁₋₆ alkoxy group, a halo-C₁₋₆ alkyl group, or a halogen atom; and
q is an integer of 0 to 4;
or a salt thereof.

6. The compound according to claim 1, wherein either
(i) X² is CR^{4b}, X³ is N, X⁴ is N, and p is 0; or
(ii) X² is N, X³ is N, X⁴ is CR^{4c}, and p is 0;
or a salt thereof.

7. The compound according to claim 1, wherein X² is CR^{4b}, X³ is C, X⁴ is CR^{4c}, X⁵ is CR^{4d}, and p is 1; or a salt thereof.

8. The compound according to claim 1, wherein X² is N, X³ is C, X⁴ is CR^{4c}, X⁵ is CR^{4d}, and p is 1; or a salt thereof.

9. The compound according to claim 1, wherein X² is CR^{4b}, X³ is C, X⁴ and X⁵ are N, and p is 1; or a salt thereof.

10. The compound according to claim 1, wherein X² is CR^{4b}, X³ is C, X⁴ is CR^{4c}, X⁵ is N, and p is 1; or a salt thereof.

11. The compound according to claim 1, wherein X² and X⁴ are N, X³ is C, X⁵ is CR^{4d}, and p is 1; or a salt thereof.

12. The compound according to claim 1, wherein X² is CR^{4b}, X³ is C, X⁴ is N, X⁵ is CR^{4d}, and p is 1; or a salt thereof.

13. The compound according to claim 2, wherein X⁶ is CR^{5′}, and [symbol] and [symbol] are both single bonds; or a salt thereof.

14. The compound according to claim 2, wherein X⁶ is CR^{5′}, and [symbol] and [symbol] are both double bonds; or a salt thereof.

15. The compound according to claim 2, wherein X⁶ is N′, and [symbol] and [symbol] are both double bonds; or a salt thereof.

16. The compound according to claim 1, wherein the moiety: 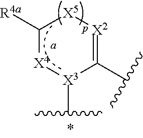
is selected from the group consisting of: 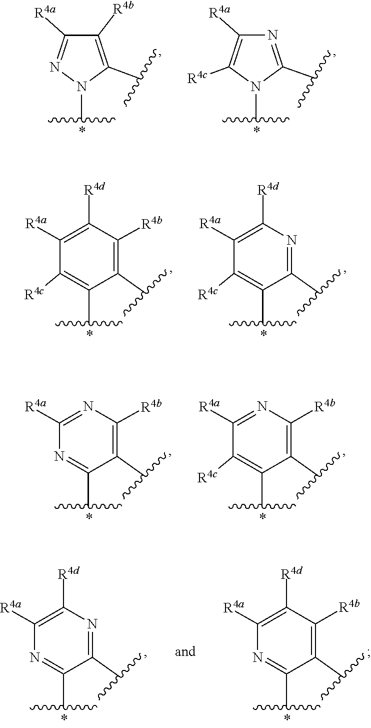
wherein
* indicates the bonding site to R^{A}; and
R^{4a}, R^{4b}, R^{4c}, and R^{4d} are as defined in claim 1;
or a salt thereof.

17. The compound according to claim 2, wherein the moiety: 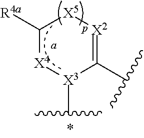
is selected from the group consisting of: 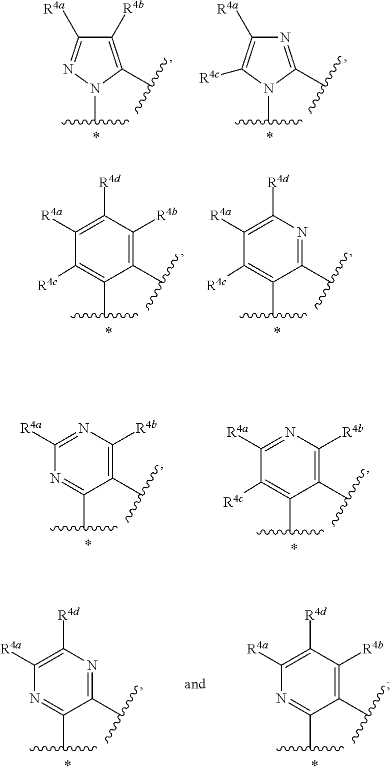
wherein * indicates the bonding site to R^{A};
R¹ is a C₁₋₆ alkyl group, a C₁₋₆ alkoxy-C₁₋₆ alkyl group, a halo-C₁₋₆ alkyl group, a cyclopropyl group, a halo-cyclopropyl group, or a mono- or di-C₁₋₆ alkylamino group;
each R² is the same or different, and is a C₁₋₆ alkyl group, a halo-C₁₋₆ alkyl group, a C₁₋₆ alkoxy-C₁₋₆ alkyl group, or a hydroxy-C₁₋₆ alkyl group;
each R³ is the same or different, and is a C₁₋₆ alkyl group, a C₁₋₆ alkoxy-C₁₋₆ alkyl group, or a halogen atom;
L¹ is a bond, —NR⁶—, or —O—; wherein R⁶ is a hydrogen atom, or a C₁₋₆ alkyl group;
L² is a bond or —CH₂—;
X¹ is N or CR^{3′}; wherein R^{3′} is a hydrogen atom;
R^{4a} is a hydrogen atom, a C₁₋₆ alkyl group, a cyclopropyl group, a C₁₋₆ alkoxy group, a halo-C₁₋₆ alkyl group, or a halogen atom;
X² is N or CR^{4b}; wherein R^{4b} is a hydrogen atom, or a halogen atom;
X⁴ is N or CR^{4c}; wherein R^{4c} is a hydrogen atom, or a halogen atom;
X⁵ is N or CR^{4d}; wherein R^{4d} is a hydrogen atom;
m is an integer of 0 to 3;
n is an integer of 0 to 3;
o is an integer of 0 to 3; and
the moiety: 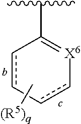
is selected from the group consisting of: 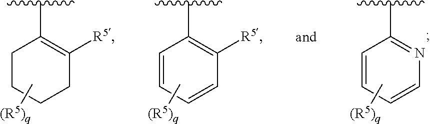
wherein
each R⁵ is the same or different, and is a halogen atom, or a C₁₋₆ alkoxy group;
R^{5′} is a hydrogen atom, or a halogen atom; and
q is an integer of 0 to 4;
or a salt thereof.

18. The compound according to claim 2, wherein the compound is a compound of formula (Ia): 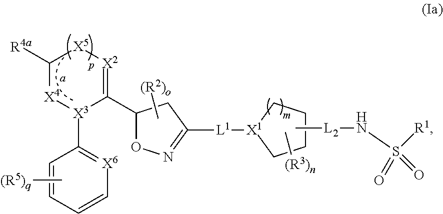
wherein
q is an integer of 0 to 4; and
the other symbols are as defined in claim 2;
or a salt thereof.

19. The compound according to claim 2, wherein
the compound is a compound of formula (Ib): 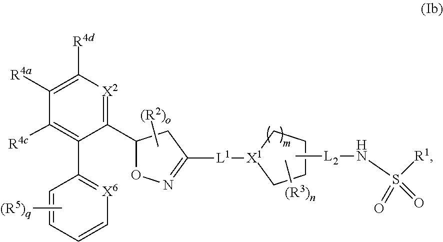
wherein
q is an integer of 0 to 4; and
the other symbols are as defined in claim 2;
or a salt thereof.

20. The compound according to claim 2, wherein the compound is a compound of formula (Ic): 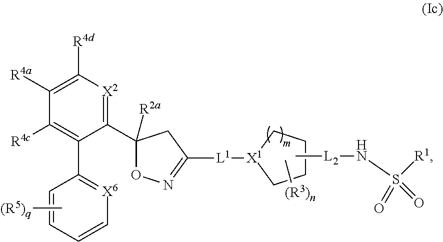
wherein
R^{2a} is a hydrogen atom, or an optionally substituted C₁₋₆ alkyl group;
q is an integer of 0 to 4; and
the other symbols are as defined in claim 2;
or a salt thereof.

21. The compound according to claim 2, wherein the compound is a compound of formula (Id): 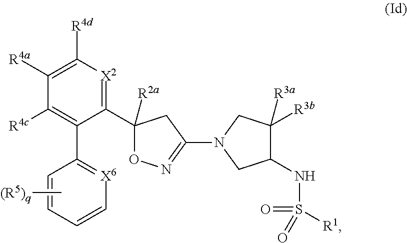
wherein
R^{2a} is a hydrogen atom, or an optionally substituted C₁₋₆ alkyl group;
R^{3a} and R^{3b} are the same or different and each is a hydrogen atom, an optionally substituted C₁₋₆ alkyl group, or a halogen atom;
q is an integer of 0 to 4; and
the other symbols are as defined in claim 2;
or a salt thereof.

22. The compound according to claim 1, wherein the compound is a compound of formula (Ie): 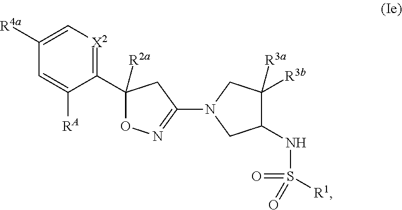
wherein
R¹ is a C₁₋₆ alkyl group, or a halo-C₁₋₆ alkyl group;
R^{2a} is a hydrogen atom, a C₁₋₆ alkyl group, or a halo-C₁₋₆ alkyl group;
R^{3a} and R^{3b} are the same or different and each is a C₁₋₆ alkyl group, or a halogen atom;
R^{4a} is a C₁₋₆ alkyl group, or a halogen atom;
X² is N or CR^{4b}; wherein R^{4b} is a halogen atom; and
R^{A} is a phenyl group, or a pyridyl group, wherein each of the phenyl group and pyridyl group is optionally substituted by one to three halogen atoms;
or a salt thereof.

23. The compound according to claim 22, wherein
R¹ is a C₁₋₆ alkyl group, or a halo-C₁₋₆ alkyl group;
R^{2a} is a hydrogen atom, a C₁₋₆ alkyl group, or a halo-C₁₋₆ alkyl group;
R^{3a} and R^{3b} are each a halogen atom;
R^{4a} is a C₁₋₆ alkyl group, or a halogen atom;
X² is N or CR^{4b}; wherein R^{4b} is a halogen atom; and
R^{A} is a phenyl group, or a pyridyl group, wherein each of the phenyl group and pyridyl group is optionally substituted by one to three halogen atoms;
or a salt thereof.

24. The compound according to claim 22, wherein
R¹ is a C₁₋₆ alkyl group, or a halo-C₁₋₆ alkyl group;
R^{2a} is a C₁₋₆ alkyl group, or a halo-C₁₋₆ alkyl group;
R^{3a} and R^{3b} are each a halogen atom;
R^{4a} is a halogen atom;
X² is N; and
R^{A} is a phenyl group optionally substituted by one to three halogen atoms;
or a salt thereof.

25. The compound according to claim 22, wherein
R¹ is a C₁₋₆ alkyl group;
R^{2a} is a halo-C₁₋₆ alkyl group;
R^{3a} and R^{3b} are each a halogen atom;
R^{4a} is a halogen atom;
X² is N; and
R^{A} is a phenyl group optionally substituted by one to three halogen atoms;
or a salt thereof.

26. The compound according to claim 1, wherein the compound is selected from the group consisting of:
N-[(3R)-4,4-difluoro-1-{(5S)-5-[5-methyl-3-(2,4,6-trifluorophenyl)pyridin-2-yl]-4,5-dihydro-1,2-oxazol-3-yl}pyrrolidin-3-yl]methanesulfonamide;
N-[(3R)-1-{(5S)-5-[3-(2,6-difluorophenyl)-5-fluoropyridin-2-yl]-5-(fluoromethyl)-4,5-dihydro-1,2-oxazol-3-yl}-4,4-difluoropyrrolidin-3-yl]methanesulfonamide;
N-[(3R)-1-{(5S)-5-[3-(2,6-difluorophenyl)-5-fluoropyridin-2-yl]-5-(fluoromethyl)-4,5-dihydro-1,2-oxazol-3-yl}-4,4-difluoropyrrolidin-3-yl]-1-fluoromethanesulfonamide; and
N-[(3R)-1-{(5S)-5-[3-(2,6-difluorophenyl)-5-fluoropyridin-2-yl]-5-methyl-4,5-dihydro-1,2-oxazol-3-yl}-4,4-difluoropyrrolidin-3-yl]methanesulfonamide,
or a salt thereof.

27. A pharmaceutical composition comprising the compound as defined in claim 1 or a salt thereof, and a pharmacologically acceptable carrier.

28.-32. (canceled)

33. A method for the prophylaxis or treatment of a disease or disorder associated with an orexin type 2 receptor in a mammal in need thereof, comprising administering to the mammal a therapeutically effective amount of the compound as defined in claim 1 or a salt thereof.

34. The method according to claim 33, wherein the disease or disorder is selected from the group consisting of narcolepsy, idiopathic hypersomnia, hypersomnia, sleep apnea syndrome, narcolepsy syndrome accompanied by narcolepsy-like symptoms, hypersomnia syndrome accompanied by daytime hypersomnia, Alzheimer's disease, obesity, insulin resistance syndrome, cardiac failure, diseases related to bone loss, sepsis, disturbance of consciousness, and side effects and complications due to anesthesia.

35. The method according to claim 33, wherein the disease or disorder is selected from the group consisting of narcolepsy, idiopathic hypersomnia, hypersomnia, and sleep apnea syndrome.

36. The method according to claim 33, wherein the disease or disorder is narcolepsy.

37.-45. (canceled)
